(19) United States
(12) Patent Application Publication
(10) Pub. No.: US 2020/0147064 A1
(43) Pub. Date: May 14, 2020

(54) Reduction of Pro-Inflammatory HDL Using a Leukotriene Inhibitor

(71) Applicant: Autoimmune Pharma LLC, Lawrence, NY (US)

(72) Inventor: Jeffrey Benison, Lawrence, NY (US)

(73) Assignee: Autoimmune Pharma LLC, Lawrence, NY (US)

(21) Appl. No.: 16/486,616

(22) PCT Filed: Feb. 16, 2018
(86) PCT No.: PCT/US2018/018497

§ 371 Date: Aug. 16, 2019

Related U.S. Application Data
(60) Provisional application No. 62/460,007, filed on Feb. 16, 2017.

(51) Int. Cl. A61K 31/444

(52) U.S. Cl. CPC A61K 31/444

(57) ABSTRACT

A method involving the administration of a therapeutically effective amount of a leukotriene inhibitor, a pharmaceutically acceptable salt, a pharmaceutically acceptable N-oxide, a pharmaceutically active metabolite, a pharmaceutically acceptable prodrug, or pharmaceutically acceptable solvate thereof to a human for reducing a level of pro-inflammatory HDL in the human. Various examples of leukotriene inhibitors, including 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid, are disclosed for administration for the reduction of pro-inflammatory HDL in a human. Reduction of pro-inflammatory HDL by the leukotriene inhibitor may include conversion of at least a portion of pro-inflammatory HDL to anti-inflammatory HDL.

### RELATED APPLICATIONS

This application claims the benefit of priority of U.S. Provisional Patent Application Ser. No. 62/460,007, filed on Feb. 16, 2017, and titled “Reduction of Pro-Inflammatory HDL Using a Leukotriene Inhibitor,” which is incorporated by reference herein in its entirety.

### BACKGROUND

A 5-lipoxygenase-activating protein (“FLAP”) is a member of the MAPEG (membrane associated proteins involved in eicosanoid and glutathione metabolism) family of proteins. Certain FLAP inhibitors, their manufacture, and their methods of use (e.g., as FLAP inhibitors and for the treatment of certain diseases) are described in the following U.S. Pat. Nos. 7,977,359, 8,710,081, 7,834,037, 8,546,431, 8,399,666, 8,841,295, 8,697,730, and 8,772,495 which are incorporated by reference herein in their entirety.

FLAP is responsible for binding arachidonic acid and transferring it to 5-lipoxygenase. 5-Lipoxygenase can then catalyze the two-step oxygenation and dehydration of arachidonic acid, converting it into the intermediate compound 5-HPETE (5-hydroperoxyeicosatetraenoic acid), and in the presence of FLAP convert the 5-HPETE to Leukotriene A₄ (LTA₄). LTA₄ is acted on by LTC₄ synthase, which conjugates LTA₄ with reduced glutathione (GSH) to form the intracellular product leukotriene C₄ (LTC₄). LTC₄ is transformed to leukotriene D₄ (LTD₄) and leukotriene E₄ (LTE₄) by the action of gamma-glutamyl-transpeptidase and dipeptidases. LTC₄ synthase plays a role as the only committed enzyme in the formation of cysteinyl leukotrienes.

Leukotrienes are biological compounds formed from arachidonic acid in the leukotriene synthesis pathway. Leukotrienes are synthesized primarily by eosinophils, neutrophils, mast cells, basophils, dendritic cells, macrophages and monocytes. Leukotrienes are agents in an inflammatory response and have been implicated in biological actions including, by way of example only, smooth muscle contraction, leukocyte activation, cytokine secretion, mucous secretion, and vascular function.

A sustained inflammatory response in a disease state can lead to conversion of normal HDL, which is anti-inflammatory, to pro-inflammatory HDL (B. J. Van Lenten, et al., Journal of Clinical Investigation, vol. 96, no. 6, pp. 2758-2767, 1995). Pro-inflammatory HDL has been implicated in the development of cardio vascular disease (M. Navab, et al; Journal of Lipid Research, vol. 50, supplement, pp. S145-S149, 2009) and may be additionally pathogenic in other diseases that are characterized by a chronic inflammatory condition. It is believed that no link has yet been made for the use of a leukotriene inhibitor for the purpose of reducing levels of pro-inflammatory HDL in a patient.

### SUMMARY OF DISCLOSURE

In one implementation, a method for reducing pro-inflammatory HDL in a human is provided. The method includes administering to the human a therapeutically effective amount of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof such that the pro-inflammatory HDL level is reduced.

In another implementation, a method for reducing pro-inflammatory HDL in a human is provided. The method includes determining that the human has a need to have pro-inflammatory HDL levels reduced; administering to the human a therapeutically effective amount of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof such that the level of pro-inflammatory HDL in the human is reduced; and confirming the conversion of pro-inflammatory HDL to anti-inflammatory HDL.

In yet another implementation, use of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof to reduce a pro-inflammatory HDL in a human such that the level of pro-inflammatory HDL is reduced is provided.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates one exemplary method of reducing pro-inflammatory HDL in a human;

FIG. 2 illustrates another example of a method of reducing pro-inflammatory HDL;

FIG. 3 illustrates a non-limiting example of a synthetic approach toward indole compounds of Formula (G), Formula (G-I) and Formula (G-II);

FIG. 4 illustrates two additional non-limiting examples of a synthetic approach toward indole compounds of Formula (G), Formula (G-I) and Formula (G-II);

FIG. 5 illustrates non-limiting examples of a synthetic strategy toward indole or indole-like scaffolds for compounds of Formula (G), Formula (G-I) and Formula (G-II);

FIG. 6 illustrates additional non-limiting examples of a synthetic strategy toward indole or indole-like scaffolds for compounds of Formula (G), Formula (G-I) and Formula (G-II);

FIG. 7 illustrates more additional non-limiting examples of a synthetic strategy toward indole or indole-like scaffolds for compounds of Formula (G), Formula (G-I) and Formula (G-II);

FIG. 8 illustrates still more additional non-limiting examples of a synthetic strategy toward indole or indole-like scaffolds for compounds of Formula (G), Formula (G-I) and Formula (G-II);

FIG. 9 illustrates yet more additional non-limiting examples of a synthetic strategy toward indole or indole-like scaffolds for compounds of Formula (G), Formula (G-I) and Formula (G-II);

FIG. 10 illustrates exemplary embodiment compounds of Formula (G), Formula (G-I) and Formula (G-II);

FIG. 11 illustrates more exemplary embodiment compounds of Formula (G), Formula (G-I) and Formula (G-II);

FIG. 12 illustrates still more exemplary embodiment compounds of Formula (G), Formula (G-I) and Formula (G-II); and

FIG. 13 illustrates yet more exemplary embodiment compounds of Formula (G), Formula (G-I) and Formula (G-II).

### DETAILED DESCRIPTION

Methods for reducing pro-inflammatory HDL in a human are described herein. In one example, these methods include the use of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof to reduce the level of pro-inflammatory HDL in the human. Examples of a reduction of pro-inflammatory HDL include, but are not limited to, a conversion of at least a portion of the pro-inflammatory HDL of the human to anti-inflammatory HDL, lowering the amount of pro-inflammatory HDL, and any combinations thereon. In one example, reduction of pro-inflammatory HDL includes converting at least a portion of the pro-inflammatory HDL of the human to anti-inflammatory HDL.

Various leukotriene inhibitors are known. Any leukotriene inhibitor that has the ability to reduce pro-inflammatory HDL in a human can be utilized (e.g., via conversion of pro-inflammatory HDL to anti-inflammatory HDL).

Examples of a leukotriene inhibitor include, but are not limited to, a compound having a structure of Formula (G) discussed further below, a compound having a structure of Formula (G-I) discussed further below, a compound having a structure of Formula (G-II) discussed further below, a 5-lipoxygenase-activating protein (FLAP) inhibitor, 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid, an lta4h inhibitor, and any combinations thereof. In one example, a leukotriene inhibitor includes a FLAP inhibitor. In another example, a leukotriene inhibitor includes a compound having a structure of Formula (G). In yet another example, a leukotriene inhibitor includes 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid. It is noted that 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid is also referred to by the trade name FIBOFLAPON and/or GSK2190915 and/or AM-803 in certain literature. In some literature, 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid is listed by the IUPAC/chemical name of 3-[3-(tert-butylthio)-1-[4-(6-ethoxypyridin-3-yl)-benzyl]-5-((5-methylpyridin-2-yl)methoxy)-1H-indol-2-yl]-2,2-dimethyl-propionic acid. It is also noted that 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid has the structure of Formula (Z1), listed below, and is a species of Formula (G), also listed below. Additional examples of leukotriene inhibitors are discussed herein.

Reduction of pro-inflammatory HDL can be advantageous for human health and may be beneficial to a human suffering from any of a variety of diseases or conditions. Examples of benefits to human health of reduction of pro-inflammatory HDL include, but are not limited to, decreased risk of development of diseases such as artherosclerosis. Examples of a disease or condition that may benefit from the reduction of pro-inflammatory HDL include, but are not limited to, a chronic inflammatory disease or condition, myositis, and any combinations thereof. Examples of a chronic inflammatory disease or condition include, but are not limited to, systemic lupus erythematosus, rheumatoid arthritis, systemic sclerosis, pulmonary arterial hypertension, asthma, gout, and any combinations thereof. Administration of a leukotriene inhibitor according to the current disclosure to a patient suffering from such a chronic inflammatory disease or condition to reduce a level of pro-inflammatory HDL in the patient may alleviate one or more symptoms of the chronic inflammatory disease and/or reduced risk of additional medical problems associated with elevated pro-inflammatory HDL. In one example, administration of a leukotriene inhibitor of the current disclosure is for reducing pro-inflammatory HDL in a patient suffering from systemic lupus erythematosus. In such an example, the patient may experience reduced risk additional medical problems associated with elevated pro-inflammatory HDL.

Systemic lupus erythematosus is a disease characterized by an auto-immune chronic inflammatory condition. The presence of pro-inflammatory HDL has been previously detected in some but not all patients and is associated with the development of cardio vascular disease in these patients.

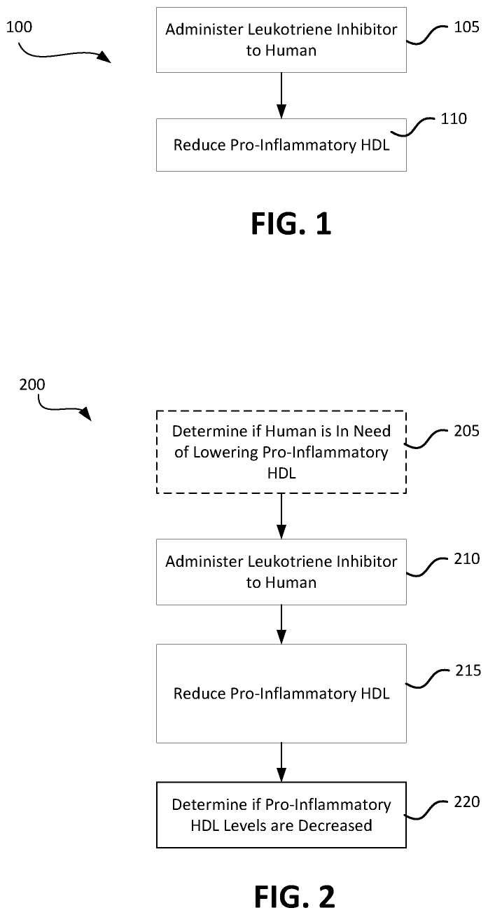

FIG. 1 illustrates one exemplary method 100 of reducing pro-inflammatory HDL in a human. At step 105, a leukotriene inhibitor is administered to a human. Suitable routes of administration include, but are not limited to, intravenous, oral, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections. It is also possible to administer a leukotriene inhibitor in a local rather than systemic manner, for example, via injection of the compound directly into an organ, often in a depot preparation or sustained release formulation. In one example, such long acting formulations may be administered by implantation (for example subcutaneously or intramuscularly). In another example, such long acting formulations may be administered by intramuscular injection. It is further contemplated that a leukotriene inhibitor may be administered in a targeted drug delivery system, for example, in a liposome coated with organ-specific antibody. In one such example, a liposome is targeted to and taken up selectively by the organ. Still further, a leukotriene inhibitor may be provided in the form of a rapid release formulation, in the form of an extended release formulation, or in the form of an intermediate release formulation. Combinations of routes of administration are contemplated as a possible way of administration.

A leukotriene inhibitor of the current disclosure may be included in a pharmaceutical composition that is administered to a human. Pharmaceutical compositions may be formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety. Example techniques for manufacturing a pharmaceutical composition include, but are not limited to, mixing, dissolving, granulating, dragee-making, levigating, emulsifying, encapsulating, entrapping, compression, formation of a thin film, formation of a gel cap, and any combinations thereof.

A leukotriene inhibitor according to this disclosure may be present in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, a leukotriene inhibitor may exist as tautomers. All tautomers are included within the scope of the compounds presented herein. Additionally, a leukotriene inhibitor described herein can exist in unsolvated as well as solvated forms with pharmaceutically acceptable solvents such as water, ethanol, and the like. The solvated forms of a leukotriene inhibitor presented herein are also considered to be disclosed herein.

A pharmaceutical composition may include additional ingredients to the leukotriene inhibitor. Examples of additional ingredients include, but are not limited to, a carrier, a stabilizer, a diluent, a dispersing agent, a suspending agent, a preserving agent, a thickening agent, a binding agent, a plasticizer (e.g., glycerol or sorbitol), a wetting agent, an emulsifying agent, a solution promoter, a solubilizing agent, a salt for regulating osmotic pressure, a buffer, an antioxidant, an excipient, a disintegrating agent, an additional active ingredient and/or therapeutically valuable substance (e.g., as in combination therapy), and any combinations thereof. In one example, such additional ingredients are inert and/or non-toxic.

Example excipients include, but are not limited to, a filler; a cellulose preparation, such as, for example, a maize starch, a wheat starch, a rice starch, a potato starch, a gelatin, a gum tragacanth, methylcellulose, a microcrystalline cellulose, hydroxypropylmethylcellulose, sodium carboxymethylcellulose; polyvinylpyrrolidone (PVP or povidone); calcium phosphate; and any combinations thereof. Example fillers include, but are not limited to, a sugar (e.g., lactose, sucrose, mannitol, sorbitol)

Example disintegrating agents include, but are not limited to, a cross-linked croscarmellose sodium, polyvinylpyrrolidone, agar, alginic acid, a salt of alginic acid (e.g., sodium alginate), and any combinations thereof.

In one example, one or more polymers may be utilized as a suspending agent. Examples of a polymer include, but are not limited to, a water-soluble polymer (e.g., cellulosic polymers, such as hydroxypropyl methylcellulose), a water-insoluble polymer (e.g., a cross-linked carboxyl-containing polymer), a mucoadhesive polymer (e.g., carboxymethylcellulose, carbomer (acrylic acid polymer), poly(methylmethacrylate), polyacrylamide, polycarbophil, acrylic acid/butyl acrylate copolymer, sodium alginate and dextran).

In one exemplary aspect, a solubilizing agents may be included to aid in the solubility of a leukotriene inhibitor according to the current disclosure and/or another active ingredient. The term “solubilizing agent” generally includes agents that result in formation of a micellar solution or a true solution of the agent. Examples of a solubilizing agent include, but are not limited to, an acceptable nonionic surfactant (e.g., polysorbate 80), an ophthalmically acceptable glycol, a polyglycol (e.g., polyethylene glycol 400)), a glycol ether, and any combinations thereof.

Examples of a buffering agent (e.g., a pH adjusting agent) include, but are not limited to, an acid (e.g., acetic, boric, citric, lactic, phosphoric and hydrochloric acids), a base (e.g., sodium hydroxide, sodium phosphate, sodium borate, sodium citrate, sodium acetate, sodium lactate and tris-hydroxymethylaminomethane), a buffers (e.g., citrate/dextrose, sodium bicarbonate and ammonium chloride). Such acids, bases and buffers may be included in an amount required to maintain pH of the composition in an acceptable range.

Examples of a salt that can be used to bring osmolality of the composition into an acceptable range include, but are not limited to, a sodium, potassium, or ammonium cation and chloride; a citrate, ascorbate, borate, phosphate, bicarbonate, sulfate, thiosulfate or bisulfite anions; sodium chloride; potassium chloride; sodium thiosulfate; sodium bisulfite; ammonium sulfate; and any combinations thereof.

Examples of a preservatives to inhibit microbial activity include, but are not limited to, a mercury-containing substance (e.g., merfen and thiomersal), stabilized chlorine dioxide, a quaternary ammonium compounds (e.g., benzalkonium chloride, cetyltrimethylammonium bromide and cetylpyridinium chloride), and any combinations thereof.

Examples of a surfactant to enhance physical stability or for other purposes include, but are not limited to, a polyoxyethylene fatty acid glyceride, a vegetable oil (e.g., polyoxyethylene (60) hydrogenated castor oil), polyoxyethylene alkylethers and alkylphenyl ethers (e.g., octoxynol 10, octoxynol 40), and any combinations thereof.

Examples of an antioxidant include, but are not limited to, ascorbic acid, sodium metabisulfite, and any combinations thereof.

In one example, a pharmaceutical composition may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

A pharmaceutical composition including a leukotriene inhibitor of the current disclosure may be administered in a variety of forms. Example forms for administration include, but are not limited to, aqueous, solid, liquid, an emulsion, semi-solid, and a suspension. Pharmaceutical compositions of multiple forms may be administered to the same human (e.g., the same one or more leukotriene inhibitors in different forms of administration). Example of a solid form include, but are not limited to, a powder, a tablet, dispersible granules, a capsule, a cachet, a suppository, and any combinations thereof. Examples of a liquid form include, but are not limited to, a solution; an emulsion; a solution including liposomes, micelles, or nanoparticles comprising a leukotriene inhibitor; and any combinations thereof. Examples of a semi-solid form include, but are not limited to, a gel, a suspension, a cream, and any combination thereof. In one example of a solution and/or suspension a first portion of an active ingredient is present in solution and a second portion of the active ingredient is present in particulate form, in suspension in a liquid matrix.

In one example an aqueous suspension composition can be packaged in single-dose non-reclosable container. In another example, a multiple-dose reclosable container can be used (e.g., including a preservative in the composition).

Various delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, a leukotriene inhibitor may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

In one example of intravenous injections, a leukotriene inhibitor as disclosed herein may be formulated in aqueous solutions (e.g., in a physiologically compatible buffers such as Hank's solution, Ringer's solution, or physiological saline buffer). For transmucosal administration, penetrants appropriate to the barrier to be permeated may be used in the formulation. Such penetrants are generally known in the art. For other parenteral injections, appropriate formulations may include aqueous or nonaqueous solutions (e.g., with physiologically compatible buffers or excipients). Such excipients are generally known in the art.

In one example of oral administration, a leukotriene inhibitor can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated. In another example a pharmaceutical preparations for oral use can be obtained by mixing one or more solid excipient with one or more leukotriene inhibitors described herein, optionally grinding the resulting mixture, and processing the mixture of granules, after adding suitable auxiliaries, if desired, to obtain tablets or dragee cores. In such an example, suitable excipients include fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In one example, dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In another example, a pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In an example for buccal or sublingual administration, one or more leukotriene inhibitors may take the form of tablets, lozenges, or gels formulated in a conventional manner. Parental injections may involve bolus injection or continuous infusion. Formulations for injection may be presented in unit dosage form, e.g., in ampoules or in multi-dose containers, with an added preservative. A pharmaceutical composition may be in a form suitable for parenteral injection as a sterile suspensions, solutions or emulsions in oily or aqueous vehicles, and may contain formulatory agents such as suspending, stabilizing and/or dispersing agents. In one example, a pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In an example of administering topically, one or more leukotriene inhibitors can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

Formulations suitable for transdermal administration may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery can be accomplished by means of iontophoretic patches and the like. Additionally, transdermal patches can provide controlled delivery of one or more leukotriene inhibitors. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In an example of administration by inhalation, one or more leukotriene inhibitors as disclosed herein may be in a form as an aerosol, a mist or a powder. Pharmaceutical compositions may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

Rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like, may also be utilized. In suppository forms, a low-melting wax such as, but not limited to, a mixture of fatty acid glycerides, optionally in combination with cocoa butter can first be melted.

A leukotriene inhibitor of the current disclosure can be administered for a variety of reasons. In one example, a leukotriene inhibitor is administered as part of a prophylactic treatment. In another example, a leukotriene inhibitor is administered as part of a therapeutic treatment. In one example of a therapeutic applications, a leukotriene inhibitor is administered to a patient already determined to have a need for lowering pro-inflammatory HDL. Such a patient may already be suffering from a disease or condition. A leukotriene inhibitor may be administered in an amount sufficient to lower pro-inflammatory HDL, which in some cases may cure or at least partially arrest the symptoms of the disease or condition. Amounts effective for this use will depend on the severity and course of the level of pro-inflammatory HDL and/or disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing a leukotriene inhibitor described herein are administered to a patient susceptible to or otherwise at risk of heightened pro-inflammatory HDL. Such a patient may also be susceptible to a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of any disease, disorder or condition, previous therapy, the patient's health status and response to the drugs, and the judgment of the treating physician.

Referring again to FIG. 1, at step 110, the level of pro-inflammatory HDL is reduced in response to the administering of a leukotriene inhibitor. Examples of a reduction of pro-inflammatory HDL include, but are not limited to, a conversion of at least a portion of the pro-inflammatory HDL of the human to anti-inflammatory HDL, lowering the level of pro-inflammatory HDL in the human, and any combinations thereon. In one example, reduction of pro-inflammatory HDL includes converting at least a portion of the pro-inflammatory HDL of the human to anti-inflammatory HDL.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's pro-inflammatory HDL level has occurred, a maintenance dose may be administered if necessary. Subsequently, the dosage or the frequency of administration, or both, can be reduced, as a function of the symptoms, to a level at which the improved (i.e., lowered) pro-inflammatory HDL level is retained. Patients can, however, require intermittent treatment on a long-term basis upon any recurrence of symptoms.

The amount of a given agent that will correspond to such an amount will vary depending upon factors such as the particular compound, level of pro-inflammatory HDL (e.g., prior to treatment, at various stages of treatment, etc.), disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment may be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

A pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In one example, a daily dosage appropriate for a leukotriene inhibitor of the current disclosure is from about 0.01 to 2.5 mg/kg per body weight. An indicated daily dosage in humans, is in the range from about 0.5 mg to about 100 mg, conveniently administered in divided doses, including, but not limited to, up to four times a day or in extended release form. Suitable unit dosage forms for oral administration comprise from about 1 to 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner. In another example, a dosage of a leukotriene inhibitor of the current disclosure is from about 20 mg to about 150 mg. In yet another example, a dosage of a leukotriene inhibitor that is 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid is about 20 mg to about 150 mg.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

FIG. 2 illustrates another example of a method 200 of reducing pro-inflammatory HDL. At step 205, it is determined if a human is in need of lowering pro-inflammatory HDL. Those skilled in the art will recognize techniques and procedures for measuring a level of pro-inflammatory HDL in a subject and/or indirectly determining that pro-inflammatory HDL levels are elevated (e.g., via presence of inflammatory symptoms, etc.). In one example, determination of a level of pro-inflammatory HDL may include determining a pro-inflammatory HDL function value.

In one example, a level or functionality of pro-inflammatory HDL can be determined using an assay methodology developed that is “cell free” (see, for example, the article McMahon et al., “A Panel of Biomarkers is Associated with Increased Risk of the Presence and Progression of Atherosclerosis in Women with Systemic Lupus Erythematosus,” Arthritis Rheumatol. 2014 January; 66(1): 130-139.), and article McMahon et al., “Dysfunctional Pro-Inflammatory High Density Lipoproteins Confer Increased Risk for Atherosclerosis in Women with Systemic Lupus Erythematosus,” Arthritis Rheum. 2009 August; 60(8): 2428-2437; and article Navab et al. “A Cell-Free Assay for Detecting HDL that is dysfunctional in preventing the formation of or inactivating oxidized Phoshpolipids,” J. Lipid Res. 2001, 42: 1308-1317, each of which is incorporated herein by reference for its disclosure of examples of such assay methodologies.) In such an example, HDL function is measured using a cell-free assay based on the ability of HDL to prevent oxidation. Normal HDL prevents oxidation of LDL and dichlorofluorescein diacetate (DCF-DA), which releases a fluorochrome upon interaction with lipid oxidation products (DCF). To determine HDL function, the change in fluorescence intensity (in fluorescence units [FU]) from oxidation of DCF/LDL in the presence or absence of test HDL is measured. LDL can be prepared from normal plasma. Techniques for preparing LDL from normal plasma are known (e.g., see Havel et al., “The Distribution and Chemical Composition of Ultracentrifugally Separated Lipoproteins in Human Serum,” J. Clin. Invest., 1955 September; 34 (9); 1345-1353, which is incorporated herein by reference for its disclosure of such a technique), and HDL can be prepared from test plasma using a dextran sulfate magnetic bead reagent. In one such methodology twenty-five microliters of LDL cholesterol (100 μg/ml) can be mixed with 6.25 μl of test HDL (100 μg HDL cholesterol/ml) in black, flat-bottomed polystyrene microtiter plates and incubated at 37° C. with rotation for 30 minutes. Twenty-five microliters of 2.0 mg/ml DCF solution can then added to each well, mixed, and incubated at 37° C. for 1 hour with rotation. Fluorescence can be determined using a fluorescence microplate reader (e.g., with a SpectraMax Gemini XS Fluorescence Microplate Reader (Molecular Devices) plate reader) at an excitation wavelength of 485 nm, emission wavelength of 530 nm, and cutoff of 515 nm, with the sensitivity of the photomultiplier set at medium. Values of DCF activated by LDL in the absence of HDL are normalized to 1.0 FU as the positive control. In assays with test HDL added, FU values >1.0 indicate HDL (in other examples, greater than or equal to 0.94 FU) that is dysfunctional and proinflammatory (piHDL); FU values <1.0 (in other examples, less than 0.94 FU) indicate that the HDL is anti-inflammatory. Each assay can be performed in a blinded manner, and the interassay and intraassay variation determined.

In another example description of a cell free assay (taken from Navab et al. “A Cell-Free Assay for Detecting HDL that is dysfunctional in preventing the formation of or inactivating oxidized Phoshpolipids,” J. Lipid Res. 2001, 42: 1308-1317), DCFH-DA (dichlorofluorescein diacetate) was dissolved in fresh m ethanol at 2.0 mg/ml and was incubated at room temperature and protected from light for 30 min. This results in the release of DCFH. On interaction with lipid oxidation products DCFH forms DCF (dichlorofluorescein), which produces intense fluorescence. Reagents were added to and incubated in 1×2.5 cm screw-cap polypropylene tubes. Fluorescence intensity was determined with a Farrand (Valhalla, N.Y.) system 3 scanning spectrofluorometer set at an excitation wavelength of 485 nm and an emission wavelength of 530 nm. A sensitivity level of 0.1 and slit widths of 2.5 and 10 nm were used for excitation and emission, respectively. For experiments investigating the protective effect of HDL on oxidation of LDL, 25 μl of LDL at 200 μg/ml was added to the tubes containing DCFH, followed by the addition of 25 dl of saline or HDL The volume was adjusted to 1.0 ml, using normal saline, and the tubes were gently vortexed and incubated at room temperature in the dark. Fluorescence readings were obtained at the indicated time points. The assay was also adapted for analyzing a large number of samples with a plate reader (Spectra Max, Gemini YS; Molecular Devices, Sunnyvale, Calif.). Round-bottom polypropylene microtiter plates (Fisher Scientific, Pittsburgh, Pa.) were utilized in place of polypropylene tubes. Combinations of PAPC plus HPODE, or Ox-PAPC plus saline or HDL, were first prepared in polypropylene tubes and subsequently aliquoted into microtiter plates at 100 μl/well. The final concentrations of PAPC, HPODE, Ox-PAPC, and HDL were as described above unless otherwise specified. Thus, unless otherwise specified each well contained DCFH (2.0 μg), PAPC (25 μg) plus HPODE (1.0 μg), or Ox-PAPC (25 μg) and HDL at the indicated concentrations. A correlation coefficient of 0.901 was obtained between the values generated with the spectrofluorometer and those obtained with the plate reader. Values for intra- and interassay variability were 4.2±1.3% and 6.3±2.1%, respectively.

At step 210, one or more leukotriene inhibitors are administered to the human (e.g., where it is determined that the human is in need of lowering pro-inflammatory HDL (therapeutic) or where it is determine that the human is not in need, but may benefit from, reduction of pro-inflammatory HDL (prophylactic)). Various aspects of administration are discussed above with respect to method 100. At step 215, a level of pro-inflammatory HDL is lowered in the human in response to the administration. At step 220, a determination is made if the pro-inflammatory HDL levels of the human have decreased. In one example, such a determination includes determining an amount of decrease of pro-inflammatory HDL (e.g., determining an amount of pro-inflammatory HDL converted to anti-inflammatory HDL, determining a change in the amount of anti-inflammatory HDL, determining a change in the amount of pro-inflammatory HDL, etc.). Comparison can be made in measurements of level and/or function of pro-inflammatory HDL taken prior to treatment. See above discussion with respect to step 205 for examples of determining level and/or function of pro-inflammatory HDL. It is noted that step 205 and/or step 220 may be omitted.

It is further contemplated that a method of reducing a level of pro-inflammatory HDL as described herein (e.g., methods 100 and 200 of FIGS. 1 and 2, respectively), can be utilized to treat a patient suffering from systemic lupus erythematosus to lower the level of pro-inflammatory HDL in the patient. In one example, the lowering of pro-inflammatory HDL can be via a conversion of pro-inflammatory HDL into anti-inflammatory HDL.

### Illustrative Biological Activity

Leukotrienes (LTs) are potent contractile and inflammatory mediators produced by release of arachidonic acid from cell membranes and conversion to leukotrienes by the action of 5-lipoxygenase, 5-lipoxygenase-activating protein, LTA₄ hydrolase and LTC₄ synthase. The leukotriene synthesis pathway, or 5-lipoxygenase pathway, involves a series of enzymatic reactions in which arachidonic acid is converted to leukotriene LTB₄, or the cysteinyl leukotrienes, LTC₄, LTD₄, and LTE₄. The pathway occurs mainly at the nuclear envelope and has been described. See, e.g., Wood, J W et al, J. Exp. Med., 178: 1935-1946, 1993; Peters-Golden, Am. J. Respir. Crit. Care Med. 157:S227-S232, 1998; Drazen, et al., ed. Five-Lipoxygenase Products in Asthma, Lung Biology in Health and Disease Series, Vol. 120, Chs. 1, 2, and 7, Marcel Dekker, Inc. NY, 1998. Protein components dedicated to the leukotriene synthesis pathway include a 5-lipoxygenase (5-LO), a 5-lipoxygenase-activating protein, a LTA₄ hydrolase, and a LTC₄ synthase. The synthesis of leukotrienes has been described in the literature, e.g., by Samuelsson et al, Science, 220, 568-575, 1983; Peters-Golden, “Cell Biology of the 5-Lipoxygenase Pathway” Am J Respir Crit Care Med 157:S227-S232 (1998). Leukotrienes are synthesized directly from arachidonic acid by different cells including eosinophils, neutrophils, basophils, lymphocytes, macrophages, monocytes and mast cells. Excess LTA₄, for example from an activated neutrophil, may enter a cell by a transcellular pathway. Most cells in the body have LTA₄ hydrolase so they can produce LTB₄. Platelets and endothelial cells have LTC₄ synthase, so can make LTC₄ when presented with LTA₄ by a transcellular pathway.

Arachidonic acid is a polyunsaturated fatty acid and is present mainly in the membranes of the body's cells. Upon presentation of inflammatory stimuli from the exterior of the cell, calcium is released and binds to phospholipase A₂ (PLA₂) and 5-LO. Cell activation results in the translocation of PLA₂ and 5-LO from the cytoplasm to the endoplasmic reticulum and/or nuclear membranes, where in the presence of FLAP, the released arachidonic acid is converted via a 5-HPETE intermediate to the epoxide LTA₄. Depending on the cell type, the LTA₄ may be immediately converted to LTC₄ by the nuclear-bound LTC₄ synthase or to LTB₄ by the action of cytosolic LTA₄ hydrolase. LTB₄ is exported from cells by an as yet uncharacterized transporter and may activate other cells, or the cell it was made in, via high affinity binding to one of two G protein-coupled receptors (GPCRs), namely BLT₁R or BLT₂R. LTC₄ is exported to the blood via the MRP-1 anion pump and rapidly converted to LTD₄ by the action of γ-glutamyl transpeptidase and LTD₄ is then converted to LTE₄ by the action of dipeptidases. LTC₄, LTD₄ and LTE₄ are collectively referred to as the cysteinyl leukotrienes (or previously as slow reacting substance of anaphylaxis, SRS-A). The cysteinyl leukotrienes activate other cells, or the cells they are made in, via high affinity binding to one of two GPCRs, namely CysLT₁R or CysLT₂R. CysLT₁ receptors are found in the human airway eosinophils, neutrophils, macrophages, mast cells, B-lymphocytes and smooth muscle and induce bronchoconstriction. Zhu et al, Am J Respir Cell Mol Biol Epub August 25 (2005). CysLT₂ receptors are located in human airway eosinophils, macrophages, mast cells and the human pulmonary vasculature Figueroa et al, Clin Exp Allergy 33:1380-1388 (2003).

### Involvement of Leukotrienes in Diseases or Conditions

The involvement of leukotrienes in disease is described in detail in the literature. See e.g., by Busse, Clin. Exp. Allergy 26:868-79, 1996; O'Byrne, Chest 111(Supp. 2): 27S-34S, 1977; Sheftell, F. D., et al., Headache, 40:158-163, 2000; Klickstein et al., J. Clin. Invest., 66:1166-1170, 1950; Davidson et al., Ann. Rheum. Dis., 42:677-679, 1983 Leukotrienes produce marked inflammatory responses in human skin. Evidence for the involvement of leukotrienes in a human disease is found in psoriasis, in which leukotrienes have been detected in psoriatic lesions (Kragballe et al., Arch. Dermatol., 119:548-552, 1983).

For example, inflammatory responses have been suggested to reflect three types of changes in the local blood vessels. The primary change is an increase in vascular diameter, which results in an increase in local blood flow and leads to an increased temperature, redness and a reduction in the velocity of blood flow, especially along the surfaces of small blood vessels. The second change is the activation of endothelial cells lining the blood vessel to express adhesion molecules that promote the binding of circulating leukocytes. The combination of slowed blood flow and induced adhesion molecules allows leukocytes to attach to the endothelium and migrate into the tissues, a process known as extravasation. These changes are initiated by cytokines and leukotrienes produced by activated macrophages. Once inflammation has begun, the first cells attracted to the site of infection are generally neutrophils. They are followed by monocytes, which differentiate into more tissue macrophages. In the latter stages of inflammation, other leukocytes, such as eosinophils and lymphocytes also enter the infected site. The third major change in the local blood vessels is an increase in vascular permeability. Instead of being tightly joined together, the endothelial cells lining the blood vessel walls become separated, leading to exit of fluid and proteins from the blood and their local accumulation in the tissue. (See Janeway, et al., Immunobiology: the immune system in health and disease, 5th ed., Garland Publishing, New York, 2001).

LTB₄ produces relatively weak contractions of isolated trachea and lung parenchyma, and these contractions are blocked in part by inhibitors of cyclooxygenase, suggesting that the contraction are secondary to the release of prostaglandins. However, LTB₄ has been shown to be a potent chemotactic agent for eosinophils and progenitors of mast cells and the LTB₄ receptor BLT1−/− knockout mouse is protected from eosinophilic inflammation and T-cell mediated allergic airway hyperreactivity. Miyahara et al. J Immunol 174:4979-4784; (Weller et al. J Exp Med 201: 1961-1971(2005).

Leukotrienes C₄ and D₄ are potent smooth muscle contractile agents, promoting bronchoconstriction in a variety of species, including humans (Dahlen et al., Nature, 288:484-486, 1980). These compounds have profound hemodynamic effects, constricting coronary blood vessels, and resulting in a reduction of cardiac output efficiency (Marone et al., in Biology of Leukotrienes, ed. By R. Levi and R. D. Krell, Ann. New York Acad. Sci. 524:321-333, 1988). Leukotrienes also act as vasoconstrictors; however, marked differences exist for different vascular beds. There are reports suggesting that leukotrienes contribute to cardiac reperfusion injury following myocardial ischemia (Barst and Mullane, Eur. J Pharmacol., 114: 383-387, 1985; Sasaki et al., Cardiovasc. Res., 22: 142-148, 1988). LTC₄ and LTD₄ directly increase vascular permeability probably by promoting retraction of capillary endothelial cells via activation of the CysLT₂ receptor and possibly other as yet undefined CysLT receptors [Lotzer et al. Arterioscler Thromb Vasc Biol 23: e32-36. (2003)]. LTB₄ enhances atherosclerotic progression in two atherosclerotic mouse models, namely low density receptor lipoprotein receptor deficient (LDLr−/−) and apolipoprotein E-deficient (ApoE−/−) mice (Aiello et al, Arterioscler Thromb Vasc Biol 22:443-449 (2002); Subbarao et al, Arterioscler Thromb Vasc Biol 24:369-375 (2004); Heller et al. Circulation 112:578-586 (2005). LTB₄ has also been shown to increase human monocyte chemoattractant protein (MCP-1) a known enhancer of atherosclerotic progression (Huang et al. Aterioscler Thromb Vasc Biol 24:1783-1788 (2004).

The role of FLAP in the leukotriene synthesis pathway is significant because FLAP in concert with 5-lipoxygenase performs the first step in the pathway for the synthesis of leukotrienes. Therefore the leukotriene synthesis pathway provides a number of targets for compounds useful in the treatment of diseases or conditions where leukotriene mediated inflammation is a major factor (Sharma J N and Mohammed L A, Inflammopharmacology. 2006 March; 14(1-2):10-6), including, by way of example, systemic lupus erythematosus, rheumatoid arthritis and type II diabetes.

### Pro-Inflammatory HDL

High density lipoprotein (HDL) is a soluble complex of proteins (apolipoproteins) and lipids that are one of the major cholesterol transport vehicles in the body (S. Lund-Katz and M. C. Phillips, Subcell Biochem. 2010; 51: 183-227). HDL also interacts with low density lipoprotein (LDL), a cholesterol transport protein known to contribute to the development of arthrosclerosis, to inhibit its oxidation and pro-inflammatory effects (Philip Barter, Eur Heart J Suppl (2005) 7 (suppl_F): F4-F8). In certain cases where HDL is exposed to a prolonged inflammatory environment it is transformed to a pro-inflammatory state and loses its ability to counteract the pathological effects of LDL (Hima Bindu G, et al, Cholesterol, Volume 2011 (2011), Article ID 274629). Therefore, the detection of pro-inflammatory IDL in diseases characterized by prolonged inflammation is a bio marker for the danger of development of arthrosclerosis (McMahon M, et al, Arthritis Rheum. 2006 August; 54(8):2541-9).

Leukotrienes are known to contribute to the inflammation in many diseases and conditions and 3-[3-tert-butylsulfanyl-1-[4-(6-ethoxy-pyridin-3-yl)-benzyl]-5-(5-methyl-1-pyridin-2-ylmethoxy)-1H-indol-2-yl]-2,2-dimethyl-propionic acid (fiboflapon) has been shown to be a potent inhibitor of leukotriene production in vivo (Stock, N S et al., J Med Chem. 2011 Dec. 8; 54(23):8013-296 (1993). As shown above, prolonged inflammation in some diseases also leads to the formation of pro-inflammatory HDL which is known to contribute to the development of coronary artery disease. Therefore, the presence of pro-inflammatory HDL can be used as a bio marker for the selection of patients to treat with a leukotriene inhibitor (e.g., a FLAP inhibitor such as fiboflapon); the diseases include, but are not limited to systemic lupus erythematosus, rheumatoid arthritis and type II diabetes.

### Certain Chemical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. In this application, the use of “or” means “and/or” unless stated otherwise. Furthermore, use of the term “including” as well as other forms, such as “include”, “includes,” and “included,” is not limiting.

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 5 carbon atoms. The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkylamine” refers to the —N(alkyl)ₓH_{y} group, where x and y are selected from the group x=1, y=1 and x=2, y=0. When x=2, the alkyl groups, taken together, can optionally form a cyclic ring system.

The term “alkenyl” refers to a type of alkyl group in which the first two atoms of the alkyl group form a double bond that is not part of an aromatic group. That is, an alkenyl group begins with the atoms —C(R)═C—R, wherein R refers to the remaining portions of the alkenyl group, which may be the same or different. Non-limiting examples of an alkenyl group include —CH═CH, —C(CH₃)═CH, —CH═CCH₃ and —C(CH₃)═CCH₃. The alkenyl moiety may be branched, straight chain, or cyclic (in which case, it would also be known as a “cycloalkenyl” group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CCH₃ and —C≡CCH₂CH₃. The “R” portion of the alkynyl moiety may be branched, straight chain, or cyclic.

An “amide” is a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound of any of Formula (G), Formula (G-I), or Formula (G-II), thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “aromatic” or “aryl” refers to an aromatic group which has at least one ring having a conjugated pi electron system and includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

The term “bond” or “single bond” refers to a chemical bond between two atoms, or two moieties when the atoms joined by the bond are considered to be part of larger substructure.

A “cyano” group refers to a —CN group.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

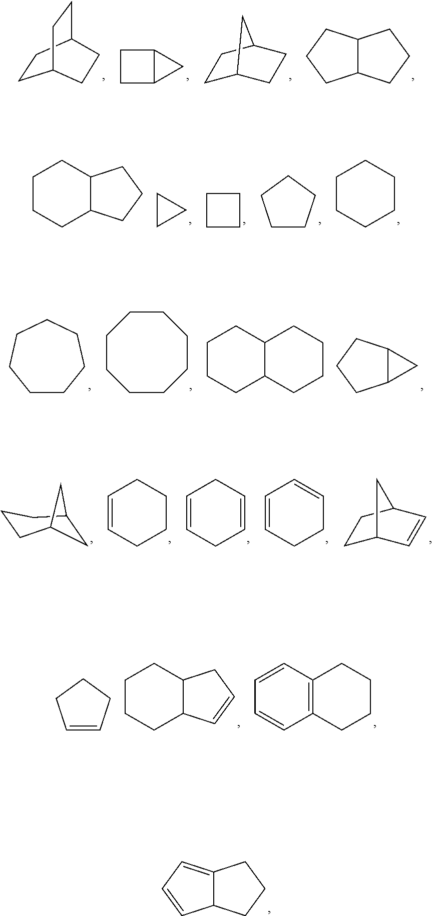

and the like.

The term “ester” refers to a chemical moiety with formula —COOR, where R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety. The term “halo” or, alternatively, “halogen” means fluoro, chloro, bromo or iodo.

The terms “haloalkyl,” “haloalkenyl,” “haloalkynyl” and “haloalkoxy” include alkyl, alkenyl, alkynyl and alkoxy structures that are substituted with one or more halo groups or with combinations thereof. The terms “fluoroalkyl” and “fluoroalkoxy” include haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

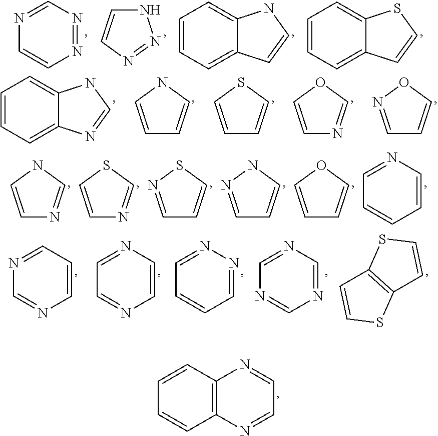

and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

A “heteroalicyclic” group refers to a cycloalkyl group that includes at least one heteroatom selected from nitrogen, oxygen and sulfur. The radicals may be fused with an aryl or heteroaryl. Illustrative examples of heterocycloalkyl groups, also referred to as non-aromatic heterocycles, include:

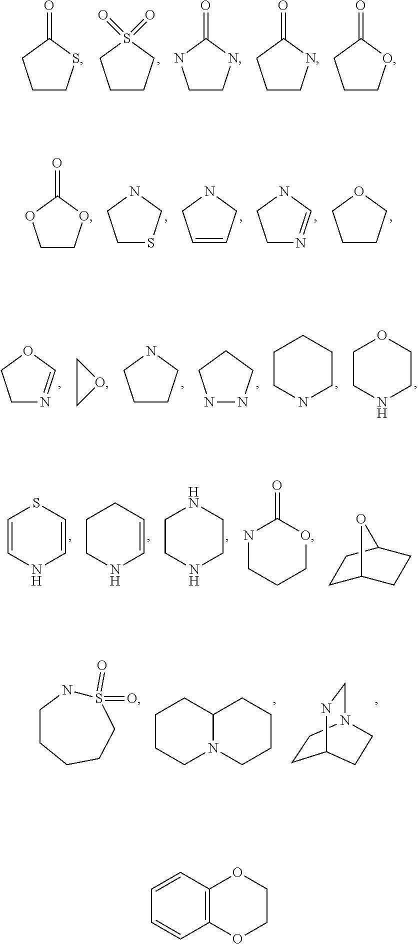

and the like. The term heteroalicyclic also includes all ring forms of the carbohydrates, including but not limited to the monosaccharides, the disaccharides and the oligosaccharides.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

An “isocyanato” group refers to a —NCO group.

An “isothiocyanato” group refers to a —NCS group.

A “mercaptyl” group refers to a (alkyl)S— group.

The term “moiety” refers to a specific segment or functional group of a molecule. Chemical moieties are often recognized chemical entities embedded in or appended to a molecule.

A “sulfinyl” group refers to a —S(═O)—R, where R is selected from the group consisting of alkyl, cycloalkyl, aryl, heteroaryl (bonded through a ring carbon) and heteroalicyclic (bonded through a ring carbon)

A “sulfonyl” group refers to a —S(═O)₂—R, where R is selected from the group consisting of alkyl, cycloalkyl, aryl, heteroaryl (bonded through a ring carbon) and heteroalicyclic (bonded through a ring carbon).

A “thiocyanato” group refers to a —CNS group.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents may may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted lower alkyl), (substituted or unsubstituted lower cycloalkyl), heteroaryl, or heteroalkyl. The protecting groups that may form the protective derivatives of the above substituents are known to those of skill in the art and may be found in references such as Greene and Wuts, above.

The compounds of Formula (G), (G-I), and (G-II) presented herein may possess one or more stereocenters and each center may exist in the R or S configuration. Such compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of any of Formula (G), Formula (G-I), or Formula (G-II), as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

### Certain Pharmaceutical Terminology

The term “acceptable” with respect to a formulation, composition or ingredient, as used herein, means having no persistent detrimental effect on the general health of the subject being treated.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “asthma” as used herein refers to any disorder of the lungs characterized by variations in pulmonary gas flow associated with airway constriction of whatever cause (intrinsic, extrinsic, or both; allergic or non-allergic). The term asthma may be used with one or more adjectives to indicate cause.

The term “cardiovascular disease,” as used herein refers to diseases affecting the heart or blood vessels or both, including but not limited to: arrhythmia; atherosclerosis and its sequelae; angina; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart or other organ or tissue; endotoxic, surgical, or traumatic shock; hypertension, valvular heart disease, heart failure, abnormal blood pressure; shock; vasoconstriction (including that associated with migraines); vascular abnormality, inflammation, insufficiency limited to a single organ or tissue. [Lotzer K et al., “The 5-lipoxygenase pathway in arterial wall biology and atherosclerosis”, Biochim Biophys Acta 2005; 1736:30-7; Helgadottir A et al., “The gene encoding 5-lipoxygenase activating protein confers risk of myocardial infarction and stroke’, Nat Genet. 2004 March; 36(3):233-9. Epub 2004 Feb. 8; [Heise C E, Evans J F et al., “Characterization of the human cysteinyl leukotriene 2 receptor”, J Biol Chem. 2000 Sep. 29; 275(39):30531-6].

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “dermatological disorder,” as used herein refers to a skin disorder. Such dermatological disorders include, but are not limited to, proliferative or inflammatory disorders of the skin such as, atopic dermatitis, bullous disorders, collagenoses, contact dermatitis eczema, Kawasaki Disease, rosacea, Sjogren-Larsso Syndrome, urticaria [Wedi B et al., “Pathophysiological role of leukotrienes in dermatological diseases: potential therapeutic implications”, BioDrugs. 2001; 15(11):729-43].

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated (e.g., reduce by some extent a level of pro-inflammatory HDL). The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a level of pro-inflammatory HDL. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “enzymatically cleavable linker,” as used herein refers to unstable or degradable linkages which may be degraded by one or more enzymes.

The terms “fibrosis” or “fibrosing disorder,” as used herein, refers to conditions that follow acute or chronic inflammation and are associated with the abnormal accumulation of cells and/or collagen and include but are not limited to fibrosis of individual organs or tissues such as the heart, kidney, joints, lung, or skin, and includes such disorders as idiopathic pulmonary fibrosis and cryptogenic fibrosing alveolitis [Charbeneau R P et al., “Eicosanoids: mediators and therapeutic targets in fibrotic lung disease”, Clin Sci (Lond). 2005 June; 108(6):479-91].

The term “iatrogenic” means a leukotriene-dependent or leukotriene-mediated condition, disorder, or disease created or worsened by medical or surgical therapy.

The term “inflammatory disorders” refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporarl arteritis); joints (arthritis: crystalline, osteo-, psoriatic, reactive, rheumatoid, Reiter's); gastrointestinal tract (Disease); skin (dermatitis); or multiple organs and tissues (systemic lupus erythematosus) [Harrison's Principles of Internal Medicine, 16^{th} Edition, Kasper D L, et al, Editors; McGraw-Hill, publishers].

The term “interstitial cystitis” refers to a disorder characterized by lower abdominal discomfort, frequent and sometimes painful urination that is not caused by anatomical abnormalites, infection, toxins, trauma or tumors [Bouchelouche K et al., “The cysteinyl leukotrine D4 receptor antagonst montelukast for the treatment of interstitial cystitis”, J Urol 2001; 166:1734].

The term “leukotriene-driven mediators,” as used herein, refers to molecules able to be produced in a patient that may result from excessive production of leukotriene stimulation of cells, such as, by way of example only, LTB₄, LTC₄, LTE₄, cysteinyl leuktorienes, monocyte inflammatory protein (MIP-1α), interleukin-8 (IL-8), interleukin-4 (IL-4), interleukin-13 (IL-13), monocyte chemoattractant protein (MCP-1), soluble intracellular adhesion molecule (sICAM; soluble ICAM), myeloperoxidase (MPO), eosinophil peroxidase (EPO), and general inflammation molecules such as interleukin-6 (11-6), C-reactive protein (CRP), and serum amyloid A protein (SAA).

The term “leukotriene-related mediators,” as used herein, refers to molecules able to be produced in a patient that may result from excessive production of leukotriene stimulation of cells, such as, by way of example only, LTB₄, LTC₄, LTE₄, cysteinyl leuktorienes, monocyte inflammatory protein (MIP-1α), interleukin-8 (IL-8), interleukin-4 (IL-4), interleukin-13 (IL-13), monocyte chemoattractant protein (MCP-1), soluble intracellular adhesion molecule (sICAM; soluble ICAM), myeloperoxidase (MPO), eosinophil peroxidase (EPO), and general inflammation molecules such as interleukin-6 (Il-6), C-reactive protein (CRP), and serum amyloid A protein (SAA).

The term “leukotriene-dependent”, as used herein, refers to conditions or disorders that would not occur, or would not occur to the same extent, in the absence of one or more leukotrienes.

The term “leukotriene-mediated”, as used herein, refers to refers to conditions or disorders that might occur in the absence of leukotrienes but can occur in the presence of one or more leukotrienes.

The term “leukotriene-responsive patient,” as used herein, refers to a patient who has been identified by either genotyping of FLAP haplotypes, or genotyping of one or more other genes in the leukotriene pathway and/or, by phenotyping of patients either by previous positive clinical response to another leukotriene modulator, including, by way of example only, zileuton (Zyflo®), montelukast (Singulair™), pranlukast (Onon™), zafirlukast (Accolate®), and/or by their profile of leukotriene-driven mediators that indicate excessive leukotriene stimulation of inflammatory cells, as likely to respond favorably to leukotriene modulator therapy.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The terms “neurogenerative disease” or “nervous system disorder,” as used herein, refers to conditions that alter the structure or function of the brain, spinal cord or peripheral nervous system, including but not limited to Alzheimer's Disease, cerebral edema, cerebral ischemia, multiple sclerosis, neuropathies, Parkinson's Disease, those found after blunt or surgical trauma (including post-surgical cognitive dysfunction and spinal cord or brain stem injury), as well as the neurological aspects of disorders such as degenerative disk disease and sciatica. The acronym “CNS” refers to disorders of the central nervous system, i.e., brain and spinal cord [Sugaya K, et al., “New anti-inflammatory treatment strategy in Alzheimer's disease”, Jpn J Pharmacol. 2000 February; 82(2):85-94; Yu G L, et al., “Montelukast, a cysteinyl leukotriene receptor-1 antagonist, dose- and time-dependently protects against focal cerebral ischemia in mice”, Pharmacology. 2005 January; 73(1):31-40. Epub 2004 Sep. 27; [Zhang W P, et al., “Neuroprotective effect of ONO-1078, a leukotriene receptor antagonist, on focal cerebral ischemia in rats’, Acta Pharmacol Sin. 2002 October; 23(10):871-7].

The terms “ocular disease” or “ophthalmic disease,” as used herein, refer to diseases which affect the eye or eyes and potentially the surrounding tissues as well. Ocular or ophthalmic diseases include, but are not limited to, conjunctivitis, retinitis, scleritis, uveitis, allergic conjuctivitis, vernal conjunctivitis, pappillary conjunctivitis [Toriyama S., “Effects of leukotriene B4 receptor antagonist on experimental autoimmune uveoretinitis in rats”, Nippon Ganka Gakkai Zasshi. 2000 June; 104(6):396-40; [Chen F, et al., “Treatment of S antigen uveoretinitis with lipoxygenase and cyclo-oxygenase inhibitors”, Ophthalmic Res. 1991; 23(2):84-91].

By “pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound of a leukotriene inhibitor (e.g., one of Formula (G), Formula (G-I), or Formula (G-II)), with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts may also be obtained by reacting a compound of a leukotriene inhibitor (e.g., one of Formula (G), Formula (G-I), or Formula (G-II)), with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of any of Formula (G), Formula (G-I), or Formula (G-II), and a co-agent, are both administered to a patient simultaneously in the form of a single entity or dosage. The term “non-fixed combination” means that the active ingredients, e.g. a compound of any of Formula (G), Formula (G-I), or Formula (G-II), and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a leukotriene inhibitor (e.g., a compound of any of Formula (G), Formula (G-I), or Formula (G-II)), with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a leukotriene inhibitor (e.g., a compound of any of Formula (G), Formula (G-I), or Formula (G-II)), which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The term “respiratory disease,” as used herein, refers to diseases affecting the organs that are involved in breathing, such as the nose, throat, larynx, trachea, bronchi, and lungs. Respiratory diseases include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia [Evans J F, “The Cysteinyl Leukotriene (CysLT) Pathway in Allergic Rhinitis”, Allergology International 2005; 54:187-90); Kemp J P., “Leukotriene receptor antagonists for the treatment of asthma”, IDrugs. 2000 April; 3(4):430-41; Riccioni G, et al., “Effect of the two different leukotriene receptor antagonists, montelukast and zafirlukast, on quality of life: a 12-week randomized study”, Allergy Asthma Proc. 2004 November-December; 25(6):445-8].

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Examples of Specific Leukotriene Inhibitor Compounds

### Compounds of Formula (Z1)

Compounds of Formula (Z1) pharmaceutically acceptable salts, pharmaceutically acceptable N-oxides, pharmaceutically active metabolites, pharmaceutically acceptable prodrugs, and pharmaceutically acceptable solvates thereof, may be used to treat patients to lower pro-inflammatory HDL.

Formula (Z1) is as follows:

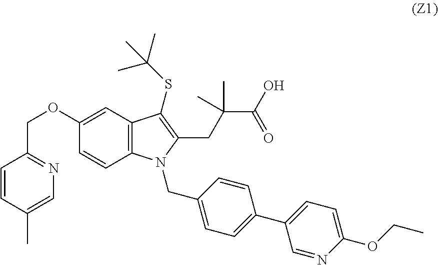 [SMILES: CCOc1ccc(-c2ccc(Cn3c(CC(C)(C)C(=O)O)c(SC(C)(C)C)c4cc(OCc5ccc(C)cn5)ccc43)cc2)cn1]

### Compounds of Formula (G), Formula (G-I) and Formula (G-II):

Compounds of Formula (G), Formula (G-I), and Formula (G-II), pharmaceutically acceptable salts, pharmaceutically acceptable N-oxides, pharmaceutically active metabolites, pharmaceutically acceptable prodrugs, and pharmaceutically acceptable solvates thereof, may be used to treat patients to lower pro-inflammatory HDL.

Formula (G-I) is as follows:

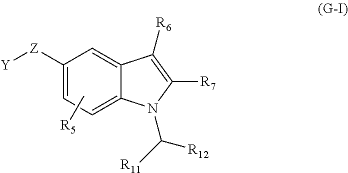

wherein,
Z is selected from N(R₁), S(O)ₘ, CR₁═CR₁, —C≡C—, C(R₁)₂[C(R₂)₂]ₙ, [C(R₂)₂]ₙC(R₁)₂O, OC(R₁)₂[C(R₂)₂]ₙ, [C(R₂)₂]ₙC(R₁)₂S(O)ₘ, S(O)ₘC(R₁)₂[C(R₂)₂]ₙ, [C(R₂)₂]ₙC(R₁)₂NR₁, NR₁C(R₁)₂[C(R₂)₂]ₙ, [C(R₂)₂]ₙO[C(R₁)₂]ₙ, [C(R₁)₂]ₙO[C(R₂)₂]ₙ, —C(O)NR₂—, —NR₂C(O)—, —NR₂C(O)O—, —OC(O)NR₂—, —S(O)₂NR₂—, —CR₁═N—N—, NR₂C(O)NR₂—, —OC(O)O—, S(O)₂NR₂, or —NR₂S(O)₂—, wherein each R₁ is independently H, CF₃, or an optionally substituted lower alkyl and two R₁ on the same carbon may join to form a carbonyl (═O); and each R₂ is independently H, OH, OMe, CF₃, or an optionally substituted lower alkyl and two R₂ on the same carbon may join to form a carbonyl (═O); m is 0, 1 or 2; each n is independently 0, 1, 2, or 3;
Y is -L₁-(substituted or unsubstituted aryl); -L₁-(substituted or unsubstituted heteroaryl); -L₁-(substituted or unsubstituted non-aromatic heterocycle), provided that when the heteroatom is directly bound to Z, the non-aromatic heterocycle is substituted; where L₁ is a bond, a substituted or unsubstituted alkyl, substituted or unsubstituted alkenyl, substituted or unsubstituted alkynyl, a substituted or unsubstituted heterocycle, a substituted or unsubstituted heteroaryl, a substituted or unsubstituted cycloalkyl, or substituted or unsubstituted aryl, —C(O), C(R₈)(OH), C(R₈)(OMe), C(═NOH), C(═NOR_{4b}), C(═O)NH, C(═O)NR_{4b}, —NHC(═O), NR_{4b}C(═O), S, S(═O), S(═O)₂, —NHC(═O)NH, or NR_{4b}C(═O)NR_{4b};
   where each substituent on Y or Z is (LₛRₛ)ⱼ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, C₁-C₆ alkyl, C₂-C₆ alkenyl, —C₁-C₆ fluoroalkyl, heteroaryl, aryl, or heterocycle; and each Rₛ is independently selected from H, halogen, —N(R₄)₂, —CN, —NO₂, N₃, —S(═O)₂NH₂, lower alkyl, lower cycloalkyl, —C₁-C₆ fluoroalkyl, heteroaryl, or heteroalkyl; where j is 0, 1, 2, 3, or 4;
   each R₃ is independently selected from H, —S(═O)₂R₈, —S(═O)₂NH₂ —C(O)R₈, —CN, —NO₂, heteroaryl, or heteroalkyl;
   each R_{3b} is independently selected from substituted or unsubstituted lower alkyl, substituted or unsubstituted lower cycloalkyl, substituted or unsubstituted phenyl or benzyl;
   each R₄ is independently selected from H, substituted or unsubstituted lower alkyl, substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7-, or 8-membered heterocyclic ring;
   each R_{4b} is independently selected from H, substituted or unsubstituted lower alkyl, substituted or unsubstituted lower cycloalkyl, substituted or unsubstituted aryl or substituted or unsubstituted benzyl; substituted or unsubstituted heteroaryl, substituted or unsubstituted heterocycle;
   R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(O), —CH(OH), -(substituted or unsubstituted C₁-C₆ alkyl), or -(substituted or unsubstituted C₂-C₆ alkenyl);
   R₇ is L₃-X-L₄-G₁, wherein,
      L₃ is a bond, or substituted or unsubstituted alkyl;
      X is a bond, O, —C(═O), —CR₉(OR₉), S, —S(═O), —S(═O)₂, —NR₉, —NR₉C(O), —C(O)NR₉, —NR₉C(O)NR₉—, or aryl;
         L₄ is a bond, or substituted or unsubstituted alkyl;
      G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O);
      or G₁ is W-G₅, where W is a substituted or unsubstituted aryl, substituted or unsubstituted heterocycle or substituted or unsubstituted heteroaryl and G₅ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈;
         each R₈ is independently selected from substituted or unsubstituted lower alkyl, substituted or unsubstituted lower cycloalkyl, phenyl or benzyl;
         each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and
         each R₁₀ is independently selected from H, —S(═O)₂R₈, —S(═O)₂NH₂ —C(O)R₈, —CN, —NO₂, heteroaryl, or heteroalkyl;
   R₅ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), —NHC(O)NH—, or —C(O)NH;
   R₁₁ is L₇-L₁₀-G₆, wherein L₇ is a bond, —O, —S, —S(═O), —S(═O)₂, —NH, —C(O), —C(O)NH, —NHC(O), (substituted or unsubstituted C₁-C₆ alkyl), or (substituted or unsubstituted C₂-C₆ alkenyl);
      L₁₀ is a bond, (substituted or unsubstituted alkyl), (substituted or unsubstituted cycloalkyl), (substituted or unsubstituted cycloalkenyl), (substituted or unsubstituted heteroaryl), (substituted or unsubstituted aryl), or (substituted or unsubstituted heterocycle);
      G₆ is H, CN, SCN, N₃, NO₂, halogen, OR₉, —C(═O)CF₃, —C(═O)R₉, —C(═O)OR₉, —SR₈, —S(═O)R₈, —S(═O)₂R₈, N(R₉)₂, tetrazolyl, —NHS(═O)₂R₈, —S(═O)₂N(R₉)₂, —C(O)NHS(═O)₂R₈, —S(═O)₂NHC(O)R₉, —C(═O)N(R₉)₂, N R₉C(O)R₉, C(R₉)₂C(═O)N(R₉)₂—C(═NR₁₀)N(R₉)₂, —NR₉C(═NR₁₀)N(R₉)₂, —NR₉C(═CR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(O)O, —NHC(O)NH—, —OC(O)O—, —OC(O)NH—, —NHC(O), —C(O)NH, —C(O)O, or —OC(O);
      or G₆ is W-G₇, wherein W is (substituted or unsubstituted heterocycle) or a (substituted or unsubstituted heteroaryl) and G₇ is H, halogen, CN, NO₂, N₃, CF₃, OCF₃, C₁-C₆ alkyl, C₃-C₆ cycloalkyl, —C₁-C₆ fluoroalkyl, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycle), or -L₅-(substituted or unsubstituted aryl), wherein L₅ is a bond, —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O);
      provided that when L₁₀ is phenyl or thiophenyl, Y is -(substituted or unsubstituted heteroaryl), -(substituted or unsubstituted aryl), and Z is [C(R₂)₂]ₙC(R₁)₂₀, then G₆ is W-G₇; and
   R₁₂ is H, (substituted or unsubstituted C₁-C₆ alkyl), (substituted or unsubstituted C₃-C₆ cycloalkyl).

For any and all of the embodiments of Formula (G), Formula (G-I), and Formula (G-II), substituents are selected from among a list of alternatives. For example, in one embodiment, the non-aromatic heterocycle of Y is selected from quinolizines, dioxines, piperidines, morpholines, thiazines, tetrahydropyridines, piperazines, oxazinanones, dihydropyrroles, dihydroimidazoles, tetrahydrofurans, dihydrooxazoles, oxiranes, pyrrolidines, pyrazolidines, dihydrothiophenones, imidazolidinones, pyrrolidinones, dihydrofuranones, dioxolanones, thiazolidines, piperidinones, tetrahydronaphthalenes, tetrahydroquinolines, tetrahydrothiophenes, and thiazepanes.

In further embodiments, the non-aromatic heterocycle of Y is selected from the group consisting of the following structures:

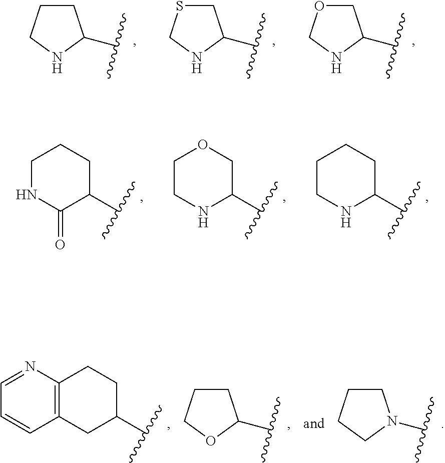

By way of example only, the non-aromatic heterocycle of Y is selected from

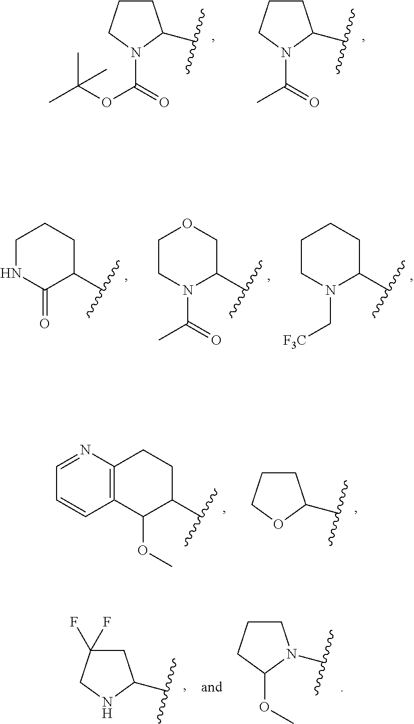

In a further or alternative embodiment, the “G” group (e.g. G₁, G₅, G₆, G₇) is any group that is used to tailor the physical and biological properties of the molecule. Such tailoring/modifications are achieved using groups which modulate acidity, basicity, lipophilicity, solubility and other physical properties of the molecule. The physical and biological properties modulated by such modifications to “G” include, by way of example only, solubility, in vivo absorption, and in vivo metabolism. In addition, in vivo metabolism may include, by way of example only, controlling in vivo PK properties, off-target activities, potential toxicities associated with cypP450 interactions, drug-drug interactions, and the like. Further, modifications to “G” allow for the tailoring of the in vivo efficacy of the compound through the modulation of, by way of example, specific and non-specific protein binding to plasma proteins and lipids and tissue distribution in vivo. Additionally, such tailoring/modifications to “G” allow for the design of compounds selective for 5-lipoxygenase-activating protein over other proteins.

In further or alternative embodiments, “G” is L₂₀-Q, wherein L₂₀ is an enzymatically cleavable linker and Q is a drug, or an affinity moiety. In further or alternative embodiments, the drug includes, by way of example only, leukotriene receptor antagonists and anti-inflammatory agents. In further or alternative embodiments, the leukotriene receptor antagonists include, but are not limited to, CysLT₁/CysLT₂ dual antagonists and CysLT₁ antagonists. In further or alternative embodiments, the affinity moiety allow for site specific binding and include, but are not limited to, antibodies, antibody fragments, DNA, RNA, siRNA, and ligands.

Formula (G-II) is as follows:

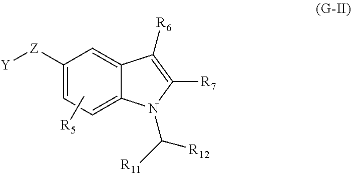

wherein,
Z is selected from N(R₁), S(O)ₘ, CR₁═CR₁, —C≡C—, C(R₁)₂[C(R₂)₂]ₙ, [C(R₂)₂]ₙC(R₁)₂₀, OC(R₁)₂[C(R₂)₂]ₙ, [C(R₂)₂]ₙC(R₁)₂S(O)ₘ, S(O)ₘC(R₁)₂[C(R₂)₂]ₙ, [C(R₂)₂]ₙC(R₁)₂NR₁, NR₁C(R₁)₂[C(R₂)₂]ₙ, [C(R₂)₂]ₙO[C(R₁)₂]ₙ, [C(R₁)₂]ₙO[C(R₂)₂]ₙ, —C(O)NR₂—, —NR₂C(O)—, —NR₂C(O)O—, —OC(O)NR₂—, —S(O)₂NR₂—, —CR₁═N—N—, NR₂C(O)NR₂—, —OC(O)O—, S(O)₂NR₂, or —NR₂S(O)₂—, wherein each R₁ is independently H, CF₃, or an optionally substituted lower alkyl and two R₁ on the same carbon may join to form a carbonyl (═O); and each R₂ is independently H, OH, OMe, CF₃, or an optionally substituted lower alkyl and two R₂ on the same carbon may join to form a carbonyl (═O); m is 0, 1 or 2; each n is independently 0, 1, 2, or 3;
Y is -L₁-(substituted or unsubstituted aryl); -L₁-(substituted or unsubstituted heteroaryl); -L₁-(substituted or unsubstituted non-aromatic heterocycle), provided that when the heteroatom is directly bound to Z, the non-aromatic heterocycle is substituted; where L₁ is a bond, a substituted or unsubstituted alkyl, substituted or unsubstituted alkenyl, substituted or unsubstituted alkynyl, a substituted or unsubstituted heterocycle, a substituted or unsubstituted heteroaryl, a substituted or unsubstituted cycloalkyl, or substituted or unsubstituted aryl, —C(O), C(R₈)(OH), C(R₈)(OMe), C(═NOH), C(═NOR_{4b}), C(═O)NH, C(═O)NR_{4b}, —NHC(═O), NR_{4b}C(═O), S, S(═O), S(═O)₂, —NHC(═O)NH, or NR_{4b}C(═O)NR_{4b};
where each substituent on Y or Z is (LₛRₛ)ⱼ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, C₁-C₆ alkyl, C₂-C₆ alkenyl, —C₁-C₆ fluoroalkyl, heteroaryl, aryl, or heterocycle; and each Rₛ is independently selected from H, halogen, —N(R₄)₂, —CN, —NO₂, N₃, —S(═O)₂NH₂, lower alkyl, lower cycloalkyl, —C₁-C₆ fluoroalkyl, heteroaryl, or heteroalkyl; where j is 0, 1, 2, 3, or 4;
each R₃ is independently selected from H, —S(═O)₂R₈, —S(═O)₂NH₂ —C(O)R₈, —CN, —NO₂, heteroaryl, or heteroalkyl;
each R_{3b} is independently selected from substituted or unsubstituted lower alkyl, substituted or unsubstituted lower cycloalkyl, substituted or unsubstituted phenyl or benzyl;
each R₄ is independently selected from H, substituted or unsubstituted lower alkyl, substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7-, or 8-membered heterocyclic ring;
each R_{4b} is independently selected from H, substituted or unsubstituted lower alkyl, substituted or unsubstituted lower cycloalkyl, substituted or unsubstituted aryl or substituted or unsubstituted benzyl; substituted or unsubstituted heteroaryl, substituted or unsubstituted heterocycle; R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(O), —CH(OH), -(substituted or unsubstituted C₁-C₆ alkyl), or -(substituted or unsubstituted C₂-C₆ alkenyl);
R₇ is L₃-X-L₄-G₁, wherein,
L₃ is a bond, or substituted or unsubstituted alkyl;
X is a bond, O, —C(═O), —CR₉(OR₉), S, —S(═O), —S(═O)₂, —NR₉, —NR₉C(O), —C(O)NR₉, —NR₉C(O)NR₉—, or aryl;
L₄ is a bond, or substituted or unsubstituted alkyl;
G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O);
or G₁ is W-G₅, where W is a substituted or unsubstituted aryl, substituted or unsubstituted heterocycle or substituted or unsubstituted heteroaryl and G₅ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈;
each R₈ is independently selected from substituted or unsubstituted lower alkyl, substituted or unsubstituted lower cycloalkyl, phenyl or benzyl;
each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and
each R₁₀ is independently selected from H, —S(═O)₂R₈, —S(═O)₂NH₂ —C(O)R₈, —CN, —NO₂, heteroaryl, or heteroalkyl;
R₅ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), —NHC(O)NH—, or —C(O)NH;
R₁₁ is L₇-L₁₀-G₆, wherein L₇ is a bond, —O, —S, —S(═O), —S(═O)₂, —NH, —C(O), —C(O)NH, —NHC(O), (substituted or unsubstituted C₁-C₆ alkyl), or (substituted or unsubstituted C₂-C₆ alkenyl);
L₁₀ is a bond, (substituted or unsubstituted alkyl), (substituted or unsubstituted cycloalkyl), (substituted or unsubstituted cycloalkenyl), (substituted or unsubstituted heteroaryl), (substituted or unsubstituted aryl), or (substituted or unsubstituted heterocycle);
G₆ is H, CN, SCN, N₃, NO₂, halogen, OR₉, —C(═O)CF₃, —C(═O)R₉, —C(═O)OR₉, —SR₈, —S(═O)R₈, —S(═O)₂R₈, N(R₉)₂, tetrazolyl, —NHS(═O)₂R₈, —S(═O)₂N(R₉)₂, —C(O)NHS(═O)₂R₈, —S(═O)₂NHC(O)R₉, —C(═O)N(R₉)₂, N R₉C(O)R₉, C(R₉)₂C(═O)N(R₉)₂—C(═NR₁₀)N(R₉)₂, —NR₉C(═NR₁₀)N(R₉)₂, —NR₉C(═CR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(O)O, —NHC(O)NH—, —OC(O)O—, —OC(O)NH—, —NHC(O), —C(O)NH, —C(O)O, or —OC(O);
or G₆ is W-G₇, wherein W is (substituted or unsubstituted heterocycle) or a (substituted or unsubstituted heteroaryl) and G₇ is H, halogen, CN, NO₂, N₃, CF₃, OCF₃, C₁-C₆ alkyl, C₃-C₆ cycloalkyl, —C₁-C₆ fluoroalkyl, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycle), or -L₅-(substituted or unsubstituted aryl), wherein L₅ is a bond, —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O);
provided that when L₁₀ is phenyl or thiophenyl, Y is -(substituted or unsubstituted heteroaryl), -(substituted or unsubstituted aryl), and Z is [C(R₂)₂]ₙC(R₁)₂₀, then G₆ is W-G₇; and R₁₂ is H, (substituted or unsubstituted C₁-C₆ alkyl), (substituted or unsubstituted C₃-C₆ cycloalkyl).

For any and all of the embodiments of Formula (G), Formula (G-I), and Formula (G-II)), substituents can be selected from among from a subset of the listed alternatives. For example, in some embodiments, Z is [C(R₂)₂]ₙC(R₁)₂₀. In further or alternative embodiments, Y is -(substituted or unsubstituted heteroaryl), -(substituted or unsubstituted aryl) and G₆ is W-G₇. In further or alternative embodiments, Y is -L₁-(substituted or unsubstituted alkyl), -L₁-(substituted or unsubstituted cycloalkyl), -L₁-(substituted or unsubstituted heterocycle), -L₁-(substituted or unsubstituted heteroaryl), -L₁-(substituted or unsubstituted non-aromatic heterocycle) provided that when the heteroatom is directly bound to Z, the non-aromatic heterocycle is substituted; -L₁-(substituted or unsubstituted aryl). In further or alternative embodiments, Y is a heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

In further or alternative embodiments, R₆ is L₂-(substituted or unsubstituted alkyl), or L₂-(substituted or unsubstituted cycloalkyl), L₂-(substituted or unsubstituted aryl), where L₂ is a bond, O, S, —S(O)₂, —C(O), —CH(OH), or substituted or unsubstituted alkyl. In further or alternative embodiments, R₇ is L₃-X-L₄-G₁; wherein, L₃ is a bond; and X is a bond, O, —CR₉(OR₉), S, —S(═O), —S(═O)₂, —NR₉, —NR₉C(O), —C(O)NR₉. In further or alternative embodiments, G₁ is tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —C₀₂R₉, —C(O)R₉, —CON(R₉)₂, —SR₈, —S(═O)R₈, —S(═O)₂R₈.

In further or alternative embodiments, the non-aromatic heterocycle of group Y can be selected from a quinolizine, a dioxine, a piperidine, a morpholine, a thiazine, a tetrahydropyridine, a piperazine, a oxazinanone, a dihydropyrrole, a dihydroimidazole, a tetrahydrofuran, a dihydrooxazole, an oxirane, a pyrrolidine, a pyrazolidine, a dihydrothiophenone, an imidazolidinone, a pyrrolidinone, a dihydrofuranone, a dioxolanone, a thiazolidine, a piperidinone, a tetrahydronaphthalene, a tetrahydroquinoline, a tetrahydrothiophene, and a thiazepane. In further or alternative embodiments, the non-aromatic heterocycle of group Y can be selected from the group consisting of:

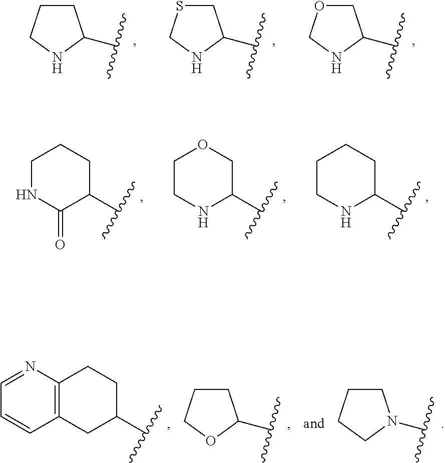

In further or alternative embodiments, “G” (e.g. G₁, G₅, G₆, G₇) is L₂₀-Q, wherein L₂₀ is an enzymatically cleavable linker and Q is a drug, or an affinity moiety. In further or alternative embodiments, the drug includes, by way of example only, leukotriene receptor antagonists and anti-inflammatory agents. In further or alternative embodiments, the leukotriene receptor antagonists include, but are not limited to, CysLT₁/CysLT₂ dual antagonists and CysLT₁ antagonists. In further or alternative embodiments, the affinity moiety allows for site specific binding and include, but are not limited to, antibodies, antibody fragments, DNA, RNA, siRNA, and ligands.

In a further or alternative embodiment, the “G” group (e.g. G₁, G₅, G₆, G₇) of any of Formula (G), Formula (G-I), or Formula (G-II), is any group that is used to tailor the physical and biological properties of the molecule. Such tailoring/modifications are achieved using groups which modulate acidity, basicity, lipophilicity, solubility and other physical properties of the molecule. The physical and biological properties modulated by such modifications to “G” include, by way of example only, solubility, in vivo absorption, and in vivo metabolism. In addition, in vivo metabolism may include, by way of example only, controlling in vivo PK properties, off-target activities, potential toxicities associated with cypP450 interactions, drug-drug interactions, and the like. Further, modifications to “G” allow for the tailoring of the in vivo efficacy of the compound through the modulation of, by way of example, specific and non-specific protein binding to plasma proteins and lipids and tissue distribution in vivo. Additionally, such tailoring/modifications to “G” allow for the design of compounds selective for 5-lipoxygenase-activating protein over other proteins. In further or alternative embodiments, “G” is L₂₀-Q, wherein L₂₀ is an enzymatically cleavable linker and Q is a drug, or an affinity moiety. In further or alternative embodiments, the drug includes, by way of example only, leukotriene receptor antagonists and anti-inflammatory agents. In further or alternative embodiments, the leukotriene receptor antagonists include, but are not limited to, CysLT₁/CysLT₂ dual antagonists and CysLT₁ antagonists. In further or alternative embodiments, the affinity moiety allows for site specific binding and include, but are not limited to, antibodies, antibody fragments, DNA, RNA, siRNA, and ligands.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

Formula (G) is as follows:

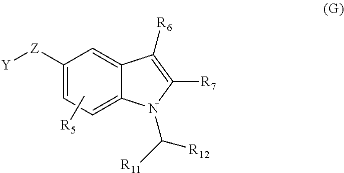

wherein,
Z is selected from [C(R₁)₂]ₘ[C(R₂)₂]ₙ, [C(R₂)₂]ₙ[C(R₁)₂]ₘO, O[C(R₁)₂]ₘ [C(R₂)₂]ₙ, [C(R₂)₂]ₙO[C(R₁)₂]ₙ, or [C(R₁)₂]ₙO[C(R₂)₂]ₙ, wherein each R₁ is independently H, CF₃, or an optionally substituted lower alkyl and two R₁ on the same carbon may join to form a carbonyl (═O); and each R₂ is independently H, OH, OMe, CF₃, or an optionally substituted lower alkyl and two R₂ on the same carbon may join to form a carbonyl (═O); m is 0, 1 or 2; each n is independently 0, 1, 2, or 3;
Y is H or -(substituted or unsubstituted aryl); or -(substituted or unsubstituted heteroaryl); where each substituent on Y or Z is (LₛRₛ)ⱼ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, substituted or unsubstituted C₁-C₆ alkyl, C₂-C₆ alkenyl, —C₁-C₆ fluoroalkyl, substituted or unsubstituted heteroaryl, substituted or unsubstituted aryl, or substituted or unsubstituted heterocycle; and each R₈ is independently selected from H, halogen, —N(R₄)₂, —CN, —NO₂, N₃, —S(═O)₂NH₂, substituted or unsubstituted lower alkyl, substituted or unsubstituted lower cycloalkyl, —C₁-C₆ fluoroalkyl, substituted or unsubstituted aryl, substituted or unsubstituted heteroaryl, or substituted or unsubstituted heteroalkyl; where j is 0, 1, 2, 3, or 4;
R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(O), —CH(OH), -(substituted or unsubstituted C₁-C₆ alkyl), or -(substituted or unsubstituted C₂-C₆ alkenyl);
R₇ is L₃-X-L₄-G₁, wherein,
   L₃ is a substituted or unsubstituted alkyl;
   X is a bond, O, —C(═O), —CR₉(OR₉), S, —S(═O), —S(═O)₂, —NR₉, —NR₉C(O), —C(O)NR₉, —NR₉C(O)NR₉—;
      L₄ is a bond, or a substituted or unsubstituted alkyl;
      G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O);
      or G₁ is W-G₅, where W is a substituted or unsubstituted aryl, substituted or unsubstituted heterocycle or substituted or unsubstituted heteroaryl and G₅ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈;
      each R₈ is independently selected from substituted or unsubstituted lower alkyl, substituted or unsubstituted lower cycloalkyl, substituted or unsubstituted phenyl or substituted or unsubstituted benzyl;
      each R₉ is independently selected from H, substituted or unsubstituted lower alkyl, substituted or unsubstituted lower cycloalkyl, substituted or unsubstituted phenyl or substituted or unsubstituted benzyl; or two R₉ groups can together form a 5-, 6-, 7-, or 8-membered heterocyclic ring; or R₈ and R₉ can together form a 5-, 6-, 7-, or 8-membered heterocyclic ring and
      each R₁₀ is independently selected from H, —S(═O)₂R₈, —S(═O)₂NH₂ —C(O)R₈, —CN, —NO₂, heteroaryl, or heteroalkyl;
R₅ is H, halogen, substituted or unsubstituted C₁-C₆ alkyl, substituted or unsubstituted O—C₁-C₆ alkyl;
R₁₁ is L₇-L₁₀-G₆, wherein L₇ is a bond, —C(O), —C(O)NH, —NHC(O), or (substituted or unsubstituted C₁-C₆ alkyl); L₁₀ is a bond, (substituted or unsubstituted alkyl), (substituted or unsubstituted cycloalkyl), (substituted or unsubstituted heteroaryl), (substituted or unsubstituted aryl), or (substituted or unsubstituted heterocycle);
   G₆ is OR₉, —C(═O)R₉, —C(═O)OR₉, —SR₈, —S(═O)R₈, —S(═O)₂R₈, N(R₉)₂, tetrazolyl, —NHS(═O)₂R₈, —S(═O)₂N(R₉)₂, —C(O)NHS(═O)₂R₈, —S(═O)₂NHC(O)R₉, —C(═O)N(R₉)₂, N R₉C(O)R₉, C(R₉)₂C(═O)N(R₉)₂—C(═NR₁₀)N(R₉)₂, —NR₉C(═NR₁₀)N(R₉)₂, —NR₉C(═CR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O)
   or G₆ is W-G₇, wherein W is (substituted or unsubstituted heterocycle), (substituted or unsubstituted aryl) or a (substituted or unsubstituted heteroaryl) and G₇ is H, halogen, CN, NO₂, N₃, CF₃, OCF₃, C₁-C₆ alkyl, C₃-C₆ cycloalkyl, —C₁-C₆ fluoroalkyl, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycle), or -L₅-(substituted or unsubstituted aryl), wherein L₅ is a bond, —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O);
   provided that R₁₁ comprises at least one (unsubstituted or substituted) aromatic moiety and at least one (unsubstituted or substituted) cyclic moiety, wherein the (unsubstituted or substituted) cyclic moiety is a (unsubstituted or substituted) heterocyclic group or a (unsubstituted or substituted) heteroaryl group and R₁₁ is not a thienyl-phenyl group; R₁₂ is H, (substituted or unsubstituted C₁-C₆ alkyl), (substituted or unsubstituted C₃-C₆ cycloalkyl);
or active metabolite, or solvate, or pharmaceutically acceptable salt, or a pharmaceutically acceptable prodrug thereof.

For any and all of the embodiments (such as, e.g. Formula (G), Formula (G-I), and Formula (G-II)), substituents can be selected from among from a subset of the listed alternatives. For example, in some embodiments, Z is [C(R₂)₂]ₙC(R₁)₂₀.

In further or alternative embodiments, Y is -(substituted or unsubstituted heteroaryl) or -(substituted or unsubstituted aryl) and G₆ is W-G₇.

In further or alternative embodiments, Y is -(substituted or unsubstituted heteroaryl).

In further or alternative embodiments, Y is selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, imidazo[1,2-a]pyridinyl and furopyridinyl, wherein Y is substituted or unsubstituted.

In further or alternative embodiments, Y is selected from the group consisting of pyridinyl or quinolinyl, wherein Y is substituted or unsubstituted.

In further or alternative embodiments, R₆ is L₂-(substituted or unsubstituted alkyl), or L₂-(substituted or unsubstituted cycloalkyl), L₂-(substituted or unsubstituted aryl), where L₂ is a bond, O, S, —S(O)₂, —C(O), or substituted or unsubstituted alkyl.

In further or alternative embodiments, X is a bond, O, —C(═O), —CR₉(OR₉), S, —S(═O), —S(═O)₂, —NR₉, —NR₉C(O), —C(O)NR₉.

In further or alternative embodiments, G₁ is tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —C₀₂R₉, —C(O)R₉, —CON(R₉)₂, —SR₈, —S(═O)R₈, or —S(═O)₂R₈.

In further or alternative embodiments, L₃ is unsubstituted alkyl; X is a bond; L₄ is a bond; and G₁ is —C(O)OR₉.

In further or alternative embodiments, R₉ is H or unsubstituted alkyl.

In further or alternative embodiments, L₁₀ is a substituted or unsubstituted aryl substituted or unsubstituted heteroaryl and G₆ is W-G₇ wherein W is substituted or unsubstituted heteroaryl, substituted or unsubstituted heterocycle.

In further or alternative embodiments, L₁₀ is a substituted or unsubstituted aryl.

In further or alternative embodiments, L₃ is unsubstituted alkyl; X is a bond; L₄ is a bond; and G₁ is —OR₉.

In further or alternative embodiments, G₁ is W-G₅, where W is a substituted or unsubstituted heterocycle or substituted or unsubstituted heteroaryl.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

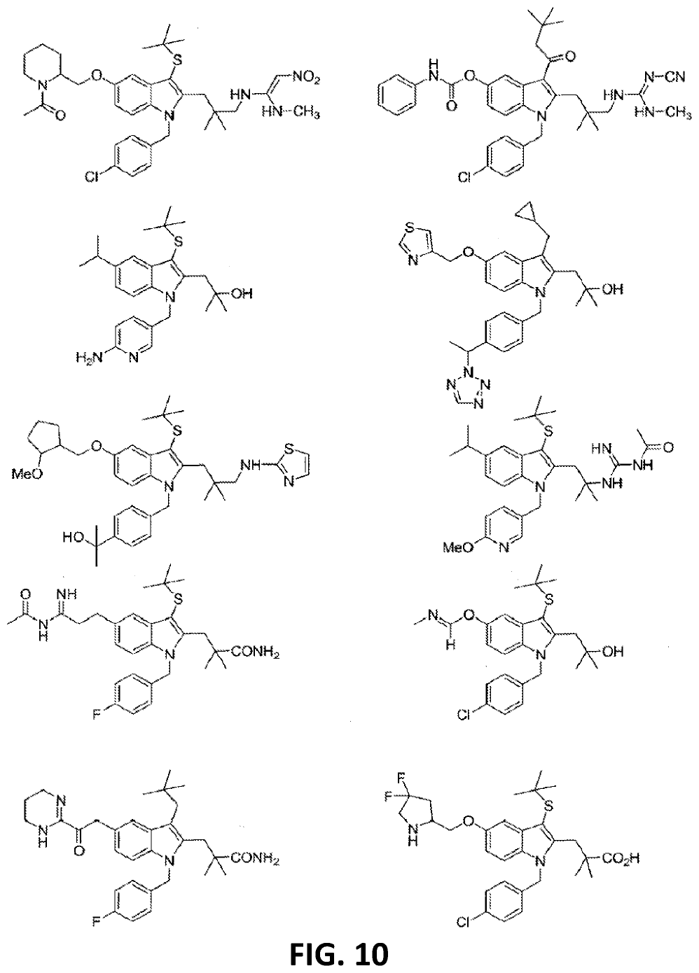

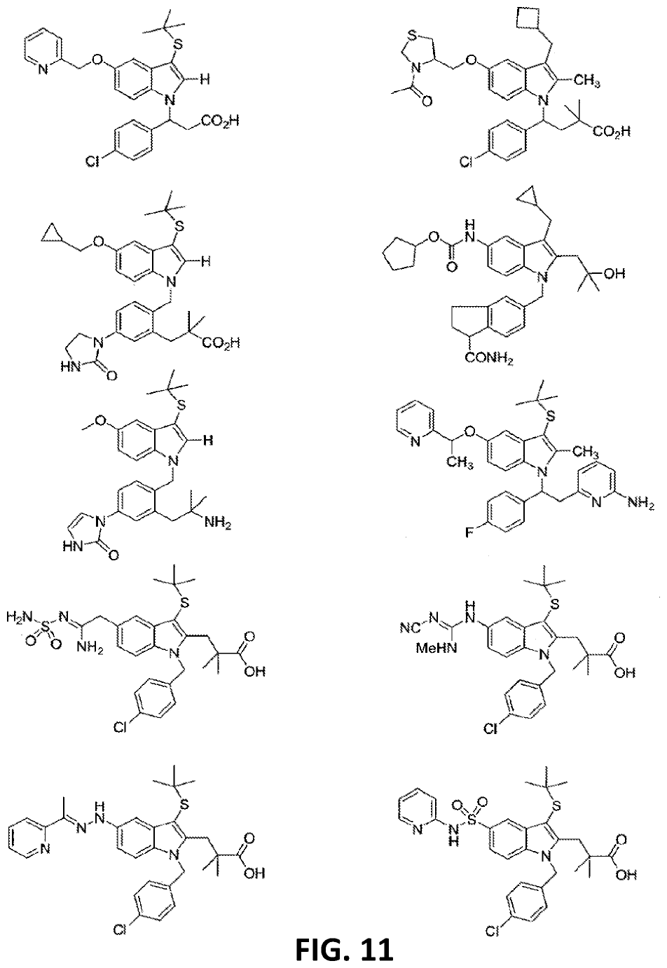

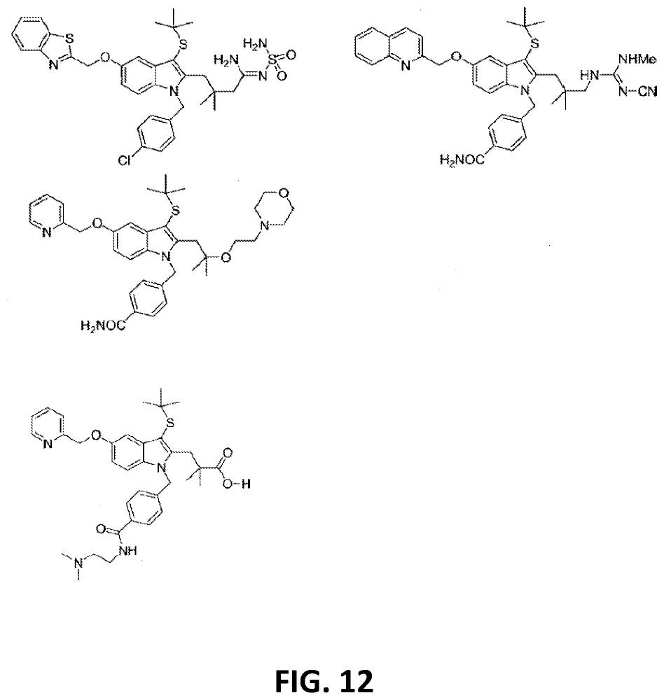

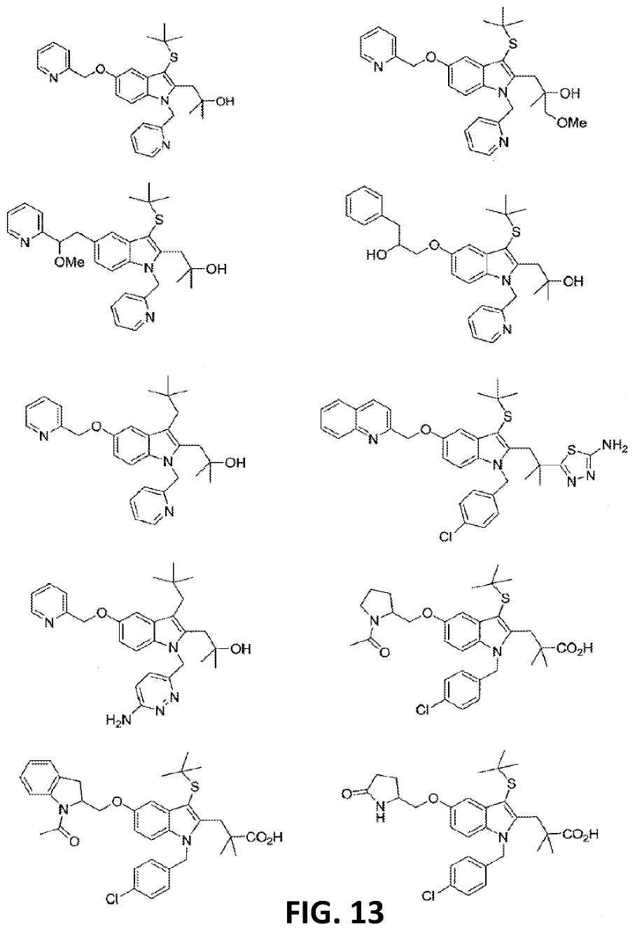

Further embodiments of Formula (G), Formula (G-I) and Formula (G-II), include, but are not limited to, compounds shown in FIGS. 10-13 and in Tables 1-4.

TABLE 1
N-(aryl-heteroaryl) indoles acid replacements

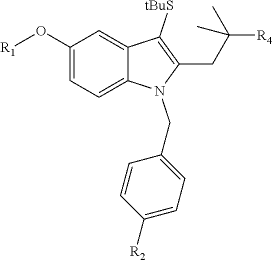

| Compound | | | | |
|---|---|---|---|---|
| # | R₁ | R₂ | R₄ | M + H |
| 1-1 | Pyridine-2- | pyridine-2-yl | C(O)NH₂ | 579 |
| | ylmethyl | | | |
| 1-2 | Pyridine-2- | 2-methoxy- | CO₂Et | 638 |
| | ylmethyl | pyridin-5-yl | | |
| 1-3 | Pyridine-2- | 2-methoxy- | C(O)(CH₂)₆OH | see |
| | ylmethyl | pyridin-5-yl | | Exp. |
| 1-4 | Pyridine-2- | pyridin-2-yl | OH | 552 |
| | ylmethyl | | | |
| 1-5 | Pyridine-2- | pyridin-3-yl | OH | 552 |
| | ylmethyl | | | |
| 1-6 | Pyridine-2- | thiazol-2-yl | OH | 558 |
| | ylmethyl | | | |
| 1-7 | Pyridine-2- | 1,3,4- | OH | 543 |
| | ylmethyl | oxadiazol- | | |
| | | 2-yl | | |
| 1-8 | Pyridine-2- | 2-methoxy- | 1,3,4-oxadiazol-2-yl | 634 |
| | ylmethyl | pyridin-5-yl | | |
| 1-9 | Pyridine-2- | 2-methoxy- | 1,3,4-oxadiazol-2-y | 649 |
| | ylmethyl | pyridin-5-yl | ylamine | |
| 1-10 | Pyridine-2- | 2-methoxy- | N-pyrazin-2-yl | 687 |
| | ylmethyl | pyridin-5-yl | propionamide | |
| 1-11 | Pyridine-2- | 2-methoxy- | N-thiazol-2-yl | 692 |
| | ylmethyl | pyridin-5-yl | propionamide | |
| 1-12 | Pyridine-2- | 2-methoxy- | N-pyridin-3-yl- | 686 |
| | ylmethyl | pyridin-5-yl | propionamide | |
| 1-13 | Pyridine-2- | 2-methoxy- | C(O)NH(CH₂CH₂NMe₂) | 680 |
| | ylmethyl | pyridin-5-yl | | |
| 1-14 | Pyridine-2- | 1,3,4- | CH₃ | 556 |
| | ylmethyl | oxadiazol-2- | | |
| | | ylamine | | |
| 1-15 | Quinolin-2- | 5-fluoro- | C(O)NHC(═NH)NH₂ | 689 |
| | ylmethyl | pyridin-2-yl | | |

TABLE 2
N-(aryl-heteroaryl) indoles and N-(aryl-heterocycle) indoles

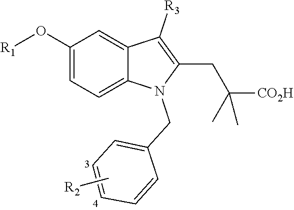

| Compound | | | | | |
|---|---|---|---|---|---|
| # | R₁ | position | R₂ | R₃ | M + H |
| 2-1 | Pyridin-2- | 4 | 2-thiazolyl | 2-methyl-2- | 586 |
| | ylmethyl | | | proplthio | |
| 2-2 | Pyridin-2- | 4 | 2-pyrimidinyl | 2-methyl-2- | 581 |
| | ylmethyl | | | propylthio | |
| 2-3 | Pyridin-2- | 4 | 3-pyridyl | 2-methyl-2- | 580 |
| | ylmethyl | | | propylthio | |
| 2-4 | Pyridin-2- | 4 | 4-pyrimidinyl | 2-methyl-2- | 581 |
| | ylmethyl | | | propylthio | |
| 2-5 | Pyridin-2- | 4 | 2-pyrazinyl | 2-methyl-2- | 581 |
| | ylmethyl | | | propylthio | |
| 2-6 | Pyridin-2- | 4 | 6-methoxy-3- | 2-methyl-2- | 611 |
| | ylmethyl | | pyridazinyl | propylthio | |
| 2-7 | Pyridin-2- | 4 | 2-amino-4- | 2-methyl-2- | 596 |
| | ylmethyl | | pyridazinyl | propylthio | |
| 2-8 | Pyridin-2- | 4 | 2-thiazolyl | 3,3- | 596 |
| | ylmethyl | | | dimethylbutanoyl | |
| 2-9 | Pyridin-2- | 4 | 2-thiazolyl | H | 498 |
| | ylmethyl | | | | |
| 2-10 | Pyridin-2- | 4 | 2-thiazolyl | acetyl | 501 |
| | ylmethyl | | | | |
| 2-11 | Pyridin-2- | 4 | 3-methoxy-6- | H | 523 |
| | ylmethyl | | pyridazinyl | | |
| 2-12 | Pyridin-2- | 4 | 3-methoxy-6- | acetyl | 565 |
| | ylmethyl | | pyridazinyl | | |
| 2-13 | Pyridin-2- | 4 | 3-methoxy-6- | ethyl | 551 |
| | ylmethyl | | pyridazinyl | | |
| 2-14 | Pyridin-2- | 4 | 2-thiazolyl | 3,3-dimethylbut- | 582 |
| | ylmethyl | | | 1-yl | |
| 2-15 | Pyridin-2- | 4 | 2-thiazolyl | cyclopropyl- | 566 |
| | ylmethyl | | | carbonyl | |
| 2-16 | Pyridin-2- | 4 | 2-thiazolyl | cyclobutyl- | 580 |
| | ylmethyl | | | carbonyl | |
| 2-17 | Pyridin-2- | 4 | 3-hydroxy-6- | 2-methyl-2- | 597 |
| | ylmethyl | | pyridazinyl | propylthio | |
| 2-18 | Pyridin-2- | 4 | 4-pyridyl | 2-methyl-2- | 580 |
| | ylmethyl | | | propylthio | |
| 2-19 | Pyridin-2- | 4 | 2-methoxypyridin-5- | 2-methyl-2- | 610 |
| | ylmethyl | | yl | propylthio | |
| 2-20 | Pyridin-2- | 4 | 2-methyl-4- | 2-methyl-2- | 595 |
| | ylmethyl | | pyridazinyl | propylthio | |
| 2-21 | Pyridin-2- | 4 | 5-methyl-thiazol-2-yl | 2-methyl-2- | 600 |
| | ylmethyl | | | propylthio | |
| 2-22 | Pyridin-2- | 4 | 2-thiazolyl | cyclobutylmethyl | 566 |
| | ylmethyl | | | | |
| 2-23 | 2-Methylthiazol- | 4 | 2-methoxy-4- | 2-methyl-2- | 631 |
| | 4-yl | | pyridazinyl | propylthio | |
| 2-24 | 2-Methylthiazol- | 4 | 2-thiazolyl | 2-methyl-2- | 606 |
| | 4-yl | | | propylthio | |
| 2-25 | 2-Methylthiazol- | 4 | 2-thiazolyl | H | 518 |
| | 4-yl | | | | |
| 2-26 | 2-Methylthiazol- | 4 | 2-thiazolyl | 3,3- | 616 |
| | 4-yl | | | dimethylbutanoyl | |
| 2-27 | 2-Methylthiazol- | 4 | 2-methoxy-4- | H | 543 |
| | 4-yl | | pyridazinyl | | |
| 2-28 | 2-Methylthiazol- | 4 | 2-methoxy-4- | 3,3- | 641 |
| | 4-yl | | pyridazinyl | dimethylbutanoyl | |
| 2-29 | Pyridin-2- | 4 | 2-thiazolyl | ethyl | 526 |
| | ylmethyl | | | | |
| 2-30 | Benzothiazol-2-yl | 4 | 2-thiazolyl | 2-methyl-2- | 667 |
| | | | | propylthio | |
| 2-31 | 2-Methylthiazol- | 4 | 2-pyrimidinyl | 2-methyl-2- | 601 |
| | 4-yl | | | propylthio | |
| 2-32 | Benzothiazol-2-yl | 4 | 2-pyrimidinyl | 2-methyl-2- | 637 |
| | | | | propylthio | |
| 2-33 | Pyridin-2- | 4 | 1-(pyridin-2- | 2-methyl-2- | 674 |
| | ylmethyl | | ylmethyl)-2-methyl- | propylthio | |
| | | | imidazol-4-yl | | |
| 2-34 | Pyridin-2- | 4 | 2,4-dimethylthiazol- | 2-methyl-2- | 614 |
| | ylmethyl | | 5-yl | propylthio | |
| 2-35 | Pyridin-2- | 4 | 5-fluoro-thiazol-2-yl | 2-methyl-2- | 604 |
| | ylmethyl | | | propylthio | |
| 2-36 | Pyridin-2- | 4- | 5-trifluoromethyl- | 2-methyl-2- | |
| | ylmethyl | | thiazol-2-yl | propylthio | |
| 2-37 | Pyridin-2- | 4 | 2-methyl-thiazol-4-yl | 2-methyl-2- | |
| | ylmethyl | | | propylthio | |
| 2-38 | Pyridin-2- | 4 | 2-methyl-thiazol-5-yl | 2-methyl-2- | |
| | ylmethyl | | | propylthio | |
| 2-39 | Pyridin-2-yl | 4 | 4-methyl-thiazol-2-yl | 2-methyl-2- | 600 |
| | ylmethyl | | | propylthio | |
| 2-40 | Pyridin-2- | 4 | isoxazol-4-yl | 2-methyl-2- | |
| | ylmethyl | | | propylthio | |
| 2-41 | Pyridin-2- | 4 | 3,5-dimethyl- | 2-methyl-2 | 600 |
| | ylmethyl | | isoxazol-4-yl | propylthio | |
| 2-42 | Pyridin-2- | 4 | 2-methyl-imidazol-4- | 2-methyl-2- | |
| | ylmethyl | | yl | propylthio | |
| 2-43 | Pyridin-2- | 4 | 1-methyl-imidazol-5- | 2-methyl-2- | 583 |
| | ylmethyl | | yl | propylthio | |
| 2-44 | Pyridin-2- | 4 | 1-methyl-imidazol-4- | 2-methyl-2- | |
| | ylmethyl | | yl | propylthio | |
| 2-45 | Pyridin-2- | 4 | Imidazol-4-yl | 2-methyl-2- | |
| | ylmethyl | | | propylthio | |
| 2-46 | Pyridin-2- | 4 | 4-methyl-imidazol-5- | 2-methyl-2- | |
| | ylmethyl | | yl | propylthio | |
| 2-47 | Pyridin-2- | 4 | 5-methoxypyridin-2- | 2-methyl-2- | 610 |
| | ylmethyl | | yl | propylthio | |
| 2-48 | Pyridin-2- | 4 | pyridine-2-yl | 2-methyl-2- | |
| | ylmethyl | | | propylthio | |
| 2-49 | Pyridin-2- | 4 | pyrazol-4-yl | 2-methyl-2- | |
| | ylmethyl | | | propylthio | |
| 2-50 | Pyridin-2- | 4 | 1-methyl-pyrazol-4- | 2-methyl-2- | |
| | ylmethyl | | yl | propylthio | |
| 2-51 | Pyridin-2- | 4 | 3-methyl-pyrazol-4- | 2-methyl-2- | |
| | ylmethyl | | yl | propylthio | |
| 2-52 | Pyridin-2- | 4 | 5-methyl-1,2,4- | 2-methyl-2- | |
| | ylmethyl | | oxadiazol-3-yl | propylthio | |
| 2-53 | Pyridin-2- | 4 | 2-methyl-1,3,4- | 2-methyl-2- | |
| | ylmethyl | | oxadiazol-5-yl | propylthio | |
| 2-54 | Pyridin-2- | 4 | 1,3,4-oxadiazol-2-yl | 2-methyl-2- | |
| | ylmethyl | | | propylthio | |
| 2-55 | Pyridin-2- | 4 | 1,3,4-thiadiazol-2-yl | 2-methyl-2- | 587 |
| | ylmethyl | | | propylthio | |
| 2-56 | Pyridin-2- | 4 | 3-methyl-pyrazol-5- | 2-methyl-2- | |
| | ylmethyl | | yl | propylthio | |
| 2-57 | Pyridin-2- | 4 | 1,2,3-thiadiazol-4-yl | 2-methyl-2- | |
| | ylmethyl | | | propylthio | |
| 2-58 | Pyridin-2- | 4 | 1-tetrazolyl | 2-methyl-2- | |
| | ylmethyl | | | propylthio | |
| 2-59 | Pyridin-2- | 4 | 2-tetrazolyl | 2-methyl-2- | |
| | ylmethyl | | | propylthio | |
| 2-60 | Pyridin-2- | 4 | 1-methyl-tetrazol-5- | 2-methyl-2- | |
| | ylmethyl | | yl | propylthio | |
| 2-61 | Pyridin-2- | 4 | 2-methyl-tetrazol-5- | 2-methyl-2- | |
| | ylmethyl | | yl | propylthio | |
| 2-62 | Pyridin-2- | 4 | 2-pyridone-5-yl | 2-methyl-2- | 596 |
| | ylmethyl | | | propylthio | |
| 2-63 | Pyridin-2- | 4 | pyridin-3-yl | 2-methyl-2- | |
| | ylmethyl | | | propylthio | |
| 2-64 | Pyridin-2- | 4 | 2-cyanopyridin-5-yl | 2-methyl-2- | 606 |
| | ylmethyl | | | propylthio | |
| 2-65 | Pyridin-2- | 4 | 2- | 2-methyl-2- | 648 |
| | ylmethyl | | trifluoromethyl- | propylthio | |
| | | | pyridin-5-yl | | |
| 2-66 | Pyridin-2- | 4 | 2- | 2-methyl-2- | |
| | ylmethyl | | acetylaminopyridin- | propylthio | |
| | | | 5-yl | | |
| 2-67 | Pyridin-2- | 4 | 2-methoxypyrimidin- | 2-methyl-2- | 611 |
| | ylmethyl | | 5-yl | propylthio | |
| 2-68 | Pyridin-2- | 4 | 2-methoxythiazol-4- | 2-methyl-2- | 616 |
| | ylmethyl | | yl | propylthio | |
| 2-69 | 3-fluoro-pyridin | 4 | 2-methoxypyridin-5- | 2-methyl-2- | |
| | 2-ylmethyl | | yl | propylthio | |
| 2-70 | 3-fluoro-pyridin- | 4 | 2-methoxypyridin-5- | 2-methyl-2- | |
| | 2-ylmethyl | | yl | propylthio | |
| 2-71 | 4-fluoro-pyridin- | 4 | 2-methoxypyridin-5- | 2-methyl-2- | |
| | 2-ylmethyl | | yl | propylthio | |
| 2-72 | 5-fluoro-pyridin | 4 | 2-methoxypyridin-5- | 2-methyl-2- | |
| | 2-ylmethyl | | yl | propylthio | |
| 2-73 | 5-methyl-pyridin- | 4 | 2-methoxypyridin-5- | 2-methyl-2- | 625 |
| | 2-ylmethyl | | yl | propylthio | |
| 2-74 | 5-cyano-pyridin- | 4 | 2-methoxypyridin-5- | 2-methyl-2- | |
| | 2-ylmethyl | | yl | propylthio | |
| 2-75 | 5-methoxy- | 4 | 2-methoxypyridin-5- | 2-methyl-2- | |
| | pyridin-2- | | yl | propylthio | |
| | ylmethyl | | | | |
| 2-76 | 5-ethyl-pyridin-2- | 4 | 4-methoxypyridin-2- | 2-methyl-2- | 638 |
| | ylmethyl | | yl | propylthio | |
| 2-77 | Quinolin-2- | 4 | 4-methoxypyridin-2- | 2-methyl-2- | 660 |
| | ylmethyl | | yl | propylthio | |
| 2-78 | 6-fluoroquinolin- | 4 | 4-methoxypyridin-2- | 2-methyl-2- | 678 |
| | 2-ylmethyl | | yl | propylthio | |
| 2-79 | Quinolin-2- | 3 | 5-fluoropyridin-2-yl | 2-methyl-2- | |
| | ylmethyl | | | propylthio | |
| 2-80 | Quinolin-2- | 3 | 2-methoxypyridin-5- | 2-methyl-2- | |
| | ylmethyl | | yl | propylthio | |
| 2-81 | Quinolin-2- | 3 | 5- | 2-methyl-2- | |
| | ylmethyl | | trifluoromethyl- | propylthio | |
| | | | pyridin-2-yl | | |
| 2-82 | 5-methyl-pyridin- | 4 | 3-fluoropyridin-2-yl | 2-methyl-2- | 612 |
| | 2-ylmethyl | | | propylthio | |
| 2-83 | Quinolin-2- | 3 | 2- | 2-methyl-2- | |
| | ylmethyl | | trifluoromethyl- | propylthio | |
| | | | pyridin-5-yl | | |
| 2-84 | 5-ethyl-pyridin-2- | 4 | 3-fluoropyridin-2-yl | 2-methyl-2- | 627 |
| | ylmethyl | | | propylthio | |
| 2-85 | Quinolin-2- | 4 | 3-fluoropyridin-2-yl | 2-methyl-2- | 648 |
| | ylmethyl | | | propylthio | |
| 2-86 | Quinolin-2- | 3 | 2-ethoxypyridin-5-yl | 2-methyl-2- | |
| | ylmethyl | | | propylthio | |
| 2-87 | Pyridin-2- | 4 | 5-carboxamido- | 2-methyl-2- | 623 |
| | ylmethyl | | pyridin-2-yl | propylthio | |
| 2-88 | Pyridin-2- | 4 | 5-cyanopyridin-2-yl | 2-methyl-2- | 605 |
| | ylmethyl | | | propylthio | |
| 2-89 | Pyridin-2- | 4 | 5-methoxythiazol-2- | 2-methyl-2- | 616 |
| | ylmethyl | | yl | propylthio | |
| 2-90 | Pyridin-2- | 4 | 2-methyl-pyridin-5-yl | 2-methyl-2- | 594 |
| | ylmethyl | | | propylthio | |
| 2-91 | Pyridin-2- | 4 | 5-trifluoromethyl- | 2-methyl-2- | 670 |
| | ylmethyl | | pyridin-2-yl | propylthio | |
| 2-92 | Pyridin-2- | 4 | 2-ethoxythiazol-4-yl | 2-methyl-2- | 631 |
| | ylmethyl | | | propylthio | |
| 2-93 | Pyridin-2- | 4 | 4-methyl-imidazol-2- | 2-methyl-2- | 583 |
| | ylmethyl | | yl | propylthio | |
| 2-94 | Pyridin-2- | 4 | 2-ethoxypyridin-5-yl | 2-methyl-2- | 624 |
| | ylmethyl | | | propylthio | |
| 2-95 | Pyridin-2- | 4 | 6-methoxypyridin-2- | 2-methyl-2- | 610 |
| | ylmethyl | | yl | propylthio | |
| 2-96 | Pyridin-2- | 4 | 5-methoxypyridin-3- | 2-methyl-2- | 610 |
| | ylmethyl | | yl | propylthio | |
| 2-97 | Pyridin-2- | 4 | 2-carboxamido- | 2-methyl-2- | 624 |
| | ylmethyl | | pyridin-5-yl | propylthio | |
| 2-98 | Pyridin-2- | 4 | 5-methyl-pyridin-2-yl | 2-methyl-2- | 594 |
| | ylmethyl | | | propylthio | |
| 2-99 | 6-Fluoro-pyridin- | 4 | 2-methoxypyridin-5- | 2-methyl-2- | 628 |
| | 2-ylmethyl | | yl | propylthio | |
| 2-100 | 6-Methoxy- | 4 | 2-methoxypyridin-5- | 2-methyl-2- | 640 |
| | pyridin-2- | | yl | propylthio | |
| | ylmethyl | | | | |
| 2-101 | 6-Methyl-pyridin- | 4 | 2-methoxypyridin-5- | 2-methyl-2- | 624 |
| | 2-ylmethyl | | yl | propylthio | |
| 2-102 | 5-Methyl-pyridin- | 4 | 2-trifluoromethyl- | 2-methyl-2- | 662 |
| | 2-ylmethyl | | pyridin-5-yl | propylthio | |
| 2-103 | 5-Methyl-pyridin- | 4 | 5-trifluoromethyl- | 2-methyl-2- | 662 |
| | 2-ylmethyl | | pyridin-2-yl | propylthio | |
| 2-104 | 6-cyclopropyl- | 4 | 2-methoxypyridin-5- | 2-methyl-2- | 650 |
| | pyridin-2- | | yl | propylthio | |
| | ylmethyl | | | | |
| 2-105 | 5-Methyl-pyridin- | 4 | 5-methyl-pyridin-2-yl | 2-methyl-2- | 608 |
| | 2-ylmethyl | | | propylthio | |
| 2-106 | 5-Methyl-pyridin- | 4 | 6-methoxy-pyridazin- | 2-methyl-2- | 625 |
| | 2-ylmethyl | | 3-yl | propylthio | |
| 2-107 | 5-Methyl-pyridin- | 4 | 2-ethoxypyridin-5-yl | 2-methyl-2- | 639 |
| | 2-ylmethyl | | | propylthio | |
| 2-108 | 5-Chloro-pyridin- | 4 | 2-methoxypyridin-5- | 2-methyl-2- | 644 |
| | 2-ylmethyl | | yl | propylthio | |
| 2-109 | S-1-(pyridin-2- | 4 | 5-trifluoromethyl- | 2-methyl-2- | 684 |
| | yl)-1-ethyl | | pyridin-2-yl | propylthio | (M + Na) |
| 2-110 | R-1-(pyridin-2- | 4 | 5-trifluoromethyl- | 2-methyl-2- | 663 |
| | yl)-1-ethyl | | pyridin-2-yl | propylthio | |
| 2-111 | S-1-(pyridin-2- | 4 | 2-methoxypyridin-5- | 2-methyl-2- | 624 |
| | yl)-1-ethyl | | yl | propylthio | |
| 2-112 | R-1-(pyridin-2- | 4 | 2-methoxypyridin-5- | 2-methyl-2- | 624 |
| | yl)-1-ethyl | | yl | propylthio | |
| 2-113 | S-1-(pyridin-2- | 4 | 6-methoxypyridin-2- | 2-methyl-2- | 625 |
| | yl)-1-ethyl | | yl | propylthio | |
| 2-114 | R-1-(pyridin-2- | 4 | 6-methoxypyridin-2- | 2-methyl-2- | 624 |
| | yl)-1-ethyl | | yl | propylthio | |
| 2-115 | S-1-(pyridin-2- | 4 | 2-ethoxythiazol-4-yl | 2-methyl-2- | 644 |
| | yl)-1-ethyl | | | propylthio | |
| 2-116 | R-1-(pyridin-2- | 4 | 2-ethoxythiazol-4-yl | 2-methyl-2- | 644 |
| | yl)-1-ethyl | | | propylthio | |
| 2-117 | 3-Methyl-pyridin- | 4 | 2-methoxypyridin-5- | 2-methyl- | 624 |
| | 2-ylmethyl | | yl | propylthio | |
| 2-118 | 3-Methyl-pyridin- | 4 | 5-trifluoromethyl- | 2-methyl-2- | 662 |
| | 2-ylmethyl | | pyridin-2-yl | propylthio | |
| 2-119 | 3,5- | 4 | 2-methoxypyridin-5- | 2-methyl-2- | 638 |
| | dimethylpyridin- | | yl | propylthio | |
| | 2-ylmethyl | | | | |
| 2-120 | 3,5- | 4 | 5-trifluoromethyl- | 2-methyl-2- | 676 |
| | dimethylpyridin- | | pyridin-2-yl | propylthio | |
| | 2-ylmethyl | | | | |
| 2-121 | Benzothiazol- | 4 | 2-methoxypyridin-5- | 2-methyl-2- | 666 |
| | 2ylmethyl | | yl | propylthio | |
| 2-122 | Benzothiazol- | 4 | 5-trifluoromethyl- | 2-methyl-2- | 666 |
| | 2ylmethyl | | pyridin-2-yl | propylthio | |
| 2-123 | Benzothiazol- | 4 | 2-methoxypyridin-5- | cyclobutylcarbonyl | 660 |
| | 2ylmethyl | | yl | yl | |
| 2-124 | Benzothiazol- | 4 | 5-trifluoromethyl- | cyclobutylmethyl | 646 |
| | 2ylmethyl | | pyridin-2-yl | | |
| 2-125 | 5-Ethylpyridin-2- | 4 | 2-methoxypyridin-5- | 2-methyl-2- | 638 |
| | ylmethyl | | yl | propylthio | |
| 2-126 | 5-Ethylpyridin-2- | 4 | 2-ethoxypyridin-5-yl | 2-methyl-2- | 652 |
| | ylmethyl | | | propylthio | |
| 2-127 | 5-Ethylpyridin-2- | 4 | 2-trifluoromethyl- | 2-methyl-2- | 676 |
| | ylmethyl | | pyridin-5-yl | propylthio | |
| 2-128 | 5-Ethylpyridin-2- | 4 | 5-trifluoromethyl- | 2-methyl-2- | 677 |
| | ylmethyl | | pyridin-2-yl | propylthio | |
| 2-129 | 5-Methylpyridin- | 4 | 2-ethoxythiazol-4-yl | 2-methyl-2- | 644 |
| | 2-ylmethyl | | | propylthio | |
| 2-130 | 5-Methylpyridin- | 4 | 2-methoxythiazol-4- | 2-methyl-2- | 630 |
| | 2-ylmethyl | | yl | propylthio | |
| 2-131 | 5-Methylpyridin- | 4 | 6-methoxypyridin-2- | 2-methyl-2- | 624 |
| | 2-ylmethyl | | yl | propylthio | |
| 2-132 | Pyridin-2- | 4 | 2-methoxypyridin-5- | cyclobutylmethyl | 590 |
| | ylmethyl | | yl | | |
| 2-133 | 5-Methylpyridin- | 4 | 2-methoxypyridin-5- | cyclobutylmethyl | 604 |
| | 2-ylmethyl | | yl | | |
| 2-134 | 5-Methylpyridin- | 4 | 2-methoxypyridin-5- | 2-methyl-prop-1- | 592 |
| | 2-ylmethyl | | yl | yl | |
| 2-135 | Quinolin-2- | 4 | 2-methoxypyridin-5- | 2-methyl-2- | 660 |
| | ylmethyl | | yl | propylthio | |
| 2-136 | Quinolin-2- | 4 | 2-trifluoromethyl- | 2-methyl-2- | 936 |
| | ylmethyl | | pyridin-5-yl | propylthio | |
| 2-137 | Quinolin-2- | 4 | 5-trifluoromethyl- | 2-methyl-2- | 698 |
| | ylmethyl | | pyridin-2-yl | propylthio | |
| 2-138 | Quinolin-2- | 4 | 6-methoxy-pyridazin- | 2-methyl-2- | 661 |
| | ylmethyl | | 3-yl | propylthio | |
| 2-139 | Quinolin-2- | 4 | 2-ethoxypyridin-5-yl | 2-methyl-2- | 674 |
| | ylmethyl | | | propylthio | |
| 2-140 | 6-fluoroquinolin- | 4 | 6-methoxypyridin-2- | 2-methyl-2- | 678 |
| | 2-ylmethyl | | yl | propylthio | |
| 2-141 | 6-fluoroquinolin- | 4 | 2-methoxypyridin-5- | 2-methyl-2- | 678 |
| | 2-ylmethyl | | yl | propylthio | |
| 2-142 | 6-fluoroquinolin- | 4 | 2-ethoxythiazol-4-yl | 2-methyl-2- | 698 |
| | 2-ylmethyl | | | propylthio | |
| 2-143 | 6-fluoroquinolin- | 4 | 5-trifluoromethyl- | 2-methyl-2- | 716 |
| | 2-ylmethyl | | pyridin-2-yl | propylthio | |
| 2-144 | 7-fluoroquinolin- | 4 | 2-trifluoromethyl- | 2-methyl-2- | 716 |
| | 2-ylmethyl | | pyridin-5-yl | propylthio | |
| 2-145 | 7-fluoroquinolin- | 4 | 5-trifluoromethyl- | 2-methyl-2- | 716 |
| | 2-ylmethyl | | pyridin-2-yl | propylthio | |
| 2-146 | 7-fluoroquinolin- | 4 | 2-methoxypyridin-5- | 2-methyl-2- | 678 |
| | 2-ylmethyl | | yl | propylthio | |
| 2-147 | 7-fluoroquinolin- | 4 | 2-ethoxypyridin-5-yl | 2-methyl-2- | 692 |
| | 2-ylmethyl | | | propylthio | |
| 2-148 | 6-fluoroquinolin- | 4 | 3-fluoropyridin-2-yl | 2-methyl-2- | 666 |
| | 2-ylmethyl | | | propylthio | |
| 2-149 | 5-methyl-pyridin- | 4 | 3- | 2-methyl-2- | 662 |
| | 2-ylmethyl | | trifluoromethyl- | propylthio | |
| | | | pyridin-2-yl | | |
| 2-150 | 5-ethyl-pyridin-2- | 4 | 3- | 2-methyl-2- | 676 |
| | ylmethyl | | trifluoromethyl- | propylthio | |
| | | | pyridin-2-yl | | |
| 2-151 | Quinolin-2- | 4 | 3- | 2-methyl-2- | 698 |
| | ylmethyl | | trifluoromethyl- | propylthio | |
| | | | pyridin-2-yl | | |
| 2-152 | Quinolin-2- | 3 | 5-methoxythiazol-2- | 2-methyl-2- | |
| | ylmethyl | | yl | propylthio | |
| 2-153 | Quinolin-2- | 3 | 3-methoxy-6- | 2-methyl-2- | |
| | ylmethyl | | pyridazinyl | propylthio | |
| 2-154 | Quinolin-2- | 3 | 5-fluoro-thiazol-2-yl | 2-methyl-2- | |
| | ylmethyl | | | propylthio | |
| 2-155 | Quinolin-2- | 3 | pyridin-2-yl | 2-methyl-2- | |
| | ylmethyl | | | propylthio | |
| 2-156 | 6-fluoroquinolin- | 4 | 3- | 2-methyl-2- | 716 |
| | 2-ylmethyl | | trifluoromethyl- | propylthio | |
| | | | pyridin-2-yl | | |
| 2-157 | 3-methylpyridin- | 4 | 2-ethoxypyridin-5-yl | 2-methyl-2- | 638 |
| | 2-ylmethyl | | | propylthio | |
| 2-158 | 3-methylpyridin- | 4 | 2- | 2-methyl-2- | 662 |
| | 2-ylmethyl | | trifluoromethyl- | propylthio | |
| | | | pyridin-5-yl | | |
| 2-159 | 3,5- | 4 | 2-ethoxypyridin-5-yl | 2-methyl-2- | 652 |
| | dimethylpyridin- | | | propylthio | |
| | 2-ylmethyl | | | | |
| 2-160 | 4-methylpyridin- | 4 | 2-methoxypyridin-5- | 2-methyl-2- | 624 |
| | 2-ylmethyl | | yl | propylthio | |
| 2-161 | 4-methylpyridin- | 4 | 2-ethoxypyridin-5-yl | 2-methyl-2- | 638 |
| | 2-ylmethyl | | | propylthio | |
| 2-162 | 4-methylpyridin- | 4 | 2- | 2-methyl-2- | 662 |
| | 2-ylmethyl | | trifluoromethyl- | propylthio | |
| | | | pyridin-5-yl | | |
| 2-163 | 5-methylpyridin- | 4 | 5- | cyclobutylmethyl | 642 |
| | 2-ylmethyl | | trifluoromethyl- | | |
| | | | pyridin-2-yl | | |
| 2-164 | 6-fluoroquinolin- | 4 | 2-ethoxypyridin-5-yl | 2-methyl-2- | 692 |
| | 2-ylmethyl | | | propylthio | |
| 2-165 | 6-fluoroquinolin- | 4 | 2- | 2-methyl-2- | 716 |
| | 2-ylmethyl | | trifluoromethyl- | propylthio | |
| | | | pyridin-5-yl | | |
| 2-166 | 6-methylquinolin- | 4 | 2-methoxypyridin-5- | 2-methyl-2- | 674 |
| | 2-ylmethyl | | yl | propylthio | |
| 2-167 | 6-methylquinolin- | 4 | 5-trifluoromethyl- | 2-methyl-2- | 712 |
| | 2-ylmethyl | | pyridin-2-yl | propylthio | |
| 2-168 | quinolin-2- | 4 | 3-methylpyridazin-6- | 2-methyl-2- | 645 |
| | ylmethyl | | yl | propylthio | |
| 2-169 | quinolin-2- | 4 | 3-ethoxypyridazin-6- | 2-methyl-2- | 675 |
| | ylmethyl | | yl | propylthio | |
| 2-170 | quinolin-2- | 4 | 2-methoxypyridin-5- | 2-methyl-1- | 628 |
| | ylmethyl | | yl | propyl | |
| 2-171 | 6-fluoroquinolin- | 4 | 3-methoxypyridazin- | 2-methyl-2- | 679 |
| | 2-ylmethyl | | 6-yl | propylthio | |
| 2-172 | Pyridine-2- | 4 | 2-methoxypyridin-5- | 2-methyl-2- | 642 |
| | ylmethyl | | yl | propylthio-S,S- | |
| | | | | dioxide | |
| 2-173 | Pyridine-2- | 4 | 2-methoxypyridin-5- | 2-methyl-2- | 626 |
| | ylmethyl | | yl | propylthio-S- | |
| | | | | oxide | |
| 2-174 | N-oxido-pyridine- | 4 | 2-methoxypyridin-5- | 2-methyl-2- | 626 |
| | 2-ylmethyl | | yl | propylthio | |
| 2-175 | Imidazo[1,2- | 4 | 2-methoxypyridin-5- | 2-methyl-2- | 649 |
| | a]pyridine-2- | | yl | propylthio | |
| | ylmethyl | | | | |
| 2-176 | Imidazo[1,2- | 4 | 2-ethoxypyridin-5-yl | 2-methyl-2- | 663 |
| | a]pyridine-2- | | | propylthio | |
| | ylmethyl | | | | |
| 2-177 | Imidazo[1,2- | 4 | 5- | 2-methyl-2- | 687 |
| | a]pyridine-2- | | trifluoromethyl- | propylthio | |
| | ylmethyl | | pyridin-2-yl | | |
| 2-178 | R-1-(pyridin-2- | 4 | 2-ethoxypyridin-5-yl | 2-methyl-2- | 638 |
| | yl)-1-ethyl | | | propylthio | |
| 2-179 | 6-fluoroquinolin- | 4 | 3-methylpyridazin-6- | 2-methyl-2- | 663 |
| | 2-ylmethyl | | yl | propylthio | |
| 2-180 | 5-methylisoxazol- | 4 | 2-methoxypyridin-5- | 2-methyl-2- | 614 |
| | 3-ylmethyl | | yl | propylthio | |
| 2-181 | 5-methylisoxazol- | 4 | 2-ethoxypyridin-5-yl | 2-methyl-2- | 628 |
| | 3-ylmethyl | | | propylthio | |
| 2-182 | 5-methylisoxazol- | 4 | 5- | 2-methyl-2- | 652 |
| | 3-ylmethyl | | trifluoromethyl- | propylthio | |
| | | | pyridin-2-yl | | |
| 2-183 | 1,3- | 4 | 2-methoxypyridin-5- | 2-methyl-2- | 627 |
| | dimethylpyrazol- | | yl | propylthio | |
| | 5-ylmethyl | | | | |
| 2-184 | 1,5- | 4 | 2-methoxypyridin-5- | 2-methyl-2- | 627 |
| | dimethylpyrazol- | | yl | propylthio | |
| | 3-ylmethyl | | | | |
| 2-185 | 6-fluoroquinolin- | 4 | 3-ethoxypyridazin-6- | 2-methyl-2- | 693 |
| | 2-ylmethyl | | yl | propylthio | |
| 2-186 | 5-ethylpyridin-2- | 4 | 3-ethoxypyridazin-6- | 2-methyl-2- | 653 |
| | ylmethyl | | yl | propylthio | |
| 2-187 | 5-ethylpyridin-2- | 4 | 3-methoxypyridazin- | 2-methyl-2- | 639 |
| | ylmethyl | | 6-yl | propylthio | |
| 2-188 | 6-fluoroquinolin- | 4 | 5-fluoropyridin-2-yl | 2-methyl-2- | 666 |
| | 2-ylmethyl | | | propylthio | |
| 2-189 | R-1-(pyridin-2- | 4 | 5-fluoropyridin-2-yl | 2-methyl-2- | 612 |
| | yl)-1-ethyl | | | propylthio | |
| 2-190 | 6-fluoroquinolin- | 4 | 2-ethoxypyridin-6-yl | 2-methyl-2- | 692 |
| | 2-ylmethyl | | | propylthio | |
| 2-191 | R-1-(pyridin-2- | 4 | 2-ethoxypyridin-6-yl | 2-methyl-2- | 638 |
| | yl)-1-ethyl | | | propylthio | |
| 2-192 | 5-methylpyridin- | 4 | 5-fluoropyridin-2-yl | 2-methyl-2- | 612 |
| | 2-ylmethyl | | | propylthio | |
| 2-193 | 5-methylpyridin- | 4- | 2-ethoxypyridin-6-yl | 2-methyl-2- | 638 |
| | 2-ylmethyl | | | propylthio | |
| 2-194 | 6-fluoroquinolin- | 4 | 2- | 2-methylprop-1- | 684 |
| | 2-ylmethyl | | trifluoromethyl- | yl | |
| | | | pyridin-5-yl | | |
| 2-195 | Pyridin-2- | 3 | 5- | 2-methyl-2- | 648 |
| | ylmethyl | | trifluoromethyl- | propylthio | |
| | | | pyridin-2-yl | | |
| 2-196 | Pyridin-2- | 3 | 2-methoxypyridin-5- | 2-methyl-2- | 610 |
| | ylmethyl | | yl | propylthio | |
| 2-197 | Quinolin-2- | 4 | 5-fluoropyridin-2-yl | 2-methyl-2- | 648 |
| | ylmethyl | | | propylthio | |
| 2-198 | Quinolin-2- | 4 | 2-ethoxypyridin-6-yl | 2-methyl-2- | 674 |
| | ylmethyl | | | propylthio | |
| 2-199 | Pyridin-2- | 4 | 2-ethoxypyridin-6-yl | 2-methyl-2- | 624 |
| | ylmethyl | | | propylthio | |
| 2-200 | 6-fluoroquinolin- | 4 | 2- | 2-methyl-2- | 716 |
| | 2-ylmethyl | | trifluoromethylpyridin- | propylthio | |
| | | | 6-yl | | |
| 2-201 | Pyridin-2- | 4 | 5-fluoropyridin-2-yl | 2-methyl-2 | 598 |
| | ylmethyl | | | propylthio | |
| 2-202 | 5-methylpyridin- | 4- | 2- | 2-methyl-2- | 662 |
| | 2-ylmethyl | | trifluoromethyl- | propylthio | |
| | | | pyridin-6-yl | | |
| 2-203 | Quinolin-2- | 4 | 2- | 2-methyl-2- | 698 |
| | ylmethyl | | trifluoromethyl- | propylthio | |
| | | | pyridin-6-yl | | |
| 2-204 | Pyridin-2- | 4 | 2- | 2-methyl-2- | 648 |
| | ylmethyl | | trifluoromethyl- | propylthio | |
| | | | pyridin-6-yl | | |
| 2-205 | Quinolin-2- | 4 | 2-thiazolyl | 2-methyl-2- | 636 |
| | ylmethyl | | | propylthio | |
| 2-206 | Pyridin-2- | 3 | 4- | 2-methyl-2- | 617 |
| | ylmethyl | | methoxytetrahydro- | propylthio | |
| | | | pyran-4-yl | | |
| 2-207 | 6-fluoroquinolin- | 4 | Pyridin-2-yl | 2-methyl-2- | 648 |
| | 2-ylmethyl | | | propylthio | |
| 2-208 | 5-Ethylpyridin-2- | 4 | Pyridin-3-yl | 2-methyl-2- | 608 |
| | ylmethyl | | | propylthio | |
| 2-209 | Quinolin-2- | 4 | Pyridin-3-yl | 2-methyl-2- | 630 |
| | ylmethyl | | | propylthio | |
| 2-210 | 6-fluoroquinolin- | 4 | Pyridin-3-yl | 2-methyl-2- | 648 |
| | 2-ylmethyl | | | propylthio | |
| 2-211 | 5-methylpyridin- | 4- | Pyridin-2-yl | 2-methyl-2- | 594 |
| | 2-ylmethyl | | | propylthio | |
| 2-212 | 5-Ethylpyridin-2- | 4 | Pyridin-2-yl | 2-methyl-2- | 608 |
| | ylmethyl | | | propylthio | |
| 2-213 | Quinolin-2- | 4 | Pyridin-2-yl | 2-methyl-2- | 630 |
| | ylmethyl | | | propylthio | |
| 2-214 | 5-methylpyridin- | 4- | Pyridin-3-yl | 2-methyl-2- | 594 |
| | 2-ylmethyl | | | propylthio | |
| 2-215 | 5-methylpyridin- | 4 | 4-methoxypyridin-2- | 2-methyl-2- | 624 |
| | 2-ylmethyl | | yl | propylthio | |
| 2-216 | Quinolin-2- | 4 | 3-methoxypyridin-2- | 2-methyl-2- | 660 |
| | ylmethyl | | yl | propylthio | |
| 2-217 | 5-methylpyridin- | 4 | 3-methoxypyridin-2- | 2-methyl-2- | 624 |
| | 2-ylmethyl | | yl | propylthio | |
| 2-218 | 5-ethylpyridin-2- | 4 | 3-methoxypyridin-2- | 2-methyl-2- | 638 |
| | ylmethyl | | yl | propylthio | |
| 2-219 | 5-methylpyridin- | 4 | 4-trifluoromethyl | 2-methyl-2- | 663 |
| | 2-ylmethyl | | pyridin-2-yl | propylthio | |
| 2-220 | 5-ethylpyridin-2- | 4 | 4-trifluoromethyl | 2-methyl-2- | 677 |
| | ylmethyl | | pyridin-2-yl | propylthio | |
| 2-221 | Quinolin-2- | 4 | 4-trifluoromethyl | 2-methyl-2- | 698 |
| | ylmethyl | | pyridin-2-yl | propylthio | |
| 2-222 | 5-methylpyridin- | 4 | 5-fluoropyridin-3-yl | 2-methyl-2- | 613 |
| | 2-ylmethyl | | | propylthio | |
| 2-223 | 5-ethylpyridin-2- | 4 | 5-fluoropyridin-3-yl | 2-methyl-2- | 626 |
| | ylmethyl | | | propylthio | |
| 2-224 | Quinolin-2- | 4 | 5-fluoropyridin-3-yl | 2-methyl-2- | 649 |
| | ylmethyl | | | propylthio | |
| 2-225 | 2,3-dimethyl | 4 | 2-methoxypyridin-5- | 2-methyl-2- | |
| | pyridin-6-yl | | yl | propylthio | |
| 2-226 | 2,3-dimethyl | 4 | 3-trifluoromethyl | 2-methyl-2- | |
| | pyridin-6-yl | | pyridin-2-yl | propylthio | |
| 2-227 | 2,3-dimethyl | 4 | 4-trifluoromethyl | 2-methyl-2- | |
| | pyridin-6-yl | | pyridin-2-yl | propylthio | |
| 2-228 | 2,3-dimethyl | 4 | 3-fluoropyridin-2-yl | 2-methyl-2- | |
| | pyridin-6-yl | | | propylthio | |
| 2-229 | 2,3-dimethyl | 4 | 5-fluoropyridin-3-y | 2-methyl-2- | |
| | pyridin-6-yl | | | propylthio | |
| 2-230 | 2,3-dimethyl | 4 | 4-methoxypyridin-2- | 2-methyl-2- | |
| | pyridin-6-yl | | yl | propylthio | |
| 2-231 | 2,3-dimethyl | 4 | Pyridine-3-yl | 2-methyl-2- | |
| | pyridin-6-yl | | | propylthio | |
| 2-232 | 5-methylpyridin- | 4 | 2-methoxypyridin-3- | 2-methyl-2- | |
| | 2-ylmethyl | | yl | propylthio | |
| 2-233 | 5-ethylpyridin-2- | 4 | 2-methoxypyridin-3- | 2-methyl-2- | |
| | ylmethyl | | yl | propylthio | |
| 2-234 | Quinolin-2- | 4 | 2-methoxypyridin-3- | 2-methyl-2- | |
| | ylmethyl | | yl | propylthio | |

TABLE 3
N-(heteroaryl-aryl) and N-(heteroaryl-heteroaryl) indoles

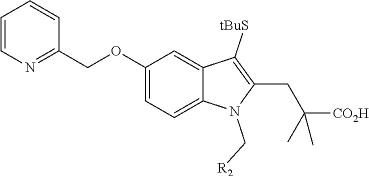

| Compound | | |
|---|---|---|
| # | R₂ | M + H |
| 3-1 | 2-(2-methoxy pyrid-5-yl)-pyrid-5-yl | 611 |
| 3-2 | 2-(4-methoxy phenyl)-pyrid-5-yl | 610 |
| 3-3 | 2-(4-trifluoromethoxy phenyl)-pyrid-5-yl | 664 |
| 3-4 | 5-(4-methoxy phenyl)-pyrid-2-yl | 610 |
| 3-5 | 5-(4-trifluoromethoxyphenyl)-pyrid-2-yl | 664 |

TABLE 4
N-(aryl-heteroaryl) indoles with non-aryl C5 substituents

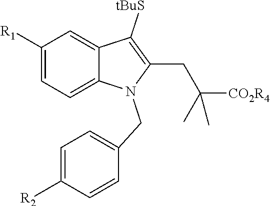

| Compound | | | | |
|---|---|---|---|---|
| # | R₁ | R₂ | R₄ | M + H |
| 4-1 | OH | 2-methoxypyridin-5- | H | 519 |
| 4-2 | isopropyl | 2-methoxypyridin-5-yl | H | 545 |

The following is a listing of names of various compounds from Tables 1-4): 3-[3-tert-butylsulfanyl-1-(4-pyridin-2-yl-benzyl)-5-(pyridin-2-ylmethoxy)-1H-indol-2-yl]-2,2-dimethyl-propionamide (Compound 1-1); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid ethyl ester (Compound 1-2); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid 6-hydroxy-hexyl ester (Compound 1-3); 1-[3-tert-butylsulfanyl-1-(4-pyridin-2-yl-benzyl)-5-(pyridin-2-ylmethoxy)-1H-indol-2-yl]-2-methyl-propan-2-ol (Compound 1-4); 1-[3-tert-butylsulfanyl-1-(4-pyridin-3-yl-benzyl)-5-(pyridin-2-ylmethoxy)-1H-indol-2-yl]-2-methyl-propan-2-ol (Compound 1-5); 1-[3-tert-butylsulfanyl-5-(pyridin-2-ylmethoxy)-1-(4-thiazol-2-yl-benzyl)-1H-indol-2-yl]-2-methyl-propan-2-ol (Compound 1-6); 1-[3-tert-butylsulfanyl-1-(4-[1,3,4]oxadiazol-2-yl-benzyl)-5-(pyridin-2-ylmethoxy)-1H-indol-2-yl]-2-methyl-propan-2-ol (Compound 1-7); 3-tert-butylsulfanyl-1-[4-(6-methoxy-pyridin-3-yl)-benzyl]-2-(2-methyl-2-[1,3,4]oxadiazol-2-yl-propyl)-5-(pyridin-2-ylmethoxy)-1H-indole (Compound 1-8); 5-{2-[3-tert-butylsulfanyl-1-[4-(6-methoxy-pyridin-3-yl)-benzyl]-5-(pyridin-2-ylmethoxy)-1H-indol-2-yl]-1,1-dimethyl-ethyl}-[1,3,4]oxadiazol-2-ylamine (Compound 1-9); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-pyrazin-2-yl-propionamide (Compound 1-10); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-thiazol-2-yl-propionamide (Compound 1-11); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-pyridin-3-yl-propionamide (Compound 1-12); 3-[3-tert-butylsulfanyl-1-[4-(6-methoxy-pyridin-3-yl)-benzyl]-5-(pyridin-2-ylmethoxy)-1H-indol-2-yl]-N-(2-dimethylamino-ethyl)-2,2-dimethyl-propionamide (Compound 1-13); 5-{4-[3-tert-butylsulfanyl-2-(2,2-dimethyl-propyl)-5-(pyridin-2-ylmethoxy)-indol-1-ylmethyl]-phenyl}-[1,3,4]oxadiazol-2-ylamine (Compound 1-14); 3-[3-tert-butylsulfanyl-1-[4-(5-fluoro-pyridin-2-yl)-benzyl]-5-(quinolin-2-ylmethoxy)-1H-indol-2-yl]-N-(2-dimethylamino-ethyl)-2,2-dimethyl-propanoylguanidine (Compound 1-15); 3-[3-tert-butylsulfanyl-5-(pyridin-2-ylmethoxy)-1-(4-thiazol-2-yl-benzyl)-1H-indol-2-yl]-2,2-dimethyl-propionic acid (Compound 2-1); 3-[3-tert-butylsulfanyl-5-(pyridin-2-ylmethoxy)-1-(4-pyrimidin-2-yl-benzyl)-1H-indol-2-yl]-2,2-dimethyl-propionic acid (Compound 2-2); -[3-tert-butylsulfanyl-1-(4-pyridin-3-yl-benzyl)-5-(pyridin-2-ylmethoxy)-1H-indol-2-yl]-2,2-dimethyl-propionic acid (Compound 2-3); 3-[3-tert-butylsulfanyl-5-(pyridin-2-ylmethoxy)-1-(4-pyrimidin-5-yl-benzyl)-1H-indol-2-yl]-2,2-dimethyl-propionic acid (Compound 2-4); 3-[3-tert-butylsulfanyl-1-(4-pyrazin-2-yl-benzyl)-5-(pyridin-2-ylmethoxy)-1H-indol-2-yl]-2,2-dimethyl-propionic acid (Compound 2-5); 3-[3-tert-butylsulfanyl-1-[4-(6-methoxy-pyridazin-3-yl)-benzyl]-5-(pyridin-2-ylmethoxy)-1H-indol-2-yl]-2,2-dimethyl-propionic acid (Compound 2-6); 3-[1-[4-(5-amino-pyrazin-2-yl)-benzyl]-3-tert-butylsulfanyl-5-(pyridin-2-ylmethoxy)-1H-indol-2-yl]-2,2-dimethyl-propionic acid (Compound 2-7); 3-[3-(3,3-dimethyl-butyryl)-5-(pyridin-2-ylmethoxy)-1-(4-thiazol-2-yl-benzyl)-1H-indol-2-yl]-2,2-dimethyl-propionic acid (Compound 2-8); 2,2-dimethyl-3-[5-(pyridin-2-ylmethoxy)-1-(4-thiazol-2-yl-benzyl)-1H-indol-2-yl]-propionic acid (Compound 2-9); 3-[3-acetyl-5-(pyridin-2-ylmethoxy)-1-(4-thiazol-2-yl-benzyl)-1H-indol-2-yl]-2,2-dimethyl-propionic acid (Compound 2-10); 3-[1-[4-(6-methoxy-pyridazin-3-yl)-benzyl]-5-(pyridin-2-ylmethoxy)-1H-indol-2-yl]-2,2-dimethyl-propionic acid (Compound 2-11); 3-[3-acetyl-1-[4-(6-methoxy-pyridazin-3-yl)-benzyl]-5-(pyridin-2-ylmethoxy)-1H-indol-2-yl]-2,2-dimethyl-propionic acid (Compound 2-12); 3-[3-ethyl-1-[4-(6-methoxy-pyridazin-3-yl)-benzyl]-5-(pyridin-2-ylmethoxy)-1H-indol-2-yl]-2,2-dimethyl-propionic acid (Compound 2-13); 3-[3-(3,3-dimethyl-butyl)-5-(pyridin-2-ylmethoxy)-1-(4-thiazol-2-yl-benzyl)-1H-indol-2-yl]-2,2-dimethyl-propionic acid (Compound 2-14); 3-[3-cyclopropanecarbonyl-5-(pyridin-2-ylmethoxy)-1-(4-thiazol-2-yl-benzyl)-1H-indol-2-yl]-2,2-dimethyl-propionic acid (Compound 2-15); 3-[3-cyclobutanecarbonyl-5-(pyridin-2-ylmethoxy)-1-(4-thiazol-2-yl-benzyl)-1H-indol-2-yl]-2,2-dimethyl-propionic acid (Compound 2-16); 3-[3-tert-butylsulfanyl-[4-(6-hydroxy-pyridazin-3-yl)-benzyl]-5-(pyridin-2-ylmethoxy)-1H-indol-2-yl]-2,2-dimethyl-propionic acid (Compound 2-17); 3-[3-tert-butylsulfanyl-1-(4-pyridin-4-yl-benzyl)-5-(pyridin-2-ylmethoxy)-1H-indol-2-yl]-2,2-dimethyl-propionic acid (Compound 2-18); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid (Compound 2-19); 3-[3-tert-butylsulfanyl-1-[4-(6-methyl-pyridazin-3-yl)-benzyl]-5-(pyridin-2-ylmethoxy)-1H-indol-2-yl]-2,2-dimethyl-propionic acid (Compound 2-20); 3-[3-tert-butylsulfanyl-1-[4-(5-methyl-thiazol-2-yl)-benzyl]-5-(pyridin-2-ylmethoxy)-1H-indol-2-yl]-2,2-dimethyl-propionic acid (Compound 2-21); 3-[3-cyclobutylmethyl-5-(pyridin-2-ylmethoxy)-1-(4-thiazol-2-yl-benzyl)-1H-indol-2-yl]-2,2-dimethyl-propionic acid (Compound 2-22); 3-[3-tert-butylsulfanyl-[4-(6-methoxy-pyridazin-3-yl)-benzyl]-5-(2-methyl-thiazol-4-ylmethoxy)-1H-indol-2-yl]-2,2-dimethyl-propionic acid (Compound 2-23); 3-[3-tert-butylsulfanyl-5-(2-methyl-thiazol-4-ylmethoxy)-1-(4-thiazol-2-yl-benzyl)-1H-indol-2-yl]-2,2-dimethyl-propionic acid (Compound 2-24); 2,2-dimethyl-3-[5-(2-methyl-thiazol-4-ylmethoxy)-1-(4-thiazol-2-yl-benzyl)-1H-indol-2-yl]-propionic acid (Compound 2-25); 3-[3-(3,3-dimethyl-butyryl)-5-(2-methyl-thiazol-4-ylmethoxy)-1-(4-thiazol-2-yl-benzyl)-1H-indol-2-yl]-2,2-dimethyl-propionic acid (Compound 2-26); 3-[1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-27); 3-[3-(3,3-dimethyl-butyryl)-1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-28); 3-[3-ethyl-5-(pyridin-2-ylmethoxy)-1-(4-thiazol-2-yl-benzyl)-1H-indol-2-yl]-2,2-dimethyl-propionic acid (Compound 2-29); 3-{5-(benzothiazol-2-ylmethoxy)-3-tert-butylsulfanyl-1-[4-(6-methoxy-pyridazin-3-yl)-benzyl]-1H-indol-2-yl}-2,2-dimethyl-propionic acid (Compound 2-30); 3-[3-tert-butylsulfanyl-5-(2-methyl-thiazol-4-ylmethoxy)-1-(4-pyrimidin-2-yl-benzyl)-1H-indol-2-yl]-2,2-dimethyl-propionic acid (Compound 2-31); 3-[5-(benzothiazol-2-ylmethoxy)-3-tert-butylsulfanyl-1-(4-pyrimidin-2-yl-benzyl)-1H-indol-2-yl]-2,2-dimethyl-propionic acid (Compound 2-32); 3-[3-tert-butylsulfanyl-[4-(2-methyl-3-pyridin-2-ylmethyl-3H-imidazol-4-yl)-benzyl]-5-(pyridin-2-ylmethoxy)-1H-indol-2-yl]-2,2-dimethyl-propionic acid (Compound 2-33); 3-[3-tert-butylsulfanyl-1-[4-(2,4-dimethyl-thiazol-5-yl)-benzyl]-5-(pyridin-2-ylmethoxy)-1H-indol-2-yl]-2,2-dimethyl-propionic acid (Compound 2-34); 3-[3-tert-butylsulfanyl-1-[4-(5-fluoro-thiazol-2-yl)-benzyl]-5-(pyridin-2-ylmethoxy)-1H-indol-2-yl]-2,2-dimethyl-propionic acid (Compound 2-35); 3-[3-tert-butylsulfanyl-1-[4-(4-methyl-thiazol-2-yl)-benzyl]-5-(pyridin-2-ylmethoxy)-1H-indol-2-yl]-2,2-dimethyl-propionic acid (Compound 2-39); 3-[3-tert-butylsulfanyl-1-[4-(3,5-dimethyl-isoxazol-4-yl)-benzyl]-5-(pyridin-2-ylmethoxy)-1H-indol-2-yl]-2,2-dimethyl-propionic acid (Compound 2-41); 3-[3-tert-butylsulfanyl-1-[4-(3-methyl-3H-imidazol-4-yl)-benzyl]-5-(pyridin-2-ylmethoxy)-1H-indol-2-yl]-2,2-dimethyl-propionic acid (Compound 2-43); 3-[3-tert-butylsulfanyl-1-[4-(5-methoxy-pyridin-2-yl)-benzyl]-5-(pyridin-2-ylmethoxy)-1H-indol-2-yl]-2,2-dimethyl-propionic acid (Compound 2-47); 3-[3-tert-butylsulfanyl-5-(pyridin-2-ylmethoxy)-1-(4-[1,3,4]thiadiazol-2-yl-benzyl)-1H-indol-2-yl]-2,2-dimethyl-propionic acid (Compound 2-55); 3-[3-tert-butylsulfanyl-1-[4-(6-hydroxy-pyridin-3-yl)-benzyl]-5-(pyridin-2-ylmethoxy)-1H-indol-2-yl]-2,2-dimethyl-propionic acid (Compound 2-62); 3-[3-tert-butylsulfanyl-1-[4-(6-cyano-pyridin-3-yl)-benzyl]-5-(pyridin-2-ylmethoxy)-1H-indol-2-yl]-2,2-dimethyl-propionic acid (Compound 2-64); 3-{3-tert-butylsulfanyl-5-(pyridin-2-ylmethoxy)-1-[4-(6-trifluoromethyl-pyridin-3-yl)-benzyl]-1H-indol-2-yl}-2,2-dimethyl-propionic acid (Compound 2-65); 3-[3-tert-butylsulfanyl-1-[4-(2-methoxy-pyrimidin-5-yl)-benzyl]-5-(pyridin-2-ylmethoxy)-1H-indol-2-yl]-2,2-dimethyl-propionic acid (Compound 2-67); 3-[3-tert-butylsulfanyl-1-[4-(2-methoxy-thiazol-4-yl)-benzyl]-5-(pyridin-2-ylmethoxy)-1H-indol-2-yl]-2,2-dimethyl-propionic acid (Compound 2-68); 3-[3-tert-butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-73); 3-{3-tert-butylsulfanyl-5-(5-ethyl-pyridin-2-ylmethoxy)-1-[4-(4-methoxy-pyridin-2-yl)-benzyl]-1H-indol-2-yl}-2,2-dimethyl-propionic acid (Compound 2-76); 3-[3-tert-butylsulfanyl-1-[4-(4-methoxy-pyridin-2-yl)-benzyl]-5-(quinolin-2-ylmethoxy)-1H-indol-2-yl]-2,2-dimethyl-propionic acid (Compound 2-77); 3-{3-tert-butylsulfanyl-5-(6-fluoro-quinolin-2-ylmethoxy)-1-[4-(4-methoxy-pyridin-2-yl)-benzyl]-1H-indol-2-yl}-2,2-dimethyl-propionic acid (Compound 2-78); 3-[3-tert-butylsulfanyl-1-[4-(3-fluoro-pyridin-2-yl)-benzyl]-5-(5-methyl-pyridin-2-ylmethoxy)-1H-indol-2-yl]-2,2-dimethyl-propionic acid (Compound 2-82); 3-{3-tert-butylsulfanyl-5-(5-ethyl-pyridin-2-ylmethoxy)-1-[4-(3-fluoro-pyridin-2-yl)-benzyl]-1H-indol-2-yl}-2,2-dimethyl-propionic acid (Compound 2-84); 3-[3-tert-butylsulfanyl-[4-(3-fluoro-pyridin-2-yl)-benzyl]-5-(quinolin-2-ylmethoxy)-1H-indol-2-yl]-2,2-dimethyl-propionic acid (Compound 2-85); 3-[3-tert-butylsulfanyl-1-[4-(5-carbamoyl-pyridin-2-yl)-benzyl]-5-(pyridin-2-ylmethoxy)-1H-indol-2-yl]-2,2-dimethyl-propionic acid (Compound 2-87); 3-[3-tert-butylsulfanyl-1-[4-(5-cyano-pyridin-2-yl)-benzyl]-5-(pyridin-2-ylmethoxy)-1H-indol-2-yl]-2,2-dimethyl-propionic acid (Compound 2-88); 3-[3-tert-butylsulfanyl-1-[4-(5-methoxy-thiazol-2-yl)-benzyl]-5-(pyridin-2-ylmethoxy)-1H-indol-2-yl]-2,2-dimethyl-propionic acid (Compound 2-89); 3-[3-tert-butylsulfanyl-1-[4-(6-methyl-pyridin-3-yl)-benzyl]-5-(pyridin-2-ylmethoxy)-1H-indol-2-yl]-2,2-dimethyl-propionic acid (Compound 2-90); 3-{3-tert-butylsulfanyl-5-(pyridin-2-ylmethoxy)-1-[4-(5-trifluoromethyl-pyridin-2-yl)-benzyl]-1H-indol-2-yl}-2,2-dimethyl-propionic acid (Compound 2-91); 3-[3-tert-butylsulfanyl-1-[4-(2-ethoxy-thiazol-4-yl)-benzyl]-5-(pyridin-2-ylmethoxy)-1H-indol-2-yl]-2,2-dimethyl-propionic acid (Compound 2-92); 3-[3-tert-butylsulfanyl-1-[4-(4-methyl-1H-imidazol-2-yl)-benzyl]-5-(pyridin-2-ylmethoxy)-1H-indol-2-yl]-2,2-dimethyl-propionic acid (Compound 2-93); 3-[3-tert-butylsulfanyl-1-[4-(6-ethoxy-pyridin-3-yl)-benzyl]-5-(pyridin-2-ylmethoxy)-1H-indol-2-yl]-2,2-dimethyl-propionic acid (Compound 2-94); 3-[3-tert-butylsulfanyl-1-[4-(6-methoxy-pyridin-2-yl)-benzyl]-5-(pyridin-2-ylmethoxy)-1H-indol-2-yl]-2,2-dimethyl-propionic acid (Compound 2-95); 3-[3-tert-butylsulfanyl-1-[4-(5-methoxy-pyridin-3-yl)-benzyl]-5-(pyridin-2-ylmethoxy)-1H-indol-2-yl]-2,2-dimethyl-propionic acid (Compound 2-96); 3-[3-tert-butylsulfanyl-1-[4-(6-carbamoyl-pyridin-3-yl)-benzyl]-5-(pyridin-2-ylmethoxy)-1H-indol-2-yl]-2,2-dimethyl-propionic acid (Compound 2-97); 3-[3-tert-butylsulfanyl-1-[4-(5-methyl-pyridin-2-yl)-benzyl]-5-(pyridin-2-ylmethoxy)-1H-indol-2-yl]-2,2-dimethyl-propionic acid (Compound 2-98); 3-{3-tert-butylsulfanyl-5-(6-fluoro-pyridin-2-ylmethoxy)-1-[4-(6-methoxy-pyridin-3-yl)-benzyl]-1H-indol-2-yl}-2,2-dimethyl-propionic acid (Compound 2-99); 3-[3-tert-butylsulfanyl-1-[4-(6-methoxy-pyridin-3-yl)-benzyl]-5-(6-methoxy-pyridin-2-ylmethoxy)-1H-indol-2-yl]-2,2-dimethyl-propionic acid (Compound 2-100); 3-[3-tert-butylsulfanyl-1-[4-(6-methoxy-pyridin-3-yl)-benzyl]-5-(6-methyl-pyridin-2-ylmethoxy)-1H-indol-2-yl]-2,2-dimethyl-propionic acid (Compound 2-101); 3-{3-tert-butylsulfanyl-5-(5-methyl-pyridin-2-ylmethoxy)-1-[4-(6-trifluoromethyl-pyridin-3-yl)-benzyl]-1H-indol-2-yl}-2,2-dimethyl-propionic acid (Compound 2-102); 3-{3-tert-butylsulfanyl-5-(5-methyl-pyridin-2-ylmethoxy)-1-[4-(5-trifluoromethyl-pyridin-2-yl)-benzyl]-1H-indol-2-yl}-2,2-dimethyl-propionic acid (Compound 2-103); 3-{3-tert-butylsulfanyl-5-(6-cyclopropyl-pyridin-2-ylmethoxy)-1-[4-(6-methoxy-pyridin-3-yl)-benzyl]-1H-indol-2-yl}-2,2-dimethyl-propionic acid (Compound 2-104); 3-[3-tert-butylsulfanyl-1-[4-(5-methyl-pyridin-2-yl)-benzyl]-5-(5-methyl-pyridin-2-ylmethoxy)-1H-indol-2-yl]-2,2-dimethyl-propionic acid (Compound 2-105); 3-[3-tert-butylsulfanyl-1-[4-(6-methoxy-pyridazin-3-yl)-benzyl]-5-(5-methyl-pyridin-2-ylmethoxy)-1H-indol-2-yl]-2,2-dimethyl-propionic acid (Compound 2-106); 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid (Compound 2-107); 3-{3-tert-butylsulfanyl-5-(5-chloro-pyridin-2-ylmethoxy)-1-[4-(6-methoxy-pyridin-3-yl)-benzyl]-1H-indol-2-yl}-2,2-dimethyl-propionic acid (Compound 2-108); 3-{3-tert-butylsulfanyl-5-((S)-1-pyridin-2-yl-ethoxy)-1-[4-(5-trifluoromethyl-pyridin-2-yl)-benzyl]-1H-indol-2-yl}-2,2-dimethyl-propionic acid (Compound 2-109); 3-{3-tert-butylsulfanyl-5-((R)-1-pyridin-2-yl-ethoxy)-1-[4-(5-trifluoromethyl-pyridin-2-yl)-benzyl]-1H-indol-2-yl}-2,2-dimethyl-propionic acid (Compound 2-110); 3-[3-tert-butylsulfanyl-[4-(6-methoxy-pyridin-3-yl)-benzyl]-5-((S)-1-pyridin-2-yl-ethoxy)-1H-indol-2-yl]-2,2-dimethyl-propionic acid (Compound 2-111); 3-[3-tert-butylsulfanyl-1-[4-(6-methoxy-pyridin-3-yl)-benzyl]-5-((R)-1-pyridin-2-yl-ethoxy)-1H-indol-2-yl]-2,2-dimethyl-propionic acid (Compound 2-112); 3-[3-tert-butylsulfanyl-1-[4-(6-methoxy-pyridin-2-yl)-benzyl]-5-((S)-1-pyridin-2-yl-ethoxy)-1H-indol-2-yl]-2,2-dimethyl-propionic acid (Compound 2-113); 3-[3-tert-butylsulfanyl-1-[4-(6-methoxy-pyridin-2-yl)-benzyl]-5-((R)-1-pyridin-2-yl-ethoxy)-1H-indol-2-yl]-2,2-dimethyl-propionic acid (Compound 2-114); 3-[3-tert-butylsulfanyl-1-[4-(2-ethoxy-thiazol-4-yl)-benzyl]-5-((S)-1-pyridin-2-yl-ethoxy)-1H-indol-2-yl]-2,2-dimethyl-propionic acid (Compound 2-115); 3-[3-tert-butylsulfanyl-[4-(2-ethoxy-thiazol-4-yl)-benzyl]-5-((R)-1-pyridin-2-yl-ethoxy)-1H-indol-2-yl]-2,2-dimethyl-propionic acid (Compound 2-116); 3-[3-tert-butylsulfanyl-1-[4-(6-methoxy-pyridin-3-yl)-benzyl]-5-(3-methyl-pyridin-2-ylmethoxy)-1H-indol-2-yl]-2,2-dimethyl-propionic acid (Compound 2-117); 3-{3-tert-butylsulfanyl-5-(3-methyl-pyridin-2-ylmethoxy)-1-[4-(5-trifluoromethyl-pyridin-2-yl)-benzyl]-1H-indol-2-yl}-2,2-dimethyl-propionic acid (Compound 2-118); 3-{3-tert-butylsulfanyl-5-(3,5-dimethyl-pyridin-2-ylmethoxy)-1-[4-(6-methoxy-pyridin-3-yl)-benzyl]-1H-indol-2-yl}-2,2-dimethyl-propionic acid (Compound 2-119); 3-{3-tert-butylsulfanyl-5-(3,5-dimethyl-pyridin-2-ylmethoxy)-1-[4-(5-trifluoromethyl-pyridin-2-yl)-benzyl]-1H-indol-2-yl}-2,2-dimethyl-propionic acid (Compound 2-120); 3-{5-(benzothiazol-2-ylmethoxy)-3-tert-butylsulfanyl-1-[4-(6-methoxy-pyridin-3-yl)-benzyl]-1H-indol-2-yl}-2,2-dimethyl-propionic acid (Compound 2-121); 3-{5-(benzothiazol-2-ylmethoxy)-3-tert-butylsulfanyl-1-[4-(5-methoxy-pyridin-2-yl)-benzyl]-1H-indol-2-yl}-2,2-dimethyl-propionic acid (Compound 2-122); 3-{5-(benzothiazol-2-ylmethoxy)-3-cyclobutanecarbonyl-1-[4-(6-methoxy-pyridin-3-yl)-benzyl]-1H-indol-2-yl}-2,2-dimethyl-propionic acid (Compound 2-123); 3-{5-(benzothiazol-2-ylmethoxy)-3-cyclobutylmethyl-1-[4-(6-methoxy-pyridin-3-yl)-benzyl]-1H-indol-2-yl}-2,2-dimethyl-propionic acid (Compound 2-124); 3-{3-tert-butylsulfanyl-5-(5-ethyl-pyridin-2-ylmethoxy)-1-[4-(6-methoxy-pyridin-3-yl)-benzyl]-1H-indol-2-yl}-2,2-dimethyl-propionic acid (Compound 2-125); 3-[3-tert-butylsulfanyl-1-[4-(6-ethoxy-pyridin-3-yl)-benzyl]-5-(5-ethyl-pyridin-2-ylmethoxy)-1H-indol-2-yl]-2,2-dimethyl-propionic acid (Compound 2-126); 3-{3-tert-butylsulfanyl-5-(5-ethyl-pyridin-2-ylmethoxy)-1-[4-(6-trifluoromethyl-pyridin-3-yl)-benzyl]-1H-indol-2-yl}-2,2-dimethyl-propionic acid (Compound 2-127); 3-{3-tert-butylsulfanyl-5-(5-ethyl-pyridin-2-ylmethoxy)-1-[4-(5-trifluoromethyl-pyridin-2-yl)-benzyl]-1H-indol-2-yl}-2,2-dimethyl-propionic acid (Compound 2-128); 3-[3-tert-butylsulfanyl-1-[4-(2-ethoxy-thiazol-4-yl)-benzyl]-5-(5-methyl-pyridin-2-ylmethoxy)-1H-indol-2-yl]-2,2-dimethyl-propionic acid (Compound 2-129); 3-[3-tert-butylsulfanyl-1-[4-(2-methoxy-thiazol-4-yl)-benzyl]-5-(5-methyl-pyridin-2-ylmethoxy)-1H-indol-2-yl]-2,2-dimethyl-propionic acid (Compound 2-130); 3-[3-tert-butylsulfanyl-1-[4-(6-methoxy-pyridin-2-yl)-benzyl]-5-(5-methyl-pyridin-2-ylmethoxy)-1H-indol-2-yl]-2,2-dimethyl-propionic acid (Compound 2-131); 3-[3-cyclobutylmethyl-1-[4-(6-methoxy-pyridin-3-yl)-benzyl]-5-(pyridin-2-ylmethoxy)-1H-indol-2-yl]-2,2-dimethyl-propionic acid (Compound 2-132); 3-[3-cyclobutylmethyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-133); 3-[3-isobutyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-134); 3-[3-tert-butylsulfanyl-1-[4-(6-methoxy-pyridin-3-yl)-benzyl]-5-(quinolin-2-ylmethoxy)-1H-indol-2-yl]-2,2-dimethyl-propionic acid (Compound 2-135); 3-{3-tert-butylsulfanyl-5-(quinolin-2-ylmethoxy)-1-[4-(6-trifluoromethyl-pyridin-3-yl)-benzyl]-1H-indol-2-yl}-2,2-dimethyl-propionic acid (Compound 2-136); 3-{3-tert-butylsulfanyl-5-(quinolin-2-ylmethoxy)-1-[4-(5-trifluoromethyl-pyridin-2-yl)-benzyl]-1H-indol-2-yl}-2,2-dimethyl-propionic acid (Compound 2-137); 3-[3-tert-butylsulfanyl-1-[4-(6-methoxy-pyridazin-3-yl)-benzyl]-5-(quinolin-2-ylmethoxy)-1H-indol-2-yl]-2,2-dimethyl-propionic acid (Compound 2-138); 3-[3-tert-butylsulfanyl-1-[4-(6-ethoxy-pyridin-3-yl)-benzyl]-5-(quinolin-2-ylmethoxy)-1H-indol-2-yl]-2,2-dimethyl-propionic acid (Compound 2-139); 3-{3-tert-butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic acid (Compound 2-140); 3-{3-tert-butylsulfanyl-5-(6-fluoro-quinolin-2-ylmethoxy)-1-[4-(6-methoxy-pyridin-3-yl)-benzyl]-1H-indol-2-yl}-2,2-dimethyl-propionic acid (Compound 2-141); 3-[3-tert-butylsulfanyl-1-[4-(2-ethoxy-thiazol-4-yl)-benzyl]-5-(6-fluoro-quinolin-2-ylmethoxy)-1H-indol-2-yl]-2,2-dimethyl-propionic acid (Compound 2-142); 3-{3-tert-butylsulfanyl-5-(6-fluoro-quinolin-2-ylmethoxy)-1-[4-(5-trifluoromethyl-pyridin-2-yl)-benzyl]-1H-indol-2-yl}-2,2-dimethyl-propionic acid (Compound 2-143); 3-{3-tert-butylsulfanyl-5-(7-fluoro-quinolin-2-ylmethoxy)-1-[4-(6-trifluoromethyl-pyridin-3-yl)-benzyl]-1H-indol-2-yl}-2,2-dimethyl-propionic acid (Compound 2-144); 3-{3-tert-butylsulfanyl-5-(7-fluoro-quinolin-2-ylmethoxy)-1-[4-(5-trifluoromethyl-pyridin-2-yl)-benzyl]-1H-indol-2-yl}-2,2-dimethyl-propionic acid (Compound 2-145); 3-{3-tert-butylsulfanyl-5-(7-fluoro-quinolin-2-ylmethoxy)-1-[4-(6-methoxy-pyridin-3-yl)-benzyl]-1H-indol-2-yl}-2,2-dimethyl-propionic acid (Compound 2-146); 3-[3-tert-butylsulfanyl-1-[4-(6-ethoxy-pyridin-3-yl)-benzyl]-5-(7-fluoro-quinolin-2-ylmethoxy)-1H-indol-2-yl]-2,2-dimethyl-propionic acid (Compound 2-147); 3-[3-tert-butylsulfanyl-[4-(3-fluoro-pyridin-2-yl)-benzyl]-5-(6-fluoro-quinolin-2-ylmethoxy)-1H-indol-2-yl]-2,2-dimethyl-propionic acid (Compound 2-148); 3-{3-tert-butylsulfanyl-5-(5-methyl-pyridin-2-ylmethoxy)-1-[4-(3-trifluoromethyl-pyridin-2-yl)-benzyl]-1H-indol-2-yl}-2,2-dimethyl-propionic acid (Compound 2-149); 3-{3-tert-butylsulfanyl-5-(5-ethyl-pyridin-2-ylmethoxy)-1-[4-(3-trifluoromethyl-pyridin-2-yl)-benzyl]-1H-indol-2-yl}-2,2-dimethyl-propionic acid (Compound 2-150); 3-{3-tert-butylsulfanyl-5-(quinolin-2-ylmethoxy)-1-[4-(3-trifluoromethyl-pyridin-2-yl)-benzyl]-1H-indol-2-yl}-2,2-dimethyl-propionic acid (Compound 2-151); 3-{3-tert-butylsulfanyl-5-(6-fluoro-quinolin-2-ylmethoxy)-1-[4-(3-trifluoromethyl-pyridin-2-yl)-benzyl]-1H-indol-2-yl}-2,2-dimethyl-propionic acid (Compound 2-156); 3-[3-tert-butylsulfanyl-1-[4-(6-ethoxy-pyridin-3-yl)-benzyl]-5-(3-methyl-pyridin-2-ylmethoxy)-1H-indol-2-yl]-2,2-dimethyl-propionic acid (Compound 2-157); 3-{3-tert-butylsulfanyl-5-(3-methyl-pyridin-2-ylmethoxy)-1-[4-(6-trifluoromethyl-pyridin-3-yl)-benzyl]-1H-indol-2-yl}-2,2-dimethyl-propionic acid (Compound 2-158); 3-{3-tert-butylsulfanyl-5-(3,5-dimethyl-pyridin-2-ylmethoxy)-1-[4-(6-ethoxy-pyridin-3-yl)-benzyl]-1H-indol-2-yl}-2,2-dimethyl-propionic acid (Compound 2-159); 3-[3-tert-butylsulfanyl-1-[4-(6-methoxy-pyridin-3-yl)-benzyl]-5-(4-methyl-pyridin-2-ylmethoxy)-1H-indol-2-yl]-2,2-dimethyl-propionic acid (Compound 2-160); 3-[3-tert-butylsulfanyl-1-[4-(6-ethoxy-pyridin-3-yl)-benzyl]-5-(4-methyl-pyridin-2-ylmethoxy)-1H-indol-2-yl]-2,2-dimethyl-propionic acid (Compound 2-161); 3-{3-tert-butylsulfanyl-5-(4-methyl-pyridin-2-ylmethoxy)-1-[4-(5-trifluoromethyl-pyridin-2-yl)-benzyl]-1H-indol-2-yl}-2,2-dimethyl-propionic acid (Compound 2-162); 3-{3-cyclobutylmethyl-5-(5-methyl-pyridin-2-ylmethoxy)-1-[4-(5-trifluoromethyl-pyridin-2-yl)-benzyl]-1H-indol-2-yl}-2,2-dimethyl-propionic acid (Compound 2-163); 3-[3-tert-butylsulfanyl-1-[4-(6-ethoxy-pyridin-3-yl)-benzyl]-5-(6-fluoro-quinolin-2-ylmethoxy)-1H-indol-2-yl]-2,2-dimethyl-propionic acid (Compound 2-164); 3-{3-tert-butylsulfanyl-5-(6-fluoro-quinolin-2-ylmethoxy)-1-[4-(6-trifluoromethyl-pyridin-3-yl)-benzyl]-1H-indol-2-yl}-2,2-dimethyl-propionic acid (Compound 2-165); 3-[3-tert-butylsulfanyl-1-[4-(6-methoxy-pyridin-3-yl)-benzyl]-5-(6-methyl-quinolin-2-ylmethoxy)-1H-indol-2-yl]-2,2-dimethyl-propionic acid (Compound 2-166); 3-{3-tert-butylsulfanyl-5-(6-methyl-quinolin-2-ylmethoxy)-1-[4-(5-trifluoromethyl-pyridin-2-yl)-benzyl]-1H-indol-2-yl}-2,2-dimethyl-propionic acid (Compound 2-167); 3-[3-tert-butylsulfanyl-1-[4-(6-methyl-pyridazin-3-yl)-benzyl]-5-(quinolin-2-ylmethoxy)-1H-indol-2-yl]-2,2-dimethyl-propionic acid (Compound 2-168); 3-[3-tert-butylsulfanyl-1-[4-(6-ethoxy-pyridazin-3-yl)-benzyl]-5-(quinolin-2-ylmethoxy)-1H-indol-2-yl]-2,2-dimethyl-propionic acid (Compound 2-169); 3-[3-isobutyl-1-[4-(6-methoxy-pyridin-3-yl)-benzyl]-5-(quinolin-2-ylmethoxy)-1H-indol-2-yl]-2,2-dimethyl-propionic acid (Compound 2-170); 3-{3-tert-butylsulfanyl-5-(6-fluoro-quinolin-2-ylmethoxy)-1-[4-(6-methoxy-pyridazin-3-yl)-benzyl]-1H-indol-2-yl}-2,2-dimethyl-propionic acid (Compound 2-171); 3-[1-[4-(6-methoxy-pyridin-3-yl)-benzyl]-3-(2-methyl-propane-2-sulfonyl)-5-(pyridin-2-ylmethoxy)-1H-indol-2-yl]-2,2-dimethyl-propionic acid (Compound 2-172); 3-[1-[4-(6-methoxy-pyridin-3-yl)-benzyl]-3-(2-methyl-propane-2-sulfinyl)-5-(pyridin-2-ylmethoxy)-1H-indol-2-yl]-2,2-dimethyl-propionic acid (Compound 2-173); 3-[3-tert-butylsulfanyl-1-[4-(6-methoxy-pyridin-3-yl)-benzyl]-5-(1-oxy-pyridin-2-ylmethoxy)-1H-indol-2-yl]-2,2-dimethyl-propionic acid (Compound 2-174); 3-{3-tert-butylsulfanyl-5-(imidazo[1,2-a]pyridin-2-ylmethoxy)-1-[4-(6-methoxy-pyridin-3-yl)-benzyl]-1H-indol-2-yl}-2,2-dimethyl-propionic acid (Compound 2-175); 3-[3-tert-butylsulfanyl-1-[4-(6-ethoxy-pyridin-3-yl)-benzyl]-5-(imidazo[1,2-a]pyridin-2-ylmethoxy)-1H-indol-2-yl]-2,2-dimethyl-propionic acid (Compound 2-176); 3-{3-tert-butylsulfanyl-5-(imidazo[1,2-a]pyridin-2-ylmethoxy)-1-[4-(5-trifluoromethyl-pyridin-2-yl)-benzyl]-1H-indol-2-yl}-2,2-dimethyl-propionic acid (Compound 2-177); 3-[3-tert-butylsulfanyl-1-[4-(6-ethoxy-pyridin-3-yl)-benzyl]-5-((R)-1-pyridin-2-yl-ethoxy)-1H-indol-2-yl]-2,2-dimethyl-propionic acid (Compound 2-178); 3-{3-tert-butylsulfanyl-5-(6-fluoro-quinolin-2-ylmethoxy)-1-[4-(6-methyl-pyridazin-3-yl)-benzyl]-1H-indol-2-yl}-2,2-dimethyl-propionic acid (Compound 2-179); 3-[3-tert-butylsulfanyl-1-[4-(6-methoxy-pyridin-3-yl)-benzyl]-5-(5-methyl-isoxazol-3-ylmethoxy)-1H-indol-2-yl]-2,2-dimethyl-propionic acid (Compound 2-180); 3-[3-tert-butylsulfanyl-1-[4-(6-ethoxy-pyridin-3-yl)-benzyl]-5-(5-methyl-isoxazol-3-ylmethoxy)-1H-indol-2-yl]-2,2-dimethyl-propionic acid (Compound 2-181); 3-{3-tert-butylsulfanyl-5-(5-methyl-isoxazol-3-ylmethoxy)-1-[4-(5-trifluoromethyl-pyridin-2-yl)-benzyl]-1H-indol-2-yl}-2,2-dimethyl-propionic acid (Compound 2-182); 3-{3-tert-Butylsulfanyl-5-(2,5-dimethyl-2H-pyrazol-3-ylmethoxy)-1-[4-(6-methoxy-pyridin-3-yl)-benzyl]-1H-indol-2-yl}-2,2-dimethyl-propionic acid (Compound 2-183); 3-{3-tert-butylsulfanyl-5-(1,5-dimethyl-1H-pyrazol-3-ylmethoxy)-1-[4-(6-methoxy-pyridin-3-yl)-benzyl]-1H-indol-2-yl}-2,2-dimethyl-propionic acid (Compound 2-184); 3-[3-tert-butylsulfanyl-1-[4-(6-ethoxy-pyridazin-3-yl)-benzyl]-5-(6-fluoro-quinolin-2-ylmethoxy)-1H-indol-2-yl]-2,2-dimethyl-propionic acid (Compound 2-185); 3-[3-tert-butylsulfanyl-1-[4-(6-ethoxy-pyridazin-3-yl)-benzyl]-5-(5-ethyl-pyridin-2-ylmethoxy)-1H-indol-2-yl]-2,2-dimethyl-propionic acid (Compound 2-186); 3-{3-tert-butylsulfanyl-5-(5-ethyl-pyridin-2-ylmethoxy)-1-[4-(6-methoxy-pyridazin-3-yl)-benzyl]-1H-indol-2-yl}-2,2-dimethyl-propionic acid (Compound 2-187); 3-[3-tert-butylsulfanyl-1-[4-(5-fluoro-pyridin-2-yl)-benzyl]-5-(6-fluoro-quinolin-2-ylmethoxy)-1H-indol-2-yl]-2,2-dimethyl-propionic acid (Compound 2-188); 3-[3-tert-butylsulfanyl-[4-(5-fluoro-pyridin-2-yl)-benzyl]-5-((R)-1-pyridin-2-yl-ethoxy)-1H-indol-2-yl]-2,2-dimethyl-propionic acid (Compound 2-189); 3-[3-tert-butylsulfanyl-1-[4-(6-ethoxy-pyridin-2-yl)-benzyl]-5-(6-fluoro-quinolin-2-ylmethoxy)-1H-indol-2-yl]-2,2-dimethyl-propionic acid (Compound 2-190); 3-[3-tert-butylsulfanyl-1-[4-(6-ethoxy-pyridin-2-yl)-benzyl]-5-((R)-1-pyridin-2-yl-ethoxy)-1H-indol-2-yl]-2,2-dimethyl-propionic acid (Compound 2-191); 3-[3-tert-butylsulfanyl-1-[4-(5-fluoro-pyridin-2-yl)-benzyl]-5-(5-methyl-pyridin-2-ylmethoxy)-1H-indol-2-yl]-2,2-dimethyl-propionic acid (Compound 2-192); 3-[3-tert-butysulfanyl-1-[4-(6-ethoxy-pyridin-2-yl)-benzyl]-5-(5-methyl-pyridin-2-ylmethoxy)-1H-indol-2-yl]-2,2-dimethyl-propionic acid (Compound 2-193); 3-{5-(6-fluoro-quinolin-2-ylmethoxy)-3-isobutyl-1-[4-(6-trifluoromethyl-pyridin-3-yl)-benzyl]-1H-indol-2-yl}-2,2-dimethyl-propionic acid (Compound 2-194); 3-{3-tert-butylsulfanyl-5-(pyridin-2-ylmethoxy)-1-[3-(5-trifluoromethyl-pyridin-2-yl)-benzyl]-1H-indol-2-yl}-2,2-dimethyl-propionic acid (Compound 2-195); 3-[3-tert-butylsulfanyl-1-[3-(6-methoxy-pyridin-3-yl)-benzyl]-5-(pyridin-2-ylmethoxy)-1H-indol-2-yl]-2,2-dimethyl-propionic acid (Compound 2-196); 3-[3-tert-butylsulfanyl-1-[4-(5-fluoro-pyridin-2-yl)-benzyl]-5-(quinolin-2-ylmethoxy)-1H-indol-2-yl]-2,2-dimethyl-propionic acid (Compound 2-197); 3-[3-tert-butylsulfanyl-1-[4-(6-ethoxy-pyridin-2-yl)-benzyl]-5-(quinolin-2-ylmethoxy)-1H-indol-2-yl]-2,2-dimethyl-propionic acid (Compound 2-198); 3-[3-tert-butylsulfanyl-1-[4-(6-ethoxy-pyridin-2-yl)-benzyl]-5-(pyridin-2-ylmethoxy)-1H-indol-2-yl]-2,2-dimethyl-propionic acid (Compound 2-199); 3-{3-tert-butylsulfanyl-5-(6-fluoro-quinolin-2-ylmethoxy)-1-[4-(6-trifluoromethyl-pyridin-2-yl)-benzyl]-1H-indol-2-yl}-2,2-dimethyl-propionic acid (Compound 2-200); 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid (Compound 2-201); 3-{3-tert-butylsulfanyl-5-(5-methyl-pyridin-2-ylmethoxy)-1-[4-(6-trifluoromethyl-pyridin-2-yl)-benzyl]-1H-indol-2-yl}-2,2-dimethyl-propionic acid (Compound 2-202); 3-{3-tert-butylsulfanyl-5-(quinolin-2-ylmethoxy)-1-[4-(6-trifluoromethyl-pyridin-2-yl)-benzyl]-1H-indol-2-yl}-2,2-dimethyl-propionic acid (Compound 2-203); 3-{3-tert-butylsulfanyl-5-(pyridin-2-ylmethoxy)-1-[4-(6-trifluoromethyl-pyridin-2-yl)-benzyl]-1H-indol-2-yl}-2,2-dimethyl-propionic acid (Compound 2-204); 3-[3-tert-butylsulfanyl-5-(quinolin-2-ylmethoxy)-1-(4-thiazol-2-yl-benzyl)-1H-indol-2-yl]-2,2-dimethyl-propionic acid (Compound 2-205); 3-[3-tert-butylsulfanyl-[3-(4-methoxy-tetrahydro-pyran-4-yl)-benzyl]-5-(pyridin-2-ylmethoxy)-1H-indol-2-yl]-2,2-dimethyl-propionic acid (Compound 2-206); 3-[3-tert-butylsulfanyl-5-(6-fluoro-quinolin-2-ylmethoxy)-1-(4-pyridin-2-yl-benzyl)-1H-indol-2-yl]-2,2-dimethyl-propionic acid (Compound 2-207); 3-[3-tert-butylsulfanyl-5-(5-ethyl-pyridin-2-ylmethoxy)-1-(4-pyridin-3-yl-benzyl)-1H-indol-2-yl]-2,2-dimethyl-propionic acid (Compound 2-208); 3-[3-tert-butylsulfanyl-1-(4-pyridin-3-yl-benzyl)-5-(quinolin-2-ylmethoxy)-1H-indol-2-yl]-2,2-dimethyl-propionic acid (Compound 2-209); 3-[3-tert-butylsulfanyl-5-(6-fluoro-quinolin-2-ylmethoxy)-1-(4-pyridin-3-yl-benzyl)-1H-indol-2-yl]-2,2-dimethyl-propionic acid (Compound 2-210); 3-[3-tert-butylsulfanyl-5-(5-methyl-pyridin-2-ylmethoxy)-1-(4-pyridin-2-yl-benzyl)-1H-indol-2-yl]-2,2-dimethyl-propionic acid (Compound 2-211); 3-[3-tert-butylsulfanyl-5-(5-ethyl-pyridin-2-ylmethoxy)-1-(4-pyridin-2-yl-benzyl)-1H-indol-2-yl]-2,2-dimethyl-propionic acid (Compound 2-212); 3-[3-tert-butylsulfanyl-1-(4-pyridin-2-yl-benzyl)-5-(quinolin-2-ylmethoxy)-1H-indol-2-yl]-2,2-dimethyl-propionic acid (Compound 2-213); 3-[3-tert-butylsulfanyl-5-(5-methyl-pyridin-2-ylmethoxy)-1-(4-pyridin-3-yl-benzyl)-1H-indol-2-yl]-2,2-dimethyl-propionic acid (Compound 2-214); 3-[3-tert-butylsulfanyl-1-[4-(4-methoxy-pyridin-2-yl)-benzyl]-5-(5-methyl-pyridin-2-ylmethoxy)-1H-indol-2-yl]-2,2-dimethyl-propionic acid (Compound 2-215; 3-[3-tert-butylsulfanyl-5-(quinolin-2-ylmethoxy)-1-(4-(3-methyoxypyridin-2-yl)-benzyl)-1H-indol-2-yl]-2,2-dimethyl-propionic acid (Compound 2-216); 3-[3-tert-butylsulfanyl-5-(5-methyl-pyridin-2-ylmethoxy)-1-(4-(3-methyoxypyridin-2-yl)-benzyl)-1H-indol-2-yl]-2,2-dimethyl-propionic acid (Compound 2-217); 3-[3-tert-butylsulfanyl-5-(5-ethyl-pyridin-2-ylmethoxy)-1-(4-(3-methyoxypyridin-2-yl)-benzyl)-1H-indol-2-yl]-2,2-dimethyl-propionic acid (Compound 2-218); 3-[3-tert-butylsulfanyl-5-(5-methyl-pyridin-2-ylmethoxy)-1-(4-(4-trifluoromethypyridin-2-yl)-benzyl)-1H-indol-2-yl]-2,2-dimethyl-propionic acid (Compound 2-219); 3-[3-tert-butylsulfanyl-5-(5-ethyl-pyridin-2-ylmethoxy)-1-(4-(4-trifluoromethypyridin-2-yl)-benzyl)-1H-indol-2-yl]-2,2-dimethyl-propionic acid (Compound 2-220); 3-[3-tert-butylsulfanyl-5-(quinolin-2-ylmethoxy)-1-(4-(4-trifluoromethypyridin-2-yl)-benzyl)-1H-indol-2-yl]-2,2-dimethyl-propionic acid (Compound 2-221); 3-[3-tert-butylsulfanyl-5-(5-methyl-pyridin-2-ylmethoxy)-1-(4-(5-fluoropyridin-3-yl)-benzyl)-H-indol-2-yl]-2,2-dimethyl-propionic acid (Compound 2-222); 3-[3-tert-butylsulfanyl-5-(5-ethyl-pyridin-2-ylmethoxy)-1-(4-(5-fluoropyridin-3-yl)-benzyl)-1H-indol-2-yl]-2,2-dimethyl-propionic acid (Compound 2-223); 3-[3-tert-butylsulfanyl-5-(quinolin-2-ylmethoxy)-1-(4-(5-fluoropyridin-3-yl)-benzyl)-1H-indol-2-yl]-2,2-dimethyl-propionic acid (Compound 2-224); 3-[3-tert-butylsulfanyl-5-(2,3-dimethyl-pyridin-6-ylmethoxy)-1-(4-(2-methoxypyridin-5-yl)-benzyl)-1H-indol-2-yl]-2,2-dimethyl-propionic acid (Compound 2-225); 3-[3-tert-butylsulfanyl-5-(2,3-dimethyl-pyridin-6-ylmethoxy)-1-(4-(3-trifluoromethylpyridin-2-yl)-benzyl)-1H-indol-2-yl]-2,2-dimethyl-propionic acid (Compound 2-226); 3-[3-tert-butylsulfanyl-5-(2,3-dimethyl-pyridin-6-ylmethoxy)-1-(4-(4-trifluoromethylpyridin-2-yl)-benzyl)-1H-indol-2-yl]-2,2-dimethyl-propionic acid (Compound 2-227); 3-[3-tert-butylsulfanyl-5-(2,3-dimethyl-pyridin-6-ylmethoxy)-1-(4-(3-fluoropyridin-2-yl)-benzyl)-1H-indol-2-yl]-2,2-dimethyl-propionic acid (Compound 2-228); 3-[3-tert-butylsulfanyl-5-(2,3-dimethyl-pyridin-6-ylmethoxy)-1-(4-(5-fluoropyridin-3-yl)-benzyl)-1H-indol-2-yl]-2,2-dimethyl-propionic acid (Compound 2-229); 3-[3-tert-butylsulfanyl-5-(2,3-dimethyl-pyridin-6-ylmethoxy)-1-(4-(4-methoxypyridin-2-yl)-benzyl)-1H-indol-2-yl]-2,2-dimethyl-propionic acid (Compound 2-230); 3-[3-tert-butylsulfanyl-5-(2,3-dimethyl-pyridin-6-ylmethoxy)-1-(4-(pyridin-2-yl)-benzyl)-1H-indol-2-yl]-2,2-dimethyl-propionic acid (Compound 2-231); 3-[3-tert-butylsulfanyl-5-(5-methyl-pyridin-2-ylmethoxy)-1-(4-(2-methoxy-pyridin-3-yl)-benzyl)-1H-indol-2-yl]-2,2-dimethyl-propionic acid (Compound 2-232); 3-[3-tert-butylsulfanyl-5-(5-ethyl-pyridin-2-ylmethoxy)-1-(4-(2-methoxy-pyridin-3-yl)-benzyl)-1H-indol-2-yl]-2,2-dimethyl-propionic acid (Compound 2-233); 3-[3-tert-butylsulfanyl-5-(quinolin-2-ylmethoxy)-1-(4-(2-methoxy-pyridin-3-yl)-benzyl)-1H-indol-2-yl]-2,2-dimethyl-propionic acid (Compound 2-234); 3-[3-tert-butylsulfanyl-1-(6′-methoxy-[2,3′]bipyridinyl-5-ylmethyl)-5-(pyridin-2-ylmethoxy)-1H-indol-2-yl]-2,2-dimethyl-propionic acid (Compound 3-1); 3-[3-tert-butylsulfanyl-1-[6-(4-methoxy-phenyl)-pyridin-3-ylmethyl]-5-(pyridin-2-ylmethoxy)-1H-indol-2-yl]-2,2-dimethyl-propionic acid (Compound 3-2); 3-{3-tert-butylsulfanyl-5-(pyridin-2-ylmethoxy)-1-[6-(4-trifluoromethoxy-phenyl)-pyridin-3-ylmethyl]-1H-indol-2-yl}-2,2-dimethyl-propionic acid (Compound 3-3); 3-[3-tert-butylsulfanyl-1-[5-(4-methoxy-phenyl)-pyridin-2-ylmethyl]-5-(pyridin-2-ylmethoxy)-1H-indol-2-yl]-2,2-dimethyl-propionic acid (Compound 3-4); 3-{3-tert-butylsulfanyl-5-(pyridin-2-ylmethoxy)-1-[5-(4-trifluoromethoxy-phenyl)-pyridin-2-ylmethyl]-1H-indol-2-yl}-2,2-dimethyl-propionic acid (Compound 3-5); 3-{3-tert-butylsulfanyl-5-isopropyl-1-[4-(6-methoxy-pyridin-3-yl)-benzyl]-1H-indol-2-yl}-2,2-dimethyl-propionic acid (Compound 4-1); 3-{3-tert-butylsulfanyl-5-hydroxy-1-[4-(6-methoxy-pyridin-3-yl)-benzyl]-1H-indol-2-yl}-2,2-dimethyl-propionic acid (Compound 4-2).

### Synthesis of Compounds

Compounds of Formula (G), Formula (G-I), and Formula (G-II), described in the prior section may be synthesized using standard synthetic techniques known to those of skill in the art or using methods known in the art in combination with methods described herein. In additions, solvents, temperatures and other reaction conditions presented herein may vary according to those of skill in the art.

The starting material used for the synthesis of the compounds of Formula (G), Formula (G-I), and Formula (G-II), described in the prior section may be synthesized or can be obtained from commercial sources, such as, but not limited to, Aldrich Chemical Co. (Milwaukee, Wis.), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Formation of Covalent Linkages by Reaction of an Electrophile with a Nucleophile

The compounds described herein can be modified using various electrophiles or nucleophiles to form new functional groups or substituents. Table 5 entitled “Examples of Covalent Linkages and Precursors Thereof” lists selected examples of covalent linkages and precursor functional groups which yield and can be used as guidance toward the variety of electrophiles and nucleophiles combinations available. Precursor functional groups are shown as electrophilic groups and nucleophilic groups.

TABLE 5
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

Use of Protecting Groups

In the reactions described, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd₀-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

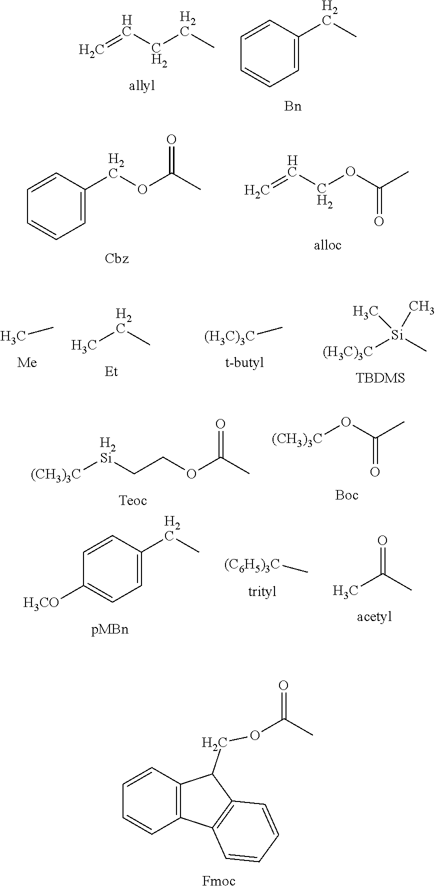

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference in their entirety.

Indole containing compounds can be prepared using standard literature procedures such as those found in Katritzky, “Handbook of Heterocyclic Chemistry” Pergamon Press, Oxford, 1986; Pindur et al, J. Heterocyclic Chem., vol 25, 1, 1987, and Robinson “The Fisher Indole Synthesis”, John Wiley & Sons, Chichester, N.Y., 1982, each of which is herein incorporated by reference in their entirety.

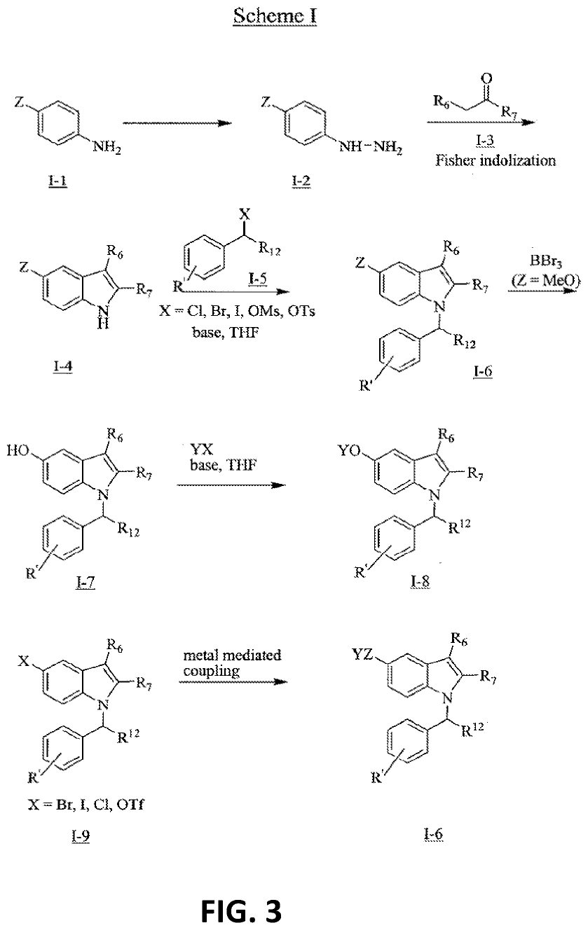

A non-limiting example of the synthetic approach toward indole compounds of Formula (G), Formula (G-I) and Formula (G-II), is shown according to reaction Scheme I shown in FIG. 3, wherein a 4-substituted anilines (I-1) can be converted to the corresponding hydrazine (I-2) using standard methodology. Reaction of hydrazine (I-2) with an appropriately substituted ketone (I-3) under standard Fisher-indolization conditions yields the indole (I-4). Indole (I-6) results from the N-alkylation of (I-4) with a benzyl halide (I-5) (or tosylate (OTs) or mesylate (OMs)) in a solvent such as tetrahydrofuran (THF) or dimethylformamide (DMF) in the presense of a base such as NaH. In the case where the 5-substituent on the indole ring is methoxy (i.e. Z is MeO) the methyl group can be removed under standard conditions, for example using BBr₃, in a solvent such as CH₂Cl₂ to afford the phenol (I-7). This phenol can be alkylated using an electrophile (YX) to provide the alkylated product (I-8). Alternatively, in the case when the 5-substituent on the indole ring is, for example, a halide or triflate (OTf; I-7) it can be coupled with a wide variety of reagents using standard metal mediated coupling reactions well known to those skilled in the art of organic synthesis to afford alternate compounds of structure (I-6). Such chemistry is described in Comprehensive Organometallic Chemistry II, vol 12, Pergamon, edited by Abel, Stone and Wilkinson. The Z substitutent of the indole (I-6) can be further modified using standard chemical procedures. In addition, when R₇ or R₆ is a bromo or iodine, standard cross coupling reactions allow the introduction of a variety of functional groups using procedures well known to those practiced in the art of organic synthesis. Furthermore, when R₇ is H, it is possible, under certain conditions, to regioselectively lithiate using a strong base such as nBuLi and then condense the anion with an electrophile to introduce substituents at C-2 (see Hasan et al, J. Org. Chem., 46, 157-164, 1981).

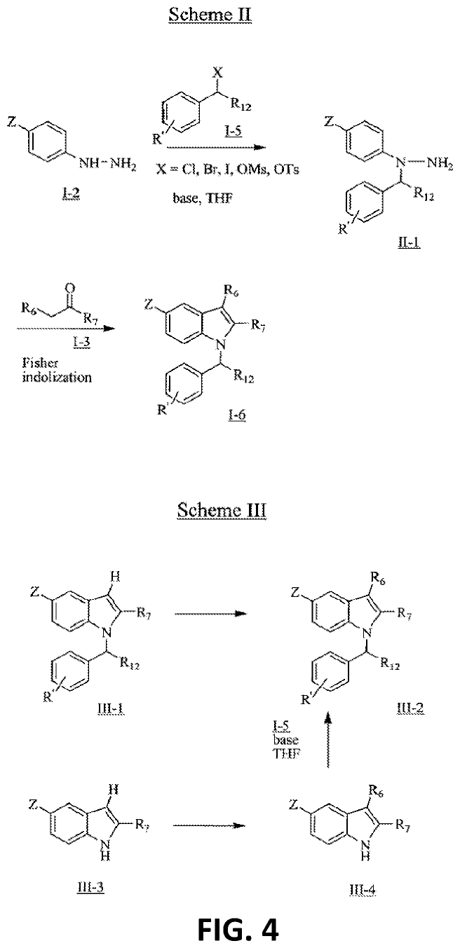

Another non-limiting example of the synthetic approach toward compounds of Formula (G), Formula (G-I) and Formula (G-II) is shown according to reaction Scheme II in FIG. 4. Commencing with the hydrazine I-2, N-alkylation with a benzyl halide (or tosylate or mesylate; I-5) using the conditions described above, provides the hydrazine derivative (II-1). Reaction with an appropriately substituted ketone (I-3) using standard Fisher indolization conditions provides the indole (I-6).

Another non-limiting example of the synthetic approach toward compounds of Formula (G), Formula (G-I) and Formula (G-II) is shown according to reaction Scheme III in FIG. 4, wherein 3-H-indoles (III-1) can be prepared directly using the procedures described above or, alternatively, they can be prepared from 3-thioindoles by treatment with moist AlCl₃ in a solvent such as CH₂Cl₂. Functionalzation at the 3-position can be achieved using a variety of reactions and procedures to allow the introduction of a wide range of substituents. By way of example only, acylation using an acid chloride (or anhydride) in the presence of a Lewis acid such as AlCl₃, allows for the introduction of acyl groups (I-6; R₆═C(O)R′) see Murakami et al. Heterocycles, v14, 1939-1941, 1980 and references cited therein. Commencing with (III-1), and using, by way of example only, sulfenic chlorides in a suitable solvent, compounds of general structure (III-2) wherein R₆ is SR″ can be prepared (Raban, J. Org. Chem., v45, 1688, 1980). Similar chemistry using indole (III-3) can be performed or, alternatively, diarlydisulfides in the presence of a base such as NaH in DMF can be used to generate (III-4) (Atkinson et al, Synthesis, 480-481, 1988). The reaction of electron deficient olefins with 3-H indoles (III-1) or (III-3) in the presence of a Lewis acid (such as Yb(OTf)₃.3H₂O) allows the installation of 3-alkyl substituents of general structure (III-2) or (III-4) (where R₆ is a substituted alkyl group; see Harrington and Kerr, Synlett, 1047-1048, 1996). Alternatively, indole (III-3) can be reacted with benzyl derivatives (I-5) in warm DMF to yield (III-4) where R₆ is a substituted benzyl group (Jacobs et al, J. Med. Chem., v36, 394-409, 1993).

### Further Synthesis of Indole and Indole-Type Compounds

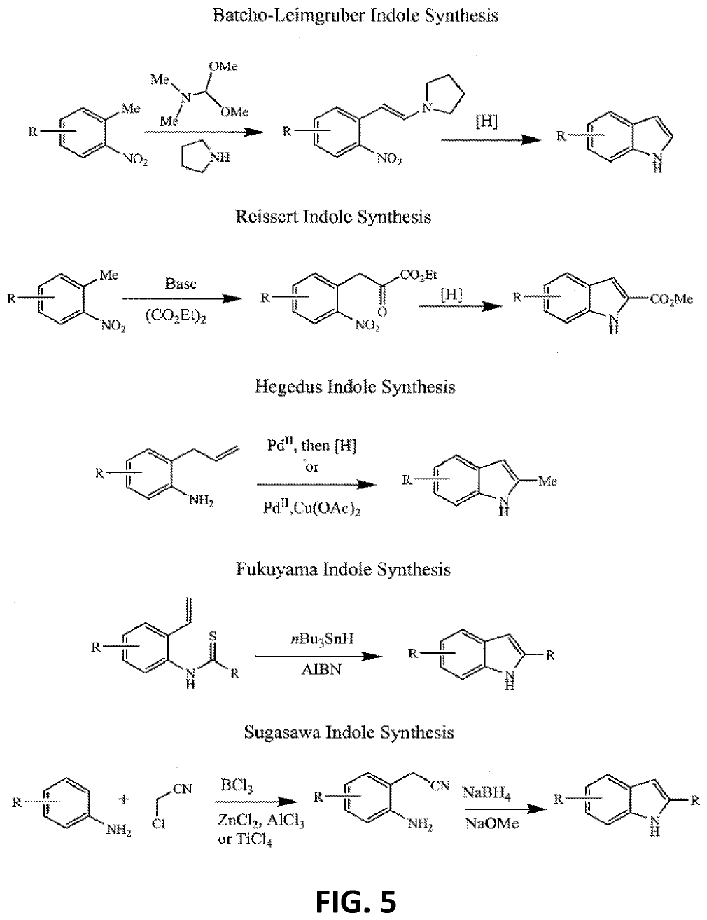

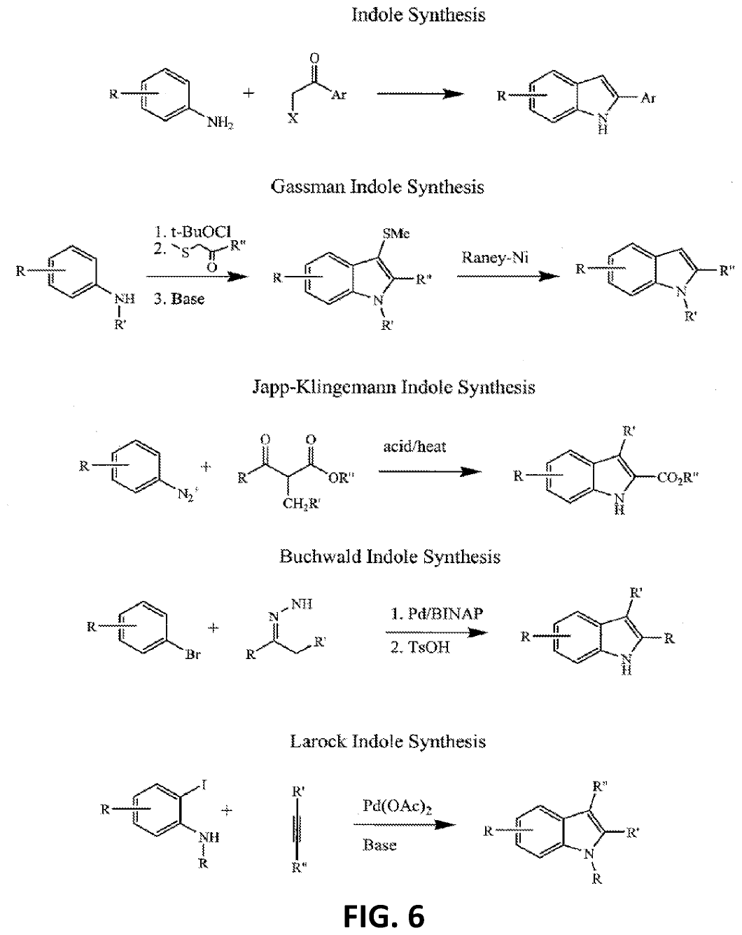

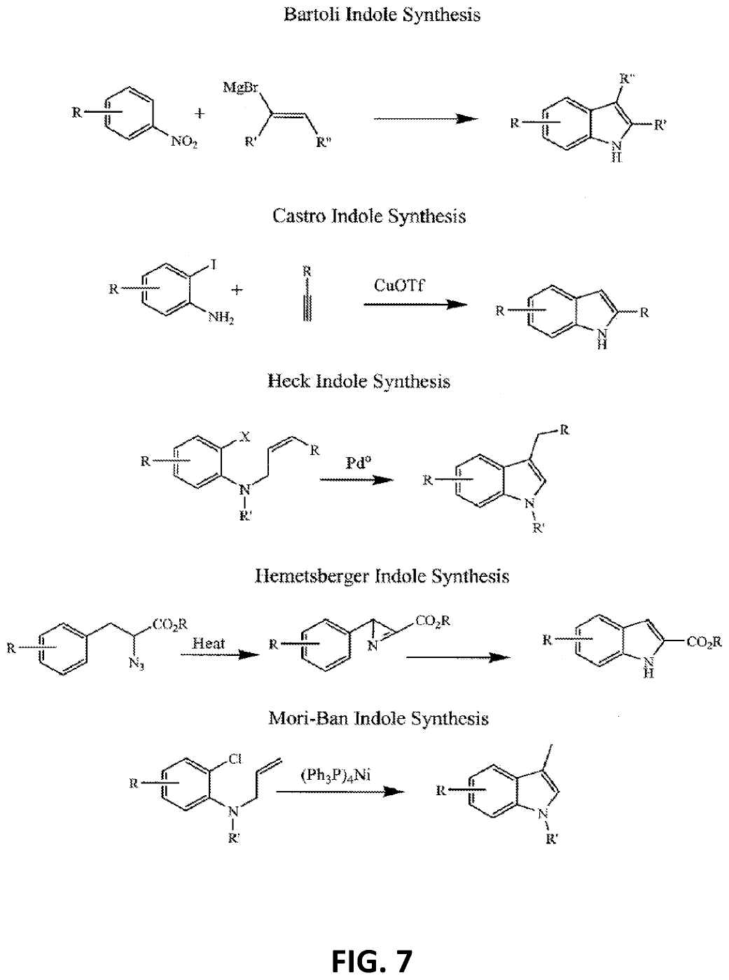

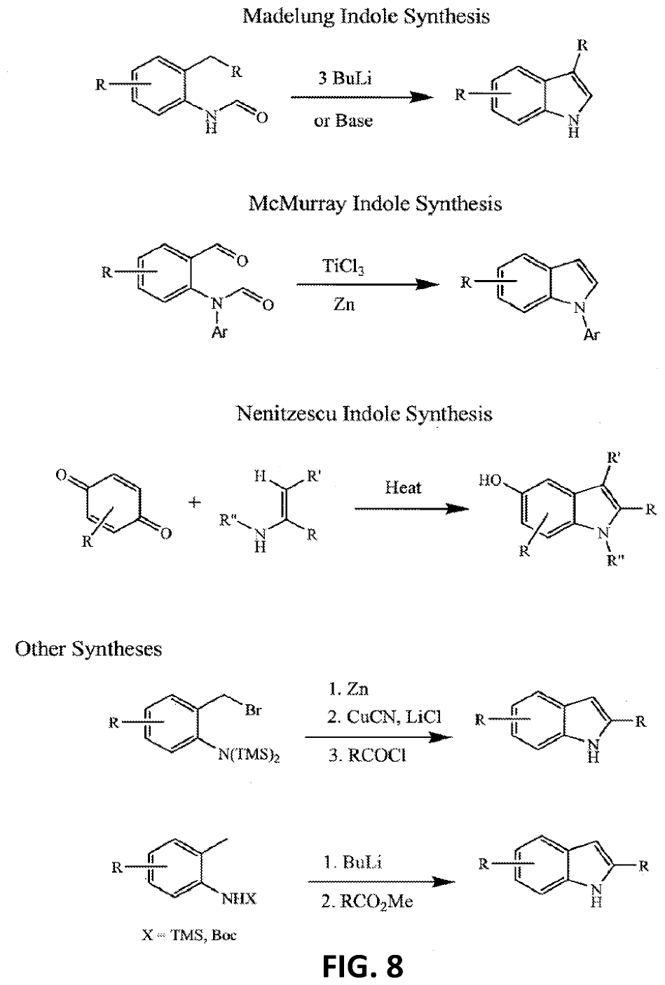

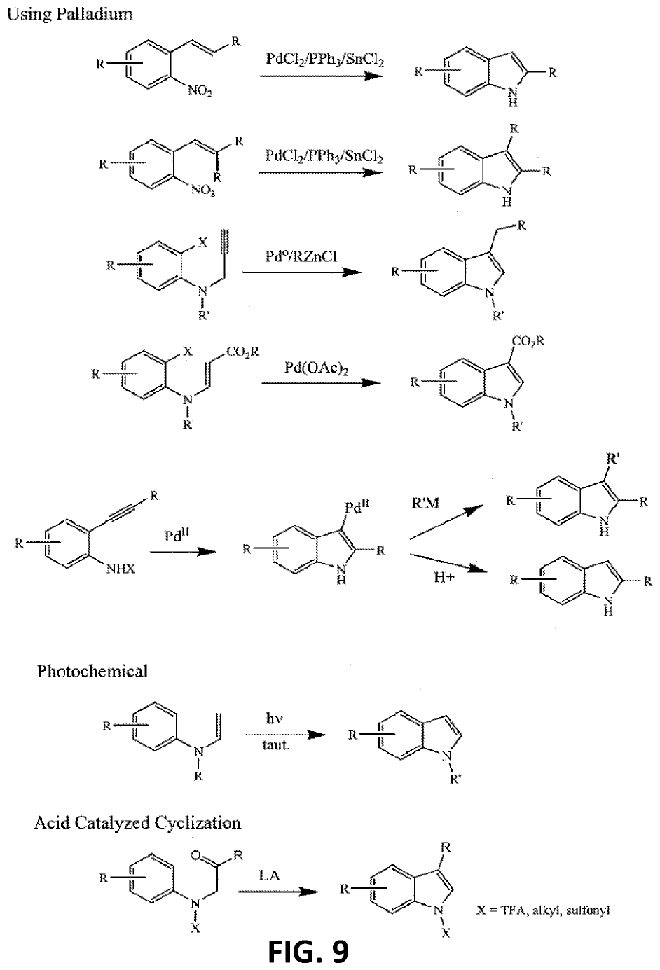

Additional non-limiting examples of the synthetic strategy toward indole or indole-like scaffolds for compounds of Formula (G), Formula (G-I) and Formula (G-II), include modifications to various syntheses of indoles, including, but not limited to; Batcho-Leimgruber Indole Synthesis, Reissert Indole Synthesis, Hegedus Indole Synthesis, Fukuyama Indole Synthesis, Sugasawa Indole Synthesis, Bischler Indole Synthesis, Gassman Indole Synthesis, Fischer Indole Synthesis, Japp-Klingemann Indole Synthesis, Buchwald Indole Synthesis, Larock Indole Synthesis, Bartoli Indole Synthesis, Castro Indole Synthesis, Hemetsberger Indole Synthesis, Mori-Ban Indole Synthesis, Madelung Indole Synthesis, Nenitzescu Indole Synthesis, and other unnamed reactions. Non-limiting examples of such synthetic methods are shown in FIGS. 5 to 9.

### Further Forms of Compounds

Compounds of Formula (G), Formula (G-I) and Formula (G-II), can be prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Alternatively, compounds of Formula (G), Formula (G-I) and Formula (G-II), can be prepared as a pharmaceutically acceptable base addition salts (which is a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

Compounds of Formula (G), Formula (G-I) and Formula (G-II), can be prepared as a pharmaceutically acceptable salts formed when an acidic proton present in the parent compound either is replaced by a metal ion, for example an alkali metal ion, an alkaline earth ion, or an aluminum ion; or coordinates with an organic base. In addition, the salt forms of the disclosed compounds can be prepared using salts of the starting materials or intermediates.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds of Formula (G), Formula (G-I) and Formula (G-II), can be conveniently prepared or formed during the processes described herein. By way of example only, hydrates of compounds of Formula (G), Formula (G-I) and Formula (G-II), can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Compounds of Formula (G), Formula (G-I) and Formula (G-II), may be in various forms, including but not limited to, amorphous forms, milled forms and nano-particulate forms. In addition, compounds of Formula (G), Formula (G-I) and Formula (G-II), include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Compounds of Formula (G), Formula (G-I) and Formula (G-II), in unoxidized form can be prepared from N-oxides of compounds of Formula (G), Formula (G-I) and/or Formula (G-II), by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

Compounds of Formula (G), Formula (G-I) and Formula (G-II), can be prepared as prodrugs. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug.

Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Additionally, prodrug derivatives of compounds of Formula (G), Formula (G-I) and Formula (G-II), can be prepared by methods known to those of ordinary skill in the art (e.g., for further details see Saulnier et al., (1994), Bioorganic and Medicinal Chemistry Letters, Vol. 4, p. 1985). By way of example only, appropriate prodrugs can be prepared by reacting a non-derivatized compound of any of Formula (G), Formula (G-I), or Formula (G-II), with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds may be a prodrug for another derivative or active compound.

Sites on the aromatic ring portion of compounds of Formula (G), Formula (G-I) and Formula (G-II), can be susceptible to various metabolic reactions, therefore incorporation of appropriate substituents on the aromatic ring structures, such as, by way of example only, halogens can reduce, minimize or eliminate this metabolic pathway.

The compounds described herein may be labeled isotopically (e.g. with a radioisotope) or by another other means, including, but not limited to, the use of chromophores or fluorescent moieties, bioluminescent labels, or chemiluminescent labels. The compounds of Formula (G), Formula (G-I) and Formula (G-II), may possess one or more stereocenters and each center may exist in the R or S configuration. The compounds presented herein include all diastereomeric, enantiomeric, and epimeric forms as well as the appropriate mixtures thereof. Compounds of Formula (G), Formula (G-I) and Formula (G-II), can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds described herein, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chiral chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981, herein incorporated by reference in its entirety.

Additionally, the compounds and methods provided herein may exist as geometric isomers. The compounds and methods provided herein include all cis, trans, syn, anti, entgegen (E), and zusammen (Z) isomers as well as the appropriate mixtures thereof. In some situations, compounds may exist as tautomers. All tautomers are included within the formulas described herein are provided by compounds and methods herein. In additional embodiments of the compounds and methods provided herein, mixtures of enantiomers and/or diastereoisomers, resulting from a single preparative step, combination, or interconversion may also be useful for the applications described herein.

### Routes of Administration

Suitable routes of administration include, but are not limited to, intravenous, oral, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

Alternately, one may administer the compound in a local rather than systemic manner, for example, via injection of the compound directly into an organ, often in a depot preparation or sustained release formulation. Such long acting formulations may be administered by implantation (for example subcutaneously or intramuscularly) or by intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation, or in the form of an intermediate release formulation.

### EXAMPLES

These examples are provided for illustrative purposes only and not to limit the scope of the claims provided herein.

### Preparation of Intermediates used in the Synthesis of Compounds of Formula (G), Formula (G-I), and Formula (G-II)

Starting materials and intermediates used in the synthesis of compounds of Formula (G), Formula (G-I), and Formula (G-II) are commercially available or can be synthesized by synthetic methods known in the art or described herein. The preparation of intermediates, such as, for example, those shown in Table 6, which are used herein and not commercially available is described below. Other intermediates not specifically mentioned herein and used in the synthesis of compounds of Formula (G), Formula (G-I), and Formula (G-II), can be prepared using the methods described herein or known in the art.

TABLE 6
Intermediates used in the Synthesis of Compounds of Formula (G), Formula (G-I), and Formula (G-II)

| Compound | | | Method for |
|---|---|---|---|
| # | Structure | Compound Name | Preparation |
| Int-5 | 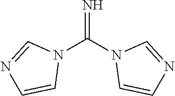 [SMILES: N=C(n1ccnc1)n1ccnc1] | C-(Di-imidazol-1-yl)- methyleneamine | Route 8, Step 1 |
| Int-10 | 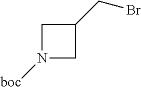 | 3-Bromomethyl-azetidine-1- carboxylic acid tert-butyl ester | Route 1, Steps 1-3a SM: 3- Azetidinecarboxylic acid (Sigma Aldrich) |
| Int-19 | 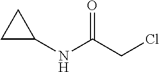 | 2-Chloro-N-cyclopropyl- acetamide | Route 2, Step 1 SM: Cyclopropylamine (Sigma Aldrich) |
| Int-20 | 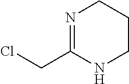 | 2-Chloromethyl-1,4,5,6- tetrahydro-pyrimidine Hydrochloride | Route 3, Steps 1-2 SM: Chloro-acetonitrile (Sigma Aldrich) |
| Int-21 | 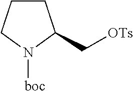 | (S)-2-(Toluene-4- sulfonyloxymethyl)- pyrrolidine-1-carboxylic acid tert-butyl ester | Route 1, Step 3c SM: (S)-(−)-1-(tert- Butoxycarbonyl)-2- pyrrolidinemethanol (Sigma Aldrich) |
| Int-22 | 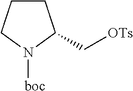 | (R)-2-(Toluene-4- sulfonyloxymethyl)- pyrrolidine-1-carboxylic acid tert-butyl ester | Route 1, Step 3c SM: (R)-(+)-1-(tert- Butoxycarbonyl)-2- pyrrolidinemethanol (Sigma Aldrich) |
| Int-23 | 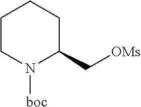 | (S)-2- Methanesulfonyloxymethyl- piperidine-1-carboxylic acid tert-butyl ester | Route 1, Step 3d SM: 1-Boc-(S)-2- piperidinemethanol (Chem Impex) |
| Int-24 | 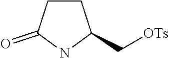 [SMILES: Cc1ccc(S(=O)(=O)OC[C@@H]2CCC(=O)N2)cc1] | Toluene-4-sulfonic acid (S)-5- oxo-pyrrolidin-2-ylmethyl ester | Route 1, Step 3c SM: (S)-(+)-5- (Hydroxymethyl)-2- pyrrolidine (Sigma Aldrich) |
| Int-25 | 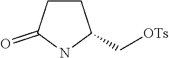 [SMILES: Cc1ccc(S(=O)(=O)OC[C@H]2CCC(=O)N2)cc1] | Toluene-4-sulfonic acid (R)-5- oxo-pyrrolidin-2-ylmethyl ester; | 6-27-Route 1, Step 3c SM: (R)-(−)-5- (Hydroxymethyl)-2- pyrrolidinone (Acros Organics) |
| Int-27 | 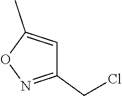 | 3-Chloromethyl-5-methyl- isoxazole Hydrochloride | Route 4, Step 4 SM: (5-Methylisoxazol- 3-yl)methanol (Acros Organics) |
| Int-28 | 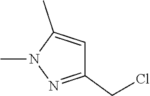 | 3-Chloromethyl-1,5-dimethyl- 1H-pyrazole Hydrochloride | Route 4, Step 4 SM: (1,5-Dimethyl-1H- pyrazol-3-yl)methanol (Acros Organics) |
| Int-29 | 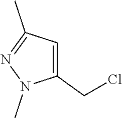 | 5-Chloromethyl-1,3-dimethyl- 1H-pyrazole Hydrochloride | Route 4, Step 4 SM: (1,3-Dimethyl-1H- pyrazol-5-yl)methanol (Acros Organics) |
| Int-30 | 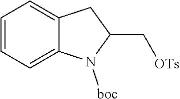 | 2-(Toluene-4- sulfonyloxymethyl)-2,3- dihydro-indole-1-carboxylic acid tert-butyl ester | Route 1, Steps 1-3c SM: Indoline-2- carboxylic Acid (Sigma Aldrich) |
| Int-31 | 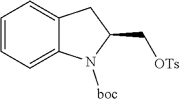 | (S)-2-(Toluene-4- sulfonyloxymethyl)-2,3- dihydro-indole-1-carboxylic acid tert-butyl ester | Route 1, Steps 1, 3c SM: (S)-(+)-2- Indolinemethanol (Sigma Aldrich) |
| Int-32 | 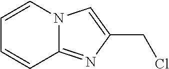 [SMILES: ClCc1cn2ccccc2n1] | 2-Chloromethyl-imidazo[1,2- a]pyridine | Route 4, Step 4 SM: Imidazo[1,2- a]pyridin-2-ylmethanol (Acros Organics) |
| Int-33 | 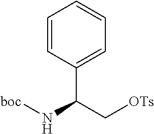 | Toluene-4-sulfonic acid (S)-2- tert-butoxycarbonylamino-2- phenyl-ethyl ester | Route 1, Steps 1, 3c SM: (S)-(+)-2- Phenylglycinol (Sigma Aldrich) |
| Int-34 | 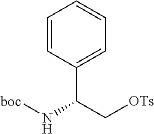 | Toluene-4-sulfonic acid (R)-2- tert-butoxycarbonylamino-2- phenyl-ethyl ester | Route 1, Step 3c SM: (R)-(−)-N-(tert- Butoxycarbonyl)-2- phenylglycinol (Sigma Aldrich) |
| Int-38 | 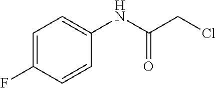 [SMILES: O=C(CCl)Nc1ccc(F)cc1] | 2-Chloro-N-(4-fluoro-phenyl)- acetamide | Route 2, Step 1 SM: 4-Fluoroaniline (Sigma Aldrich) |
| Int-39 | 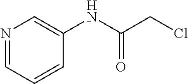 [SMILES: O=C(CCl)Nc1cccnc1] | 2-Chloro-N-pyridin-3-yl- acetamide | Route 2, Step 1 SM: 3-Aminopyridine (Sigma Aldrich) |
| Int-44 | 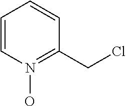 | 2-Chloromethyl-pyridin-1-ol | Route 4, Step 1 SM: 2-Chloromethyl- pyridine Hydrochloride (Sigma Aldrich) |
| Int-45 | 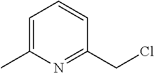 | 2-Chloromethyl-6-methyl- pyridine Hydrochloride | Route 4, Step 4 SM: 6-Methyl-2- pyridinemethanol (Sigma Aldrich) |
| Int-46 | 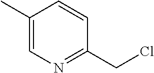 | 2-Chloromethyl-5-methyl- pyridine Hydrochloride | Route 4, Steps 1-4 SM: 2,5-Lutidine (Sigma Aldrich) |
| Int-47 | 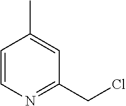 | 2-Chloromethyl-4-methyl- pyridine Hydrochloride | Route 4, Steps 1-4 SM: 2,4-Lutidine (Sigma Aldrich) |
| Int-48 | 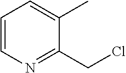 | 2-Chloromethyl-3-methyl- pyridine Hydrochloride | Route 4, Steps 1-4 SM: 2,3-Lutidine (Sigma Aldrich) |
| Int-49 | 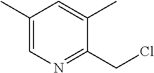 | 2-Chloromethyl-3,5-dimethyl- pyridine Hydrochloride | Route 4, Steps 1-4 SM: 2,3,5-Collidine (Sigma Aldrich) |
| Int-50 | 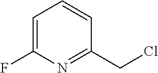 | 2-Chloromethyl-6-fluoro- pyridine Hydrochloride | Route 5, Step 3c SM: 2-Fluoro-6- methylpyridine (Oakwood Product) |
| Int-51 | 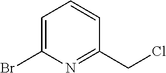 | 2-Chloromethyl-6-bromo- pyridine Hydrochloride | Route 4, Step 4 SM: (6-Bromo-pyridin- 2-yl)-methanol (Sigma Aldrich) |
| Int-52 | 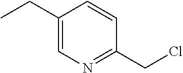 | 2-Chloromethyl-5-ethyl- pyridine | Route 4, Steps 1-4 SM: 5-Ethyl-2- methylpyridine (Sigma Aldrich) |
| Int-53 | 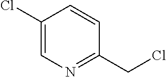 | 2-Chloromethyl-5-chloro- pyridine | Route 1, Step 2; Route 4, Step 4 SM: 5-Chloropyridine-2- carboxylic Acid (Matrix Scientific) |
| Int-54 | 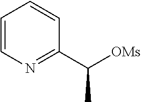 | Methanesulfonic acid (S)-1- pyridin-2-yl-ethyl ester | Route 1, Step 3 SM: (R)-alpha-Methyl- 2-pyridinemethanol (Sigma Aldrich) |
| Int-55 | 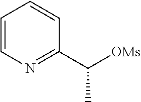 | Methanesulfonic acid (R)-1- pyridin-2-yl-ethyl ester | Route 1, Step 3 SM: (S)-alpha-Methyl- 2-pyridinemethanol (Sigma Aldrich) |
| Int-57 | 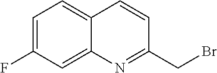 [SMILES: Fc1ccc2ccc(CBr)nc2c1] | 2-Bromomethyl-7-fluoro- quinoline | Route 5, Step 3a SM: 7-Fluoro-2- methylquinoline (Sigma Aldrich) |
| Int-58 | 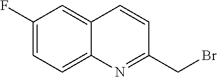 [SMILES: Fc1ccc2nc(CBr)ccc2c1] | 2-Bromomethyl-6-fluoro- quinoline | Route 5, Step 3a SM: 6-Fluoro-2- methylquinoline (Sigma Aldrich) |
| Int-59 | 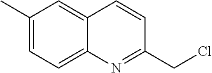 | 2-Chloromethyl-6-methyl- quinoline | Route 4, Steps 1-4 SM: 2,6- Dimethylquinoline (Sigma Aldrich) |
| Int-60 | 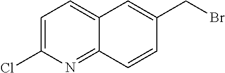 [SMILES: Clc1ccc2cc(CBr)ccc2n1] | 2-Chloro-6-bromomethyl- quinoline | Route 5, Steps 1-3a SM: Cinnamoyl chloride (Sigma Alrich) and p- toluidine (Sigma Aldrich) |
| Int-71 | 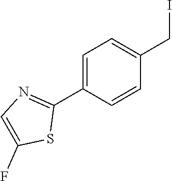 [SMILES: Fc1cnc(-c2ccc(CI)cc2)s1] | 5-Fluoro-2-(4-iodomethyl- phenyl)-thiazole | Route 6, Step 1-2a; Route 1, Step 3b |
| Int-72 | 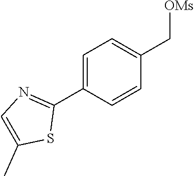 [SMILES: Cc1cnc(-c2ccc(COS(C)(=O)=O)cc2)s1] | Methanesulfonic acid 4-(5- methyl-thiazol-2-yl)-benzyl ester | Route 6, Step 1-2b; Route 1, Step 3d |
| Int-73 | 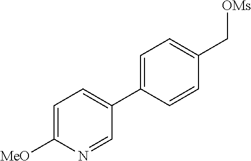 [SMILES: COc1ccc(-c2ccc(COS(C)(=O)=O)cc2)cn1] | Methanesulfonic acid 4-(6- methoxy-pyridin-3-yl)-benzyl ester | Route 6, Step 1; Route 1, Step 3d |
| Int-74 | 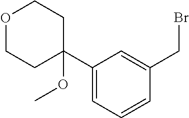 [SMILES: COC1(c2cccc(CBr)c2)CCOCC1] | 4-(3-Bromomethyl-phenyl)-4- methoxy-tetrahydro-pyran | Route 9, Step 1; Route 5, Step 3a |
| Int-75 | 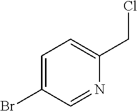 | 5-Bromo-2-chloromethyl- pyridine | Route 4, Step 4 (5-Bromo-pyridin-2-yl)- methanol (Biofine International) |
| Int-76 | 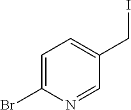 | 2-Bromo-5-iodomethyl- pyridine | Route 1, Step 3b (6-Bromo-pyridin-3-yl)- methanol (Biofine International) |
| Int-118 | 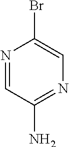 | 5-Bromo-pyrazin-2-ylamine | Route 5, Step 3b SM: Aminopyrazine (Lancaster) |
| Int-135 | 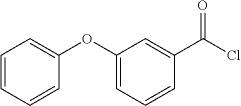 [SMILES: O=C(Cl)c1cccc(Oc2ccccc2)c1] | 3-Phenoxy-benzoyl chloride | Route 7, Step 1 SM: 3-Phenoxy-benzoic acid (Sigma Aldrich) |
| Int-136 | 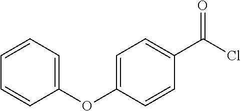 [SMILES: O=C(Cl)c1ccc(Oc2ccccc2)cc1] | 4-Phenoxy-benzoyl chloride | Route 7, Step 1 SM: 4-Phenoxy-benzoic acid (Sigma Aldrich) |
| Int-140 | 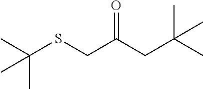 | 1-tert-Butylsulfanyl-4,4- dimethyl-pentan-2-one | Route 10, Steps 1-2 |

### Route 1:

### Step 1: BOC Protection (Int-10)

3-Azetidinecarboxylic acid (Sigma Aldrich, 0.25 g, 2.5 mmol) was dissolved in tBuOH (5 mL) and 1N NaOH (2.7 mL, 2.7 mmol). Di-tert-butyl dicarbonate (0.59 g, 2.7 mmol) was added, and the reaction was stirred overnight at room temperature. The reaction was diluted with water, acidified slowly to pH 4 with 1N HCl, and the mixture was extracted with EtOAc until all product was removed from the aqueous layer by ninhydrin stain. The combined organic layers were dried, filtered, and concentrated to give the desired product.

### Step 2: Borane Reduction (Int-10)

Acid from Step 1 (0.7 g, 3.5 mmol) was dissolved in THF and cooled to 0° C. under N₂. Borane-THF complex was added to the solution, and the reaction was stirred at room temperature overnight. The reaction was cooled to 0° C. and quenched with water. The mixture was extracted 3 times with EtOAc, the combined organic layers were dried over MgSO₄, filtered, and concentrated. The crude material was filtered through a plug of silica gel and eluted with EtOAc to give the desired compound.

### Step 3a: Br₂ Bromide Formation (Int-10)

Triphenylphosphine (1.7 g, 6.5 mmol) was dissolved in DMF and cooled to 0° C. Bromine (0.31 mL, 5.9 mmol) as added slowly, and the solution was stirred for 30 minutes. Alcohol from Step 2 (0.32 g, 2.0 mmol) was added in DMF and the reaction was stirred at room temperature overnight. The mixture was diluted with water, extracted 3 times with EtOAc, and the combined organic layers were dried over MgSO₄, filtered, and concentrated. The crude material was filtered through a plug of silica gel and eluted with EtOAc to give the desired compound.

### Step 3b: I₂ Iodide Formation (Int-73)

(6-Bromo-pyridin-3-yl)-methanol (0.5 g, 2.7 mmol) was dissolved in toluene (20 mL). Triphenylphosphine (0.9 g, 3.5 mmol) and imidazole (0.4 g, 6.0 mmol) were added, followed by a solution of iodine (0.88 g, 3.5 mmol) in toluene dropwise. The reaction was stirred at room temperature for 15 minutes, and then poured into saturated aq. Na₂CO₃. The organic layer was washed with aq. sodium thiosulfate, water, then dried over MgSO₄, filtered, and concentrated. The crude material was purified on silica gel (EtOAc:hexanes gradient) to give the desired product.

### Step 3c: Tosylation (Int-21)

(S)-(−)-1-(tert-Butoxycarbonyl)-2-pyrrolidinemethanol (1.0 g, 5.0 mmol) was dissolved in pyridine (3 mL), and toluenesulfonyl chloride (1.0 g, 5.5 mmol) was added. The reaction was stirred overnight at room temperature, and diluted with water and extracted with EtOAc. The combined organic layers were washed with water, dried over MgSO₄, filtered, and concentrated. The residue was purified on silica gel (0 to 10% EtOAc in hexanes) to give the desired product.

### Step 3d: Mesylation (Int-55)

(R)-alpha-Methyl-2-pyridinemethanol (1.0 g, 8.1 mmol) was dissolved in CH₂Cl₂ (20 mL) and cooled to 0° C. Triethylamine (1.7 mL, 12.2 mmol) was added, followed by methanesulfonyl chloride (0.66 mL, 8.4 mmol) dropwise. The reaction was stirred for 30 minutes, and then diluted with CH₂Cl₂, washed with water, dried over MgSO₄, filtered, and concentrated to obtain the desired product.

### Route 2:

### Step 1: Amide Formation (Int-19)

Cyclopropylamine (0.35 mL, 5.0 mmol) and triethylamine (0.7 mL, 5.1 mmol) were dissolved in CH₂Cl₂ (10 mL). The reaction was cooled to −10° C. and chloroacetyl chloride (0.4 mL, 5.0 mmol) was added dropwise. The reaction was stirred at −10° C. for 1 hour, then at room temperature for 2 hours, followed by a quench with water. The aqueous layer was extracted with CH₂Cl₂, and the organic layers were dried, filtered, and concentrated to give the desired product.

### Route 3:

### Step 1: Imine Formation (Int-20)

Chloroacetonitrile (0.5 g, 6.6 mmol) was dissolved in Et₂O (10 mL) and cooled to 0° C. EtOH (0.43 mL, 7.3 mmol) was added, followed by 4N HCl in 1,4-dioxane (15 mL, 59.6 mmol). The reaction was stirred at 0° C. for 4 days, and then concentrated to give the desired product as a white solid.

### Step 2: Cyclization (Int-20)

Imine from Step 1 (0.3 g, 2.0 mmol) was dissolved in EtOH (4 mL) and cooled to 0° C. 1,3-Diaminopropane (0.17 mL, 2.0 mmol) was added, followed by iPr₂NEt (0.35 mL, 2.0 mmol). The reaction was stirred at 0° C. for 2 hours, and then 4N HCl in 1,4-dioxane (0.5 mL, 2 mmol) was added. The mixture was filtered, and the filtrate was concentrated to give the desired product.

### Route 4:

Step 1: mCPBA Oxidation (Int-46)

2,5-Lutidine (5.0 g, 46.7 mmol) was dissolved in CHCl₃ (125 mL) and cooled to 0° C. m-Chloroperoxybenzoic acid (70%; 13.9 g, 55.2 mmol) was added, and the reaction was stirred overnight at room temperature. The mixture was washed with saturated aq. Na₂CO₃, dried over Na₂SO₄, filtered, and concentrated to give the desired product.

### Step 2: Acetylation (Int-46)

The N-oxide from Step 1 (46.7 mmol) was dissolved in acetic anhydride (25 mL) and heated to reflux at 100° C. for one hour. The mixture was cooled to room temperature, and ethanol (46.7 mmol) was slowly added to quench the reaction. The solution was evaporated to dryness and purified on silica gel to give the desired product.

### Step 3: Hydrolysis (Int-46)

Acetate from Step 2 (46.7 mmol) was dissolved in concentrated HCl (20 mL) and refluxed for 1 hour. The reaction was cooled and evaporated to dryness to give an orange solid, which was used directly in the next reaction.

### Step 4: SOCl₂ Chloride Formation (Int-46)

Alcohol from Step 3 (1.0 g, 8.1 mmol) was dissolved in thionyl chloride (3 mL) and stirred at room temperature for 30 minutes under N₂. The mixture was evaporated to dryness to give the desired product as a hydrochloride salt, which was used directly in subsequent reactions.

### Route 5:

### Step 1: Condensation (Int-60)

p-Toluidine (10 g, 60.0 mmol) and triethylamine (8.4 mL, 60.3 mmol) were dissolved in CH₂Cl₂ (200 mL) at room temperature. Cinnamoyl chloride (6.5 g, 60.7 mmol) was added, and the reaction was stirred for 1 hour. The reaction was washed with water, dried, filtered, and concentrated. To the residue was added aluminum chloride (5 g, 37.5 mmol), which was heated neat. After 45 minutes, ice was added to form a precipitate. The mixture was stirred overnight at room temperature. The precipitate was then filtered and dissolved in CH₂Cl₂, washed with 1N HCl, brine, dried over MgSO₄, filtered, and concentrated. The residue was recrystallized from ethanol to give the desired quinolinone product.

### Step 2: POCl₃ Chloride Formation (Int-60)

Quinolinone from Step 1 (3.12 g, 19.6 mmol) was heated to 90° C. in POCl₃ (10 mL). Once no starting material remained, the reaction was cooled and concentrated. The residue was diluted with EtOAc and saturated aq. NaHCO₃, and the aqueous layer was extracted with EtOAc. The combined organics were dried, filtered, and concentrated to give the chloroquinoline product.

### Step 3a: NBS Bromide Formation (Alkyl) (Int-60)

Quinoline from Step 2 (19.6 mmol) was heated to 80° C. for 1 hour in benzene (200 mL) with NBS (3.6 g, 20.2 mmol) and catalytic benzoyl peroxide. The reaction mixture was concentrated and purified on silica gel to give the desired product.

### Step 3b: NBS Bromide Formation (Aryl) (Int-118)

2-Aminopyrazine (4 g, 42 mmol) was dissolved in water (2 mL) and DMSO (70 mL), and NBS (7.5 g, 42 mmol) was added over 1 hour at 0° C. The reaction was warmed to room temperature and stirred overnight. The mixture was poured onto ice and extracted 4 times with EtOAc. The combined organic layers were washed with 5% Na₂CO₃, water, and brine, dried over MgSO₄, filtered, and concentrated. The residue was purified on silica gel to give the desired product.

### Step 3c: NCS Chloride Formation (Int-50)

2-Fluoro-6-methylpyridine (1.11 g, 10 mmol), NCS (2.0 g, 15 mmol), and catalytic benzoyl peroxide were dissolved in benzene and heated to reflux overnight. The reaction was concentrated and diluted with water and EtOAc. The organic layer was washed with saturated aq. NaHCO₃, dried, filtered, and concentrated. The residue was purified on silica gel to give the desired product.

### Route 6:

### Step 1: Suzuki Coupling (Int-71)

To (4-Hydroxymethylphenyl)boronic acid (Combi-Blocks; 1.0 g, 6.6 mmol) in DME/H₂O (16 mL, 2:1) was added 2-bromothiazole (1.2 g, 7.2 mmol) and K₂CO₃ (2.7 g, 19.7 mmol). The reaction was degassed with N₂ for 20 minutes. Pd(PPh₃)₄ (0.76 g, 0.7 mmol) was added and the reaction was further degassed for 10 minutes. The reaction was then heated to 90° C. overnight under N₂. LCMS confirmed the formation of the product. The reaction was partitioned between water and EtOAc and the aqueous layer was extracted twice with EtOAc. The combined organic layers were dried over MgSO₄, filtered, concentrated, and purified on silica gel (EtOAc:hexanes gradient) to give the desired product.

### Step 2a: F-Alkylation (Int-71)

Thiazole from Step 1 (0.35 g, 1.8 mmol) was dissolved in THF (15 mL) and cooled to −78° C. under N₂. n-Butyllithium (1.6M; 4.6 mL, 7.3 mmol) was added dropwise, followed by NFSi (1.2 g, 3.7 mmol). The reaction was quenched at −78° C. with saturated aq. NH₄Cl, and diluted with EtOAc and water. The aqueous layer was extracted twice with EtOAc, and the combined organics were dried over MgSO₄, filtered, and concentrated. The residue was purified on silica gel to give the desired compound.

### Step 2b: Me-Alkylation (Int-72)

Thiazole from Step 1 (0.33 g, 1.7 mmol) was dissolved in THF (15 mL) and cooled to −78° C. under N₂. n-Butyllithium (1.6M; 4.3 mL, 6.7 mmol) was added dropwise, followed by iodomethane (0.16, 2.6 mmol). The reaction was quenched at −78° C. with saturated aq. NH₄Cl, and diluted with EtOAc and water. The aqueous layer was extracted twice with EtOAc, and the combined organics were dried over MgSO₄, filtered, and concentrated. The residue was purified on silica gel to give the desired compound.

### Route 7:

### Step 1: Acid Chloride Formation (Int 135)

3-Phenoxy-benzoic acid (0.50 g, 0.23 mmol) was dissolved in CH₂Cl₂. Oxalyl chloride (0.32 g, 0.25 mmol) was added, followed by 1-2 drops of DMF. The reaction was stirred at room temperature, and then concentrated to give the desired acid chloride.

### Route 8:

### Step 1: Alkylation (Int-5)

To imidazole (0.41 g, 6.0 mmol) in CH₂Cl₂ was added bromoacetonitrile (0.21 g, 2.0 mmol), and the reaction was refluxed for 30 minutes. The mixture was cooled to room temperature and filtered, and the filtrate was concentrated to give the desired product.

### Route 9:

### Step 1: Methylation (Int-74)

To 4-m-Tolyl-tetrahydro-pyran-4-ol (2.5 g, 13.0 mmol) in THF (50 mL) was added sodium hydride (60%; 0.8 g, 20.0 mmol) at room temperature. Iodomethane (1.25 mL, 20 mmol) was added, and the reaction was stirred for 1 hour. The mixture was quenched with water, and the aqueous layer was extracted with EtOAc. The combined organic layers were washed with water, dried over MgSO₄, filtered, and concentrated. The residue was purified on silica gel to give the desired compound.

### Route 10:

### Step 1: Bromination

To 4,4-Dimethyl-pentan-2-one (3.7 mL, 26.3 mmol) in MeOH (2.8 mL) at 0° C. was added bromine (1.34 mL, 26.3 mmol) in a single stream. The reaction was warmed slowly to 10° C. for 30 minutes to initiate the reaction, and then stirred at room temperature for an additional 15 minutes. The reaction was diluted with water and diethyl ether, and the aqueous layer was extracted with diethyl ether three times. The combined organic layers were dried over MgSO₄, filtered, and concentrated to give the desired product as a colourless liquid.

### Step 2: Thiol Addition

Bromide from Step 1 (26.3 mmol) was dissolved in THF (50 mL), and the mixture was cooled to 0° C. 2-Methyl-2-propanethiol (2.45 mL, 21.6 mmol) was added, followed by triethylamine (7.9 mL, 56.8 mmol). The reaction was stirred at room temperature for 18 hours, then diluted with water. The aqueous layer was extracted with diethyl ether, and the combined organic layers were dried over MgSO₄, filtered, and concentrated to give the desired product. Synthesis of Compounds of Formula (G), Formula (G-I) and Formula (G-II)

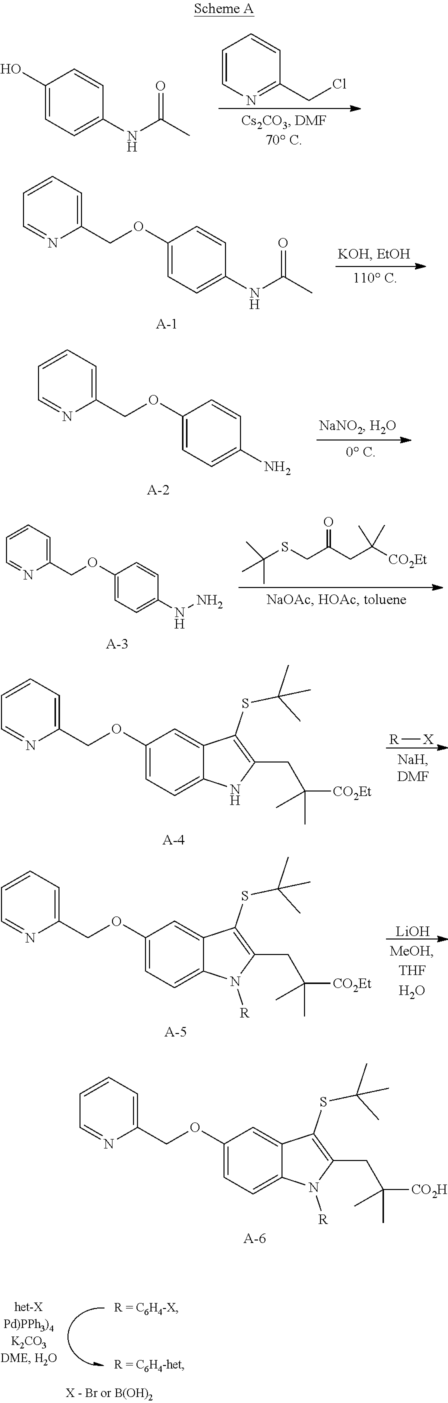

### Example 1: Preparation of Compound 1-2, Compound 2-19, Compound 2-21, Compound 2-35, Compound 2-62, Compound 2-89, Compound 2-195, Compound 2-196, Compound 2-206, Compound 3-1, Compound 3-2, Compound 3-3, Compound 3-4, Compound 3-5, and Compound 4-1

Compound 1-2, Compound 2-19, Compound 2-21, Compound 2-35, Compound 2-62, Compound 2-89, Compound 2-195, Compound 2-196, Compound 2-206, Compound 3-1, Compound 3-2, Compound 3-3, Compound 3-4, Compound 3-5, and Compound 4-1, were prepared as outlined in Scheme A. A detailed illustrative example of the reaction conditions shown in Scheme A is described for the synthesis of 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid (Compound 2-19).

### Step 1: N-[4-(Pyridin-2-ylmethoxy)-phenyl]-acetamide

A mixture of 4-acetamidophenol (Sigma-Aldrich; 73.6 g), 2-chloromethylpyridine hydrochloride (80 g) and cesium carbonate (320 g) in DMF (1 L) was stirred at 70° C. for 2 days. The mixture was cooled, poured into water (2 L) and extracted with EtOAC (×6). The organic layers were washed with brine, dried (MgSO₄) and filtered to give a tan solid (A-1, 114 g) which was used as such in the next step.

### Step 2: 4-(Pyridin-2-ylmethoxy)-phenylamine Hydrochloride

A-1 (114 g) was dissolved in EtOH (1 L) and to this was added KOH (50 g) in water (200 mL). The solution was heated to 110° C. for 2 days, KOH (20 g in 100 mL water) was added and heating continued for a further 2 days. The solution was cooled, the EtOH was removed in vacuo and the residue partitioned between EtOAc and water. After extraction of the water with EtOAc (×3), the organic layers were washed with brine, dried (MgSO₄) and filtered. To this solution was added saturated HCl in EtOAc and a precipitated formed immediately. Collection of the solids by filtration followed by drying under vacuum provided the title compound (A-2, 95 g) as a pink solid.

### Step 3: [4-(Pyridin-2-ylmethoxy)-phenyl]-hydrazine Dihydrochloride

A-2 (95 g) was dissolved in water (1 L) at 0° C. and to this was added NaNO₂ (26 g) in water (100 mL). The diazonium salt was allowed to form over 45 minutes and then it was poured slowly over 15 minutes into a rapidly stirred mixture of Na₂S204 (350 g) in water (1 L) and ether (1 L) at 0° C. Stirring continued for 40 minutes then mixture was made basic using conc. KOH. After extraction using EtOAc (×2) the organic layers were washed with water, then brine, dried (MgSO₄) and filtered. To this solution was added saturated HCl in EtOAc and a precipitated formed immediately. Collection of the solids by filtration followed by drying under vacuum provided the title compound as a tan solid (A-3, 75 g).

### Step 4: 3-[3-tert-Butylsulfanyl-5-(pyridin-2-ylmethoxy)-1H-indol-2-yl]-2,2-dimethyl-propionic Acid Ethyl Ester

A-3 (75 g), ethyl 5-(t-butylthio)-2,2-dimethyl-4-oxo-pentanoate (prepared according to the procedures described in U.S. Pat. No. 5,288,743 issued Feb. 22, 1994; 64 g), NaOAc (40 g) in toluene (800 mL) and HOAc (400 mL) was stirred at room temperature for 3 days. The mixture was poured into water and made basic with solid Na₂CO₃. The mixture was extracted with EtOAc (×3), then washed with water (×2), brine, dried (MgSO₄), filtered and concentrated to give a dark red-black oil. Column chromatography of the mother liquor (silica gel packed in hexanes; eluting with hexane then hexane-EtOAc 9:1 rising to 4:1) afforded 68 g of the title compound (A-4), as a yellow solid.

### Step 5: 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic Acid Ethyl Ester

3-[3-tert-Butylsulfanyl-5-(pyridin-2-ylmethoxy)-1H-indol-2-yl]-2,2-dimethyl-propionic acid ethyl ester (A-4; 20.0 g, 45.4 mmol) was dissolved in DMF (150 mL) and cooled to −10° C. under N₂. Sodium hydride (60% dispersion in mineral oil; 2.0 g, 50.0 mmol) was added portionwise, and the reaction was stirred at −10° C. for 45 minutes until the foam had disappeared. To this dark brown-reddish solution was added methanesulfonic acid 4-(6-methoxy-pyridin-3-yl)-benzyl ester (Int-72; 16.0 g, 54.5 mmol) in DMF dropwise. The reaction was then stirred at −10° C. for 1 hour and allowed to warm to room temperature slowly. After 16 hours, LCMS confirmed the formation of the product. The reaction was quenched with saturated NH₄Cl and diluted with methyl tert-butyl ether (MTBE) and water. The aqueous phase was extracted twice with MTBE. The combined organic layers were dried over MgSO₄, filtered, and concentrated, and the crude product was purified by column chromatography to give the desired product (A-5).

### Step 6: 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic Acid

A-5 (21.5 g, 33.7 mmol) was dissolved in THF (100 mL) and MeOH (100 mL) and stirred until it became a clear solution. 3N LiOH aqueous solution (56 mL, 168.5 mmol) was added and the reaction was refluxed at 80° C. for 2 hours. LCMS confirmed the formation of the product, so the reaction was cooled to room temperature and partitioned between EtOAc and water. The pH of the aqueous solution was adjusted to pH 1 with 10% HCl, and the aqueous phase was extracted three times with EtOAc. The combined organic layers were washed with water, dried over MgSO₄, filtered, and concentrated to give the desired free acid (A-6).

Mass spectrometry data of Compound 1-2, Compound 2-19, Compound 2-21, Compound 2-35, Compound 2-62, Compound 2-89, Compound 2-195, Compound 2-196, Compound 2-206, Compound 3-1, Compound 3-2, Compound 3-3, Compound 3-4, Compound 3-5, and Compound 4-1 is shown in Tables 1-4.

Notes:

For Compound 1-2, step 6 was not performed.
For Compound 2-62, after Step 6, the 6-methoxy-pyridin-3-yl in the precursor was hydrolyzed with potassium hydroxide to give the 6-hydroxy-pyridin-3-yl in the final product.
For Compound 2-89, during Step 6, the 5-fluorothiazoyl in the precursor was also hydrolyzed to give the 5-methoxythiazolyl in the final product.
For Compound 2-195, after Step 5, a Suzuki cross-coupling reaction was performed to give compound A-5b, as described in Example 5, Step 2.
For Compound 2-196, after Step 5, a Suzuki cross-coupling reaction was performed to give compound A-5b, as described in Example 5, Step 2.
For Compound 3-1, after Step 5, a Suzuki cross-coupling reaction was performed to give compound A-5b, as described in Example 5, Step 2.
For Compound 3-2, after Step 5, a Suzuki cross-coupling reaction was performed to give compound A-5b, as described in Example 5, Step 2.
For Compound 3-3, after Step 5, a Suzuki cross-coupling reaction was performed to give compound A-5b, as described in Example 5, Step 2.
For Compound 3-4, after Step 5, a Suzuki cross-coupling reaction was performed to give compound A-5b, as described in Example 5, Step 2.
For Compound 3-5, after Step 5, a Suzuki cross-coupling reaction was performed to give compound A-5b, as described in Example 5, Step 2.
For Compound 4-1, i) during Step 1, 1-(4-isopropylphenyl)hydrazine was used in place of 4-methoxyphenylhydrazine, and 1-iodo-4-bromomethylbenzene was used in place of 4-chlorobenzylchloride, ii) Steps 3 and 4 were not performed; the product from Step 2 (C-2) was used directly in Step 5, iii) after the hydrolysis in Step 5, a Suzuki cross-coupling reaction was performed to give the final compound, as described in Example 5, Step 2.

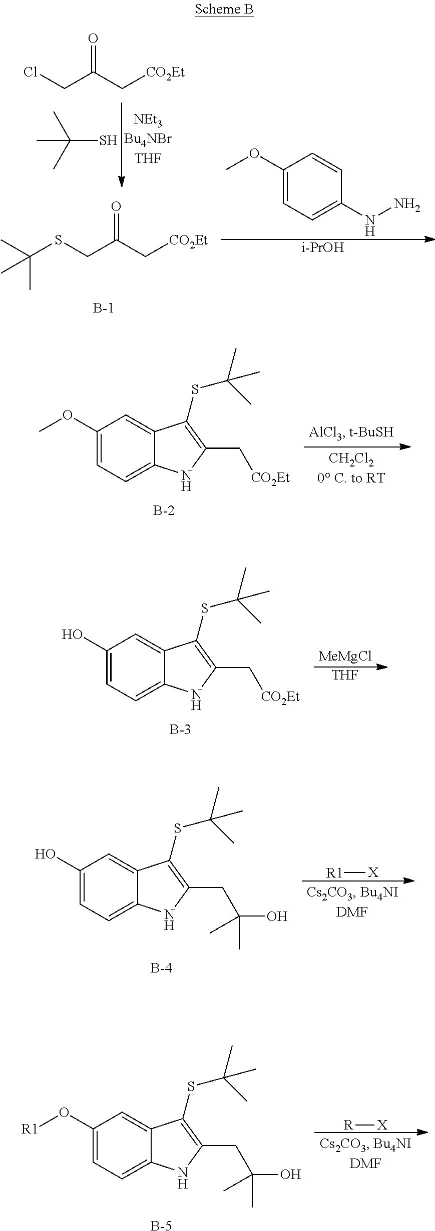

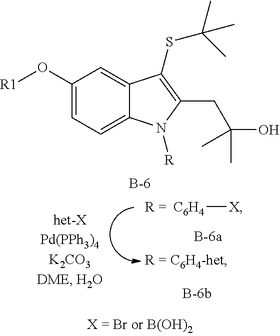

### Example 2: Preparation of Compound 1-4, Compound 1-5, and Compound 1-6

Compound 1-4, Compound 1-5, and Compound 1-6 were prepared as outlined in Scheme B. A detailed illustrative example of the reaction conditions shown in Scheme B is described for the synthesis of 1-[3-tert-butylsulfanyl-1-(4-chloro-benzyl)-5-(pyridin-2-ylmethoxy)-1H-indol-2-yl]-2-methyl-propan-2-ol.

### Step 1: 4-tert-Butylsulfanyl-3-oxo-butyric Acid Ethyl Ester

Ethyl 4-chloroacetoacetate (7.5 mL, 51.9 mmol), 2-methyl-2-propanethiol (5.6 mL, 49.7 mmol), triethylamine (10.8 mL, 77.4 mmol), and catalytic tetrabutylammonium bromide were dissolved in THF (250 mL) and stirred at room temperature overnight. Silica gel was added, and the mixture was concentrated and filtered over a plug of silica gel to obtain the desired product (B-1), which was used without further purification.

### Step 2: (3-tert-Butylsulfanyl-5-methoxy-1H-indol-2-yl)-acetic Acid Ethyl Ester

4-Methoxyphenylhydrazine hydrochloride (7.7 g, 44.1 mmol) and B-1 (7.4 g, 33.9 mmol) were dissolved in 2-propanol (150 mL) and heated to reflux for 24 hours. The reaction mixture was concentrated and partitioned between EtOAc and saturated aq. NaHCO₃. The aqueous layer was extracted with EtOAc, and the combined organic layers were washed with brine, dried over MgSO₄, filtered, and concentrated. The residue was purified on silica gel (0 to 30% EtOAc in hexanes) to give the desired product (B-2).

### Step 3: (3-tert-Butylsulfanyl-5-hydroxy-1H-indol-2-yl)-acetic Acid Ethyl Ester

Aluminum chloride (7.5 g 56.0 mmol) was suspended in tert-butylthiol (21 mL, 186.7 mmol) at 0° C. B-2 (6.0 g, 18.7 mmol) was added in CH₂Cl₂ (21 mL), and the reaction was allowed to warm to room temperature. After 2 hours, the reaction was complete by TLC analysis, so the solution poured into ice and acidified with 10% HCl aqueous solution. The aqueous layer was extracted three times with EtOAc, the combined organics were dried over MgSO₄, filtered, and concentrated to give the desired product (B-3).

### Step 4: 3-tert-Butylsulfanyl-2-(2-hydroxy-2-methyl-propyl)-1H-indol-5-ol

B-3 (2.2 g, 7.0 mmol) was dissolved in THF (70 mL) and cooled to 0° C. Methylmagnesium chloride (3M; 14 mL, 42.0 mmol) was added dropwise, and the reaction was stirred for 1 hour at room temperature. The reaction was quenched with aq. NH₄Cl and extracted with EtOAc. The combined organic layers were dried over MgSO₄, filtered, concentrated, and purified on silica gel to give the desired product (B-4).

### Step 5: 1-[3-tert-Butylsulfanyl-5-(pyridin-2-ylmethoxy)-1H-indol-2-yl]-2-methyl-propan-2-ol

To B-4 (0.18 g, 0.61 mmol) in DMF (6 mL) was added cesium carbonate (1.0 g, 3.1 mmol). The reaction was stirred at room temperature for 30 minutes, and then 2-chloromethylpyridine hydrochloride (0.11 g, 0.67 mmol) and tetrabutylammonium iodide (0.05 g, 0.13 mmol) were added, and the reaction was stirred at room temperature for an additional 16 hours. The reaction was partitioned between water and diethyl ether, and the aqueous layer was extracted with diethyl ether. The combined organic layers were washed with water, dried over MgSO₄, filtered, and concentrated. The residue was purified on silica gel to give the desired product (B-5).

### Step 6: 1-[3-tert-Butylsulfanyl-1-(4-chloro-benzyl)-5-(pyridin-2-ylmethoxy)-1H-indol-2-yl]-2-methyl-propan-2-ol

To B-5 (0.05 g, 0.13 mmol) in DMF (3 mL) was added cesium carbonate (0.21 g, 0.65 mmol). The reaction was stirred at room temperature for 30 minutes, and then 1-chloro-4-chloromethylbenzene (0.03 g, 0.20 mmol) and tetrabutylammonium iodide (0.05 g, 0.13 mmol) were added, and the reaction was stirred at room temperature overnight. The reaction was partitioned between water and EtOAc, and the aqueous layer was extracted with EtOAc. The combined organics were washed with water, dried over MgSO₄, filtered, concentrated, and purified on silica gel (EtOAc:hexanes gradient) to give the desired compound (B-6).

Mass spectrometry data of Compound 1-4, Compound 1-5, and Compound 1-6, is shown in Tables 1-4.

Notes:

For Compound 1-4, Compound 1-5, and Compound 1-6, after Step 6, a Suzuki cross-coupling reaction was performed to give compound B-6b, as described in Example 5, Step 2.

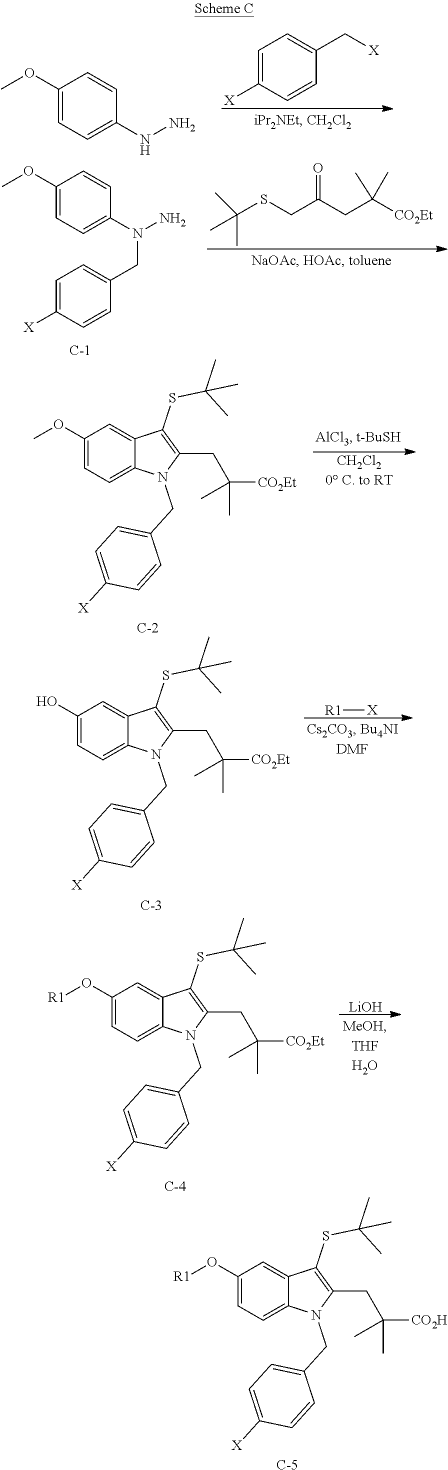

### Example 3 (X=Cl): (S)-2-[3-tert-Butylsulfanyl-2-(2-carboxy-2-methyl-propyl)-1-(4-chloro-benzyl)-1H-indol-5-yloxymethyl]-pyrrolidine-1-carboxylic acid tert-butyl Ester

### Step 1: N-(4-Chloro-benzyl)-N-(4-methoxy-phenyl)-hydrazine Hydrochloride

A solution of 4-Methoxyphenylhydrazine hydrochloride (10.0 g, 57.3 mmol), 4-chlorobenzylchloride (9.2 g, 57.2 mmol), tetrabutylammonium bromide (3.7 g, 11.5 mmol), and diisopropylethylamine (20 mL, 115 mmol) in CH₂Cl₂ (250 mL) was stirred at room temperature for several days. The reaction mixture was diluted with water and the organic layer was dried over MgSO₄, filtered, and concentrated. The residue was taken up in toluene (200 mL) and diethyl ether (100 mL), and 1 equivalent of 4N HCl in dioxane was added at 0° C. The mixture was stirred at room temperature for 2 hours, and then evaporated to dryness to give the desired product (C-1; X=Cl) as a purple solid.

### Step 2: 3-[1-(4-Chloro-benzyl)-3-tert-butylsulfanyl-5-methoxy-1H-indol-2-yl]-2,2-dimethyl-propionic Acid Ethyl Ester

C-1 (˜16 g, 57.3 mmol), ethyl 5-(t-butylthio)-2,2-dimethyl-4-oxo-pentanoate (prepared according to the procedures described in U.S. Pat. No. 5,288,743 issued Feb. 22, 1994; 14.8 g, 57.3 mmol), NaOAc (5.2 g) in toluene (120 mL) and HOAc (66 mL) was stirred at room temperature in the dark for 5 days. The mixture was partitioned between EtOAc and water, and the organic layer was stirred with solid NaHCO₃, filtered, and evaporated. The residue was purified on silica gel (0 to 55% CH₂Cl₂ in hexanes), and the isolated product was recrystallized from hexanes to give the desired product (C-2; X=Cl).

### Step 3: 3-[1-(4-Chloro-benzyl)-3-tert-butylsulfanyl-5-hydroxy-1H-indol-2-yl]-2,2-dimethyl-propionic Acid Ethyl Ester

Aluminum chloride (0.820 g 6.15 mmol) was suspended in tert-butylthiol (1.8 mL, 16 mmol) and cooled to 0° C. C-2 (1.0 g, 2.0 mmol) was added in CH₂Cl₂ (2.4 mL), and the reaction was allowed to warm to room temperature. After 3 hours, the reaction was complete by TLC analysis, so the solution was diluted with CH₂Cl₂ and washed with 10% ice-cooled HCl aqueous solution. The aqueous layer was extracted three times with CH₂Cl₂, the combined organics were dried over MgSO₄, filtered, and concentrated to give the desired product (C-3; X=Cl) as a colourless foam.

### Step 4: (S)-2-[3-tert-Butylsulfanyl-1-(4-chloro-benzyl)-2-(2-ethoxycarbonyl-2-methyl-propyl)-1H-indol-5-yloxymethyl]-pyrrolidine-1-carboxylic acid tert-butyl Ester

To 3-[1-(4-Chloro-benzyl)-3-tert-butylsulfanyl-5-hydroxy-1H-indol-2-yl]-2,2-dimethyl-propionic acid ethyl ester (C-3; 0.5 g, 1.05 mmol) in DMF (2.5 mL) was added N—BOC—(S)-2-(toluene-4-sulfonyloxymethyl)pyrrolidine (0.39 g, 1.10 mmol), and Cs₂CO₃ (0.69 g, 2.1 mmol). The reaction was stirred at 45° C. for 2 hours, and then catalytic potassium iodide was added and the reaction was heated to 60° C. overnight. The reaction mixture was diluted with EtOAc, washed with water, dried over Na₂SO₄, filtered, and concentrated. The residue was purified on silica get (0 to 15% EtOAc in hexanes) to give the desired product (C-4; X=Cl).

### Step 5: (S)-2-[3-tert-Butylsulfanyl-2-(2-carboxy-2-methyl-propyl)-1-(4-chloro-benzyl)-1H-indol-5-yloxymethyl]-pyrrolidine-1-carboxylic Acid tert-butyl Ester (1-1)

The ester from Step 4 (0.16 g, 0.26 mmol) was dissolved in MeOH (1 mL), THF (1 mL), and water (1 mL). Lithium hydroxide (0.6 g, 1.43 mmol) was added, and the reaction was heated for 12 hours until no starting material was seen by TLC analysis. The reaction was diluted with water, acidified to pH 5 with citric acid, and extracted with EtOAc. The combined organic layers were washed with water, dried over MgSO₄, filtered, and concentrated. The residue was purified on silica gel (0 to 40% EtOAc in hexanes) to give the desired product (C-5; X=Cl).

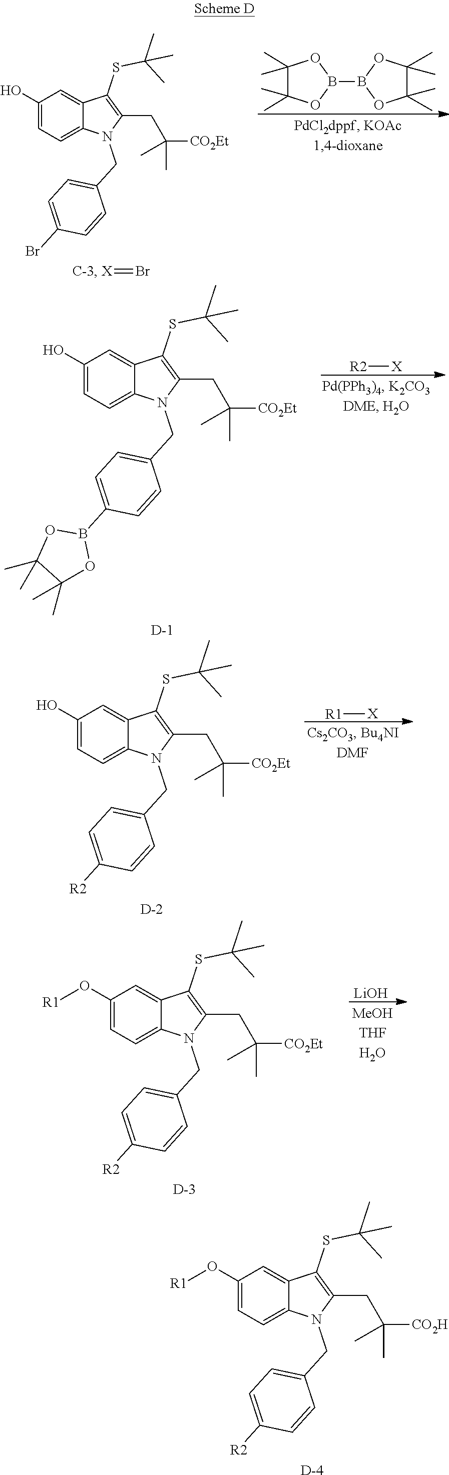

### Example 4: Preparation of Compound 2-23, Compound 2-24, Compound 2-31, Compound 2-32, Compound 2-33, Compound 2-76, Compound 2-77, Compound 2-78, Compound 2-79, Compound 2-80, Compound 2-81, Compound 2-82, Compound 2-84, Compound 2-85, Compound 2-99, Compound 2-100, Compound 2-101, Compound 2-104, Compound 2-108, Compound 2-122, Compound 2-135, Compound 2-141, Compound 2-148, Compound 2-149, Compound 2-150, Compound 2-151, Compound 2-156, Compound 2-183, Compound 2-184, Compound 2-188, Compound 2-189, Compound 2-190, Compound 2-191, Compound 2-192, Compound 2-193, Compound 2-197, Compound 2-198, Compound 2-199, Compound 2-200, Compound 2-201, Compound 2-202, Compound 2-203, Compound 2-204, Compound 2-205; Compound 2-207, Compound 2-208, Compound 2-209, Compound 2-210, Compound 2-211, Compound 2-212, Compound 2-213, Compound 2-214, Compound 2-215, Compound 2-216, Compound 2-217, Compound 2-218, Compound 2-219, Compound 2-220, Compound 2-221, Compound 2-222, Compound 2-223, Compound 2-224, Compound 2-225, Compound 2-226, Compound 2-227, Compound 2-228, Compound 2-229, Compound 2-230, Compound 2-231, Compound 2-232, Compound 2-233, Compound 2-234, and Compound 4-2

Compound 2-23, Compound 2-24, Compound 2-31, Compound 2-32, Compound 2-33, Compound 2-76, Compound 2-77, Compound 2-78, Compound 2-79, Compound 2-80, Compound 2-81, Compound 2-82, Compound 2-84, Compound 2-85, Compound 2-99, Compound 2-100, Compound 2-101, Compound 2-104, Compound 2-108, Compound 2-122, Compound 2-135, Compound 2-141, Compound 2-148, Compound 2-149, Compound 2-150, Compound 2-151, Compound 2-156, Compound 2-183, Compound 2-184, Compound 2-188, Compound 2-189, Compound 2-190, Compound 2-191, Compound 2-192, Compound 2-193, Compound 2-197, Compound 2-198, Compound 2-199, Compound 2-200, Compound 2-201, Compound 2-202, Compound 2-203, Compound 2-204, Compound 2-205; Compound 2-207, Compound 2-208, Compound 2-209, Compound 2-210, Compound 2-211, Compound 2-212, Compound 2-213, Compound 2-214, Compound 2-215, Compound 2-216, Compound 2-217, Compound 2-218, Compound 2-219, Compound 2-220, Compound 2-221, Compound 2-222, Compound 2-223, Compound 2-224, Compound 2-225, Compound 2-226, Compound 2-227, Compound 2-228, Compound 2-229, Compound 2-230, Compound 2-231, Compound 2-232, Compound 2-233, Compound 2-234, and Compound 4-2, were prepared as shown in Scheme D. A detailed illustrative example of the reaction conditions shown in Scheme D is described for the synthesis of 3-{3-tert-Butylsulfanyl-5-(6-fluoro-quinolin-2-ylmethoxy)-1-[4-(6-methoxy-pyridin-3-yl)-benzyl]-1H-indol-2-yl}-2,2-dimethyl-propionic acid (Compound 2-141).

### Step 1: 3-{3-tert-Butylsulfanyl-5-hydroxy-1-[4-(4,4,5,5-tetramethyl-[1,3,2]dioxaborolan-2-yl)-benzyl]-1H-indol-2-yl}-2,2-dimethyl-propionic Acid Ethyl Ester

The phenol from Example 3, Step 3 (C-3, X=Br; 35.0 g, 67.5 mmol), bis(pinacolato)diboron (Combi-Blocks; 25.0 g, 98.4 mmol), and KOAc (19.9 g, 209.1 mmol) was dissolved in 1,4-dioxane (350 mL) and degassed with N₂ for 30 minutes. PdCl₂dppf (2.5 g, 3.1 mmol) was added, and the reaction mixture was degassed an additional 30 minutes with N₂. The reaction was heated at 85° C. overnight. The reaction mixture was partitioned between water and EtOAc, the aqueous layer was extracted three times with EtOAc, the combined organic layers were washed with water, brine, dried over MgSO₄, filtered, and concentrated. The crude material was purified on silica gel (15% EtOAc in hexanes) to give the desired product (D-1, 33.5 g).

### Step 2: 3-{3-tert-Butylsulfanyl-5-hydroxy-1-[4-(6-methoxy-pyridin-3-yl)-benzyl]-1H-indol-2-yl}-2,2-dimethyl-propionic Acid Ethyl Ester

D-1 (25.34 g, 44.8 mmol), 5-bromo-2-methoxypyridine (Combi-blocks; 10.9 g, 70.3 mmol), and K₂CO₃ (15.5 g, 112.1 mmol) were dissolved in DME (300 mL) and water (150 mL) and degassed with N₂ for 30 minutes. Pd(PPh₃)₄ (1.6 g, 1.4 mmol) was added, and the reaction mixture was degassed with N₂ for an additional 15 minutes. The solution was heated to 80° C. overnight, and then cooled to room temperature and diluted with EtOAc and water. The aqueous layer was extracted 3 times with EtOAc, the combined organic layers were washed with water, brine, dried over MgSO₄, filtered, and concentrated. The crude material was purified on silica gel (0 to 8% EtOAc in hexanes) to give the desired product (D-2, 23.7 g).

### Step 3: 3-{3-tert-Butylsulfanyl-5-(6-fluoro-quinolin-2-ylmethoxy)-1-[4-(6-methoxy-pyridin-3-yl)-benzyl]-1H-indol-2-yl}-2,2-dimethyl-propionic Acid Ethyl Ester

To 3-{3-tert-Butylsulfanyl-5-hydroxy-1-[4-(6-methoxy-pyridin-3-yl)-benzyl]-1H-indol-2-yl}-2,2-dimethyl-propionic acid ethyl ester (D-2; 6.5 g, 11.9 mmol) in MeCN (75 mL) was added 2-bromomethyl-6-fluoro-quinoline (3.14 g, 13.1 mmol), and Cs₂CO₃ (9.7 g, 29.8 mmol). The reaction was stirred at room temperature overnight, after which LCMS showed the reaction was complete. The reaction mixture was partitioned between EtOAc and water, the aqueous layer was extracted with EtOAc, and the combined organic layers were dried over MgSO₄, filtered, and concentrated. The residue was purified on silica gel (0 to 25% EtOAc in hexanes) to give the desired product (D-3, 7.6 g).

### Step 4: 3-{3-tert-Butylsulfanyl-5-(6-fluoro-quinolin-2-ylmethoxy)-1-[4-(6-methoxy-pyridin-3-yl)-benzyl]-1H-indol-2-yl}-2,2-dimethyl-propionic Acid

D-3 (6.58 g, 9.3 mmol) was dissolved in MeOH (36 mL), THF (75 mL), and water (36 mL). Lithium hydroxide (2.42 g, 57.7 mmol) was added, and the reaction was heated at 60° C. for 6 hours until no starting material was seen by TLC analysis. The reaction was diluted with water, acidified to pH 5 with citric acid, and extracted with EtOAc. The combined organic layers were washed with water, dried over MgSO₄, filtered, and concentrated. The residue was triturated with hexane:EtOAc (9:1) overnight, and filtered to give the desired product (D-4, 5.9 g).

Mass spectrometry data for Compound 2-23, Compound 2-24, Compound 2-31, Compound 2-32, Compound 2-33, Compound 2-76, Compound 2-77, Compound 2-78, Compound 2-79, Compound 2-80, Compound 2-81, Compound 2-82, Compound 2-84, Compound 2-85, Compound 2-99, Compound 2-100, Compound 2-101, Compound 2-104, Compound 2-108, Compound 2-122, Compound 2-135, Compound 2-141, Compound 2-148, Compound 2-149, Compound 2-150, Compound 2-151, Compound 2-156, Compound 2-183, Compound 2-184, Compound 2-188, Compound 2-189, Compound 2-190, Compound 2-191, Compound 2-192, Compound 2-193, Compound 2-197, Compound 2-198, Compound 2-199, Compound 2-200, Compound 2-201, Compound 2-202, Compound 2-203, Compound 2-204, Compound 2-205; Compound 2-207, Compound 2-208, Compound 2-209, Compound 2-210, Compound 2-211, Compound 2-212, Compound 2-213, Compound 2-214, Compound 2-215, and Compound 4-2, is shown in Tables 1-4.

Notes:

For Compound 2-33, during Step 3, the imidazole was also alkylated to give the final product.
For Compound 2-79, during Step 4, the ethyl ester of the precursor was also hydrolyzed to give the acid in the final product.
For Compound 2-80, after Step 3, the ketone in the precursor was reduced with sodium borohydride to give the alcohol in the final product.
For Compound 2-100, during Step 4, the 6-fluoropyridinyl in the precursor was also hydrolyzed to give the 6-methoxypyridinyl in the final product.
For Compound 2-104, after Step 3, a Suzuki cross-coupling reaction was performed on the 6-bromopyridinyl in the precursor to give the 6-cyclopropylpyridinyl in the final product, as described in Example 5, Step 2.
For Compound 4-2, Step 3 was not performed.

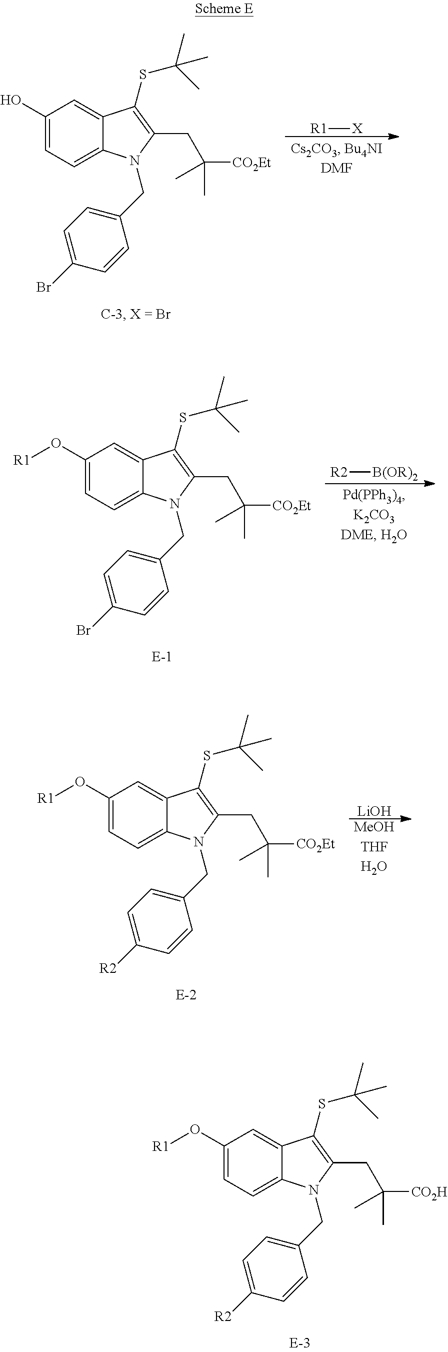

### Example 5: Preparation of Compound 2-30, Compound 2-64, Compound 2-73, Compound 2-87, Compound 2-88, Compound 2-97, Compound 2-107, and Compound 2-121

Compound 2-30, Compound 2-64, Compound 2-73, Compound 2-87, Compound 2-88, Compound 2-97, Compound 2-107, and Compound 2-121, were prepared as shown in Scheme E. A detailed illustrative example of the reaction conditions shown in Scheme E is described for the synthesis of 3-[3-tert-butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-73).

### Step 1: 3-[1-(4-Bromo-benzyl)-3-tert-butylsulfanyl-5-(6-fluoro-quinolin-2-ylmethoxy)-1H-indol-2-yl]-2,2-dimethyl-propionic Acid Ethyl Ester

To 3-[1-(4-Bromo-benzyl)-3-tert-butylsulfanyl-5-hydroxy-1H-indol-2-yl]-2,2-dimethyl-propionic acid ethyl ester (C-3; 0.25 g, 0.48 mmol) in DMF (2 mL) was added 2-chloromethyl-5-methyl-pyridine hydrochloride (0.13 g, 0.72 mmol), Cs₂CO₃ (0.39 g, 1.21 mmol), and catalytic tetrabutylammonium iodide. The reaction was stirred at room temperature overnight, after which LCMS showed the reaction was complete. The reaction mixture was partitioned between EtOAc and water, the aqueous layer was extracted with EtOAc, and the combined organic layers were dried over MgSO₄, filtered, and concentrated. The crude material was purified on silica gel (0 to 15% EtOAc in hexanes) to give an additional the desired product (E-1, 0.30 g).

### Step 2: 3-{3-tert-Butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic Acid Ethyl Ester

E-1 (0.06 g, 0.10 mmol), 2-methoxy-pyridine-5-boronic acid (0.02 g, 0.14 mmol), and K₂CO₃ (0.03 g, 0.24 mmol) were dissolved in DME (1 mL) and water (0.5 mL) and degassed with N₂ for 10 minutes. Pd(PPh₃)₄ (0.01 g, 0.01 mmol) was added, and the reaction mixture was degassed with N₂ for an additional 10 minutes. The solution was heated to 80° C. for 4 hours, and then cooled to room temperature and diluted with EtOAc and water. The aqueous layer was extracted 3 times with EtOAc, the combined organic layers were washed with water, brine, dried over MgSO₄, filtered, and concentrated. The crude material was purified on silica gel (0 to 50% EtOAc in hexanes) to give the desired product (E-2).

### Step 3: 3-{3-tert-Butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic acid

E-2 (0.22 g, 0.31 mmol) was dissolved in MeOH (0.1 mL), THF (0.1 mL), and water (0.1 mL). Lithium hydroxide, 1N aqueous solution (0.1 mL) was added, and the reaction was heated at 60° C. for 4 hours until no starting material was seen by LCMS. The reaction was diluted with water and EtOAc, acidified to pH 5 with citric acid, and extracted with EtOAc. The combined organic layers were washed with water, dried over MgSO₄, filtered, and concentrated to give the desired product (F-4).

Mass spectrometry data for Compound 2-30, Compound 2-64, Compound 2-73, Compound 2-87, Compound 2-88, Compound 2-97, Compound 2-107, and Compound 2-121, is shown in Table 1-4.

Notes:

For Compound 2-64, Steps 2 and 3 were performed in the reverse order.
For Compound 2-87, during Step 3, the 5-cyanopyridyl in the precursor was also hydrolyzed to give the 5-carbamoylpyridyl in the final product.
For Compound 2-88, Steps 2 and 3 were performed in the reverse order.
For Compound 2-97, during Step 3, the 6-cyanopyridyl in the precursor was also hydrolyzed to give the 6-carbamoylpyridyl in the final product.

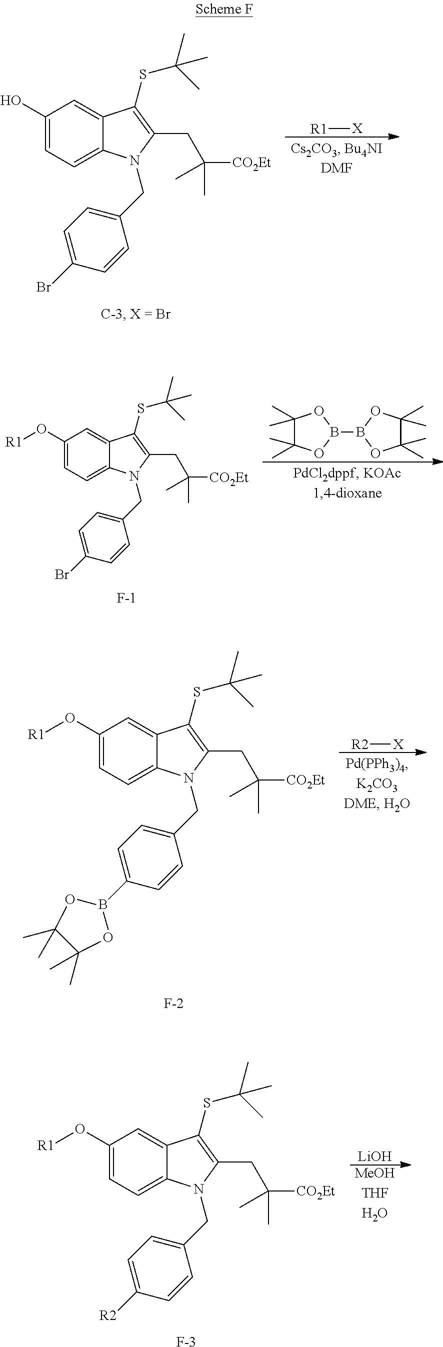

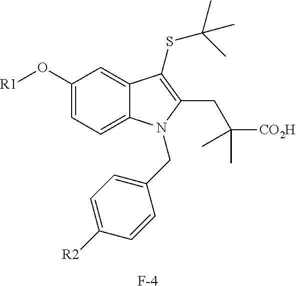

### Example 6: Preparation of Compound 2-1, Compound 2-2, Compound 2-3, Compound 2-4, Compound 2-5, Compound 2-6, Compound 2-7, Compound 2-17, Compound 2-18, Compound 2-20, Compound 2-34, Compound 2-39, Compound 2-41, Compound 2-43, Compound 2-47, Compound 2-55, Compound 2-65, Compound 2-67, Compound 2-68, Compound 2-90, Compound 2-91, Compound 2-92, Compound 2-93, Compound 2-94, Compound 2-95, Compound 2-96, Compound 2-98, Compound 2-102, Compound 2-103, Compound 2-105, Compound 2-106, Compound 2-109, Compound 2-110, Compound 2-111, Compound 2-112, Compound 2-113, Compound 2-114, Compound 2-115, Compound 2-116, Compound 2-117, Compound 2-118, Compound 2-119, Compound 2-120, Compound 2-125, Compound 2-126, Compound 2-127, Compound 2-128, Compound 2-129, Compound 2-130, Compound 2-131, Compound 2-136, Compound 2-137, Compound 2-138, Compound 2-139, Compound 2-140, Compound 2-142, Compound 2-143, Compound 2-144, Compound 2-145, Compound 2-146, Compound 2-147, Compound 2-157, Compound 2-158, Compound 2-159, Compound 2-160, Compound 2-161, Compound 2-162, Compound 2-164, Compound 2-165, Compound 2-166, Compound 2-167, Compound 2-168, Compound 2-169, Compound 2-171, Compound 2-172, Compound 2-173, Compound 2-174, Compound 2-175, Compound 2-176, Compound 2-177, Compound 2-178, Compound 2-179, Compound 2-180, Compound 2-181, Compound 2-182, Compound 2-185, Compound 2-186, and Compound 2-187

Compound 2-1, Compound 2-2, Compound 2-3, Compound 2-4, Compound 2-5, Compound 2-6, Compound 2-7, Compound 2-17, Compound 2-18, Compound 2-20, Compound 2-34, Compound 2-39, Compound 2-41, Compound 2-43, Compound 2-47, Compound 2-55, Compound 2-65, Compound 2-67, Compound 2-68, Compound 2-90, Compound 2-91, Compound 2-92, Compound 2-93, Compound 2-94, Compound 2-95, Compound 2-96, Compound 2-98, Compound 2-102, Compound 2-103, Compound 2-105, Compound 2-106, Compound 2-109, Compound 2-110, Compound 2-111, Compound 2-112, Compound 2-113, Compound 2-114, Compound 2-115, Compound 2-116, Compound 2-117, Compound 2-118, Compound 2-119, Compound 2-120, Compound 2-125, Compound 2-126, Compound 2-127, Compound 2-128, Compound 2-129, Compound 2-130, Compound 2-131, Compound 2-136, Compound 2-137, Compound 2-138, Compound 2-139, Compound 2-140, Compound 2-142, Compound 2-143, Compound 2-144, Compound 2-145, Compound 2-146, Compound 2-147, Compound 2-157, Compound 2-158, Compound 2-159, Compound 2-160, Compound 2-161, Compound 2-162, Compound 2-164, Compound 2-165, Compound 2-166, Compound 2-167, Compound 2-168, Compound 2-169, Compound 2-171, Compound 2-172, Compound 2-173, Compound 2-174, Compound 2-175, Compound 2-176, Compound 2-177, Compound 2-178, Compound 2-179, Compound 2-180, Compound 2-181, Compound 2-182, Compound 2-185, Compound 2-186, and Compound 2-187, were prepared as shown in Scheme F. A detailed illustrative example of the reaction conditions shown in Scheme F is described for the synthesis of 3-{3-tert-butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic acid (Compound 2-140).

### Step 1: 3-[1-(4-Bromo-benzyl)-3-tert-butylsulfanyl-5-(6-fluoro-quinolin-2-ylmethoxy)-1H-indol-2-yl]-2,2-dimethyl-propionic Acid Ethyl Ester

To 3-[1-(4-Bromo-benzyl)-3-tert-butylsulfanyl-5-hydroxy-1H-indol-2-yl]-2,2-dimethyl-propionic acid ethyl ester (C-3; 2.0 g, 3.9 mmol) in MeCN (25 mL) was added 2-bromomethyl-6-fluoro-quinoline (1.0 g, 4.2 mmol), and Cs₂CO₃ (2.5 g, 7.7 mmol). The reaction was stirred at room temperature overnight, after which LCMS showed the reaction was complete. The reaction mixture was partitioned between EtOAc and water, the aqueous layer was extracted with EtOAc, and the combined organic layers were dried over MgSO₄, filtered, and concentrated. The residue was recrystallized in EtOAc:hexane to give the desired product (F-1, 1.9 g). The filtrate was concentrated and purified on silica gel (0 to 15% EtOAc in hexanes) to give an additional 1 g of F-1.

### Step 2: 3-{3-tert-Butylsulfanyl-5-(6-fluoro-quinolin-2-ylmethoxy)-1-[4-(4,4,5,5-tetramethyl-[1,3,2]dioxaborolan-2-yl)-benzyl]-1H-indol-2-yl}-2,2-dimethyl-propionic Acid Ethyl Ester

F-1 (1.0 g, 1.5 mmol), bis(pinacolato)diboron (Combi-Blocks; 1.1 g, 4.3 mmol), and KOAc (0.44 g, 4.5 mmol) was dissolved in 1,4-dioxane (15 mL) and degassed with N₂ for 10 minutes in a sealed vessel. PdCl₂dppf (0.13 g, 0.16 mmol) was added, and the reaction mixture was degassed an additional 10 minutes with N₂. The vessel was sealed and the reaction was heated at 95° C. overnight. The reaction mixture was partitioned between water and EtOAc, the aqueous layer was extracted three times with EtOAc, the combined organic layers were washed with water, brine, dried over MgSO₄, filtered, and concentrated. The crude material was purified on silica gel (0 to 20% EtOAc in hexanes) to give the desired product (F-2).

### Step 3: 3-{3-tert-Butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic Acid Ethyl Ester

F-2 (0.25 g, 0.35 mmol), 2-bromo-6-methoxypyridine (0.09 g, 0.48 mmol), and K₂CO₃ (0.15 g, 1.05 mmol) were dissolved in DME (3.5 mL) and water (1.8 mL) and degassed with N₂ for 10 minutes. Pd(PPh₃)₄ (0.06 g, 0.05 mmol) was added, and the reaction mixture was degassed with N₂ for an additional 10 minutes. The solution was heated to 85° C. for 4 hours, and then cooled to room temperature and diluted with EtOAc and water. The aqueous layer was extracted 3 times with EtOAc, the combined organic layers were washed with water, brine, dried over MgSO₄, filtered, and concentrated. The crude material was purified on silica gel (0 to 25% EtOAc in hexanes) to give the desired product (F-3).

### Step 4: 3-{3-tert-Butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic Acid

F-3 (0.22 g, 0.31 mmol) was dissolved in MeOH (1.5 mL), THF (3 mL), and water (1.5 mL). Lithium hydroxide (0.08 g, 1.9 mmol) was added, and the reaction was heated at 60° C. for 3.5 hours until no starting material was seen by TLC analysis. The reaction was diluted with water, acidified to pH 5 with citric acid, and extracted with EtOAc. The combined organic layers were washed with water, dried over MgSO₄, filtered, and concentrated to give the desired product (F-4).

Mass spectrometry data for Compound 2-1, Compound 2-2, Compound 2-3, Compound 2-4, Compound 2-5, Compound 2-6, Compound 2-7, Compound 2-17, Compound 2-18, Compound 2-20, Compound 2-34, Compound 2-39, Compound 2-41, Compound 2-43, Compound 2-47, Compound 2-55, Compound 2-65, Compound 2-67, Compound 2-68, Compound 2-90, Compound 2-91, Compound 2-92, Compound 2-93, Compound 2-94, Compound 2-95, Compound 2-96, Compound 2-98, Compound 2-102, Compound 2-103, Compound 2-105, Compound 2-106, Compound 2-109, Compound 2-110, Compound 2-111, Compound 2-112, Compound 2-113, Compound 2-114, Compound 2-115, Compound 2-116, Compound 2-117, Compound 2-118, Compound 2-119, Compound 2-120, Compound 2-125, Compound 2-126, Compound 2-127, Compound 2-128, Compound 2-129, Compound 2-130, Compound 2-131, Compound 2-136, Compound 2-137, Compound 2-138, Compound 2-139, Compound 2-140, Compound 2-142, Compound 2-143, Compound 2-144, Compound 2-145, Compound 2-146, Compound 2-147, Compound 2-157, Compound 2-158, Compound 2-159, Compound 2-160, Compound 2-161, Compound 2-162, Compound 2-164, Compound 2-165, Compound 2-166, Compound 2-167, Compound 2-168, Compound 2-169, Compound 2-171, Compound 2-172, Compound 2-173, Compound 2-174, Compound 2-175, Compound 2-176, Compound 2-177, Compound 2-178, Compound 2-179, Compound 2-180, Compound 2-181, Compound 2-182, Compound 2-185, Compound 2-186, and Compound 2-187, is shown in Tables 1-4.

Notes:

For Compound 2-17, during Step 4, the 6-methoxypyridazinyl in the precursor was also hydrolyzed to give the 6-hydroxypyridazinyl in the final product.
For Compound 2-172, after Step 2, the 3-tert-butylsulfanyl in the precursor was oxidized with m-chloroperoxybenzoic acid to give the 2-methylpropane-2-sulfonyl in the final product.
For Compound 2-173, after Step 2, the 3-tert-butylsulfanyl moiety in the precursor was oxidized with m-chloroperoxybenzoic acid to give the 2-methylpropane-2-sulfinyl moiety in the final product.

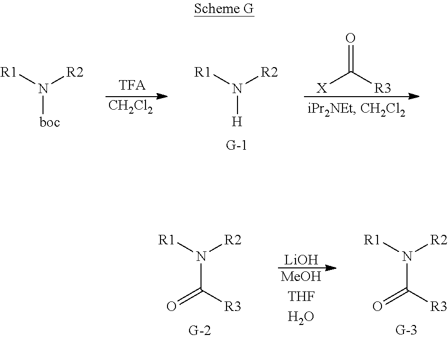

### Example 7: 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic Acid

### Step 1: 3-{3-tert-Butylsulfanyl-5-[(S)-1-(2,3-dihydro-1H-indol-2-yl)methoxy]-1-[4-(6-methoxy-pyridazin-3-yl)-benzyl]-1H-indol-2-yl}-2,2-dimethyl-propionic Acid Ethyl Ester

(S)-2-{3-tert-Butylsulfanyl-2-(2-ethoxycarbonyl-2-methyl-propyl)-1-[4-(6-methoxy-pyridazin-3-yl)-benzyl]-1H-indol-5-yloxymethyl}-2,3-dihydro-indole-1-carboxylic acid tert-butyl ester (0.23 g, 0.30 mmol) was dissolved in CH₂Cl₂ (1.5 mL). TFA (1.5 mL) was added and the reaction was stirred at room temperature for 10 minutes until no starting material was seen by TLC analysis. The solution was concentrated in vacuo, and the crude product (G-1) was used without further purification.

### Step 2: 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic Acid Ethyl Ester

G-1 (0.30 mmol) was dissolved in CH₂Cl₂ (1 mL). Diisopropylethylamine (0.5 mL) was added, followed by acetic anhydride (33 uL, 0.35 mmol), and the reaction was stirred at room temperature until no starting material was seen by LCMS. The reaction was diluted with CH₂Cl₂ and MeOH, concentrated, redissolved in CH₂Cl₂ and washed with water, dried over Na₂SO₄, filtered, and concentrated. The residue was purified on silica gel to give the desired product (G-2).

### Step 3: 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic Acid

G-2 (0.05 g, 0.07 mmol) was dissolved in MeOH (0.5 mL), THF (0.5 mL), and water (0.5 mL). Lithium hydroxide (0.03 g, 0.7 mmol) was added, and the reaction was heated at 60° C. for 6 hours until no starting material was seen by TLC analysis. The reaction was diluted with water, acidified to pH 5 with citric acid, and extracted with EtOAc. The combined organic layers were washed with water, dried over MgSO₄, filtered, and concentrated. The residue was purified on silica gel to give the desired product (G-3).

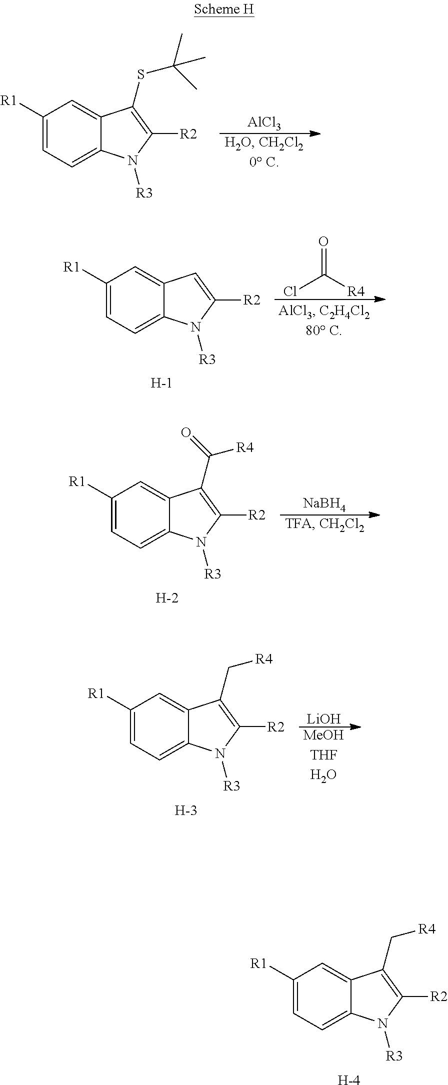

### Example 8: Preparation of Compound 2-8, Compound 2-9, Compound 2-10, Compound 2-11, Compound 2-12, Compound 2-13, Compound 2-14, Compound 2-15, Compound 2-16, Compound 2-22, Compound 2-25, Compound 2-26, Compound 2-27, Compound 2-28, Compound 2-29, Compound 2-123, Compound 2-124; Compound 2-132, Compound 2-133, Compound 2-134; Compound 2-163, Compound 2-170, and Compound 2-194

Compound 2-8, Compound 2-9, Compound 2-10, Compound 2-11, Compound 2-12, Compound 2-13, Compound 2-14, Compound 2-15, Compound 2-16, Compound 2-22, Compound 2-25, Compound 2-26, Compound 2-27, Compound 2-28, Compound 2-29, Compound 2-123, Compound 2-124; Compound 2-132, Compound 2-133, Compound 2-134; Compound 2-163, Compound 2-170, and Compound 2-194, were prepared as shown in Scheme H. A detailed illustrative example of the reaction conditions shown in Scheme H is described for the synthesis of 3-{5-(benzothiazol-2-ylmethoxy)-3-cyclobutylmethyl-1-[4-(6-methoxy-pyridin-3-yl)-benzyl]-1H-indol-2-yl}-2,2-dimethyl-propionic acid (Compound 2-124).

### Step 1: 3-{5-(Benzothiazol-2-ylmethoxy)-1-[4-(6-methoxy-pyridin-3-yl)-benzyl]-1H-indol-2-yl}-2,2-dimethyl-propionic Acid Ethyl Ester

Aluminum chloride (0.18 g, 1.37 mmol) was suspended in CH₂Cl₂ (1 mL), and water (19 uL, 1.0 mmol) was added slowly at room temperature. The mixture was stirred for 5 minutes, and then cooled to 0° C. 3-{5-(Benzothiazol-2-ylmethoxy)-3-tert-butylsulfanyl-1-[4-(6-methoxy-pyridin-3-yl)-benzyl]-1H-indol-2-yl}-2,2-dimethyl-propionic acid ethyl ester (0.12 g, 0.17 mmol) was added in CH₂Cl₂ (1 mL), and the reaction was stirred at room temperature for 2 hours. Once no starting material was observed by tlc, water was added and the mixture was extracted with CH₂Cl₂. The combined organic layers were washed with water, dried over MgSO₄, filtered, and concentrated. The residue was purified to give the desired product (H-1).

### Step 2: 3-{5-(Benzothiazol-2-ylmethoxy)-3-cyclobutanecarbonyl-1-[4-(6-methoxy-pyridin-3-yl)-benzyl]-1H-indol-2-yl}-2,2-dimethyl-propionic Acid Ethyl Ester

To H-1 (0.10 g, 0.17 mmol) in dichloroethane (5 mL) was added cyclobutanecarbonyl chloride (57 uL, 0.50 mmol) and aluminum chloride (0.09 g, 0.66 mmol). The reaction was heated under N₂ for 1.5 hours, and then cooled to room temperature and quenched with saturated aq. potassium sodium tartrate. The mixture was extracted with EtOAc, and the combined organic layers were dried over MgSO₄, filtered, concentrated, and purified on silica gel to give the desired product (H-2).

### Step 3: 3-{5-(Benzothiazol-2-ylmethoxy)-3-cyclobutylmethyl-1-[4-(6-methoxy-pyridin-3-yl)-benzyl]-1H-indol-2-yl}-2,2-dimethyl-propionic Acid Ethyl Ester

H-2 (0.05 g, 0.08 mmol) was suspended in CH₂Cl₂, and sodium borohydride (0.03 g, 0.8 mmol) was added dropwise in TFA (1 mL) and CH₂Cl₂ (1 mL). The mixture was stirred at room temperature for 4 hours, and then quenched with water and basified with solid NaOH pellets. The mixture was extracted with CH₂Cl₂, and the combined organics were dried over MgSO₄, filtered, and concentrated. The residue was purified on silica gel to give the desired product (H-3).

### Step 4: 3-{5-(Benzothiazol-2-ylmethoxy)-3-cyclobutylmethyl-1-[4-(6-methoxy-pyridin-3-yl)-benzyl]-1H-indol-2-yl}-2,2-dimethyl-propionic Acid

H-3 (0.03 g, 0.04 mmol) was dissolved in MeOH (0.5 mL) and THF (0.5 mL). Aq. lithium hydroxide (1N, 0.5 mL) was added, and the reaction was heated at 60° C. for 4 hours until no starting material was seen by LCMS. The reaction was diluted with water, acidified to pH 5 with citric acid, and extracted with EtOAc. The combined organic layers were washed with water, dried over MgSO₄, filtered, and concentrated to give the desired product (H-4).

Mass spectrometry data for Compound 2-8, Compound 2-9, Compound 2-10, Compound 2-11, Compound 2-12, Compound 2-13, Compound 2-14, Compound 2-15, Compound 2-16, Compound 2-22, Compound 2-25, Compound 2-26, Compound 2-27, Compound 2-28, Compound 2-29, Compound 2-123, Compound 2-124; Compound 2-132, Compound 2-133, Compound 2-134; Compound 2-163, Compound 2-170, and Compound 2-194, is shown in Tables 1-4.

Notes:

For Compound 2-8, only Steps 1, 2, and 4 were performed.
For Compound 2-9, only Steps 1 and 4 were performed.
For Compound 2-10, only Steps 1, 2, and 4 were performed.
For Compound 2-11, only Steps 1 and 4 were performed.
For Compound 2-12, only Steps 1, 2, and 4 were performed.
For Compound 2-15, only Steps 1, 2, and 4 were performed.
For Compound 2-16, only Steps 1, 2, and 4 were performed.
For Compound 2-25, only Steps 1 and 4 were performed.
For Compound 2-26, only Steps 1, 2, and 4 were performed.
For Compound 2-27, only Steps 1 and 4 were performed.
For Compound 2-28, only Steps 1, 2, and 4 were performed.
For Compound 2-123, only Steps 1, 2, and 4 were performed.

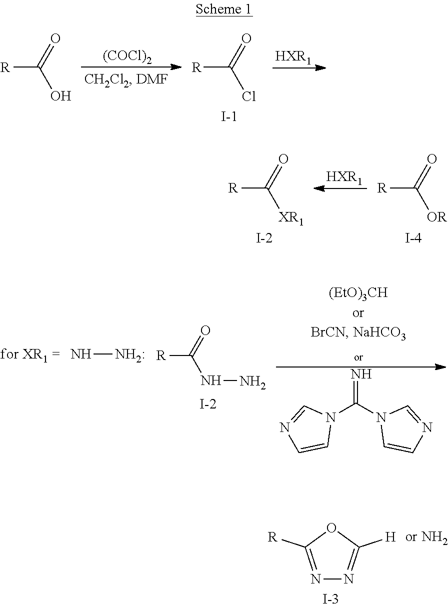

### Example 9: Preparation of Compound 1-1, Compound 1-3, Compound 1-7, Compound 1-8, Compound 1-9, Compound 1-10, Compound 1-11, Compound 1-12, Compound 1-13, Compound 1-14 and Compound 1-15

Compound 1-1, Compound 1-3, Compound 1-7, Compound 1-8, Compound 1-9, Compound 1-10, Compound 1-11, Compound 1-12, Compound 1-13, Compound 1-14 and Compound 1-15, were prepared as outlined in Scheme I. A detailed illustrative example of the reaction conditions shown in Scheme H is described for the synthesis of 3-[3-tert-butylsulfanyl-1-(4-chloro-benzyl)-5-isopropyl-1H-indol-2-yl]-N-(2-hydroxy-ethyl)-2,2-dimethyl-propionamide.

### Step 1: 3-[3-tert-Butylsulfanyl-1-(4-chloro-benzyl)-5-isopropyl-1H-indol-2-yl]-2,2-dimethyl-propionyl Chloride

To 3-[3-tert-butylsulfanyl-1-(4-chloro-benzyl)-5-isopropyl-1H-indol-2-yl]-2,2-dimethyl-propionic acid (prepared according to the procedures described in U.S. Pat. No. 5,081,138 issued Jan. 14, 1992; 0.25 g, 0.53 mmol) suspended in CH₂Cl₂ (5 mL) was added oxalyl chloride (48 uL, 0.56 mmol) and catalytic DMF. The reaction was stirred at room temperature for 3 hours, and then concentrated to give I-1, which was used without further purification.

### Step 2: 3-[3-tert-Butylsulfanyl-1-(4-chloro-benzyl)-5-isopropyl-1H-indol-2-yl]-N-(2-hydroxy-ethyl)-2,2-dimethyl-propionamide

To I-1 (0.18 mmol) in CH₂Cl₂ was added triethylamine (0.1 mL, 0.70 mmol) and 2-aminoethanol (10 uL, 0.19 mmol). The reaction was stirred for 2 days at room temperature, and then concentrated and purified on silica gel (EtOAc:hexanes gradient) to give the desired product (I-2).

### Step 3: 5-{4-[3-tert-Butylsulfanyl-2-(2,2-dimethyl-propyl)-5-(pyridin-2-ylmethoxy)-indol-1-ylmethyl]-phenyl}-[1,3,4]oxadiazol-2-ylamine

To 4-[3-tert-Butylsulfanyl-2-(2,2-dimethyl-propyl)-5-(pyridin-2-ylmethoxy)-indol-1-ylmethyl]-benzoic acid hydrazide (0.05 g, 0.10 mmol) in DMF (1 mL) was added C-(Di-imidazol-1-yl)-methyleneamine (0.08 g, 0.50 mmol), and the reaction was heated at 85° C. for 3 hours. The mixture was cooled to room temperature and partitioned between water and EtOAc. The aqueous layer was extracted with EtOAc, and the combined organic layers were dried over MgSO₄, filtered, and concentrated. The residue was purified on silica gel (EtOAc:hexane gradient) to give the desired product.

Mass spectrometry data for Compound 1-1, Compound 1-7, Compound 1-8, Compound 1-9, Compound 1-10, Compound 1-11, Compound 1-12, Compound 1-13, and Compound 1-14, is shown in Tables 1-4. NMR data for Compound 1-3 is shown below.

Notes:

For Compound 1-3, ¹H NMR (CDCl₃) δ 8.6 (d, 1H), 8.31 (d, 1H), 7.70 (m, 2H), 7.57 (d, 1H), 7.38 (d, 2H), 7.32 (d, 1H), 7.20 (m, 1H), 7.08 (d, 1H), 6.80 (m, 4H), 5.41 (s, 2H), 5.27 (s, 2H), 3.96 (t, 5H), 3.57 (t, 2H), 3.27 (s, 2H), 1.57-1.20 (m, 23H).
For Compound 1-7, during Step 3, hydrazide I-2 was converted to 1,3,4-oxadiazol-2-yl I-3 using triethyl orthoformate.
For Compound 1-8, i) hydrazide I-2 was made directly from ester I-4, ii) during Step 3, hydrazide I-2 was converted to 1,3,4-oxadiazol-2-yl I-3 using triethyl orthoformate.
For Compound 1-9, i) hydrazide I-2 was made directly from ester I-4, ii) during Step 3, hydrazide I-2 was converted to 1,3,4-oxadiazol-2-ylamine I-3 using cyanogen bromide and sodium bicarbonate.
For Compound 1-14, during Step 3, hydrazide I-2 was converted to 1,3,4-oxadiazol-2-ylamine I-3 using C-(di-imidazol-1-yl)-methyleneamine.

### Example 14: Exemplary Pharmaceutical Compositions that Could be Utilized

### Example 14a: Parenteral Composition

To prepare a parenteral pharmaceutical composition suitable for administration by injection, 100 mg of a water-soluble salt of a compound of any of Formula (G), Formula (G-I), or Formula (G-II), is dissolved in DMSO and then mixed with 10 mL of 0.9% sterile saline. The mixture is incorporated into a dosage unit form suitable for administration by injection.

### Example 14b: Oral Composition

To prepare a pharmaceutical composition for oral delivery, 100 mg of a compound of any of Formula (G), Formula (G-I), or Formula (G-II), is mixed with 750 mg of starch. The mixture is incorporated into an oral dosage unit for, such as a hard gelatin capsule, which is suitable for oral administration.

### Example 14c: Sublingual (Hard Lozenge) Composition

To prepare a pharmaceutical composition for buccal delivery, such as a hard lozenge, mix 100 mg of a compound of any of Formula (G), Formula (G-I), or Formula (G-II), with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 14d: Inhalation Composition

To prepare a pharmaceutical composition for inhalation delivery, 20 mg of a compound of any of Formula (G), Formula (G-I), or Formula (G-II), is mixed with 50 mg of anhydrous citric acid and 100 mL of 0.9% sodium chloride solution. The mixture is incorporated into an inhalation delivery unit, such as a nebulizer, which is suitable for inhalation administration.

### Example 14e: Rectal Gel Composition

To prepare a pharmaceutical composition for rectal delivery, 100 mg of a compound of any of Formula (G), Formula (G-I), or Formula (G-II), is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 14f: Topical Gel Composition

To prepare a pharmaceutical topical gel composition, 100 mg of a compound of any of Formula (G), Formula (G-I), or Formula (G-II), is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 14g: Ophthalmic Solution Composition

To prepare a pharmaceutical opthalmic solution composition, 100 mg of a compound of any of Formula (G), Formula (G-I), or Formula (G-II), is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Additional Examples of Methods of Reducing Pro-Inflammatory HDL

In the following example methods listed below (identified as example methods 15-1 to 15-34), any one or more features from one method is contemplated to be able to be combined with any one or more features of another method where such combination is possible without detriment to the method. Thus, multiple combinations are possible that are not shown expressly.

15-1. An example method for reducing pro-inflammatory HDL in a human comprising administering to the human a therapeutically effective amount of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof such that the pro-inflammatory HDL level is reduced.
15-2. An example method according to example method 15-1, wherein the reduction of pro-inflammatory HDL includes at least a portion of the pro-inflammatory HDL being converted to anti-inflammatory HDL.
15-3. An example method according to example method 15-2, further comprising: determining prior to said administering that the human has a need to have pro-inflammatory HDL levels reduced.
15-4. An example method according to example method 15-3, further comprising: determining after said administering if an amount of pro-inflammatory HDL in the human has been converted to an amount of anti-inflammatory HDL.
15-5. An example method according to example method 15-4, further comprising: administering to the human a pharmaceutically acceptable excipient.
15-6. An example method according to example method 15-3, further comprising: administering to the human a pharmaceutically acceptable excipient.
15-7. An example method according to example method 15-2, further comprising: determining after said administering if an amount of pro-inflammatory HDL in the human has been converted to an amount of anti-inflammatory HDL.
15-8. An example method according to example method 15-7, further comprising: administering to the human a pharmaceutically acceptable excipient.
15-9. An example method according to example method 15-2, further comprising: administering to the human a pharmaceutically acceptable excipient.
15-10. An example method according to example method 15-1, further comprising: determining prior to said administering that the human has a need to have pro-inflammatory HDL levels reduced.
15-11. An example method according to example method 15-10, further comprising: determining after said administering if an amount of pro-inflammatory HDL in the human has been converted to an amount of anti-inflammatory HDL.
15-12. An example method according to example method 15-11, further comprising: administering to the human a pharmaceutically acceptable excipient.
15-13. An example method according to example method 15-10, further comprising: administering to the human a pharmaceutically acceptable excipient.
15-14. An example method according to example method 15-1, further comprising: determining after said administering if an amount of pro-inflammatory HDL in the human has been converted to an amount of anti-inflammatory HDL.
15-15. An example method according to example method 15-14, administering to the human a pharmaceutically acceptable excipient.
15-16. An example method according to one of example methods 15-1 to 15-14, wherein the leukotriene inhibitor is a 5-lipoxygenase-activating protein inhibitor.
15-17. An example method according to one of example methods 15-1 to 15-14, wherein the leukotriene inhibitor is a compound having the structure of Formula (G):

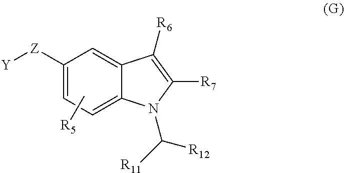

wherein,
   Z is selected from [C(R₁)₂]ₘ[C(R₂)₂]ₙ, [C(R₂)₂]ₙ[C(R₁)₂]ₘO, O[C(R₁)₂]ₘ [C(R₂)₂]ₙ, [C(R₂)₂]ₙO[C(R₁)₂]ₙ, or [C(R₁)₂]ₙO[C(R₂)₂]ₙ, wherein each R₁ is independently H, CF₃, or an optionally substituted lower alkyl and two R₁ on the same carbon may join to form a carbonyl (═O); and each R₂ is independently H, OH, OMe, CF₃, or an optionally substituted lower alkyl and two R₂ on the same carbon may join to form a carbonyl (═O); m is 0, 1 or 2; each n is independently 0, 1, 2, or 3;
   Y is H or -(substituted or unsubstituted aryl); or -(substituted or unsubstituted heteroaryl);
      where each substituent on Y or Z is (LₛRₛ)ⱼ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, substituted or unsubstituted C₁-C₆ alkyl, C₂-C₆ alkenyl, —C₁-C₆ fluoroalkyl, substituted or unsubstituted heteroaryl, substituted or unsubstituted aryl, or substituted or unsubstituted heterocycle; and each R₈ is independently selected from H, halogen, —N(R₄)₂, —CN, —NO₂, N₃, —S(═O)₂NH₂, substituted or unsubstituted lower alkyl, substituted or unsubstituted lower cycloalkyl, —C₁-C₆ fluoroalkyl, substituted or unsubstituted aryl, substituted or unsubstituted heteroaryl, or substituted or unsubstituted heteroalkyl; where j is 0, 1, 2, 3, or 4;
   R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(O), —CH(OH), -(substituted or unsubstituted C₁-C₆ alkyl), or -(substituted or unsubstituted C₂-C₆ alkenyl);
   R₇ is L₃-X-L₄-G₁, wherein,
      L₃ is a substituted or unsubstituted alkyl;
      X is a bond, O, —C(═O), —CR₉(OR₉), S, —S(═O), —S(═O)₂, —NR₉, —NR₉C(O), —C(O)NR₉, —NR₉C(O)NR₉—;
      L₄ is a bond, or a substituted or unsubstituted alkyl;
      G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O);
      or G₁ is W-G₅, where W is a substituted or unsubstituted aryl, substituted or unsubstituted heterocycle or substituted or unsubstituted heteroaryl and G₅ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈;
      each R₈ is independently selected from substituted or unsubstituted lower alkyl, substituted or unsubstituted lower cycloalkyl, substituted or unsubstituted phenyl or substituted or unsubstituted benzyl;
      each R₉ is independently selected from H, substituted or unsubstituted lower alkyl, substituted or unsubstituted lower cycloalkyl, substituted or unsubstituted phenyl or substituted or unsubstituted benzyl; or two R₉ groups can together form a 5-, 6-, 7-, or 8-membered heterocyclic ring; or R₈ and R₉ can together form a 5-, 6-, 7-, or 8-membered heterocyclic ring and
      each R₁₀ is independently selected from H, —S(═O)₂R₈, —S(═O)₂NH₂ —C(O)R₈, —CN, —NO₂, heteroaryl, or heteroalkyl;
   R₅ is H, halogen, substituted or unsubstituted C₁-C₆ alkyl, substituted or unsubstituted O—C₁-C₆ alkyl;
   R₁₁ is L₇-L₁₀-G₆, wherein L₇ is a bond, —C(O), —C(O)NH, —NHC(O), or (substituted or unsubstituted C₁-C₆ alkyl); L₁₀ is a bond, (substituted or unsubstituted alkyl), (substituted or unsubstituted cycloalkyl), (substituted or unsubstituted heteroaryl), (substituted or unsubstituted aryl), or (substituted or unsubstituted heterocycle); G₆ is OR₉, —C(═O)R₉, —C(═O)OR₉, —SR₈, —S(═O)R₈, —S(═O)₂R₈, N(R₉)₂, tetrazolyl, —NHS(═O)₂R₈, —S(═O)₂N(R₉)₂, —C(O)NHS(═O)₂R₈, —S(═O)₂NHC(O)R₉, —C(═O)N(R₉)₂, N R₉C(O)R₉, C(R₉)₂C(═O)N(R₉)₂—C(═NR₁₀)N(R₉)₂, —NR₉C(═NR₁₀)N(R₉)₂, —NR₉C(═CR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O)
      or G₆ is W-G₇, wherein W is (substituted or unsubstituted heterocycle), (substituted or unsubstituted aryl) or a (substituted or unsubstituted heteroaryl) and G₇ is H, halogen, CN, NO₂, N₃, CF₃, OCF₃, C₁-C₆ alkyl, C₃-C₆ cycloalkyl, —C₁-C₆ fluoroalkyl, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycle), or -L₅-(substituted or unsubstituted aryl), wherein L₅ is a bond, —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O);
      provided that R₁₁ comprises at least one (unsubstituted or substituted) aromatic moiety and at least one (unsubstituted or substituted) cyclic moiety, wherein the (unsubstituted or substituted) cyclic moiety is a (unsubstituted or substituted) heterocyclic group or a (unsubstituted or substituted) heteroaryl group and R₁₁ is not a thienyl-phenyl group;
   R₁₂ is H, (substituted or unsubstituted C₁-C₆ alkyl), (substituted or unsubstituted C₃-C₆ cycloalkyl);
   or active metabolite, or solvate, or pharmaceutically acceptable salt, or a pharmaceutically acceptable prodrug thereof.
      15-18. An example method according to one of example methods 15-17, wherein Z is [C(R₂)₂]ₙC(R₁)₂O.
      15-19. An example method according to one of example methods 15-18, wherein Y is -(substituted or unsubstituted heteroaryl) or -(substituted or unsubstituted aryl) and G₆ is W-G₇.
      15-20. An example method according to one of example methods 15-17, wherein Y is -(substituted or unsubstituted heteroaryl).
      15-21. An example method according to one of example methods 15-20, wherein Y is selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, imidazo[1,2-a]pyridinyl, thiophenopyridinyl and furopyridinyl, wherein Y is substituted or unsubstituted.
      15-22. An example method according to one of example method 15-21, wherein Y is selected from the group consisting of pyridinyl or quinolinyl, wherein Y is substituted or unsubstituted.
      15-23. An example method according to one of example method 15-17, wherein R₆ is L₂-(substituted or unsubstituted alkyl), or L₂-(substituted or unsubstituted cycloalkyl), L₂-(substituted or unsubstituted aryl), where L₂ is a bond, O, S, —S(O)₂, —C(O), or substituted or unsubstituted alkyl.
      15-24. An example method according to one of example method 15-17, wherein X is a bond, 0, —C(═O), —CR₉(OR₉), S, —S(═O), —S(═O)₂, —NR₉, —NR₉C(O), —C(O)NR₉.
      15-25. An example method according to one of example method 15-17, wherein G₁ is tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈.
      15-26. An example method according to one of example method 15-17, wherein L₃ is unsubstituted alkyl; X is a bond; L₄ is a bond; and G₁ is —C(O)OR₉.
      15-27. An example method according to one of example method 15-26, wherein R₉ is H or unsubstituted alkyl.
      15-28. An example method according to one of example method 15-17, wherein L₁₀ is a substituted or unsubstituted aryl substituted or unsubstituted heteroaryl and G₆ is W-G₇ wherein W is substituted or unsubstituted heteroaryl, substituted or unsubstituted heterocycle.
      15-29. An example method according to one of example method 15-28, wherein L₁₀ is a substituted or unsubstituted aryl.
      15-30. An example method according to one of example method 15-17, wherein L₃ is unsubstituted alkyl; X is a bond; L₄ is a bond; and G₁ is —OR₉.
      15-31. An example method according to one of example method 15-17, wherein G₁ is W-G₅, where W is a substituted or unsubstituted heterocycle or substituted or unsubstituted heteroaryl.
      15-32. An example method according to one of example methods 15-1 to 15-14, wherein the leukotriene inhibitor is a compound having the structure of Formula (Z1):

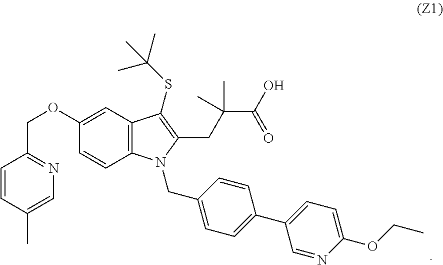 [SMILES: CCOc1ccc(-c2ccc(Cn3c(CC(C)(C)C(=O)O)c(SC(C)(C)C)c4cc(OCc5ccc(C)cn5)ccc43)cc2)cn1]

15-33. An example method according to one of example methods 15-1 to 15-14, wherein the leukotriene inhibitor is selected from the group consisting of 3-[3-tert-butylsulfanyl-1-(4-pyridin-2-yl-benzyl)-5-(pyridin-2-ylmethoxy)-1H-indol-2-yl]-2,2-dimethyl-propionamide (Compound 1-1); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid ethyl ester (Compound 1-2); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid 6-hydroxy-hexyl ester (Compound 1-3); 1-[3-tert-butylsulfanyl-1-(4-pyridin-2-yl-benzyl)-5-(pyridin-2-ylmethoxy)-1H-indol-2-yl]-2-methyl-propan-2-ol (Compound 1-4); 1-[3-tert-butylsulfanyl-1-(4-pyridin-3-yl-benzyl)-5-(pyridin-2-ylmethoxy)-1H-indol-2-yl]-2-methyl-propan-2-ol (Compound 1-5); 1-[3-tert-butylsulfanyl-5-(pyridin-2-ylmethoxy)-1-(4-thiazol-2-yl-benzyl)-1H-indol-2-yl]-2-methyl-propan-2-ol (Compound 1-6); 1-[3-tert-butylsulfanyl-1-(4-[1,3,4]oxadiazol-2-yl-benzyl)-5-(pyridin-2-ylmethoxy)-1H-indol-2-yl]-2-methyl-propan-2-ol (Compound 1-7); 3-tert-butylsulfanyl-1-[4-(6-methoxy-pyridin-3-yl)-benzyl]-2-(2-methyl-2-[1,3,4]oxadiazol-2-yl-propyl)-5-(pyridin-2-ylmethoxy)-1H-indole (Compound 1-8); 5-{2-[3-tert-butylsulfanyl-1-[4-(6-methoxy-pyridin-3-yl)-benzyl]-5-(pyridin-2-ylmethoxy)-1H-indol-2-yl]-1,1-dimethyl-ethyl}-[1,3,4]oxadiazol-2-ylamine (Compound 1-9); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-pyrazin-2-yl-propionamide (Compound 1-10); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-thiazol-2-yl-propionamide (Compound 1-11); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-pyridin-3-yl-propionamide (Compound 1-12); 3-[3-tert-butylsulfanyl-1-[4-(6-methoxy-pyridin-3-yl)-benzyl]-5-(pyridin-2-ylmethoxy)-1H-indol-2-yl]-N-(2-dimethylamino-ethyl)-2,2-dimethyl-propionamide (Compound 1-13); 5-{4-[3-tert-butylsulfanyl-2-(2,2-dimethyl-propyl)-5-(pyridin-2-ylmethoxy)-indol-1-ylmethyl]-phenyl}-[1,3,4]oxadiazol-2-ylamine (Compound 1-14); 3-[3-tert-butylsulfanyl-1-[4-(5-fluoro-pyridin-2-yl)-benzyl]-5-(quinolin-2-ylmethoxy)-1H-indol-2-yl]-N-(2-dimethylamino-ethyl)-2,2-dimethyl-propanoylguanidine (Compound 1-15); 3-[3-tert-butylsulfanyl-5-(pyridin-2-ylmethoxy)-1-(4-thiazol-2-yl-benzyl)-1H-indol-2-yl]-2,2-dimethyl-propionic acid (Compound 2-1); 3-[3-tert-butylsulfanyl-5-(pyridin-2-ylmethoxy)-1-(4-pyrimidin-2-yl-benzyl)-1H-indol-2-yl]-2,2-dimethyl-propionic acid (Compound 2-2); -[3-tert-butylsulfanyl-1-(4-pyridin-3-yl-benzyl)-5-(pyridin-2-ylmethoxy)-1H-indol-2-yl]-2,2-dimethyl-propionic acid (Compound 2-3); 3-[3-tert-butylsulfanyl-5-(pyridin-2-ylmethoxy)-1-(4-pyrimidin-5-yl-benzyl)-1H-indol-2-yl]-2,2-dimethyl-propionic acid (Compound 2-4); 3-[3-tert-butylsulfanyl-1-(4-pyrazin-2-yl-benzyl)-5-(pyridin-2-ylmethoxy)-1H-indol-2-yl]-2,2-dimethyl-propionic acid (Compound 2-5); 3-[3-tert-butylsulfanyl-1-[4-(6-methoxy-pyridazin-3-yl)-benzyl]-5-(pyridin-2-ylmethoxy)-1H-indol-2-yl]-2,2-dimethyl-propionic acid (Compound 2-6); 3-[1-[4-(5-amino-pyrazin-2-yl)-benzyl]-3-tert-butylsulfanyl-5-(pyridin-2-ylmethoxy)-1H-indol-2-yl]-2,2-dimethyl-propionic acid (Compound 2-7); 3-[3-(3,3-dimethyl-butyryl)-5-(pyridin-2-ylmethoxy)-1-(4-thiazol-2-yl-benzyl)-1H-indol-2-yl]-2,2-dimethyl-propionic acid (Compound 2-8); 2,2-dimethyl-3-[5-(pyridin-2-ylmethoxy)-1-(4-thiazol-2-yl-benzyl)-1H-indol-2-yl]-propionic acid (Compound 2-9); 3-[3-acetyl-5-(pyridin-2-ylmethoxy)-1-(4-thiazol-2-yl-benzyl)-1H-indol-2-yl]-2,2-dimethyl-propionic acid (Compound 2-10); 3-[1-[4-(6-methoxy-pyridazin-3-yl)-benzyl]-5-(pyridin-2-ylmethoxy)-1H-indol-2-yl]-2,2-dimethyl-propionic acid (Compound 2-11); 3-[3-acetyl-1-[4-(6-methoxy-pyridazin-3-yl)-benzyl]-5-(pyridin-2-ylmethoxy)-1H-indol-2-yl]-2,2-dimethyl-propionic acid (Compound 2-12); 3-[3-ethyl-1-[4-(6-methoxy-pyridazin-3-yl)-benzyl]-5-(pyridin-2-ylmethoxy)-1H-indol-2-yl]-2,2-dimethyl-propionic acid (Compound 2-13); 3-[3-(3,3-dimethyl-butyl)-5-(pyridin-2-ylmethoxy)-1-(4-thiazol-2-yl-benzyl)-1H-indol-2-yl]-2,2-dimethyl-propionic acid (Compound 2-14); 3-[3-cyclopropanecarbonyl-5-(pyridin-2-ylmethoxy)-1-(4-thiazol-2-yl-benzyl)-1H-indol-2-yl]-2,2-dimethyl-propionic acid (Compound 2-15); 3-[3-cyclobutanecarbonyl-5-(pyridin-2-ylmethoxy)-1-(4-thiazol-2-yl-benzyl)-1H-indol-2-yl]-2,2-dimethyl-propionic acid (Compound 2-16); 3-[3-tert-butylsulfanyl-1-[4-(6-hydroxy-pyridazin-3-yl)-benzyl]-5-(pyridin-2-ylmethoxy)-1H-indol-2-yl]-2,2-dimethyl-propionic acid (Compound 2-17); 3-[3-tert-butylsulfanyl-1-(4-pyridin-4-yl-benzyl)-5-(pyridin-2-ylmethoxy)-1H-indol-2-yl]-2,2-dimethyl-propionic acid (Compound 2-18); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid (Compound 2-19); 3-[3-tert-butylsulfanyl-1-[4-(6-methyl-pyridazin-3-yl)-benzyl]-5-(pyridin-2-ylmethoxy)-1H-indol-2-yl]-2,2-dimethyl-propionic acid (Compound 2-20); 3-[3-tert-butylsulfanyl-1-[4-(5-methyl-thiazol-2-yl)-benzyl]-5-(pyridin-2-ylmethoxy)-1H-indol-2-yl]-2,2-dimethyl-propionic acid (Compound 2-21); 3-[3-cyclobutylmethyl-5-(pyridin-2-ylmethoxy)-1-(4-thiazol-2-yl-benzyl)-1H-indol-2-yl]-2,2-dimethyl-propionic acid (Compound 2-22); 3-[3-tert-butylsulfanyl-1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-23); 3-[3-tert-butylsulfanyl-5-(2-methyl-thiazol-4-ylmethoxy)-1-(4-thiazol-2-yl-benzyl)-1H-indol-2-yl]-2,2-dimethyl-propionic acid (Compound 2-24); 2,2-dimethyl-3-[5-(2-methyl-thiazol-4-ylmethoxy)-1-(4-thiazol-2-yl-benzyl)-1H-indol-2-yl]-propionic acid (Compound 2-25); 3-[3-(3,3-dimethyl-butyryl)-5-(2-methyl-thiazol-4-ylmethoxy)-1-(4-thiazol-2-yl-benzyl)-1H-indol-2-yl]-2,2-dimethyl-propionic acid (Compound 2-26); 3-[1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-27); 3-[3-(3,3-dimethyl-butyryl)-1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-28); 3-[3-ethyl-5-(pyridin-2-ylmethoxy)-1-(4-thiazol-2-yl-benzyl)-1H-indol-2-yl]-2,2-dimethyl-propionic acid (Compound 2-29); 3-{5-(benzothiazol-2-ylmethoxy)-3-tert-butylsulfanyl-1-[4-(6-methoxy-pyridazin-3-yl)-benzyl]-1H-indol-2-yl}-2,2-dimethyl-propionic acid (Compound 2-30); 3-[3-tert-butylsulfanyl-5-(2-methyl-thiazol-4-ylmethoxy)-1-(4-pyrimidin-2-yl-benzyl)-1H-indol-2-yl]-2,2-dimethyl-propionic acid (Compound 2-31); 3-[5-(benzothiazol-2-ylmethoxy)-3-tert-butylsulfanyl-1-(4-pyrimidin-2-yl-benzyl)-1H-indol-2-yl]-2,2-dimethyl-propionic acid (Compound 2-32); 3-[3-tert-butylsulfanyl-1-[4-(2-methyl-3-pyridin-2-ylmethyl-3H-imidazol-4-yl)-benzyl]-5-(pyridin-2-ylmethoxy)-1H-indol-2-yl]-2,2-dimethyl-propionic acid (Compound 2-33); 3-[3-tert-butylsulfanyl-1-[4-(2,4-dimethyl-thiazol-5-yl)-benzyl]-5-(pyridin-2-ylmethoxy)-1H-indol-2-yl]-2,2-dimethyl-propionic acid (Compound 2-34); 3-[3-tert-butylsulfanyl-1-[4-(5-fluoro-thiazol-2-yl)-benzyl]-5-(pyridin-2-ylmethoxy)-1H-indol-2-yl]-2,2-dimethyl-propionic acid (Compound 2-35); 3-[3-tert-butylsulfanyl-1-[4-(4-methyl-thiazol-2-yl)-benzyl]-5-(pyridin-2-ylmethoxy)-1H-indol-2-yl]-2,2-dimethyl-propionic acid (Compound 2-39); 3-[3-tert-butylsulfanyl-1-[4-(3,5-dimethyl-isoxazol-4-yl)-benzyl]-5-(pyridin-2-ylmethoxy)-1H-indol-2-yl]-2,2-dimethyl-propionic acid (Compound 2-41); 3-[3-tert-butylsulfanyl-1-[4-(3-methyl-3H-imidazol-4-yl)-benzyl]-5-(pyridin-2-ylmethoxy)-1H-indol-2-yl]-2,2-dimethyl-propionic acid (Compound 2-43); 3-[3-tert-butylsulfanyl-1-[4-(5-methoxy-pyridin-2-yl)-benzyl]-5-(pyridin-2-ylmethoxy)-1H-indol-2-yl]-2,2-dimethyl-propionic acid (Compound 2-47); 3-[3-tert-butylsulfanyl-5-(pyridin-2-ylmethoxy)-1-(4-[1,3,4]thiadiazol-2-yl-benzyl)-1H-indol-2-yl]-2,2-dimethyl-propionic acid (Compound 2-55); 3-[3-tert-butylsulfanyl-1-[4-(6-hydroxy-pyridin-3-yl)-benzyl]-5-(pyridin-2-ylmethoxy)-1H-indol-2-yl]-2,2-dimethyl-propionic acid (Compound 2-62); 3-[3-tert-butylsulfanyl-1-[4-(6-cyano-pyridin-3-yl)-benzyl]-5-(pyridin-2-ylmethoxy)-1H-indol-2-yl]-2,2-dimethyl-propionic acid (Compound 2-64); 3-{3-tert-butylsulfanyl-5-(pyridin-2-ylmethoxy)-1-[4-(6-trifluoromethyl-pyridin-3-yl)-benzyl]-1H-indol-2-yl}-2,2-dimethyl-propionic acid (Compound 2-65); 3-[3-tert-butylsulfanyl-1-[4-(2-methoxy-pyrimidin-5-yl)-benzyl]-5-(pyridin-2-ylmethoxy)-1H-indol-2-yl]-2,2-dimethyl-propionic acid (Compound 2-67); 3-[3-tert-butylsulfanyl-1-[4-(2-methoxy-thiazol-4-yl)-benzyl]-5-(pyridin-2-ylmethoxy)-1H-indol-2-yl]-2,2-dimethyl-propionic acid (Compound 2-68); 3-[3-tert-butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-73); 3-{3-tert-butylsulfanyl-5-(5-ethyl-pyridin-2-ylmethoxy)-1-[4-(4-methoxy-pyridin-2-yl)-benzyl]-1H-indol-2-yl}-2,2-dimethyl-propionic acid (Compound 2-76); 3-[3-tert-butylsulfanyl-1-[4-(4-methoxy-pyridin-2-yl)-benzyl]-5-(quinolin-2-ylmethoxy)-1H-indol-2-yl]-2,2-dimethyl-propionic acid (Compound 2-77); 3-{3-tert-butylsulfanyl-5-(6-fluoro-quinolin-2-ylmethoxy)-1-[4-(4-methoxy-pyridin-2-yl)-benzyl]-1H-indol-2-yl}-2,2-dimethyl-propionic acid (Compound 2-78); 3-[3-tert-butylsulfanyl-1-[4-(3-fluoro-pyridin-2-yl)-benzyl]-5-(5-methyl-pyridin-2-ylmethoxy)-1H-indol-2-yl]-2,2-dimethyl-propionic acid (Compound 2-82); 3-{3-tert-butylsulfanyl-5-(5-ethyl-pyridin-2-ylmethoxy)-1-[4-(3-fluoro-pyridin-2-yl)-benzyl]-1H-indol-2-yl}-2,2-dimethyl-propionic acid (Compound 2-84); 3-[3-tert-butylsulfanyl-1-[4-(3-fluoro-pyridin-2-yl)-benzyl]-5-(quinolin-2-ylmethoxy)-1H-indol-2-yl]-2,2-dimethyl-propionic acid (Compound 2-85); 3-[3-tert-butylsulfanyl-1-[4-(5-carbamoyl-pyridin-2-yl)-benzyl]-5-(pyridin-2-ylmethoxy)-1H-indol-2-yl]-2,2-dimethyl-propionic acid (Compound 2-87); 3-[3-tert-butylsulfanyl-1-[4-(5-cyano-pyridin-2-yl)-benzyl]-5-(pyridin-2-ylmethoxy)-1H-indol-2-yl]-2,2-dimethyl-propionic acid (Compound 2-88); 3-[3-tert-butylsulfanyl-1-[4-(5-methoxy-thiazol-2-yl)-benzyl]-5-(pyridin-2-ylmethoxy)-1H-indol-2-yl]-2,2-dimethyl-propionic acid (Compound 2-89); 3-[3-tert-butylsulfanyl-1-[4-(6-methyl-pyridin-3-yl)-benzyl]-5-(pyridin-2-ylmethoxy)-1H-indol-2-yl]-2,2-dimethyl-propionic acid (Compound 2-90); 3-{3-tert-butylsulfanyl-5-(pyridin-2-ylmethoxy)-1-[4-(5-trifluoromethyl-pyridin-2-yl)-benzyl]-1H-indol-2-yl}-2,2-dimethyl-propionic acid (Compound 2-91); 3-[3-tert-butylsulfanyl-1-[4-(2-ethoxy-thiazol-4-yl)-benzyl]-5-(pyridin-2-ylmethoxy)-1H-indol-2-yl]-2,2-dimethyl-propionic acid (Compound 2-92); 3-[3-tert-butylsulfanyl-1-[4-(4-methyl-1H-imidazol-2-yl)-benzyl]-5-(pyridin-2-ylmethoxy)-1H-indol-2-yl]-2,2-dimethyl-propionic acid (Compound 2-93); 3-[3-tert-butylsulfanyl-1-[4-(6-ethoxy-pyridin-3-yl)-benzyl]-5-(pyridin-2-ylmethoxy)-1H-indol-2-yl]-2,2-dimethyl-propionic acid (Compound 2-94); 3-[3-tert-butylsulfanyl-1-[4-(6-methoxy-pyridin-2-yl)-benzyl]-5-(pyridin-2-ylmethoxy)-1H-indol-2-yl]-2,2-dimethyl-propionic acid (Compound 2-95); 3-[3-tert-butylsulfanyl-1-[4-(5-methoxy-pyridin-3-yl)-benzyl]-5-(pyridin-2-ylmethoxy)-1H-indol-2-yl]-2,2-dimethyl-propionic acid (Compound 2-96); 3-[3-tert-butylsulfanyl-1-[4-(6-carbamoyl-pyridin-3-yl)-benzyl]-5-(pyridin-2-ylmethoxy)-1H-indol-2-yl]-2,2-dimethyl-propionic acid (Compound 2-97); 3-[3-tert-butylsulfanyl-1-[4-(5-methyl-pyridin-2-yl)-benzyl]-5-(pyridin-2-ylmethoxy)-1H-indol-2-yl]-2,2-dimethyl-propionic acid (Compound 2-98); 3-{3-tert-butylsulfanyl-5-(6-fluoro-pyridin-2-ylmethoxy)-1-[4-(6-methoxy-pyridin-3-yl)-benzyl]-1H-indol-2-yl}-2,2-dimethyl-propionic acid (Compound 2-99); 3-[3-tert-butylsulfanyl-1-[4-(6-methoxy-pyridin-3-yl)-benzyl]-5-(6-methoxy-pyridin-2-ylmethoxy)-1H-indol-2-yl]-2,2-dimethyl-propionic acid (Compound 2-100); 3-[3-tert-butylsulfanyl-1-[4-(6-methoxy-pyridin-3-yl)-benzyl]-5-(6-methyl-pyridin-2-ylmethoxy)-1H-indol-2-yl]-2,2-dimethyl-propionic acid (Compound 2-101); 3-{3-tert-butylsulfanyl-5-(5-methyl-pyridin-2-ylmethoxy)-1-[4-(6-trifluoromethyl-pyridin-3-yl)-benzyl]-1H-indol-2-yl}-2,2-dimethyl-propionic acid (Compound 2-102); 3-{3-tert-butylsulfanyl-5-(5-methyl-pyridin-2-ylmethoxy)-1-[4-(5-trifluoromethyl-pyridin-2-yl)-benzyl]-1H-indol-2-yl}-2,2-dimethyl-propionic acid (Compound 2-103); 3-{3-tert-butylsulfanyl-5-(6-cyclopropyl-pyridin-2-ylmethoxy)-1-[4-(6-methoxy-pyridin-3-yl)-benzyl]-1H-indol-2-yl}-2,2-dimethyl-propionic acid (Compound 2-104); 3-[3-tert-butylsulfanyl-1-[4-(5-methyl-pyridin-2-yl)-benzyl]-5-(5-methyl-pyridin-2-ylmethoxy)-1H-indol-2-yl]-2,2-dimethyl-propionic acid (Compound 2-105); 3-[3-tert-butylsulfanyl-1-[4-(6-methoxy-pyridazin-3-yl)-benzyl]-5-(5-methyl-pyridin-2-ylmethoxy)-1H-indol-2-yl]-2,2-dimethyl-propionic acid (Compound 2-106); 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid (Compound 2-107); 3-{3-tert-butylsulfanyl-5-(5-chloro-pyridin-2-ylmethoxy)-1-[4-(6-methoxy-pyridin-3-yl)-benzyl]-1H-indol-2-yl}-2,2-dimethyl-propionic acid (Compound 2-108); 3-{3-tert-butylsulfanyl-5-((S)-1-pyridin-2-yl-ethoxy)-1-[4-(5-trifluoromethyl-pyridin-2-yl)-benzyl]-1H-indol-2-yl}-2,2-dimethyl-propionic acid (Compound 2-109); 3-{3-tert-butylsulfanyl-5-((R)-1-pyridin-2-yl-ethoxy)-1-[4-(5-trifluoromethyl-pyridin-2-yl)-benzyl]-1H-indol-2-yl}-2,2-dimethyl-propionic acid (Compound 2-110); 3-[3-tert-butylsulfanyl-1-[4-(6-methoxy-pyridin-3-yl)-benzyl]-5-((S)-1-pyridin-2-yl-ethoxy)-1H-indol-2-yl]-2,2-dimethyl-propionic acid (Compound 2-111); 3-[3-tert-butylsulfanyl-1-[4-(6-methoxy-pyridin-3-yl)-benzyl]-5-((R)-1-pyridin-2-yl-ethoxy)-1H-indol-2-yl]-2,2-dimethyl-propionic acid (Compound 2-112); 3-[3-tert-butylsulfanyl-1-[4-(6-methoxy-pyridin-2-yl)-benzyl]-5-((S)-1-pyridin-2-yl-ethoxy)-1H-indol-2-yl]-2,2-dimethyl-propionic acid (Compound 2-113); 3-[3-tert-butylsulfanyl-1-[4-(6-methoxy-pyridin-2-yl)-benzyl]-5-((R)-1-pyridin-2-yl-ethoxy)-1H-indol-2-yl]-2,2-dimethyl-propionic acid (Compound 2-114); 3-[3-tert-butylsulfanyl-1-[4-(2-ethoxy-thiazol-4-yl)-benzyl]-5-((S)-1-pyridin-2-yl-ethoxy)-1H-indol-2-yl]-2,2-dimethyl-propionic acid (Compound 2-115); 3-[3-tert-butylsulfanyl-1-[4-(2-ethoxy-thiazol-4-yl)-benzyl]-5-((R)-1-pyridin-2-yl-ethoxy)-1H-indol-2-yl]-2,2-dimethyl-propionic acid (Compound 2-116); 3-[3-tert-butylsulfanyl-1-[4-(6-methoxy-pyridin-3-yl)-benzyl]-5-(3-methyl-pyridin-2-ylmethoxy)-1H-indol-2-yl]-2,2-dimethyl-propionic acid (Compound 2-117); 3-{3-tert-butylsulfanyl-5-(3-methyl-pyridin-2-ylmethoxy)-1-[4-(5-trifluoromethyl-pyridin-2-yl)-benzyl]-1H-indol-2-yl}-2,2-dimethyl-propionic acid (Compound 2-118); 3-{3-tert-butylsulfanyl-5-(3,5-dimethyl-pyridin-2-ylmethoxy)-1-[4-(6-methoxy-pyridin-3-yl)-benzyl]-1H-indol-2-yl}-2,2-dimethyl-propionic acid (Compound 2-119); 3-{3-tert-butylsulfanyl-5-(3,5-dimethyl-pyridin-2-ylmethoxy)-1-[4-(5-trifluoromethyl-pyridin-2-yl)-benzyl]-1H-indol-2-yl}-2,2-dimethyl-propionic acid (Compound 2-120); 3-{5-(benzothiazol-2-ylmethoxy)-3-tert-butylsulfanyl-1-[4-(6-methoxy-pyridin-3-yl)-benzyl]-1H-indol-2-yl}-2,2-dimethyl-propionic acid (Compound 2-121); 3-{5-(benzothiazol-2-ylmethoxy)-3-tert-butylsulfanyl-1-[4-(5-methoxy-pyridin-2-yl)-benzyl]-1H-indol-2-yl}-2,2-dimethyl-propionic acid (Compound 2-122); 3-{5-(benzothiazol-2-ylmethoxy)-3-cyclobutanecarbonyl-1-[4-(6-methoxy-pyridin-3-yl)-benzyl]-1H-indol-2-yl}-2,2-dimethyl-propionic acid (Compound 2-123); 3-{5-(benzothiazol-2-ylmethoxy)-3-cyclobutylmethyl-1-[4-(6-methoxy-pyridin-3-yl)-benzyl]-1H-indol-2-yl}-2,2-dimethyl-propionic acid (Compound 2-124); 3-{3-tert-butylsulfanyl-5-(5-ethyl-pyridin-2-ylmethoxy)-1-[4-(6-methoxy-pyridin-3-yl)-benzyl]-1H-indol-2-yl}-2,2-dimethyl-propionic acid (Compound 2-125); 3-[3-tert-butylsulfanyl-1-[4-(6-ethoxy-pyridin-3-yl)-benzyl]-5-(5-ethyl-pyridin-2-ylmethoxy)-1H-indol-2-yl]-2,2-dimethyl-propionic acid (Compound 2-126); 3-{3-tert-butylsulfanyl-5-(5-ethyl-pyridin-2-ylmethoxy)-1-[4-(6-trifluoromethyl-pyridin-3-yl)-benzyl]-1H-indol-2-yl}-2,2-dimethyl-propionic acid (Compound 2-127); 3-{3-tert-butylsulfanyl-5-(5-ethyl-pyridin-2-ylmethoxy)-1-[4-(5-trifluoromethyl-pyridin-2-yl)-benzyl]-1H-indol-2-yl}-2,2-dimethyl-propionic acid (Compound 2-128); 3-[3-tert-butylsulfanyl-1-[4-(2-ethoxy-thiazol-4-yl)-benzyl]-5-(5-methyl-pyridin-2-ylmethoxy)-1H-indol-2-yl]-2,2-dimethyl-propionic acid (Compound 2-129); 3-[3-tert-butylsulfanyl-1-[4-(2-methoxy-thiazol-4-yl)-benzyl]-5-(5-methyl-pyridin-2-ylmethoxy)-1H-indol-2-yl]-2,2-dimethyl-propionic acid (Compound 2-130); 3-[3-tert-butylsulfanyl-1-[4-(6-methoxy-pyridin-2-yl)-benzyl]-5-(5-methyl-pyridin-2-ylmethoxy)-1H-indol-2-yl]-2,2-dimethyl-propionic acid (Compound 2-131); 3-[3-cyclobutylmethyl-1-[4-(6-methoxy-pyridin-3-yl)-benzyl]-5-(pyridin-2-ylmethoxy)-1H-indol-2-yl]-2,2-dimethyl-propionic acid (Compound 2-132); 3-[3-cyclobutylmethyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-133); 3-[3-isobutyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-134); 3-[3-tert-butylsulfanyl-1-[4-(6-methoxy-pyridin-3-yl)-benzyl]-5-(quinolin-2-ylmethoxy)-1H-indol-2-yl]-2,2-dimethyl-propionic acid (Compound 2-135); 3-{3-tert-butylsulfanyl-5-(quinolin-2-ylmethoxy)-1-[4-(6-trifluoromethyl-pyridin-3-yl)-benzyl]-1H-indol-2-yl}-2,2-dimethyl-propionic acid (Compound 2-136); 3-{3-tert-butylsulfanyl-5-(quinolin-2-ylmethoxy)-1-[4-(5-trifluoromethyl-pyridin-2-yl)-benzyl]-H-indol-2-yl}-2,2-dimethyl-propionic acid (Compound 2-137); 3-[3-tert-butylsulfanyl-1-[4-(6-methoxy-pyridazin-3-yl)-benzyl]-5-(quinolin-2-ylmethoxy)-1H-indol-2-yl]-2,2-dimethyl-propionic acid (Compound 2-138); 3-[3-tert-butylsulfanyl-1-[4-(6-ethoxy-pyridin-3-yl)-benzyl]-5-(quinolin-2-ylmethoxy)-1H-indol-2-yl]-2,2-dimethyl-propionic acid (Compound 2-139); 3-{3-tert-butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic acid (Compound 2-140); 3-{3-tert-butylsulfanyl-5-(6-fluoro-quinolin-2-ylmethoxy)-1-[4-(6-methoxy-pyridin-3-yl)-benzyl]-1H-indol-2-yl}-2,2-dimethyl-propionic acid (Compound 2-141); 3-[3-tert-butylsulfanyl-1-[4-(2-ethoxy-thiazol-4-yl)-benzyl]-5-(6-fluoro-quinolin-2-ylmethoxy)-1H-indol-2-yl]-2,2-dimethyl-propionic acid (Compound 2-142); 3-{3-tert-butylsulfanyl-5-(6-fluoro-quinolin-2-ylmethoxy)-1-[4-(5-trifluoromethyl-pyridin-2-yl)-benzyl]-11H-indol-2-yl}-2,2-dimethyl-propionic acid (Compound 2-143); 3-{3-tert-butylsulfanyl-5-(7-fluoro-quinolin-2-ylmethoxy)-1-[4-(6-trifluoromethyl-pyridin-3-yl)-benzyl]-1H-indol-2-yl}-2,2-dimethyl-propionic acid (Compound 2-144); 3-{3-tert-butylsulfanyl-5-(7-fluoro-quinolin-2-ylmethoxy)-1-[4-(5-trifluoromethyl-pyridin-2-yl)-benzyl]-1H-indol-2-yl}-2,2-dimethyl-propionic acid (Compound 2-145); 3-{3-tert-butylsulfanyl-5-(7-fluoro-quinolin-2-ylmethoxy)-1-[4-(6-methoxy-pyridin-3-yl)-benzyl]-1H-indol-2-yl}-2,2-dimethyl-propionic acid (Compound 2-146); 3-[3-tert-butylsulfanyl-1-[4-(6-ethoxy-pyridin-3-yl)-benzyl]-5-(7-fluoro-quinolin-2-ylmethoxy)-1H-indol-2-yl]-2,2-dimethyl-propionic acid (Compound 2-147); 3-[3-tert-butylsulfanyl-1-[4-(3-fluoro-pyridin-2-yl)-benzyl]-5-(6-fluoro-quinolin-2-ylmethoxy)-1H-indol-2-yl]-2,2-dimethyl-propionic acid (Compound 2-148); 3-{3-tert-butylsulfanyl-5-(5-methyl-pyridin-2-ylmethoxy)-1-[4-(3-trifluoromethyl-pyridin-2-yl)-benzyl]-1H-indol-2-yl}-2,2-dimethyl-propionic acid (Compound 2-149); 3-{3-tert-butylsulfanyl-5-(5-ethyl-pyridin-2-ylmethoxy)-1-[4-(3-trifluoromethyl-pyridin-2-yl)-benzyl]-1H-indol-2-yl}-2,2-dimethyl-propionic acid (Compound 2-150); 3-{3-tert-butylsulfanyl-5-(quinolin-2-ylmethoxy)-1-[4-(3-trifluoromethyl-pyridin-2-yl)-benzyl]-1H-indol-2-yl}-2,2-dimethyl-propionic acid (Compound 2-151); 3-{3-tert-butylsulfanyl-5-(6-fluoro-quinolin-2-ylmethoxy)-1-[4-(3-trifluoromethyl-pyridin-2-yl)-benzyl]-1H-indol-2-yl}-2,2-dimethyl-propionic acid (Compound 2-156); 3-[3-tert-butylsulfanyl-1-[4-(6-ethoxy-pyridin-3-yl)-benzyl]-5-(3-methyl-pyridin-2-ylmethoxy)-1H-indol-2-yl]-2,2-dimethyl-propionic acid (Compound 2-157); 3-{3-tert-butylsulfanyl-5-(3-methyl-pyridin-2-ylmethoxy)-1-[4-(6-trifluoromethyl-pyridin-3-yl)-benzyl]-1H-indol-2-yl}-2,2-dimethyl-propionic acid (Compound 2-158); 3-{3-tert-butylsulfanyl-5-(3,5-dimethyl-pyridin-2-ylmethoxy)-1-[4-(6-ethoxy-pyridin-3-yl)-benzyl]-1H-indol-2-yl}-2,2-dimethyl-propionic acid (Compound 2-159); 3-[3-tert-butylsulfanyl-1-[4-(6-methoxy-pyridin-3-yl)-benzyl]-5-(4-methyl-pyridin-2-ylmethoxy)-1H-indol-2-yl]-2,2-dimethyl-propionic acid (Compound 2-160); 3-[3-tert-butylsulfanyl-1-[4-(6-ethoxy-pyridin-3-yl)-benzyl]-5-(4-methyl-pyridin-2-ylmethoxy)-1H-indol-2-yl]-2,2-dimethyl-propionic acid (Compound 2-161); 3-{3-tert-butylsulfanyl-5-(4-methyl-pyridin-2-ylmethoxy)-1-[4-(5-trifluoromethyl-pyridin-2-yl)-benzyl]-1H-indol-2-yl}-2,2-dimethyl-propionic acid (Compound 2-162); 3-{3-cyclobutylmethyl-5-(5-methyl-pyridin-2-ylmethoxy)-1-[4-(5-trifluoromethyl-pyridin-2-yl)-benzyl]-1H-indol-2-yl}-2,2-dimethyl-propionic acid (Compound 2-163); 3-[3-tert-butylsulfanyl-1-[4-(6-ethoxy-pyridin-3-yl)-benzyl]-5-(6-fluoro-quinolin-2-ylmethoxy)-1H-indol-2-yl]-2,2-dimethyl-propionic acid (Compound 2-164); 3-{3-tert-butylsulfanyl-5-(6-fluoro-quinolin-2-ylmethoxy)-1-[4-(6-trifluoromethyl-pyridin-3-yl)-benzyl]-1H-indol-2-yl}-2,2-dimethyl-propionic acid (Compound 2-165); 3-[3-tert-butylsulfanyl-1-[4-(6-methoxy-pyridin-3-yl)-benzyl]-5-(6-methyl-quinolin-2-ylmethoxy)-1H-indol-2-yl]-2,2-dimethyl-propionic acid (Compound 2-166); 3-{3-tert-butylsulfanyl-5-(6-methyl-quinolin-2-ylmethoxy)-1-[4-(5-trifluoromethyl-pyridin-2-yl)-benzyl]-1H-indol-2-yl}-2,2-dimethyl-propionic acid (Compound 2-167); 3-[3-tert-butylsulfanyl-1-[4-(6-methyl-pyridazin-3-yl)-benzyl]-5-(quinolin-2-ylmethoxy)-1H-indol-2-yl]-2,2-dimethyl-propionic acid (Compound 2-168); 3-[3-tert-butylsulfanyl-1-[4-(6-ethoxy-pyridazin-3-yl)-benzyl]-5-(quinolin-2-ylmethoxy)-1H-indol-2-yl]-2,2-dimethyl-propionic acid (Compound 2-169); 3-[3-isobutyl-1-[4-(6-methoxy-pyridin-3-yl)-benzyl]-5-(quinolin-2-ylmethoxy)-1H-indol-2-yl]-2,2-dimethyl-propionic acid (Compound 2-170); 3-{3-tert-butylsulfanyl-5-(6-fluoro-quinolin-2-ylmethoxy)-1-[4-(6-methoxy-pyridazin-3-yl)-benzyl]-1H-indol-2-yl}-2,2-dimethyl-propionic acid (Compound 2-171); 3-[1-[4-(6-methoxy-pyridin-3-yl)-benzyl]-3-(2-methyl-propane-2-sulfonyl)-5-(pyridin-2-ylmethoxy)-1H-indol-2-yl]-2,2-dimethyl-propionic acid (Compound 2-172); 3-[1-[4-(6-methoxy-pyridin-3-yl)-benzyl]-3-(2-methyl-propane-2-sulfinyl)-5-(pyridin-2-ylmethoxy)-1H-indol-2-yl]-2,2-dimethyl-propionic acid (Compound 2-173); 3-[3-tert-butylsulfanyl-1-[4-(6-methoxy-pyridin-3-yl)-benzyl]-5-(1-oxy-pyridin-2-ylmethoxy)-1H-indol-2-yl]-2,2-dimethyl-propionic acid (Compound 2-174); 3-{3-tert-butylsulfanyl-5-(imidazo[1,2-a]pyridin-2-ylmethoxy)-1-[4-(6-methoxy-pyridin-3-yl)-benzyl]-1H-indol-2-yl}-2,2-dimethyl-propionic acid (Compound 2-175); 3-[3-tert-butylsulfanyl-1-[4-(6-ethoxy-pyridin-3-yl)-benzyl]-5-(imidazo[1,2-a]pyridin-2-ylmethoxy)-1H-indol-2-yl]-2,2-dimethyl-propionic acid (Compound 2-176); 3-{3-tert-butylsulfanyl-5-(imidazo[1,2-a]pyridin-2-ylmethoxy)-1-[4-(5-trifluoromethyl-pyridin-2-yl)-benzyl]-1H-indol-2-yl}-2,2-dimethyl-propionic acid (Compound 2-177); 3-[3-tert-butylsulfanyl-1-[4-(6-ethoxy-pyridin-3-yl)-benzyl]-5-((R)-1-pyridin-2-yl-ethoxy)-1H-indol-2-yl]-2,2-dimethyl-propionic acid (Compound 2-178); 3-{3-tert-butylsulfanyl-5-(6-fluoro-quinolin-2-ylmethoxy)-1-[4-(6-methyl-pyridazin-3-yl)-benzyl]-1H-indol-2-yl}-2,2-dimethyl-propionic acid (Compound 2-179); 3-[3-tert-butylsulfanyl-1-[4-(6-methoxy-pyridin-3-yl)-benzyl]-5-(5-methyl-isoxazol-3-ylmethoxy)-1H-indol-2-yl]-2,2-dimethyl-propionic acid (Compound 2-180); 3-[3-tert-butylsulfanyl-1-[4-(6-ethoxy-pyridin-3-yl)-benzyl]-5-(5-methyl-isoxazol-3-ylmethoxy)-1H-indol-2-yl]-2,2-dimethyl-propionic acid (Compound 2-181); 3-{3-tert-butylsulfanyl-5-(5-methyl-isoxazol-3-ylmethoxy)-1-[4-(5-trifluoromethyl-pyridin-2-yl)-benzyl]-1H-indol-2-yl}-2,2-dimethyl-propionic acid (Compound 2-182); 3-{3-tert-Butylsulfanyl-5-(2,5-dimethyl-2H-pyrazol-3-ylmethoxy)-1-[4-(6-methoxy-pyridin-3-yl)-benzyl]-1H-indol-2-yl}-2,2-dimethyl-propionic acid (Compound 2-183); 3-{3-tert-butylsulfanyl-5-(1,5-dimethyl-1H-pyrazol-3-ylmethoxy)-1-[4-(6-methoxy-pyridin-3-yl)-benzyl]-1H-indol-2-yl}-2,2-dimethyl-propionic acid (Compound 2-184); 3-[3-tert-butylsulfanyl-1-[4-(6-ethoxy-pyridazin-3-yl)-benzyl]-5-(6-fluoro-quinolin-2-ylmethoxy)-1H-indol-2-yl]-2,2-dimethyl-propionic acid (Compound 2-185); 3-[3-tert-butylsulfanyl-1-[4-(6-ethoxy-pyridazin-3-yl)-benzyl]-5-(5-ethyl-pyridin-2-ylmethoxy)-1H-indol-2-yl]-2,2-dimethyl-propionic acid (Compound 2-186); 3-{3-tert-butylsulfanyl-5-(5-ethyl-pyridin-2-ylmethoxy)-1-[4-(6-methoxy-pyridazin-3-yl)-benzyl]-1H-indol-2-yl}-2,2-dimethyl-propionic acid (Compound 2-187); 3-[3-tert-butylsulfanyl-1-[4-(5-fluoro-pyridin-2-yl)-benzyl]-5-(6-fluoro-quinolin-2-ylmethoxy)-1H-indol-2-yl]-2,2-dimethyl-propionic acid (Compound 2-188); 3-[3-tert-butylsulfanyl-1-[4-(5-fluoro-pyridin-2-yl)-benzyl]-5-((R)-1-pyridin-2-yl-ethoxy)-1H-indol-2-yl]-2,2-dimethyl-propionic acid (Compound 2-189); 3-[3-tert-butylsulfanyl-1-[4-(6-ethoxy-pyridin-2-yl)-benzyl]-5-(6-fluoro-quinolin-2-ylmethoxy)-1H-indol-2-yl]-2,2-dimethyl-propionic acid (Compound 2-190); 3-[3-tert-butylsulfanyl-1-[4-(6-ethoxy-pyridin-2-yl)-benzyl]-5-((R)-1-pyridin-2-yl-ethoxy)-1H-indol-2-yl]-2,2-dimethyl-propionic acid (Compound 2-191); 3-[3-tert-butylsulfanyl-1-[4-(5-fluoro-pyridin-2-yl)-benzyl]-5-(5-methyl-pyridin-2-ylmethoxy)-1H-indol-2-yl]-2,2-dimethyl-propionic acid (Compound 2-192); 3-[3-tert-butysulfanyl-1-[4-(6-ethoxy-pyridin-2-yl)-benzyl]-5-(5-methyl-pyridin-2-ylmethoxy)-1H-indol-2-yl]-2,2-dimethyl-propionic acid (Compound 2-193); 3-{5-(6-fluoro-quinolin-2-ylmethoxy)-3-isobutyl-1-[4-(6-trifluoromethyl-pyridin-3-yl)-benzyl]-1H-indol-2-yl}-2,2-dimethyl-propionic acid (Compound 2-194); 3-{3-tert-butylsulfanyl-5-(pyridin-2-ylmethoxy)-1-[3-(5-trifluoromethyl-pyridin-2-yl)-benzyl]-1H-indol-2-yl}-2,2-dimethyl-propionic acid (Compound 2-195); 3-[3-tert-butylsulfanyl-1-[3-(6-methoxy-pyridin-3-yl)-benzyl]-5-(pyridin-2-ylmethoxy)-1H-indol-2-yl]-2,2-dimethyl-propionic acid (Compound 2-196); 3-[3-tert-butylsulfanyl-1-[4-(5-fluoro-pyridin-2-yl)-benzyl]-5-(quinolin-2-ylmethoxy)-1H-indol-2-yl]-2,2-dimethyl-propionic acid (Compound 2-197); 3-[3-tert-butylsulfanyl-1-[4-(6-ethoxy-pyridin-2-yl)-benzyl]-5-(quinolin-2-ylmethoxy)-1H-indol-2-yl]-2,2-dimethyl-propionic acid (Compound 2-198); 3-[3-tert-butylsulfanyl-1-[4-(6-ethoxy-pyridin-2-yl)-benzyl]-5-(pyridin-2-ylmethoxy)-1H-indol-2-yl]-2,2-dimethyl-propionic acid (Compound 2-199); 3-{3-tert-butylsulfanyl-5-(6-fluoro-quinolin-2-ylmethoxy)-1-[4-(6-trifluoromethyl-pyridin-2-yl)-benzyl]-1H-indol-2-yl}-2,2-dimethyl-propionic acid (Compound 2-200); 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid (Compound 2-201); 3-{3-tert-butylsulfanyl-5-(5-methyl-pyridin-2-ylmethoxy)-1-[4-(6-trifluoromethyl-pyridin-2-yl)-benzyl]-1H-indol-2-yl}-2,2-dimethyl-propionic acid (Compound 2-202); 3-{3-tert-butylsulfanyl-5-(quinolin-2-ylmethoxy)-1-[4-(6-trifluoromethyl-pyridin-2-yl)-benzyl]-1H-indol-2-yl}-2,2-dimethyl-propionic acid (Compound 2-203); 3-{3-tert-butylsulfanyl-5-(pyridin-2-ylmethoxy)-1-[4-(6-trifluoromethyl-pyridin-2-yl)-benzyl]-1H-indol-2-yl}-2,2-dimethyl-propionic acid (Compound 2-204); 3-[3-tert-butylsulfanyl-5-(quinolin-2-ylmethoxy)-1-(4-thiazol-2-yl-benzyl)-1H-indol-2-yl]-2,2-dimethyl-propionic acid (Compound 2-205); 3-[3-tert-butylsulfanyl-1-[3-(4-methoxy-tetrahydro-pyran-4-yl)-benzyl]-5-(pyridin-2-ylmethoxy)-1H-indol-2-yl]-2,2-dimethyl-propionic acid (Compound 2-206); 3-[3-tert-butylsulfanyl-5-(6-fluoro-quinolin-2-ylmethoxy)-1-(4-pyridin-2-yl-benzyl)-1H-indol-2-yl]-2,2-dimethyl-propionic acid (Compound 2-207); 3-[3-tert-butylsulfanyl-5-(5-ethyl-pyridin-2-ylmethoxy)-1-(4-pyridin-3-yl-benzyl)-1H-indol-2-yl]-2,2-dimethyl-propionic acid (Compound 2-208); 3-[3-tert-butylsulfanyl-1-(4-pyridin-3-yl-benzyl)-5-(quinolin-2-ylmethoxy)-1H-indol-2-yl]-2,2-dimethyl-propionic acid (Compound 2-209); 3-[3-tert-butylsulfanyl-5-(6-fluoro-quinolin-2-ylmethoxy)-1-(4-pyridin-3-yl-benzyl)-1H-indol-2-yl]-2,2-dimethyl-propionic acid (Compound 2-210); 3-[3-tert-butylsulfanyl-5-(5-methyl-pyridin-2-ylmethoxy)-1-(4-pyridin-2-yl-benzyl)-1H-indol-2-yl]-2,2-dimethyl-propionic acid (Compound 2-211); 3-[3-tert-butylsulfanyl-5-(5-ethyl-pyridin-2-ylmethoxy)-1-(4-pyridin-2-yl-benzyl)-1H-indol-2-yl]-2,2-dimethyl-propionic acid (Compound 2-212); 3-[3-tert-butylsulfanyl-1-(4-pyridin-2-yl-benzyl)-5-(quinolin-2-ylmethoxy)-1H-indol-2-yl]-2,2-dimethyl-propionic acid (Compound 2-213); 3-[3-tert-butylsulfanyl-5-(5-methyl-pyridin-2-ylmethoxy)-1-(4-pyridin-3-yl-benzyl)-1H-indol-2-yl]-2,2-dimethyl-propionic acid (Compound 2-214); 3-[3-tert-butylsulfanyl-1-[4-(4-methoxy-pyridin-2-yl)-benzyl]-5-(5-methyl-pyridin-2-ylmethoxy)-1H-indol-2-yl]-2,2-dimethyl-propionic acid (Compound 2-215; 3-[3-tert-butylsulfanyl-5-(quinolin-2-ylmethoxy)-1-(4-(3-methyoxypyridin-2-yl)-benzyl)-1H-indol-2-yl]-2,2-dimethyl-propionic acid (Compound 2-216); 3-[3-tert-butylsulfanyl-5-(5-methyl-pyridin-2-ylmethoxy)-1-(4-(3-methyoxypyridin-2-yl)-benzyl)-1H-indol-2-yl]-2,2-dimethyl-propionic acid (Compound 2-217); 3-[3-tert-butylsulfanyl-5-(5-ethyl-pyridin-2-ylmethoxy)-1-(4-(3-methyoxypyridin-2-yl)-benzyl)-1H-indol-2-yl]-2,2-dimethyl-propionic acid (Compound 2-218); 3-[3-tert-butylsulfanyl-5-(5-methyl-pyridin-2-ylmethoxy)-1-(4-(4-trifluoromethypyridin-2-yl)-benzyl)-1H-indol-2-yl]-2,2-dimethyl-propionic acid (Compound 2-219); 3-[3-tert-butylsulfanyl-5-(5-ethyl-pyridin-2-ylmethoxy)-1-(4-(4-trifluoromethypyridin-2-yl)-benzyl)-1H-indol-2-yl]-2,2-dimethyl-propionic acid (Compound 2-220); 3-[3-tert-butylsulfanyl-5-(quinolin-2-ylmethoxy)-1-(4-(4-trifluoromethypyridin-2-yl)-benzyl)-1H-indol-2-yl]-2,2-dimethyl-propionic acid (Compound 2-221); 3-[3-tert-butylsulfanyl-5-(5-methyl-pyridin-2-ylmethoxy)-1-(4-(5-fluoropyridin-3-yl)-benzyl)-1H-indol-2-yl]-2,2-dimethyl-propionic acid (Compound 2-222); 3-[3-tert-butylsulfanyl-5-(5-ethyl-pyridin-2-ylmethoxy)-1-(4-(5-fluoropyridin-3-yl)-benzyl)-1H-indol-2-yl]-2,2-dimethyl-propionic acid (Compound 2-223); 3-[3-tert-butylsulfanyl-5-(quinolin-2-ylmethoxy)-1-(4-(5-fluoropyridin-3-yl)-benzyl)-1H-indol-2-yl]-2,2-dimethyl-propionic acid (Compound 2-224); 3-[3-tert-butylsulfanyl-5-(2,3-dimethyl-pyridin-6-ylmethoxy)-1-(4-(2-methoxypyridin-5-yl)-benzyl)-1H-indol-2-yl]-2,2-dimethyl-propionic acid (Compound 2-225); 3-[3-tert-butylsulfanyl-5-(2,3-dimethyl-pyridin-6-ylmethoxy)-1-(4-(3-trifluoromethylpyridin-2-yl)-benzyl)-1H-indol-2-yl]-2,2-dimethyl-propionic acid (Compound 2-226); 3-[3-tert-butylsulfanyl-5-(2,3-dimethyl-pyridin-6-ylmethoxy)-1-(4-(4-trifluoromethylpyridin-2-yl)-benzyl)-1H-indol-2-yl]-2,2-dimethyl-propionic acid (Compound 2-227); 3-[3-tert-butylsulfanyl-5-(2,3-dimethyl-pyridin-6-ylmethoxy)-1-(4-(3-fluoropyridin-2-yl)-benzyl)-1H-indol-2-yl]-2,2-dimethyl-propionic acid (Compound 2-228); 3-[3-tert-butylsulfanyl-5-(2,3-dimethyl-pyridin-6-ylmethoxy)-1-(4-(5-fluoropyridin-3-yl)-benzyl)-1H-indol-2-yl]-2,2-dimethyl-propionic acid (Compound 2-229); 3-[3-tert-butylsulfanyl-5-(2,3-dimethyl-pyridin-6-ylmethoxy)-1-(4-(4-methoxypyridin-2-yl)-benzyl)-1H-indol-2-yl]-2,2-dimethyl-propionic acid (Compound 2-230); 3-[3-tert-butylsulfanyl-5-(2,3-dimethyl-pyridin-6-ylmethoxy)-1-(4-(pyridin-2-yl)-benzyl)-1H-indol-2-yl]-2,2-dimethyl-propionic acid (Compound 2-231); 3-[3-tert-butylsulfanyl-5-(5-methyl-pyridin-2-ylmethoxy)-1-(4-(2-methoxy-pyridin-3-yl)-benzyl)-1H-indol-2-yl]-2,2-dimethyl-propionic acid (Compound 2-232); 3-[3-tert-butylsulfanyl-5-(5-ethyl-pyridin-2-ylmethoxy)-1-(4-(2-methoxy-pyridin-3-yl)-benzyl)-1H-indol-2-yl]-2,2-dimethyl-propionic acid (Compound 2-233); 3-[3-tert-butylsulfanyl-5-(quinolin-2-ylmethoxy)-1-(4-(2-methoxy-pyridin-3-yl)-benzyl)-1H-indol-2-yl]-2,2-dimethyl-propionic acid (Compound 2-234); 3-[3-tert-butylsulfanyl-1-(6′-methoxy-[2,3′]bipyridinyl-5-ylmethyl)-5-(pyridin-2-ylmethoxy)-1H-indol-2-yl]-2,2-dimethyl-propionic acid (Compound 3-1); 3-[3-tert-butylsulfanyl-1-[6-(4-methoxy-phenyl)-pyridin-3-ylmethyl]-5-(pyridin-2-ylmethoxy)-1H-indol-2-yl]-2,2-dimethyl-propionic acid (Compound 3-2); 3-{3-tert-butylsulfanyl-5-(pyridin-2-ylmethoxy)-1-[6-(4-trifluoromethoxy-phenyl)-pyridin-3-ylmethyl]-1H-indol-2-yl}-2,2-dimethyl-propionic acid (Compound 3-3); 3-[3-tert-butylsulfanyl-1-[5-(4-methoxy-phenyl)-pyridin-2-ylmethyl]-5-(pyridin-2-ylmethoxy)-1H-indol-2-yl]-2,2-dimethyl-propionic acid (Compound 3-4); 3-{3-tert-butylsulfanyl-5-(pyridin-2-ylmethoxy)-1-[5-(4-trifluoromethoxy-phenyl)-pyridin-2-ylmethyl]-1H-indol-2-yl}-2,2-dimethyl-propionic acid (Compound 3-5); 3-{3-tert-butylsulfanyl-5-isopropyl-1-[4-(6-methoxy-pyridin-3-yl)-benzyl]-1H-indol-2-yl}-2,2-dimethyl-propionic acid (Compound 4-1); 3-{3-tert-butylsulfanyl-5-hydroxy-1-[4-(6-methoxy-pyridin-3-yl)-benzyl]-1H-indol-2-yl}-2,2-dimethyl-propionic acid (Compound 4-2).
15-34. An example method according to one of example methods 15-1 to 15-14, wherein the leukotriene inhibitor is 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid.

In any of the example methods (e.g., example methods 15-1 to 15-34) the human to which a leukotriene inhibitor is administered may be suffering from a chronic inflammatory disease or condition, such as for example, a disease or condition selected from systemic lupus erythematosus, rheumatoid arthritis, systemic sclerosis, pulmonary arterial hypertension, asthma, gout, sleep apnea, and any combinations thereof. In any of the example methods (e.g., example methods 15-1 to 15-34), a leukotriene inhibitor may be present in the form of a salt, such as for example a sodium salt.

In general, the systems, methods, compounds, compositions, etc. of the present invention have been exemplified by various exemplary embodiments and implementations as shown in the accompanying drawings and as described above. However, it should be understood that the presentation of these embodiments and implementations should not be construed as requiring that: 1) these embodiments and implementations stand in isolation from one another; 2) that individual components, features, aspects, and/or functionalities described relative to each one of the embodiments and implementations cannot be used independently of the corresponding embodiment or implementation; and 3) that individual components, features, aspects, and/or functionalities described cannot be used individually in connection with other embodiments and implementations, either described herein or derivable therefrom, alone and/or in any combination with one another. On the contrary, those skilled in the art will appreciate that the individual components, features, aspects, and functionalities of a particular embodiment or implementation can, as appropriate under the circumstances, be utilized alone and in any subcombination with other components, features, aspects, and/or functionalities of that particular embodiment or implementation and with any other embodiment or implementation, including the specific examples described herein.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method for reducing pro-inflammatory HDL in a human comprising administering to the human a therapeutically effective amount of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof such that the pro-inflammatory HDL level is reduced.

2. A method according to claim 1, wherein the reduction of pro-inflammatory HDL includes at least a portion of the pro-inflammatory HDL being converted to anti-inflammatory HDL.

3. A method according to claim 1, wherein the leukotriene inhibitor is a 5-lipoxygenase-activating protein inhibitor.

4. A method according to claim 1, further comprising: determining prior to said administering that the human has a need to have pro-inflammatory HDL levels reduced.

5. A method according to claim 1, further comprising: determining after said administering if an amount of pro-inflammatory HDL in the human has been converted to an amount of anti-inflammatory HDL.

6. (canceled)

7. A method according to claim 1, wherein the leukotriene inhibitor is a compound having the structure of Formula (G): 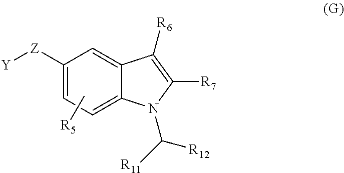
wherein,
Z is selected from [C(R₁)₂]ₘ[C(R₂)₂]ₙ, [C(R₂)₂]ₙ[C(R₁)₂]ₘO, O[C(R₁)₂]ₘ [C(R₂)₂]ₙ, [C(R₂)₂]ₙO[C(R₁)₂]ₙ, or [C(R₁)₂]ₙO[C(R₂)₂]ₙ, wherein each R₁ is independently H, CF₃, or an optionally substituted lower alkyl and two R₁ on the same carbon may join to form a carbonyl (═O); and each R₂ is independently H, OH, OMe, CF₃, or an optionally substituted lower alkyl and two R₂ on the same carbon may join to form a carbonyl (═O); m is 0, 1 or 2; each n is independently 0, 1, 2, or 3;
Y is H or -(substituted or unsubstituted aryl); or -(substituted or unsubstituted heteroaryl);
where each substituent on Y or Z is (LₛRₛ)ⱼ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, substituted or unsubstituted C₁-C₆ alkyl, C₂-C₆ alkenyl, —C₁-C₆ fluoroalkyl, substituted or unsubstituted heteroaryl, substituted or unsubstituted aryl, or substituted or unsubstituted heterocycle; and each R₅ is independently selected from H, halogen, —N(R₄)₂, —CN, —NO₂, N₃, —S(═O)₂NH₂, substituted or unsubstituted lower alkyl, substituted or unsubstituted lower cycloalkyl, —C₁-C₆ fluoroalkyl, substituted or unsubstituted aryl, substituted or unsubstituted heteroaryl, or substituted or unsubstituted heteroalkyl; where j is 0, 1, 2, 3, or 4;
R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(O), —CH(OH), -(substituted or unsubstituted C₁-C₆ alkyl), or -(substituted or unsubstituted C₂-C₆ alkenyl);
R₇ is L₃-X-L₄-G₁, wherein,
L₃ is a substituted or unsubstituted alkyl;
X is a bond, O, —C(═O), —CR₉(OR₉), S, —S(═O), —S(═O)₂, —NR₉, —NR₉C(O), —C(O)NR₉, —NR₉C(O)NR₉—;
L₄ is a bond, or a substituted or unsubstituted alkyl;
G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O);
or G₁ is W-G₅, where W is a substituted or unsubstituted aryl, substituted or unsubstituted heterocycle or substituted or unsubstituted heteroaryl and G₅ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈;
each R₈ is independently selected from substituted or unsubstituted lower alkyl, substituted or unsubstituted lower cycloalkyl, substituted or unsubstituted phenyl or substituted or unsubstituted benzyl;
each R₉ is independently selected from H, substituted or unsubstituted lower alkyl, substituted or unsubstituted lower cycloalkyl, substituted or unsubstituted phenyl or substituted or unsubstituted benzyl; or two R₉ groups can together form a 5-, 6-, 7-, or 8-membered heterocyclic ring; or R₈ and R₉ can together form a 5-, 6-, 7-, or 8-membered heterocyclic ring and
each R₁₀ is independently selected from H, —S(═O)₂R₈, —S(═O)₂NH₂ —C(O)R₈, —CN, —NO₂, heteroaryl, or heteroalkyl;
R₅ is H, halogen, substituted or unsubstituted C₁-C₆ alkyl, substituted or unsubstituted O—C₁-C₆ alkyl;
R₁₁ is L₇-L₁₀-G₆, wherein L₇ is a bond, —C(O), —C(O)NH, —NHC(O), or (substituted or unsubstituted C₁-C₆ alkyl); L₁₀ is a bond, (substituted or unsubstituted alkyl), (substituted or unsubstituted cycloalkyl), (substituted or unsubstituted heteroaryl), (substituted or unsubstituted aryl), or (substituted or unsubstituted heterocycle);
G₆ is OR₉, —C(═O)R₉, —C(═O)OR₉, —SR₈, —S(═O)R₈, —S(═O)₂R₈, N(R₉)₂, tetrazolyl, —NHS(═O)₂R₈, —S(═O)₂N(R₉)₂, —C(O)NHS(═O)₂R₈, —S(═O)₂NHC(O)R₉, —C(═O)N(R₉)₂, N R₉C(O)R₉, C(R₉)₂C(═O)N(R₉)₂—C(═NR₁₀)N(R₉)₂, —NR₉C(═NR₁₀)N(R₉)₂, —NR₉C(═CR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O)
or G₆ is W-G₇, wherein W is (substituted or unsubstituted heterocycle), (substituted or unsubstituted aryl) or a (substituted or unsubstituted heteroaryl) and G₇ is H, halogen, CN, NO₂, N₃, CF₃, OCF₃, C₁-C₆ alkyl, C₃-C₆ cycloalkyl, —C₁-C₆ fluoroalkyl, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycle), or -L₅-(substituted or unsubstituted aryl), wherein L₅ is a bond, —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O);
provided that R₁₁ comprises at least one (unsubstituted or substituted) aromatic moiety and at least one (unsubstituted or substituted) cyclic moiety, wherein the (unsubstituted or substituted) cyclic moiety is a (unsubstituted or substituted) heterocyclic group or a (unsubstituted or substituted) heteroaryl group and R₁₁ is not a thienyl-phenyl group;
R₁₂ is H, (substituted or unsubstituted C₁-C₆ alkyl), (substituted or unsubstituted C₃-C₆ cycloalkyl);
or active metabolite, or solvate, or pharmaceutically acceptable salt, or a pharmaceutically acceptable prodrug thereof.

8. A method according to claim 7, wherein Z is [C(R₂)₂]ₙC(R₁)₂O.

9. A method according to claim 8, wherein Y is -(substituted or unsubstituted heteroaryl) or -(substituted or unsubstituted aryl) and G₆ is W-G₇.

10. A method according to claim 7, wherein Y is -(substituted or unsubstituted heteroaryl).

11. (canceled)

12. (canceled)

13. A method according to claim 7, wherein R₆ is L₂-(substituted or unsubstituted alkyl), or L₂-(substituted or unsubstituted cycloalkyl), L₂-(substituted or unsubstituted aryl), where L₂ is a bond, O, S, —S(O)₂, —C(O), or substituted or unsubstituted alkyl.

14. (canceled)

15. (canceled)

16. (canceled)

17. (canceled)

18. (canceled)

19. (canceled)

20. (canceled)

21. (canceled)

22. A method according to claim 1, wherein the leukotriene inhibitor is a compound having the structure of Formula (Z1):

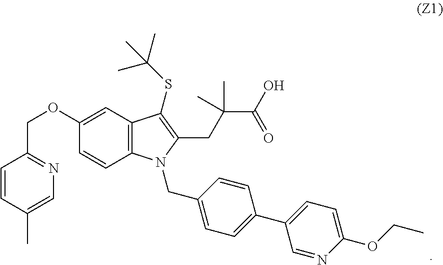 [SMILES: CCOc1ccc(-c2ccc(Cn3c(CC(C)(C)C(=O)O)c(SC(C)(C)C)c4cc(OCc5ccc(C)cn5)ccc43)cc2)cn1]

23. A method according to claim 1, wherein the leukotriene inhibitor is selected from the group consisting of 3-[3-tert-butylsulfanyl-1-(4-pyridin-2-yl-benzyl)-5-(pyridin-2-ylmethoxy)-1H-indol-2-yl]-2,2-dimethyl-propionamide (Compound 1-1); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid ethyl ester (Compound 1-2); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid 6-hydroxy-hexyl ester (Compound 1-3); 1-[3-tert-butylsulfanyl-1-(4-pyridin-2-yl-benzyl)-5-(pyridin-2-ylmethoxy)-1H-indol-2-yl]-2-methyl-propan-2-ol (Compound 1-4); 1-[3-tert-butylsulfanyl-1-(4-pyridin-3-yl-benzyl)-5-(pyridin-2-ylmethoxy)-1H-indol-2-yl]-2-methyl-propan-2-ol (Compound 1-5); 1-[3-tert-butylsulfanyl-5-(pyridin-2-ylmethoxy)-1-(4-thiazol-2-yl-benzyl)-1H-indol-2-yl]-2-methyl-propan-2-ol (Compound 1-6); 1-[3-tert-butylsulfanyl-1-(4-[1,3,4]oxadiazol-2-yl-benzyl)-5-(pyridin-2-ylmethoxy)-1H-indol-2-yl]-2-methyl-propan-2-ol (Compound 1-7); 3-tert-butylsulfanyl-1-[4-(6-methoxy-pyridin-3-yl)-benzyl]-2-(2-methyl-2-[1,3,4]oxadiazol-2-yl-propyl)-5-(pyridin-2-ylmethoxy)-1H-indole (Compound 1-8); 5-{2-[3-tert-butylsulfanyl-1-[4-(6-methoxy-pyridin-3-yl)-benzyl]-5-(pyridin-2-ylmethoxy)-1H-indol-2-yl]-1,1-dimethyl-ethyl}-[1,3,4]oxadiazol-2-ylamine (Compound 1-9); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-pyrazin-2-yl-propionamide (Compound 1-10); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-thiazol-2-yl-propionamide (Compound 1-11); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-N-pyridin-3-yl-propionamide (Compound 1-12); 3-[3-tert-butylsulfanyl-1-[4-(6-methoxy-pyridin-3-yl)-benzyl]-5-(pyridin-2-ylmethoxy)-1H-indol-2-yl]-N-(2-dimethylamino-ethyl)-2,2-dimethyl-propionamide (Compound 1-13); 5-{4-[3-tert-butylsulfanyl-2-(2,2-dimethyl-propyl)-5-(pyridin-2-ylmethoxy)-indol-1-ylmethyl]-phenyl}-[1,3,4]oxadiazol-2-ylamine (Compound 1-14); 3-[3-tert-butylsulfanyl-1-[4-(5-fluoro-pyridin-2-yl)-benzyl]-5-(quinolin-2-ylmethoxy)-1H-indol-2-yl]-N-(2-dimethylamino-ethyl)-2,2-dimethyl-propanoylguanidine (Compound 1-15); 3-[3-tert-butylsulfanyl-5-(pyridin-2-ylmethoxy)-1-(4-thiazol-2-yl-benzyl)-1H-indol-2-yl]-2,2-dimethyl-propionic acid (Compound 2-1); 3-[3-tert-butylsulfanyl-5-(pyridin-2-ylmethoxy)-1-(4-pyrimidin-2-yl-benzyl)-1H-indol-2-yl]-2,2-dimethyl-propionic acid (Compound 2-2); -[3-tert-butylsulfanyl-1-(4-pyridin-3-yl-benzyl)-5-(pyridin-2-ylmethoxy)-1H-indol-2-yl]-2,2-dimethyl-propionic acid (Compound 2-3); 3-[3-tert-butylsulfanyl-5-(pyridin-2-ylmethoxy)-1-(4-pyrimidin-5-yl-benzyl)-1H-indol-2-yl]-2,2-dimethyl-propionic acid (Compound 2-4); 3-[3-tert-butylsulfanyl-1-(4-pyrazin-2-yl-benzyl)-5-(pyridin-2-ylmethoxy)-1H-indol-2-yl]-2,2-dimethyl-propionic acid (Compound 2-5); 3-[3-tert-butylsulfanyl-1-[4-(6-methoxy-pyridazin-3-yl)-benzyl]-5-(pyridin-2-ylmethoxy)-1H-indol-2-yl]-2,2-dimethyl-propionic acid (Compound 2-6); 3-[1-[4-(5-amino-pyrazin-2-yl)-benzyl]-3-tert-butylsulfanyl-5-(pyridin-2-ylmethoxy)-1H-indol-2-yl]-2,2-dimethyl-propionic acid (Compound 2-7); 3-[3-(3,3-dimethyl-butyryl)-5-(pyridin-2-ylmethoxy)-1-(4-thiazol-2-yl-benzyl)-1H-indol-2-yl]-2,2-dimethyl-propionic acid (Compound 2-8); 2,2-dimethyl-3-[5-(pyridin-2-ylmethoxy)-1-(4-thiazol-2-yl-benzyl)-1H-indol-2-yl]-propionic acid (Compound 2-9); 3-[3-acetyl-5-(pyridin-2-ylmethoxy)-1-(4-thiazol-2-yl-benzyl)-1H-indol-2-yl]-2,2-dimethyl-propionic acid (Compound 2-10); 3-[1-[4-(6-methoxy-pyridazin-3-yl)-benzyl]-5-(pyridin-2-ylmethoxy)-1H-indol-2-yl]-2,2-dimethyl-propionic acid (Compound 2-11); 3-[3-acetyl-1-[4-(6-methoxy-pyridazin-3-yl)-benzyl]-5-(pyridin-2-ylmethoxy)-1H-indol-2-yl]-2,2-dimethyl-propionic acid (Compound 2-12); 3-[3-ethyl-1-[4-(6-methoxy-pyridazin-3-yl)-benzyl]-5-(pyridin-2-ylmethoxy)-1H-indol-2-yl]-2,2-dimethyl-propionic acid (Compound 2-13); 3-[3-(3,3-dimethyl-butyl)-5-(pyridin-2-ylmethoxy)-1-(4-thiazol-2-yl-benzyl)-1H-indol-2-yl]-2,2-dimethyl-propionic acid (Compound 2-14); 3-[3-cyclopropanecarbonyl-5-(pyridin-2-ylmethoxy)-1-(4-thiazol-2-yl-benzyl)-1H-indol-2-yl]-2,2-dimethyl-propionic acid (Compound 2-15); 3-[3-cyclobutanecarbonyl-5-(pyridin-2-ylmethoxy)-1-(4-thiazol-2-yl-benzyl)-1H-indol-2-yl]-2,2-dimethyl-propionic acid (Compound 2-16); 3-[3-tert-butylsulfanyl-1-[4-(6-hydroxy-pyridazin-3-yl)-benzyl]-5-(pyridin-2-ylmethoxy)-1H-indol-2-yl]-2,2-dimethyl-propionic acid (Compound 2-17); 3-[3-tert-butylsulfanyl-1-(4-pyridin-4-yl-benzyl)-5-(pyridin-2-ylmethoxy)-1H-indol-2-yl]-2,2-dimethyl-propionic acid (Compound 2-18); 3-[3-tert-butylsulfanyl-1-[4-(6-methoxy-pyridin-3-yl)-benzyl]-5-(pyridin-2-ylmethoxy)-1H-indol-2-yl]-2,2-dimethyl-propionic acid (Compound 2-19); 3-[3-tert-butylsulfanyl-1-[4-(6-methyl-pyridazin-3-yl)-benzyl]-5-(pyridin-2-ylmethoxy)-1H-indol-2-yl]-2,2-dimethyl-propionic acid (Compound 2-20); 3-[3-tert-butylsulfanyl-1-[4-(5-methyl-thiazol-2-yl)-benzyl]-5-(pyridin-2-ylmethoxy)-1H-indol-2-yl]-2,2-dimethyl-propionic acid (Compound 2-21); 3-[3-cyclobutylmethyl-5-(pyridin-2-ylmethoxy)-1-(4-thiazol-2-yl-benzyl)-1H-indol-2-yl]-2,2-dimethyl-propionic acid (Compound 2-22); 3-[3-tert-butylsulfanyl-1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-23); 3-[3-tert-butylsulfanyl-5-(2-methyl-thiazol-4-ylmethoxy)-1-(4-thiazol-2-yl-benzyl)-1H-indol-2-yl]-2,2-dimethyl-propionic acid (Compound 2-24); 2,2-dimethyl-3-[5-(2-methyl-thiazol-4-ylmethoxy)-1-(4-thiazol-2-yl-benzyl)-1H-indol-2-yl]-propionic acid (Compound 2-25); 3-[3-(3,3-dimethyl-butyryl)-5-(2-methyl-thiazol-4-ylmethoxy)-1-(4-thiazol-2-yl-benzyl)-1H-indol-2-yl]-2,2-dimethyl-propionic acid (Compound 2-26); 3-[1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-27); 3-[3-(3,3-dimethyl-butyryl)-1-[4-(6-methoxy-pyridazin-3-yl)-benzyl]-5-(2-methyl-thiazol-4-ylmethoxy)-1H-indol-2-yl]-2,2-dimethyl-propionic acid (Compound 2-28); 3-[3-ethyl-5-(pyridin-2-ylmethoxy)-1-(4-thiazol-2-yl-benzyl)-1H-indol-2-yl]-2,2-dimethyl-propionic acid (Compound 2-29); 3-{5-(benzothiazol-2-ylmethoxy)-3-tert-butylsulfanyl-1-[4-(6-methoxy-pyridazin-3-yl)-benzyl]-1H-indol-2-yl}-2,2-dimethyl-propionic acid (Compound 2-30); 3-[3-tert-butylsulfanyl-5-(2-methyl-thiazol-4-ylmethoxy)-1-(4-pyrimidin-2-yl-benzyl)-1H-indol-2-yl]-2,2-dimethyl-propionic acid (Compound 2-31); 3-[5-(benzothiazol-2-ylmethoxy)-3-tert-butylsulfanyl-1-(4-pyrimidin-2-yl-benzyl)-1H-indol-2-yl]-2,2-dimethyl-propionic acid (Compound 2-32); 3-[3-tert-butylsulfanyl-1-[4-(2-methyl-3-pyridin-2-ylmethyl-3H-imidazol-4-yl)-benzyl]-5-(pyridin-2-ylmethoxy)-1H-indol-2-yl]-2,2-dimethyl-propionic acid (Compound 2-33); 3-[3-tert-butylsulfanyl-1-[4-(2,4-dimethyl-thiazol-5-yl)-benzyl]-5-(pyridin-2-ylmethoxy)-1H-indol-2-yl]-2,2-dimethyl-propionic acid (Compound 2-34); 3-[3-tert-butylsulfanyl-1-[4-(5-fluoro-thiazol-2-yl)-benzyl]-5-(pyridin-2-ylmethoxy)-1H-indol-2-yl]-2,2-dimethyl-propionic acid (Compound 2-35); 3-[3-tert-butylsulfanyl-1-[4-(4-methyl-thiazol-2-yl)-benzyl]-5-(pyridin-2-ylmethoxy)-1H-indol-2-yl]-2,2-dimethyl-propionic acid (Compound 2-39); 3-[3-tert-butylsulfanyl-1-[4-(3,5-dimethyl-isoxazol-4-yl)-benzyl]-5-(pyridin-2-ylmethoxy)-1H-indol-2-yl]-2,2-dimethyl-propionic acid (Compound 2-41); 3-[3-tert-butylsulfanyl-1-[4-(3-methyl-3H-imidazol-4-yl)-benzyl]-5-(pyridin-2-ylmethoxy)-1H-indol-2-yl]-2,2-dimethyl-propionic acid (Compound 2-43); 3-[3-tert-butylsulfanyl-1-[4-(5-methoxy-pyridin-2-yl)-benzyl]-5-(pyridin-2-ylmethoxy)-1H-indol-2-yl]-2,2-dimethyl-propionic acid (Compound 2-47); 3-[3-tert-butylsulfanyl-5-(pyridin-2-ylmethoxy)-1-(4-[1,3,4]thiadiazol-2-yl-benzyl)-1H-indol-2-yl]-2,2-dimethyl-propionic acid (Compound 2-55); 3-[3-tert-butylsulfanyl-1-[4-(6-hydroxy-pyridin-3-yl)-benzyl]-5-(pyridin-2-ylmethoxy)-1H-indol-2-yl]-2,2-dimethyl-propionic acid (Compound 2-62); 3-[3-tert-butylsulfanyl-1-[4-(6-cyano-pyridin-3-yl)-benzyl]-5-(pyridin-2-ylmethoxy)-1H-indol-2-yl]-2,2-dimethyl-propionic acid (Compound 2-64); 3-{3-tert-butylsulfanyl-5-(pyridin-2-ylmethoxy)-1-[4-(6-trifluoromethyl-pyridin-3-yl)-benzyl]-1H-indol-2-yl}-2,2-dimethyl-propionic acid (Compound 2-65); 3-[3-tert-butylsulfanyl-1-[4-(2-methoxy-pyrimidin-5-yl)-benzyl]-5-(pyridin-2-ylmethoxy)-1H-indol-2-yl]-2,2-dimethyl-propionic acid (Compound 2-67); 3-[3-tert-butylsulfanyl-1-[4-(2-methoxy-thiazol-4-yl)-benzyl]-5-(pyridin-2-ylmethoxy)-1H-indol-2-yl]-2,2-dimethyl-propionic acid (Compound 2-68); 3-[3-tert-butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-73); 3-{3-tert-butylsulfanyl-5-(5-ethyl-pyridin-2-ylmethoxy)-1-[4-(4-methoxy-pyridin-2-yl)-benzyl]-1H-indol-2-yl}-2,2-dimethyl-propionic acid (Compound 2-76); 3-[3-tert-butylsulfanyl-1-[4-(4-methoxy-pyridin-2-yl)-benzyl]-5-(quinolin-2-ylmethoxy)-1H-indol-2-yl]-2,2-dimethyl-propionic acid (Compound 2-77); 3-{3-tert-butylsulfanyl-5-(6-fluoro-quinolin-2-ylmethoxy)-1-[4-(4-methoxy-pyridin-2-yl)-benzyl]-1H-indol-2-yl}-2,2-dimethyl-propionic acid (Compound 2-78); 3-[3-tert-butylsulfanyl-1-[4-(3-fluoro-pyridin-2-yl)-benzyl]-5-(5-methyl-pyridin-2-ylmethoxy)-1H-indol-2-yl]-2,2-dimethyl-propionic acid (Compound 2-82); 3-{3-tert-butylsulfanyl-5-(5-ethyl-pyridin-2-ylmethoxy)-1-[4-(3-fluoro-pyridin-2-yl)-benzyl]-1H-indol-2-yl}-2,2-dimethyl-propionic acid (Compound 2-84); 3-[3-tert-butylsulfanyl-1-[4-(3-fluoro-pyridin-2-yl)-benzyl]-5-(quinolin-2-ylmethoxy)-1H-indol-2-yl]-2,2-dimethyl-propionic acid (Compound 2-85); 3-[3-tert-butylsulfanyl-1-[4-(5-carbamoyl-pyridin-2-yl)-benzyl]-5-(pyridin-2-ylmethoxy)-1H-indol-2-yl]-2,2-dimethyl-propionic acid (Compound 2-87); 3-[3-tert-butylsulfanyl-1-[4-(5-cyano-pyridin-2-yl)-benzyl]-5-(pyridin-2-ylmethoxy)-1H-indol-2-yl]-2,2-dimethyl-propionic acid (Compound 2-88); 3-[3-tert-butylsulfanyl-1-[4-(5-methoxy-thiazol-2-yl)-benzyl]-5-(pyridin-2-ylmethoxy)-1H-indol-2-yl]-2,2-dimethyl-propionic acid (Compound 2-89); 3-[3-tert-butylsulfanyl-1-[4-(6-methyl-pyridin-3-yl)-benzyl]-5-(pyridin-2-ylmethoxy)-1H-indol-2-yl]-2,2-dimethyl-propionic acid (Compound 2-90); 3-{3-tert-butylsulfanyl-5-(pyridin-2-ylmethoxy)-1-[4-(5-trifluoromethyl-pyridin-2-yl)-benzyl]-1H-indol-2-yl}-2,2-dimethyl-propionic acid (Compound 2-91); 3-[3-tert-butylsulfanyl-1-[4-(2-ethoxy-thiazol-4-yl)-benzyl]-5-(pyridin-2-ylmethoxy)-1H-indol-2-yl]-2,2-dimethyl-propionic acid (Compound 2-92); 3-[3-tert-butylsulfanyl-1-[4-(4-methyl-1H-imidazol-2-yl)-benzyl]-5-(pyridin-2-ylmethoxy)-1H-indol-2-yl]-2,2-dimethyl-propionic acid (Compound 2-93); 3-[3-tert-butylsulfanyl-1-[4-(6-ethoxy-pyridin-3-yl)-benzyl]-5-(pyridin-2-ylmethoxy)-1H-indol-2-yl]-2,2-dimethyl-propionic acid (Compound 2-94); 3-[3-tert-butylsulfanyl-1-[4-(6-methoxy-pyridin-2-yl)-benzyl]-5-(pyridin-2-ylmethoxy)-1H-indol-2-yl]-2,2-dimethyl-propionic acid (Compound 2-95); 3-[3-tert-butylsulfanyl-1-[4-(5-methoxy-pyridin-3-yl)-benzyl]-5-(pyridin-2-ylmethoxy)-1H-indol-2-yl]-2,2-dimethyl-propionic acid (Compound 2-96); 3-[3-tert-butylsulfanyl-1-[4-(6-carbamoyl-pyridin-3-yl)-benzyl]-5-(pyridin-2-ylmethoxy)-1H-indol-2-yl]-2,2-dimethyl-propionic acid (Compound 2-97); 3-[3-tert-butylsulfanyl-1-[4-(5-methyl-pyridin-2-yl)-benzyl]-5-(pyridin-2-ylmethoxy)-1H-indol-2-yl]-2,2-dimethyl-propionic acid (Compound 2-98); 3-{3-tert-butylsulfanyl-5-(6-fluoro-pyridin-2-ylmethoxy)-1-[4-(6-methoxy-pyridin-3-yl)-benzyl]-1H-indol-2-yl}-2,2-dimethyl-propionic acid (Compound 2-99); 3-[3-tert-butylsulfanyl-1-[4-(6-methoxy-pyridin-3-yl)-benzyl]-5-(6-methoxy-pyridin-2-ylmethoxy)-1H-indol-2-yl]-2,2-dimethyl-propionic acid (Compound 2-100); 3-[3-tert-butylsulfanyl-1-[4-(6-methoxy-pyridin-3-yl)-benzyl]-5-(6-methyl-pyridin-2-ylmethoxy)-1H-indol-2-yl]-2,2-dimethyl-propionic acid (Compound 2-101); 3-{3-tert-butylsulfanyl-5-(5-methyl-pyridin-2-ylmethoxy)-1-[4-(6-trifluoromethyl-pyridin-3-yl)-benzyl]-1H-indol-2-yl}-2,2-dimethyl-propionic acid (Compound 2-102); 3-{3-tert-butylsulfanyl-5-(5-methyl-pyridin-2-ylmethoxy)-1-[4-(5-trifluoromethyl-pyridin-2-yl)-benzyl]-1H-indol-2-yl}-2,2-dimethyl-propionic acid (Compound 2-103); 3-{3-tert-butylsulfanyl-5-(6-cyclopropyl-pyridin-2-ylmethoxy)-1-[4-(6-methoxy-pyridin-3-yl)-benzyl]-1H-indol-2-yl}-2,2-dimethyl-propionic acid (Compound 2-104); 3-[3-tert-butylsulfanyl-1-[4-(5-methyl-pyridin-2-yl)-benzyl]-5-(5-methyl-pyridin-2-ylmethoxy)-1H-indol-2-yl]-2,2-dimethyl-propionic acid (Compound 2-105); 3-[3-tert-butylsulfanyl-1-[4-(6-methoxy-pyridazin-3-yl)-benzyl]-5-(5-methyl-pyridin-2-ylmethoxy)-1H-indol-2-yl]-2,2-dimethyl-propionic acid (Compound 2-106); 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid (Compound 2-107); 3-{3-tert-butylsulfanyl-5-(5-chloro-pyridin-2-ylmethoxy)-1-[4-(6-methoxy-pyridin-3-yl)-benzyl]-1H-indol-2-yl}-2,2-dimethyl-propionic acid (Compound 2-108); 3-{3-tert-butylsulfanyl-5-((S)-1-pyridin-2-yl-ethoxy)-1-[4-(5-trifluoromethyl-pyridin-2-yl)-benzyl]-1H-indol-2-yl}-2,2-dimethyl-propionic acid (Compound 2-109); 3-{3-tert-butylsulfanyl-5-((R)-1-pyridin-2-yl-ethoxy)-1-[4-(5-trifluoromethyl-pyridin-2-yl)-benzyl]-1H-indol-2-yl}-2,2-dimethyl-propionic acid (Compound 2-110); 3-[3-tert-butylsulfanyl-1-[4-(6-methoxy-pyridin-3-yl)-benzyl]-5-((S)-1-pyridin-2-yl-ethoxy)-1H-indol-2-yl]-2,2-dimethyl-propionic acid (Compound 2-111); 3-[3-tert-butylsulfanyl-1-[4-(6-methoxy-pyridin-3-yl)-benzyl]-5-((R)-1-pyridin-2-yl-ethoxy)-1H-indol-2-yl]-2,2-dimethyl-propionic acid (Compound 2-112); 3-[3-tert-butylsulfanyl-1-[4-(6-methoxy-pyridin-2-yl)-benzyl]-5-((S)-1-pyridin-2-yl-ethoxy)-1H-indol-2-yl]-2,2-dimethyl-propionic acid (Compound 2-113); 3-[3-tert-butylsulfanyl-1-[4-(6-methoxy-pyridin-2-yl)-benzyl]-5-((R)-1-pyridin-2-yl-ethoxy)-1H-indol-2-yl]-2,2-dimethyl-propionic acid (Compound 2-114); 3-[3-tert-butylsulfanyl-1-[4-(2-ethoxy-thiazol-4-yl)-benzyl]-5-((S)-1-pyridin-2-yl-ethoxy)-1H-indol-2-yl]-2,2-dimethyl-propionic acid (Compound 2-115); 3-[3-tert-butylsulfanyl-1-[4-(2-ethoxy-thiazol-4-yl)-benzyl]-5-((R)-1-pyridin-2-yl-ethoxy)-1H-indol-2-yl]-2,2-dimethyl-propionic acid (Compound 2-116); 3-[3-tert-butylsulfanyl-1-[4-(6-methoxy-pyridin-3-yl)-benzyl]-5-(3-methyl-pyridin-2-ylmethoxy)-1H-indol-2-yl]-2,2-dimethyl-propionic acid (Compound 2-117); 3-{3-tert-butylsulfanyl-5-(3-methyl-pyridin-2-ylmethoxy)-1-[4-(5-trifluoromethyl-pyridin-2-yl)-benzyl]-1H-indol-2-yl}-2,2-dimethyl-propionic acid (Compound 2-118); 3-{3-tert-butylsulfanyl-5-(3,5-dimethyl-pyridin-2-ylmethoxy)-1-[4-(6-methoxy-pyridin-3-yl)-benzyl]-1H-indol-2-yl}-2,2-dimethyl-propionic acid (Compound 2-119); 3-{3-tert-butylsulfanyl-5-(3,5-dimethyl-pyridin-2-ylmethoxy)-1-[4-(5-trifluoromethyl-pyridin-2-yl)-benzyl]-1H-indol-2-yl}-2,2-dimethyl-propionic acid (Compound 2-120); 3-{5-(benzothiazol-2-ylmethoxy)-3-tert-butylsulfanyl-1-[4-(6-methoxy-pyridin-3-yl)-benzyl]-1H-indol-2-yl}-2,2-dimethyl-propionic acid (Compound 2-121); 3-{5-(benzothiazol-2-ylmethoxy)-3-tert-butylsulfanyl-1-[4-(5-methoxy-pyridin-2-yl)-benzyl]-1H-indol-2-yl}-2,2-dimethyl-propionic acid (Compound 2-122); 3-{5-(benzothiazol-2-ylmethoxy)-3-cyclobutanecarbonyl-1-[4-(6-methoxy-pyridin-3-yl)-benzyl]-1H-indol-2-yl}-2,2-dimethyl-propionic acid (Compound 2-123); 3-{5-(benzothiazol-2-ylmethoxy)-3-cyclobutylmethyl-1-[4-(6-methoxy-pyridin-3-yl)-benzyl]-1H-indol-2-yl}-2,2-dimethyl-propionic acid (Compound 2-124); 3-{3-tert-butylsulfanyl-5-(5-ethyl-pyridin-2-ylmethoxy)-1-[4-(6-methoxy-pyridin-3-yl)-benzyl]-1H-indol-2-yl}-2,2-dimethyl-propionic acid (Compound 2-125); 3-[3-tert-butylsulfanyl-1-[4-(6-ethoxy-pyridin-3-yl)-benzyl]-5-(5-ethyl-pyridin-2-ylmethoxy)-1H-indol-2-yl]-2,2-dimethyl-propionic acid (Compound 2-126); 3-{3-tert-butylsulfanyl-5-(5-ethyl-pyridin-2-ylmethoxy)-1-[4-(6-trifluoromethyl-pyridin-3-yl)-benzyl]-1H-indol-2-yl}-2,2-dimethyl-propionic acid (Compound 2-127); 3-{3-tert-butylsulfanyl-5-(5-ethyl-pyridin-2-ylmethoxy)-1-[4-(5-trifluoromethyl-pyridin-2-yl)-benzyl]-1H-indol-2-yl}-2,2-dimethyl-propionic acid (Compound 2-128); 3-[3-tert-butylsulfanyl-1-[4-(2-ethoxy-thiazol-4-yl)-benzyl]-5-(5-methyl-pyridin-2-ylmethoxy)-1H-indol-2-yl]-2,2-dimethyl-propionic acid (Compound 2-129); 3-[3-tert-butylsulfanyl-1-[4-(2-methoxy-thiazol-4-yl)-benzyl]-5-(5-methyl-pyridin-2-ylmethoxy)-1H-indol-2-yl]-2,2-dimethyl-propionic acid (Compound 2-130); 3-[3-tert-butylsulfanyl-1-[4-(6-methoxy-pyridin-2-yl)-benzyl]-5-(5-methyl-pyridin-2-ylmethoxy)-1H-indol-2-yl]-2,2-dimethyl-propionic acid (Compound 2-131); 3-[3-cyclobutylmethyl-1-[4-(6-methoxy-pyridin-3-yl)-benzyl]-5-(pyridin-2-ylmethoxy)-1H-indol-2-yl]-2,2-dimethyl-propionic acid (Compound 2-132); 3-[3-cyclobutylmethyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-133); 3-[3-isobutyl-1-[4-(6-methoxy-pyridin-3-yl)-benzyl]-5-(5-methyl-pyridin-2-ylmethoxy)-1H-indol-2-yl]-2,2-dimethyl-propionic acid (Compound 2-134); 3-[3-tert-butylsulfanyl-1-[4-(6-methoxy-pyridin-3-yl)-benzyl]-5-(quinolin-2-ylmethoxy)-1H-indol-2-yl]-2,2-dimethyl-propionic acid (Compound 2-135); 3-{3-tert-butylsulfanyl-5-(quinolin-2-ylmethoxy)-1-[4-(6-trifluoromethyl-pyridin-3-yl)-benzyl]-1H-indol-2-yl}-2,2-dimethyl-propionic acid (Compound 2-136); 3-{3-tert-butylsulfanyl-5-(quinolin-2-ylmethoxy)-1-[4-(5-trifluoromethyl-pyridin-2-yl)-benzyl]-1H-indol-2-yl}-2,2-dimethyl-propionic acid (Compound 2-137); 3-[3-tert-butylsulfanyl-1-[4-(6-methoxy-pyridazin-3-yl)-benzyl]-5-(quinolin-2-ylmethoxy)-1H-indol-2-yl]-2,2-dimethyl-propionic acid (Compound 2-138); 3-[3-tert-butylsulfanyl-1-[4-(6-ethoxy-pyridin-3-yl)-benzyl]-5-(quinolin-2-ylmethoxy)-1H-indol-2-yl]-2,2-dimethyl-propionic acid (Compound 2-139); 3-{3-tert-butylsulfanyl-5-(6-fluoro-quinolin-2-ylmethoxy)-1-[4-(6-methoxy-pyridin-2-yl)-benzyl]-1H-indol-2-yl}-2,2-dimethyl-propionic acid (Compound 2-140); 3-{3-tert-butylsulfanyl-5-(6-fluoro-quinolin-2-ylmethoxy)-1-[4-(6-methoxy-pyridin-3-yl)-benzyl]-1H-indol-2-yl}-2,2-dimethyl-propionic acid (Compound 2-141); 3-[3-tert-butylsulfanyl-1-[4-(2-ethoxy-thiazol-4-yl)-benzyl]-5-(6-fluoro-quinolin-2-ylmethoxy)-1H-indol-2-yl]-2,2-dimethyl-propionic acid (Compound 2-142); 3-{3-tert-butylsulfanyl-5-(6-fluoro-quinolin-2-ylmethoxy)-1-[4-(5-trifluoromethyl-pyridin-2-yl)-benzyl]-1H-indol-2-yl}-2,2-dimethyl-propionic acid (Compound 2-143); 3-{3-tert-butylsulfanyl-5-(7-fluoro-quinolin-2-ylmethoxy)-1-[4-(6-trifluoromethyl-pyridin-3-yl)-benzyl]-1H-indol-2-yl}-2,2-dimethyl-propionic acid (Compound 2-144); 3-{3-tert-butylsulfanyl-5-(7-fluoro-quinolin-2-ylmethoxy)-1-[4-(5-trifluoromethyl-pyridin-2-yl)-benzyl]-1H-indol-2-yl}-2,2-dimethyl-propionic acid (Compound 2-145); 3-{3-tert-butylsulfanyl-5-(7-fluoro-quinolin-2-ylmethoxy)-1-[4-(6-methoxy-pyridin-3-yl)-benzyl]-1H-indol-2-yl}-2,2-dimethyl-propionic acid (Compound 2-146); 3-[3-tert-butylsulfanyl-1-[4-(6-ethoxy-pyridin-3-yl)-benzyl]-5-(7-fluoro-quinolin-2-ylmethoxy)-1H-indol-2-yl]-2,2-dimethyl-propionic acid (Compound 2-147); 3-[3-tert-butylsulfanyl-1-[4-(3-fluoro-pyridin-2-yl)-benzyl]-5-(6-fluoro-quinolin-2-ylmethoxy)-1H-indol-2-yl]-2,2-dimethyl-propionic acid (Compound 2-148); 3-{3-tert-butylsulfanyl-5-(5-methyl-pyridin-2-ylmethoxy)-1-[4-(3-trifluoromethyl-pyridin-2-yl)-benzyl]-1H-indol-2-yl}-2,2-dimethyl-propionic acid (Compound 2-149); 3-{3-tert-butylsulfanyl-5-(5-ethyl-pyridin-2-ylmethoxy)-1-[4-(3-trifluoromethyl-pyridin-2-yl)-benzyl]-1H-indol-2-yl}-2,2-dimethyl-propionic acid (Compound 2-150); 3-{3-tert-butylsulfanyl-5-(quinolin-2-ylmethoxy)-1-[4-(3-trifluoromethyl-pyridin-2-yl)-benzyl]-1H-indol-2-yl}-2,2-dimethyl-propionic acid (Compound 2-151); 3-{3-tert-butylsulfanyl-5-(6-fluoro-quinolin-2-ylmethoxy)-1-[4-(3-trifluoromethyl-pyridin-2-yl)-benzyl]-1H-indol-2-yl}-2,2-dimethyl-propionic acid (Compound 2-156); 3-[3-tert-butylsulfanyl-1-[4-(6-ethoxy-pyridin-3-yl)-benzyl]-5-(3-methyl-pyridin-2-ylmethoxy)-1H-indol-2-yl]-2,2-dimethyl-propionic acid (Compound 2-157); 3-{3-tert-butylsulfanyl-5-(3-methyl-pyridin-2-ylmethoxy)-1-[4-(6-trifluoromethyl-pyridin-3-yl)-benzyl]-1H-indol-2-yl}-2,2-dimethyl-propionic acid (Compound 2-158); 3-{3-tert-butylsulfanyl-5-(3,5-dimethyl-pyridin-2-ylmethoxy)-1-[4-(6-ethoxy-pyridin-3-yl)-benzyl]-1H-indol-2-yl}-2,2-dimethyl-propionic acid (Compound 2-159); 3-[3-tert-butylsulfanyl-1-[4-(6-methoxy-pyridin-3-yl)-benzyl]-5-(4-methyl-pyridin-2-ylmethoxy)-1H-indol-2-yl]-2,2-dimethyl-propionic acid (Compound 2-160); 3-[3-tert-butylsulfanyl-1-[4-(6-ethoxy-pyridin-3-yl)-benzyl]-5-(4-methyl-pyridin-2-ylmethoxy)-1H-indol-2-yl]-2,2-dimethyl-propionic acid (Compound 2-161); 3-{3-tert-butylsulfanyl-5-(4-methyl-pyridin-2-ylmethoxy)-1-[4-(5-trifluoromethyl-pyridin-2-yl)-benzyl]-1H-indol-2-yl}-2,2-dimethyl-propionic acid (Compound 2-162); 3-{3-cyclobutylmethyl-5-(5-methyl-pyridin-2-ylmethoxy)-1-[4-(5-trifluoromethyl-pyridin-2-yl)-benzyl]-1H-indol-2-yl}-2,2-dimethyl-propionic acid (Compound 2-163); 3-[3-tert-butylsulfanyl-1-[4-(6-ethoxy-pyridin-3-yl)-benzyl]-5-(6-fluoro-quinolin-2-ylmethoxy)-1H-indol-2-yl]-2,2-dimethyl-propionic acid (Compound 2-164); 3-{3-tert-butylsulfanyl-5-(6-fluoro-quinolin-2-ylmethoxy)-1-[4-(6-trifluoromethyl-pyridin-3-yl)-benzyl]-1H-indol-2-yl}-2,2-dimethyl-propionic acid (Compound 2-165); 3-[3-tert-butylsulfanyl-1-[4-(6-methoxy-pyridin-3-yl)-benzyl]-5-(6-methyl-quinolin-2-ylmethoxy)-1H-indol-2-yl]-2,2-dimethyl-propionic acid (Compound 2-166); 3-{3-tert-butylsulfanyl-5-(6-methyl-quinolin-2-ylmethoxy)-1-[4-(5-trifluoromethyl-pyridin-2-yl)-benzyl]-1H-indol-2-yl}-2,2-dimethyl-propionic acid (Compound 2-167); 3-[3-tert-butylsulfanyl-1-[4-(6-methyl-pyridazin-3-yl)-benzyl]-5-(quinolin-2-ylmethoxy)-1H-indol-2-yl]-2,2-dimethyl-propionic acid (Compound 2-168); 3-[3-tert-butylsulfanyl-1-[4-(6-ethoxy-pyridazin-3-yl)-benzyl]-5-(quinolin-2-ylmethoxy)-1H-indol-2-yl]-2,2-dimethyl-propionic acid (Compound 2-169); 3-[3-isobutyl-1-[4-(6-methoxy-pyridin-3-yl)-benzyl]-5-(quinolin-2-ylmethoxy)-1H-indol-2-yl]-2,2-dimethyl-propionic acid (Compound 2-170); 3-{3-tert-butylsulfanyl-5-(6-fluoro-quinolin-2-ylmethoxy)-1-[4-(6-methoxy-pyridazin-3-yl)-benzyl]-1H-indol-2-yl}-2,2-dimethyl-propionic acid (Compound 2-171); 3-[1-[4-(6-methoxy-pyridin-3-yl)-benzyl]-3-(2-methyl-propane-2-sulfonyl)-5-(pyridin-2-ylmethoxy)-1H-indol-2-yl]-2,2-dimethyl-propionic acid (Compound 2-172); 3-[1-[4-(6-methoxy-pyridin-3-yl)-benzyl]-3-(2-methyl-propane-2-sulfinyl)-5-(pyridin-2-ylmethoxy)-1H-indol-2-yl]-2,2-dimethyl-propionic acid (Compound 2-173); 3-[3-tert-butylsulfanyl-1-[4-(6-methoxy-pyridin-3-yl)-benzyl]-5-(1-oxy-pyridin-2-ylmethoxy)-1H-indol-2-yl]-2,2-dimethyl-propionic acid (Compound 2-174); 3-{3-tert-butylsulfanyl-5-(imidazo[1,2-a]pyridin-2-ylmethoxy)-1-[4-(6-methoxy-pyridin-3-yl)-benzyl]-1H-indol-2-yl}-2,2-dimethyl-propionic acid (Compound 2-175); 3-[3-tert-butylsulfanyl-1-[4-(6-ethoxy-pyridin-3-yl)-benzyl]-5-(imidazo[1,2-a]pyridin-2-ylmethoxy)-1H-indol-2-yl]-2,2-dimethyl-propionic acid (Compound 2-176); 3-{3-tert-butylsulfanyl-5-(imidazo[1,2-a]pyridin-2-ylmethoxy)-1-[4-(5-trifluoromethyl-pyridin-2-yl)-benzyl]-1H-indol-2-yl}-2,2-dimethyl-propionic acid (Compound 2-177); 3-[3-tert-butylsulfanyl-1-[4-(6-ethoxy-pyridin-3-yl)-benzyl]-5-((R)-1-pyridin-2-yl-ethoxy)-1H-indol-2-yl]-2,2-dimethyl-propionic acid (Compound 2-178); 3-{3-tert-butylsulfanyl-5-(6-fluoro-quinolin-2-ylmethoxy)-1-[4-(6-methyl-pyridazin-3-yl)-benzyl]-1H-indol-2-yl}-2,2-dimethyl-propionic acid (Compound 2-179); 3-[3-tert-butylsulfanyl-1-[4-(6-methoxy-pyridin-3-yl)-benzyl]-5-(5-methyl-isoxazol-3-ylmethoxy)-1H-indol-2-yl]-2,2-dimethyl-propionic acid (Compound 2-180); 3-[3-tert-butylsulfanyl-1-[4-(6-ethoxy-pyridin-3-yl)-benzyl]-5-(5-methyl-isoxazol-3-ylmethoxy)-1H-indol-2-yl]-2,2-dimethyl-propionic acid (Compound 2-181); 3-{3-tert-butylsulfanyl-5-(5-methyl-isoxazol-3-ylmethoxy)-1-[4-(5-trifluoromethyl-pyridin-2-yl)-benzyl]-1H-indol-2-yl}-2,2-dimethyl-propionic acid (Compound 2-182); 3-{3-tert-Butylsulfanyl-5-(2,5-dimethyl-2H-pyrazol-3-ylmethoxy)-1-[4-(6-methoxy-pyridin-3-yl)-benzyl]-1H-indol-2-yl}-2,2-dimethyl-propionic acid (Compound 2-183); 3-{3-tert-butylsulfanyl-5-(1,5-dimethyl-1H-pyrazol-3-ylmethoxy)-1-[4-(6-methoxy-pyridin-3-yl)-benzyl]-1H-indol-2-yl}-2,2-dimethyl-propionic acid (Compound 2-184); 3-[3-tert-butylsulfanyl-1-[4-(6-ethoxy-pyridazin-3-yl)-benzyl]-5-(6-fluoro-quinolin-2-ylmethoxy)-1H-indol-2-yl]-2,2-dimethyl-propionic acid (Compound 2-185); 3-[3-tert-butylsulfanyl-1-[4-(6-ethoxy-pyridazin-3-yl)-benzyl]-5-(5-ethyl-pyridin-2-ylmethoxy)-1H-indol-2-yl]-2,2-dimethyl-propionic acid (Compound 2-186); 3-{3-tert-butylsulfanyl-5-(5-ethyl-pyridin-2-ylmethoxy)-1-[4-(6-methoxy-pyridazin-3-yl)-benzyl]-1H-indol-2-yl}-2,2-dimethyl-propionic acid (Compound 2-187); 3-[3-tert-butylsulfanyl-1-[4-(5-fluoro-pyridin-2-yl)-benzyl]-5-(6-fluoro-quinolin-2-ylmethoxy)-1H-indol-2-yl]-2,2-dimethyl-propionic acid (Compound 2-188); 3-[3-tert-butylsulfanyl-1-[4-(5-fluoro-pyridin-2-yl)-benzyl]-5-((R)-1-pyridin-2-yl-ethoxy)-1H-indol-2-yl]-2,2-dimethyl-propionic acid (Compound 2-189); 3-[3-tert-butylsulfanyl-1-[4-(6-ethoxy-pyridin-2-yl)-benzyl]-5-(6-fluoro-quinolin-2-ylmethoxy)-1H-indol-2-yl]-2,2-dimethyl-propionic acid (Compound 2-190); 3-[3-tert-butylsulfanyl-1-[4-(6-ethoxy-pyridin-2-yl)-benzyl]-5-((R)-1-pyridin-2-yl-ethoxy)-1H-indol-2-yl]-2,2-dimethyl-propionic acid (Compound 2-191); 3-[3-tert-butylsulfanyl-1-[4-(5-fluoro-pyridin-2-yl)-benzyl]-5-(5-methyl-pyridin-2-ylmethoxy)-1H-indol-2-yl]-2,2-dimethyl-propionic acid (Compound 2-192); 3-[3-tert-butysulfanyl-1-[4-(6-ethoxy-pyridin-2-yl)-benzyl]-5-(5-methyl-pyridin-2-ylmethoxy)-1H-indol-2-yl]-2,2-dimethyl-propionic acid (Compound 2-193); 3-{5-(6-fluoro-quinolin-2-ylmethoxy)-3-isobutyl-1-[4-(6-trifluoromethyl-pyridin-3-yl)-benzyl]-1H-indol-2-yl}-2,2-dimethyl-propionic acid (Compound 2-194); 3-{3-tert-butylsulfanyl-5-(pyridin-2-ylmethoxy)-1-[3-(5-trifluoromethyl-pyridin-2-yl)-benzyl]-1H-indol-2-yl}-2,2-dimethyl-propionic acid (Compound 2-195); 3-[3-tert-butylsulfanyl-1-[3-(6-methoxy-pyridin-3-yl)-benzyl]-5-(pyridin-2-ylmethoxy)-1H-indol-2-yl]-2,2-dimethyl-propionic acid (Compound 2-196); 3-[3-tert-butylsulfanyl-1-[4-(5-fluoro-pyridin-2-yl)-benzyl]-5-(quinolin-2-ylmethoxy)-1H-indol-2-yl]-2,2-dimethyl-propionic acid (Compound 2-197); 3-[3-tert-butylsulfanyl-1-[4-(6-ethoxy-pyridin-2-yl)-benzyl]-5-(quinolin-2-ylmethoxy)-1H-indol-2-yl]-2,2-dimethyl-propionic acid (Compound 2-198); 3-[3-tert-butylsulfanyl-1-[4-(6-ethoxy-pyridin-2-yl)-benzyl]-5-(pyridin-2-ylmethoxy)-1H-indol-2-yl]-2,2-dimethyl-propionic acid (Compound 2-199); 3-{3-tert-butylsulfanyl-5-(6-fluoro-quinolin-2-ylmethoxy)-1-[4-(6-trifluoromethyl-pyridin-2-yl)-benzyl]-1H-indol-2-yl}-2,2-dimethyl-propionic acid (Compound 2-200); 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid (Compound 2-201); 3-{3-tert-butylsulfanyl-5-(5-methyl-pyridin-2-ylmethoxy)-1-[4-(6-trifluoromethyl-pyridin-2-yl)-benzyl]-1H-indol-2-yl}-2,2-dimethyl-propionic acid (Compound 2-202); 3-{3-tert-butylsulfanyl-5-(quinolin-2-ylmethoxy)-1-[4-(6-trifluoromethyl-pyridin-2-yl)-benzyl]-1H-indol-2-yl}-2,2-dimethyl-propionic acid (Compound 2-203); 3-{3-tert-butylsulfanyl-5-(pyridin-2-ylmethoxy)-1-[4-(6-trifluoromethyl-pyridin-2-yl)-benzyl]-1H-indol-2-yl}-2,2-dimethyl-propionic acid (Compound 2-204); 3-[3-tert-butylsulfanyl-5-(quinolin-2-ylmethoxy)-1-(4-thiazol-2-yl-benzyl)-1H-indol-2-yl]-2,2-dimethyl-propionic acid (Compound 2-205); 3-[3-tert-butylsulfanyl-1-[3-(4-methoxy-tetrahydro-pyran-4-yl)-benzyl]-5-(pyridin-2-ylmethoxy)-1H-indol-2-yl]-2,2-dimethyl-propionic acid (Compound 2-206); 3-[3-tert-butylsulfanyl-5-(6-fluoro-quinolin-2-ylmethoxy)-1-(4-pyridin-2-yl-benzyl)-1H-indol-2-yl]-2,2-dimethyl-propionic acid (Compound 2-207); 3-[3-tert-butylsulfanyl-5-(5-ethyl-pyridin-2-ylmethoxy)-1-(4-pyridin-3-yl-benzyl)-1H-indol-2-yl]-2,2-dimethyl-propionic acid (Compound 2-208); 3-[3-tert-butylsulfanyl-1-(4-pyridin-3-yl-benzyl)-5-(quinolin-2-ylmethoxy)-1H-indol-2-yl]-2,2-dimethyl-propionic acid (Compound 2-209); 3-[3-tert-butylsulfanyl-5-(6-fluoro-quinolin-2-ylmethoxy)-1-(4-pyridin-3-yl-benzyl)-1H-indol-2-yl]-2,2-dimethyl-propionic acid (Compound 2-210); 3-[3-tert-butylsulfanyl-5-(5-methyl-pyridin-2-ylmethoxy)-1-(4-pyridin-2-yl-benzyl)-1H-indol-2-yl]-2,2-dimethyl-propionic acid (Compound 2-211); 3-[3-tert-butylsulfanyl-5-(5-ethyl-pyridin-2-ylmethoxy)-1-(4-pyridin-2-yl-benzyl)-1H-indol-2-yl]-2,2-dimethyl-propionic acid (Compound 2-212); 3-[3-tert-butylsulfanyl-1-(4-pyridin-2-yl-benzyl)-5-(quinolin-2-ylmethoxy)-1H-indol-2-yl]-2,2-dimethyl-propionic acid (Compound 2-213); 3-[3-tert-butylsulfanyl-5-(5-methyl-pyridin-2-ylmethoxy)-1-(4-pyridin-3-yl-benzyl)-1H-indol-2-yl]-2,2-dimethyl-propionic acid (Compound 2-214); 3-[3-tert-butylsulfanyl-1-[4-(4-methoxy-pyridin-2-yl)-benzyl]-5-(5-methyl-pyridin-2-ylmethoxy)-1H-indol-2-yl]-2,2-dimethyl-propionic acid (Compound 2-215; 3-[3-tert-butylsulfanyl-5-(quinolin-2-ylmethoxy)-1-(4-(3-methyoxypyridin-2-yl)-benzyl)-1H-indol-2-yl]-2,2-dimethyl-propionic acid (Compound 2-216); 3-[3-tert-butylsulfanyl-5-(5-methyl-pyridin-2-ylmethoxy)-1-(4-(3-methyoxypyridin-2-yl)-benzyl)-1H-indol-2-yl]-2,2-dimethyl-propionic acid (Compound 2-217); 3-[3-tert-butylsulfanyl-5-(5-ethyl-pyridin-2-ylmethoxy)-1-(4-(3-methyoxypyridin-2-yl)-benzyl)-1H-indol-2-yl]-2,2-dimethyl-propionic acid (Compound 2-218); 3-[3-tert-butylsulfanyl-5-(5-methyl-pyridin-2-ylmethoxy)-1-(4-(4-trifluoromethypyridin-2-yl)-benzyl)-1H-indol-2-yl]-2,2-dimethyl-propionic acid (Compound 2-219); 3-[3-tert-butylsulfanyl-5-(5-ethyl-pyridin-2-ylmethoxy)-1-(4-(4-trifluoromethypyridin-2-yl)-benzyl)-1H-indol-2-yl]-2,2-dimethyl-propionic acid (Compound 2-220); 3-[3-tert-butylsulfanyl-5-(quinolin-2-ylmethoxy)-1-(4-(4-trifluoromethypyridin-2-yl)-benzyl)-1H-indol-2-yl]-2,2-dimethyl-propionic acid (Compound 2-221); 3-[3-tert-butylsulfanyl-5-(5-methyl-pyridin-2-ylmethoxy)-1-(4-(5-fluoropyridin-3-yl)-benzyl)-1H-indol-2-yl]-2,2-dimethyl-propionic acid (Compound 2-222); 3-[3-tert-butylsulfanyl-5-(5-ethyl-pyridin-2-ylmethoxy)-1-(4-(5-fluoropyridin-3-yl)-benzyl)-1H-indol-2-yl]-2,2-dimethyl-propionic acid (Compound 2-223); 3-[3-tert-butylsulfanyl-5-(quinolin-2-ylmethoxy)-1-(4-(5-fluoropyridin-3-yl)-benzyl)-1H-indol-2-yl]-2,2-dimethyl-propionic acid (Compound 2-224); 3-[3-tert-butylsulfanyl-5-(2,3-dimethyl-pyridin-6-ylmethoxy)-1-(4-(2-methoxypyridin-5-yl)-benzyl)-1H-indol-2-yl]-2,2-dimethyl-propionic acid (Compound 2-225); 3-[3-tert-butylsulfanyl-5-(2,3-dimethyl-pyridin-6-ylmethoxy)-1-(4-(3-trifluoromethylpyridin-2-yl)-benzyl)-1H-indol-2-yl]-2,2-dimethyl-propionic acid (Compound 2-226); 3-[3-tert-butylsulfanyl-5-(2,3-dimethyl-pyridin-6-ylmethoxy)-1-(4-(4-trifluoromethylpyridin-2-yl)-benzyl)-1H-indol-2-yl]-2,2-dimethyl-propionic acid (Compound 2-227); 3-[3-tert-butylsulfanyl-5-(2,3-dimethyl-pyridin-6-ylmethoxy)-1-(4-(3-fluoropyridin-2-yl)-benzyl)-1H-indol-2-yl]-2,2-dimethyl-propionic acid (Compound 2-228); 3-[3-tert-butylsulfanyl-5-(2,3-dimethyl-pyridin-6-ylmethoxy)-1-(4-(5-fluoropyridin-3-yl)-benzyl)-1H-indol-2-yl]-2,2-dimethyl-propionic acid (Compound 2-229); 3-[3-tert-butylsulfanyl-5-(2,3-dimethyl-pyridin-6-ylmethoxy)-1-(4-(4-methoxypyridin-2-yl)-benzyl)-1H-indol-2-yl]-2,2-dimethyl-propionic acid (Compound 2-230); 3-[3-tert-butylsulfanyl-5-(2,3-dimethyl-pyridin-6-ylmethoxy)-1-(4-(pyridin-2-yl)-benzyl)-1H-indol-2-yl]-2,2-dimethyl-propionic acid (Compound 2-231); 3-[3-tert-butylsulfanyl-5-(5-methyl-pyridin-2-ylmethoxy)-1-(4-(2-methoxy-pyridin-3-yl)-benzyl)-1H-indol-2-yl]-2,2-dimethyl-propionic acid (Compound 2-232); 3-[3-tert-butylsulfanyl-5-(5-ethyl-pyridin-2-ylmethoxy)-1-(4-(2-methoxy-pyridin-3-yl)-benzyl)-1H-indol-2-yl]-2,2-dimethyl-propionic acid (Compound 2-233); 3-[3-tert-butylsulfanyl-5-(quinolin-2-ylmethoxy)-1-(4-(2-methoxy-pyridin-3-yl)-benzyl)-1H-indol-2-yl]-2,2-dimethyl-propionic acid (Compound 2-234); 3-[3-tert-butylsulfanyl-1-(6′-methoxy-[2,3′]bipyridinyl-5-ylmethyl)-5-(pyridin-2-ylmethoxy)-1H-indol-2-yl]-2,2-dimethyl-propionic acid (Compound 3-1); 3-[3-tert-butylsulfanyl-1-[6-(4-methoxy-phenyl)-pyridin-3-ylmethyl]-5-(pyridin-2-ylmethoxy)-1H-indol-2-yl]-2,2-dimethyl-propionic acid (Compound 3-2); 3-{3-tert-butylsulfanyl-5-(pyridin-2-ylmethoxy)-1-[6-(4-trifluoromethoxy-phenyl)-pyridin-3-ylmethyl]-1H-indol-2-yl}-2,2-dimethyl-propionic acid (Compound 3-3); 3-[3-tert-butylsulfanyl-1-[5-(4-methoxy-phenyl)-pyridin-2-ylmethyl]-5-(pyridin-2-ylmethoxy)-1H-indol-2-yl]-2,2-dimethyl-propionic acid (Compound 3-4); 3-{3-tert-butylsulfanyl-5-(pyridin-2-ylmethoxy)-1-[5-(4-trifluoromethoxy-phenyl)-pyridin-2-ylmethyl]-1H-indol-2-yl}-2,2-dimethyl-propionic acid (Compound 3-5); 3-{3-tert-butylsulfanyl-5-isopropyl-1-[4-(6-methoxy-pyridin-3-yl)-benzyl]-1H-indol-2-yl}-2,2-dimethyl-propionic acid (Compound 4-1); 3-{3-tert-butylsulfanyl-5-hydroxy-1-[4-(6-methoxy-pyridin-3-yl)-benzyl]-1H-indol-2-yl}-2,2-dimethyl-propionic acid (Compound 4-2).

24. A method according to claim 1, wherein the leukotriene inhibitor is 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid.

25. A method according to claim 1, wherein the human is suffering from a chronic inflammatory disease or condition.

26. A method according to claim 25, wherein the disease or condition is selected from the group consisting of systemic lupus erythematosus, rheumatoid arthritis, systemic sclerosis, pulmonary arterial hypertension, asthma, gout, sleep apnea, and any combinations thereof.

27. A method according to claim 1, wherein the leukotriene inhibitor is present in the form of a salt.

28. A method according to claim 1, wherein the leukotriene inhibitor is present in the form of a sodium salt.

29. A method for reducing pro-inflammatory HDL in a human comprising:
determining that the human has a need to have pro-inflammatory HDL levels reduced;
administering to the human a therapeutically effective amount of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof such that the level of pro-inflammatory HDL in the human is reduced;
confirming the conversion of pro-inflammatory HDL to anti-inflammatory HDL.

30. (canceled)

31. Use of a leukotriene inhibitor, a pharmaceutically acceptable salt, a pharmaceutically acceptable N-oxide, a pharmaceutically active metabolite, a pharmaceutically acceptable prodrug, or a pharmaceutically acceptable solvate thereof to reduce a pro-inflammatory HDL in a human such that the level of pro-inflammatory HDL is reduced.

32. The use according to claim 31, wherein the leukotriene inhibitor is 3-[3-tert-butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid.
